SEQUENCE LISTING (337 sequences)

SEQ ID NO: 1 - DNA, 72 bp, Artificial Sequence
  Pel B leader
atgaaatacctgctgccgaccgcagccgctggtctgctgctgctcgcggcccagccggccatggccgccggt

SEQ ID NO: 2 - protein, 24 aa, Artificial Sequence
  Pel B leader
MKYLLPTAAAGLLLLAAQPAMAAG

SEQ ID NO: 3 - DNA, 72 bp, Artificial Sequence
  Pel B leader amber stop
atgaaatacctgctgccgaccgcagccgctggtctgctgctgctcgcggcctagccggccatggccgccggt

SEQ ID NO: 4 - protein, 17 aa, Artificial Sequence
  Pel B leader amber stop
MKYLLPTAAAGLLLLAA

SEQ ID NO: 5 - DNA, 69 bp, Artificial Sequence
  OmpA leader
atgaaaaagacagctatcgcgattgcagtggcactggctggtttcgctaccgtagcccaggcggccgca

SEQ ID NO: 6 - protein, 23 aa, Artificial Sequence
  OmpA leader
MKKTAIAIAVALAGFATVAQAAA

SEQ ID NO: 7 - DNA, 69 bp, Artificial Sequence
  OmpA leader amber stop
atgaaaaagacagctatcgcgattgcagtggcactggctggtttcgctaccgtagcctaggcggccgca

SEQ ID NO: 8 - protein, 19 aa, Artificial Sequence
  OmpA leader amber stop
MKKTAIAIAVALAGFATVA

SEQ ID NO: 9 - protein, 35 aa, Artificial Sequence
  GCN4 zipper
GRMKQLEDKVEELLSKNYHLENEVARLKKLVGERG

SEQ ID NO: 10 - protein, 123 aa, Artificial Sequence
  2G12 VH domain
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSP

SEQ ID NO: 11 - protein, 107 aa, Artificial Sequence
  2G12 VL domain 1
DVVMTQSPSTLSASVGDTITITCRASQSIETWLAWYQQKPGKAPKLLIYKASTLKTGVPSRFSGSGSGTEFTLTISGLQFDDFATYHCQHYAGYSATFGQGTRVEIK

SEQ ID NO: 12 - protein, 108 aa, Artificial Sequence
  2G12 VL domain 2
AGVVMTQSPSTLSASVGDTITITCRASQSIETWLAWYQQKPGKAPKLLIYKASTLKTGVPSRFSGSGSGTEFTLTISGLQFDDFATYHCQHYAGYSATFGQGTRVEIK

SEQ ID NO: 13 - DNA, 3513 bp, Artificial Sequence
  pCAL G13 vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcccagccggccatggccgccggtgcctaactctggctggtttcgctaccgtaaccggtttaattaataaggaggatataattatgaaaaagacagctatcgcgattgcagtggcactggctggtttcgctaccgtagcccaggcggccgcacgcgtctggttgaatctggtggggtctggaattctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 14 - DNA, 3513 bp, Artificial Sequence
  pCAL A1 vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcccagccggccatggccgccggtgcctaactctggctggtttcgctaccgtaaccggtttaattaataaggaggatataattatgaaaaagacagctatcgcgattgcagtggcactggctggtttcgctaccgtagcccaggcggccgcacgcgtctggttgaatctggtggggtctggaattctgcgatcgcggccaggccggccgcaccatcaccatcaccatggcgcatacccgtacgacgttccggactacgcttctactagttagaagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 15 - DNA, 54 bp, Artificial Sequence
  Linker 1
ggtggttcgtctggatcttcctcctctggtggcggtggctcgggcggtggtggc

SEQ ID NO: 16 - protein, 18 aa, Artificial Sequence
  Linker 1
GGSSGSSSSGGGGSGGGG

SEQ ID NO: 17 - DNA, 54 bp, Artificial Sequence
  Linker 2
ggaggatccggcagcagcagcagcggcggcggcggcgggagctccggcggcgga

SEQ ID NO: 18 - protein, 18 aa, Artificial Sequence
  Linker 2
GGSGSSSSGGGGGSSGGG

SEQ ID NO: 19 - DNA, 48 bp, Artificial Sequence
  L216
ggaggatccggcagcagcagcagcggcggcgggagctccggcggcgga

SEQ ID NO: 20 - protein, 16 aa, Artificial Sequence
  L216
GGSGSSSSGGGSSGGG

SEQ ID NO: 21 - DNA, 51 bp, Artificial Sequence
  L217
ggaggatccggcagcagcagcagcggcggcggcgggagctccggcggcgga

SEQ ID NO: 22 - protein, 17 aa, Artificial Sequence
  L217
GGSGSSSSGGGGSSGGG

SEQ ID NO: 23 - DNA, 57 bp, Artificial Sequence
  L219
ggaggatccagcggcagcagcagcagcggcggcggcggcgggagctccggcggcgga

SEQ ID NO: 24 - protein, 19 aa, Artificial Sequence
  L219
GGSSGSSSSGGGGGSSGGG

SEQ ID NO: 25 - DNA, 60 bp, Artificial Sequence
  L220
ggaggatccagcggcggcagcagcagcagcggcggcggcggcgggagctccggcggcgga

SEQ ID NO: 26 - protein, 20 aa, Artificial Sequence
  L220
GGSSGGSSSSGGGGGSSGGG

SEQ ID NO: 27 - DNA, 87 bp, Artificial Sequence
  BamHISacI linker / BamHISacI(+)
gatccggtggcggcagcgaaggtggtggcagcgaaggtggcggtagcgaaggtggcggcagcgaaggcggcggtagcggtgggagct

SEQ ID NO: 28 - protein, 29 aa, Artificial Sequence
  BamHISacI linker
DPVAAAKVVAAKVAVAKVAAAKAAVAVGA

SEQ ID NO: 29 - DNA, 6819 bp, Artificial Sequence
  vector with 2G12 Fab Cys19 construct
ggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctagggccaggccggccgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaaatacctattgcctacggcagccgctggattgttattactcgctgcccaaccagccatggccgaagttcagctggttgaatctggtggtggtctggttaaagctggtggttctctgtgcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcggcagcagccaccatcaccatcaccatggcgcatacccgtacgacgttccggactacgcttcttagctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 30 - DNA, 6802 bp, Artificial Sequence
  vector with 2G12 scFab deltaC2Cys19 construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatctggtggtggatccggtggcggcagcgaaggtggtggcagcgaaggtggcggtagcgaaggtggcggcagcgaaggcggcggtagcggtgggagctccggtgaagttcagctggttgaatctggtggtggtctggttaaagctggtggttctctgtgcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcacaaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatctggcagcagcggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 31 - DNA, 6121 bp, Artificial Sequence
  vector with 2G12 scFv Cys19 construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaaggtggttcgtctggatcttcctcctctggtggcggtggctcgggcggtggtggcgaagttcagctggttgaatctggtggtggtctggttaaagctggtggttctctgtgcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcacaaaatgcgtgttgaagacaccgctatctactactgcgctcgtaaaggttctgaccgtctgtctgacaacgacccgttcgacgcttggggtccgggtaccgttgttaccgtttctccgggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 32 - DNA, 4765 bp, Artificial Sequence
  2G12 pCAL G13 vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcccagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcccaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 33 - DNA, 4765 bp, Artificial Sequence
  3-ALA 2G12 pCAL G13 vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcccagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcccaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtgcggcggacgcggacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 34 - DNA, 4765 bp, Artificial Sequence
  2G12 pCAL A1 vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcccagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcccaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttagaagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 35 - DNA, 5882 bp, Artificial Sequence
  2G12 pCAL IT*
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcggtacccgataaaagcggcttcctgacaggaggccgttttgttttgcagcccacctcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcctagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcctaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 36 - DNA, 5882 bp, Artificial Sequence
  2G12 pCAL ITPO vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcggtacccgataaaagcggcttcctgacaggaggccgttttgttttgcagcccacctcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcccagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcccaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 37 - DNA, 3 bp, Artificial Sequence
  Amber stop
tag

SEQ ID NO: 38 - DNA, 6840 bp, Artificial Sequence
  2G12 Fab hinge in petDuet
ggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctagggccaggccggccgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaaatacctattgcctacggcagccgctggattgttattactcgctgcccaaccagccatggccgaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaaagctgcgataaaacccatacctgcccgccgtgcccgcaccatcaccatcaccatggcgcatacccgtacgacgttccggactacgcttcttagctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 39 - DNA, 6121 bp, Artificial Sequence
  vector with 2G12 domain exchanged scFv
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaaggtggttcgtctggatcttcctcctctggtggcggtggctcgggcggtggtggcgaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcacaaaatgcgtgttgaagacaccgctatctactactgcgctcgtaaaggttctgaccgtctgtctgacaacgacccgttcgacgcttggggtccgggtaccgttgttaccgtttctccgggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 40 - DNA, 6916 bp, Artificial Sequence
  vector w/ 2G12 domain exchanged scFv tandem
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaaggtggttcgtctggatcttcctcctctggtggcggtggctcgggcggtggtggcgaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcacaaaatgcgtgttgaagacaccgctatctactactgcgctcgtaaaggttctgaccgtctgtctgacaacgacccgttcgacgcttggggtccgggtaccgttgttaccgtttctccgggaggatccggcagcagcagcagcggcggcggcggcgggagctccggcggcggagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcacaaaatgcgtgttgaagacaccgctatctactactgcgctcgtaaaggttctgaccgtctgtctgacaacgacccgttcgacgcttggggtccgggtaccgttgttaccgtttctccgggtggttcgtctggatcttcctcctctggtggcggtggctcgggcggtggtggcgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaaggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 41 - DNA, 6166 bp, Artificial Sequence
  vector w/ 2G12 domain exchanged scFv hinge(E)
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaaggtggttcgtctggatcttcctcctctggtggcggtggctcgggcggtggtggcgaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcacaaaatgcgtgttgaagacaccgctatctactactgcgctcgtaaaggttctgaccgtctgtctgacaacgacccgttcgacgcttggggtccgggtaccgttgttaccgtttctccggaaccgaaaagctgcgataaaacccatacctgcccgccgtgcccgggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 42 - DNA, 6163 bp, Artificial Sequence
  vector w/ 2G12 domain exchanged scFv hinge deltaE
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaaggtggttcgtctggatcttcctcctctggtggcggtggctcgggcggtggtggcgaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcacaaaatgcgtgttgaagacaccgctatctactactgcgctcgtaaaggttctgaccgtctgtctgacaacgacccgttcgacgcttggggtccgggtaccgttgttaccgtttctccgccgaaaagctgcgataaaacccatacctgcccgccgtgcccgggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 43 - protein, 424 aa, Artificial Sequence
  M13 gene III protein (cp3; g3p; minor coat protein; g.i. 59799327)
MKKLLFAIPLVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 44 - DNA, 7 bp, Artificial Sequence
  Sap-I cleavage site
gctcttc

SEQ ID NO: 45 - DNA, 7 bp, Artificial Sequence
  reverse complement Sap-I cleavage site
gaagagc

SEQ ID NO: 46 - DNA, 923 bp, Artificial Sequence
  pComb3x vector containing the Ac8-encoding sequence - sequence through the Ab framework into the start of gene III
atgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgcttcttaggagggtggtggctctgaggg

SEQ ID NO: 47 - DNA, 70 bp, Artificial Sequence
  pCAL_0
agcggaagagcgcccaatacgcaaaccgcctctccccgcgcgttggccgattcattaatgcagctggcac

SEQ ID NO: 48 - DNA, 85 bp, Artificial Sequence
  pCAL_1
gacaggtttcccgactggaaagcgggcagtgagcgcaacgcaattaatgtgagttagctcactcattaggcaccccaggctttac

SEQ ID NO: 49 - DNA, 85 bp, Artificial Sequence
  pCAL_2
actttatgcttccggctcgtatgttgtgtggaattgtgagcggataacaattgaattaaggaggatataattatgaaatacctgc

SEQ ID NO: 50 - DNA, 85 bp, Artificial Sequence
  pCAL_3
tgccgaccgcagccgctggtctgctgctgctcgcggcccagccggccatggccgccggtgcctaactctggctggtttcgctacc

SEQ ID NO: 51 - DNA, 85 bp, Artificial Sequence
  pCAL_4
gtaaccggtttaattaataaggaggatataattatgaaaaagacagctatcgcgattgcagtggcactggctggtttcgctaccg

SEQ ID NO: 52 - DNA, 85 bp, Artificial Sequence
  pCAL_5
tagcccaggcggccgcacgcgtctggttgaatctggtggggtctggaattctgcgatcgcggccaggccggccgcaccatcacca

SEQ ID NO: 53 - DNA, 44 bp, Artificial Sequence
  pCAL_6
tcaccatggcgcatacccgtacgacgttccggactacgcttcta

SEQ ID NO: 54 - DNA, 70 bp, Artificial Sequence
  pCAL_7
ctagtagaagcgtagtccggaacgtcgtacgggtatgcgccatggtgatggtgatggtgcggccggcctg

SEQ ID NO: 55 - DNA, 85 bp, Artificial Sequence
  pCAL_8
gccgcgatcgcagaattccagaccccaccagattcaaccagacgcgtgcggccgcctgggctacggtagcgaaaccagccagtgc

SEQ ID NO: 56 - DNA, 85 bp, Artificial Sequence
  pCAL_9
cactgcaatcgcgatagctgtctttttcataattatatcctccttattaattaaaccggttacggtagcgaaaccagccagagtt

SEQ ID NO: 57 - DNA, 85 bp, Artificial Sequence
  pCAL_10
aggcaccggcggccatggccggctgggccgcgagcagcagcagaccagcggctgcggtcggcagcaggtatttcataattatatc

SEQ ID NO: 58 - DNA, 85 bp, Artificial Sequence
  pCAL_11
ctccttaattcaattgttatccgctcacaattccacacaacatacgagccggaagcataaagtgtaaagcctggggtgcctaatg

SEQ ID NO: 59 - DNA, 85 bp, Artificial Sequence
  pCAL_12
agtgagctaactcacattaattgcgttgcgctcactgcccgctttccagtcgggaaacctgtcgtgccagctgcattaatgaatc

SEQ ID NO: 60 - DNA, 45 bp, Artificial Sequence
  pCAL_13
ggccaacgcgcggggagaggcggtttgcgtattgggcgctcttcc

SEQ ID NO: 61 - DNA, 34 bp, Artificial Sequence
  SpeIG3-F
ggtggtggttctggtactagttaggagggtggtg

SEQ ID NO: 62 - DNA, 52 bp, Artificial Sequence
  PvuINheIG3-R
gggaagggcgatcgttagctagcttaagactccttattacgcagtatgttag

SEQ ID NO: 63 - DNA, 34 bp, Artificial Sequence
  SpeG3A-F
ggtggtggttctggtactagttagaagggtggtg

SEQ ID NO: 64 - DNA, 645 bp, Artificial Sequence
  NT encoding 3-ALA 2G12 and 2G12 Fab light chain
gccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgc

SEQ ID NO: 65 - DNA, 684 bp, Artificial Sequence
  NT encoding 2G12 Fab  heavy chain
gaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcga

SEQ ID NO: 66 - DNA, 27 bp, Artificial Sequence
  nt sequence encoding HA tag
tacccgtacgacgttccggactacgct

SEQ ID NO: 67 - protein, 9 aa, Artificial Sequence
  AA sequence - HA tag
YPYDVPDYA

SEQ ID NO: 68 - DNA, 6840 bp, Artificial Sequence; identical to SEQ ID NO: 38
  2G12 heavy and light chain in pET Duet vector (Fab)

SEQ ID NO: 69 - DNA, 37 bp, Artificial Sequence
  pCALVL-F
ccatggccgccggtgttgttatgacccagtctccgtc

SEQ ID NO: 70 - DNA, 38 bp, Artificial Sequence
  pCALCK-R
ctccttattaattaattagcattcaccacggttgaaag

SEQ ID NO: 71 - DNA, 40 bp, Artificial Sequence
  pCALVH-F primer
gcccaggcggccgcagaagttcagctggttgaatctggtg

SEQ ID NO: 72 - DNA, 41 bp, Artificial Sequence
  pCALCH-R
ctggccgcgatcgcaggcaagatttcggttcaactttcttg

SEQ ID NO: 73 - protein, 228 aa, Artificial Sequence
  2G12 domain exchanged Fab heavy chain (VH-CH1)
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLR

SEQ ID NO: 74 - protein, 215 aa, Artificial Sequence
  2G12 and 3-ALA light chain (domain exchanged Fab)
AGVVMTQSPSTLSASVGDTITITCRASQSIETWLAWYQQKPGKAPKLLIYKASTLKTGVPSRFSGSGSGTEFTLTISGLQFDDFATYHCQHYAGYSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 40 bp, Artificial Sequence
  3Ala-R
tcgaacgggtccgcgtccgccgcacggtcagaacctttac

SEQ ID NO: 76 - DNA, 40 bp, Artificial Sequence
  3Ala-F
gttctgaccgtgcggcggacgcggacccgttcgacgcttg

SEQ ID NO: 77 - DNA, 24 bp, Artificial Sequence
  oligonucleotide pool A / primer A
gcccaggcggccgcagaagttcag

SEQ ID NO: 78 - DNA, 58 bp, Artificial Sequence
  primer E /oligonucleotide pool E
cctttggtcgacgccggagaaacggtaacaacggtacccggaccccaagcgtcgaacg

SEQ ID NO: 79 - DNA, 6145 bp, Artificial Sequence
  pAC8 sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaaaagacagctatcgcgattgcagtggcactggctggtttcgctaccgtggcccaggcggccgagatagtcctcacgcagtctccaggcaccctgtctttgtctccaggggaaagagccaccctctcctgcagggccagtcagagtgttagtagcgcctacttagcctggtaccagcagaaacctggccaggctcccaggctcctcatctatggtgcatccagcagggccactggcatcccagacaggttcagtggcagtgggtctgggacagacttcactctcaccatcagcagactggaacctgaagattttgcagtgtattactgtcagcagtatggtaggtcacccactttcggcggagggaccaaggtggagatcaaaggtggttcgtctagatcttcctcctctggtggcggtggctcgggcggtggtggccaggtccagctcgtccagtcaggggctgaggtgaagaagcctgggtcctcggtgaaggtctcctgcaaggcttctggaggttccttcagcagctatgctatcaactgggtgcgacaggcccctggacaagggcttgagtggatgggagggctcatgcctatctttgggacaacaaactacgcacagaagttccaggacagactcacgattaccgcggacgtatccacgagtacagcctacatgcagctgagcggcctgacatatgaagacacggccatgtattactgtgcgagagttgcctatatgttggaacctaccgtcactgcagggggtttggacgtctggggccaagggaccacggtcaccgtgagctcagcttccaccaagggcggccaggccggccagcaccatcaccatcaccatggcgcatacccgtacgacgttccggactacgcttcttaggcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 80 - DNA, 41 bp, Artificial Sequence
  LacITerm-F1 Primer
ggcgccgctcttcgagcgacaccatcgaatggcgcaaaacc

SEQ ID NO: 81 - DNA, 41 bp, Artificial Sequence
  LacITerm-R1 Primer
cttttatcgggtaccgctcactgcccgctttccagtcggga

SEQ ID NO: 82 - DNA, 58 bp, Artificial Sequence
  Term-R Primer
gctaggtgggctgcaaaacaaaacggcctcctgtcaggaagccgcttttatcgggtac

SEQ ID NO: 83 - DNA, 41 bp, Artificial Sequence
  LacITerm-F2 Primer
aaagcgggcagtgagcggtacccgataaaagcggcttcctg

SEQ ID NO: 84 - DNA, 41 bp, Artificial Sequence
  TermPO-R Primer
cacattaattgcgttgaggtgggctgcaaaacaaaacggcc

SEQ ID NO: 85 - DNA, 41 bp, Artificial Sequence
  TermPO-F Primer
gttttgcagcccacctcaacgcaattaatgtgagttagctc

SEQ ID NO: 86 - DNA, 30 bp, Artificial Sequence
  SgrAIPelB-R Primer
cataacaacaccggcggccatggccggctg

SEQ ID NO: 87 - DNA, 20 bp, Artificial Sequence
  SeqpCALTerm-F Primer
taaccgtattaccgcctttg

SEQ ID NO: 88 - DNA, 20 bp, Artificial Sequence
  SeqpCALTerm-R Primer
tgccagctgcattaatgaat

SEQ ID NO: 89 - DNA, 21 bp, Artificial Sequence
  SeqpCALIT-R Primer
cataactcacattaattgcgt

SEQ ID NO: 90 - DNA, 20 bp, Artificial Sequence
  SeqITPO-F2 Primer
gttgcccgtctcactggtga

SEQ ID NO: 91 - DNA, 25 bp, Artificial Sequence
  KasI-F Primer
ccaccctggcgcccaatacgcaaac

SEQ ID NO: 92 - DNA, 37 bp, Artificial Sequence
  AmbPelB-R Primer
gcggccatggccggctaggccgcgagcagcagcagac

SEQ ID NO: 93 - DNA, 37 bp, Artificial Sequence
  AmbPelB-F Primer
tgctgctcgcggcctagccggccatggccgccggtgt

SEQ ID NO: 94 - DNA, 36 bp, Artificial Sequence
  AmbOmpA-R Primer
gaacttctgcggccgcctaggctacggtagcgaaac

SEQ ID NO: 95 - DNA, 20 bp, Artificial Sequence
  SeqHCFR1-R Primer
ttagaaacaccgcaagacag

SEQ ID NO: 96 - DNA, 20 bp, Artificial Sequence
  SeqpCAL-F Primer
atataattatgaaatacctg

SEQ ID NO: 97 - DNA, 20 bp, Artificial Sequence
  SeqITPO-F4 Primer
gcgtggaccgcttgctgcaa

SEQ ID NO: 98 - DNA, 45 bp, Artificial Sequence
  CALX24H1S-F primer ; F1
gccgctgtgccatcgctcagtaacgcggccgcagaagttcagctg

SEQ ID NO: 99 - DNA, 39 bp, Artificial Sequence
  R1
ggcggcgctcttcagttagaaacaccgcaagacaggatc

SEQ ID NO: 100 - DNA, 35 bp, Artificial Sequence
  F2
ggcggcgctcttctcgtgttccgggtggtggtctg

SEQ ID NO: 101 - DNA, 37 bp, Artificial Sequence
  R2
ggcggcgctcttcagtagatagcggtgtcttcaacac

SEQ ID NO: 102 - DNA, 34 bp, Artificial Sequence
  F3
ggcggcgctcttcgggtccgggtaccgttgttac

SEQ ID NO: 103 - DNA, 44 bp, Artificial Sequence
  R3
gccgctgtgccatcgctcagtaacgtcgacgccggagaaacggt

SEQ ID NO: 104 - DNA, 39 bp, Artificial Sequence
  H1F Primer
aacttccgtatctctgctnntnnkatgaactgggttcgt

SEQ ID NO: 105 - DNA, 39 bp, Artificial Sequence
  Reference sequence used to design H1F Primer
aacttccgtatctctgctcacaccatgaactgggttcgt

SEQ ID NO: 106 - DNA, 39 bp, Artificial Sequence
  H1R Primer
acgacgaacccagttcatmnnannagcagagatacggaa

SEQ ID NO: 107 - DNA, 39 bp, Artificial Sequence
  Reference sequence used to design H1R Primer
acgacgaacccagttcatggtgtgagcagagatacggaa

SEQ ID NO: 108 - DNA, 60 bp, Artificial Sequence
  H3F Primer
tactactgcgctcgtaaannktctgaccgtnntnnkgacnnknnkccgttcgacgcttgg

SEQ ID NO: 109 - DNA, 60 bp, Artificial Sequence
  Reference sequence used to design H3F Primer
tactactgcgctcgtaaaggttctgaccgtctgtctgacaacgacccgttcgacgcttgg

SEQ ID NO: 110 - DNA, 60 bp, Artificial Sequence
  H3R Primer
accccaagcgtcgaacggmnnmnngtcmnnannacggtcagamnntttacgagcgcagta

SEQ ID NO: 111 - DNA, 60 bp, Artificial Sequence
  Reference sequence used to design H3R Primer
accccaagcgtcgaacgggtcgttgtcagacagacggtcagaacctttacgagcgcagta

SEQ ID NO: 112 - DNA, 24 bp, Artificial Sequence
  CALX24 primer
gccgctgtgccatcgctcagtaac

SEQ ID NO: 113 - DNA, 23 bp, Artificial Sequence
  OmpA-F Primer
gtggcactggctggtttcgctac

SEQ ID NO: 114 - DNA, 48 bp, Artificial Sequence
  VLL1-R Primer
ggaggaagatccagacgaaccacctttgatttcaacacgggtaccctg

SEQ ID NO: 115 - DNA, 46 bp, Artificial Sequence
  L1VH-F Primer
ggtggctcgggcggtggtggcgaagttcagctggttgaatctggtg

SEQ ID NO: 116 - DNA, 46 bp, Artificial Sequence
  VHL2-R Primer
ctgctgctgctgccggatcctcccggagaaacggtaacaacggtac

SEQ ID NO: 117 - DNA, 46 bp, Artificial Sequence
  L2VH-F Primer
ggcgggagctccggcggcggagaagttcagctggttgaatctggtg

SEQ ID NO: 118 - DNA, 47 bp, Artificial Sequence
  VHL1-R Primer
ggaggaagatccagacgaaccacccggagaaacggtaacaacggtac

SEQ ID NO: 119 - DNA, 44 bp, Artificial Sequence
  L1VL-F Primer
ggtggctcgggcggtggtggcgttgttatgacccagtctccgtc

SEQ ID NO: 120 - DNA, 42 bp, Artificial Sequence
  VLSfi-R Primer
gtgctggccggcctggcctttgatttcaacacgggtaccctg

SEQ ID NO: 121 - DNA, 28 bp, Artificial Sequence
  Sfi6His-R Primer
gtgatggtgctggccggcctggcctttg

SEQ ID NO: 122 - DNA, 54 bp, Artificial Sequence
  Linker 1(-) (L1 prime)
gccaccaccgcccgagccaccgccaccagaggcggcagatccagacgaaccacc

SEQ ID NO: 123 - DNA, 54 bp, Artificial Sequence
  Linker 2(-) (L2 prime)
tccgccgccggagctcccgccgccgccgccgctgctgctgctgccggatcctcc

SEQ ID NO: 124 - DNA, 6819 bp, Artificial Sequence
  2G12 Fab in pET Duet vector
ggggaattgtgagcggataacaattcccctctagaaataattttgtttaactttaagaaggagatataccatgaaaaagacagctatcgcgattgcagtggcactggctggtttcgctaccgtggcccaggcggccgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctagggccaggccggccgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaaatacctattgcctacggcagccgctggattgttattactcgctgcccaaccagccatggccgaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcggcagcagccaccatcaccatcaccatggcgcatacccgtacgacgttccggactacgcttcttagctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 125 - DNA, 58 bp, Artificial Sequence
  VHSfi-R Primer
ccatggtgatggtgatggtgctggccggcctggcccggagaaacggtaacaacggtac

SEQ ID NO: 126 - DNA, 24 bp, Artificial Sequence
  AgeI-F Primer
ccctgaaaaccggtgttccgtctc

SEQ ID NO: 127 - DNA, 30 bp, Artificial Sequence
  Cys19-R Primer
caccgcaagacaggcacagagaaccaccag

SEQ ID NO: 128 - DNA, 30 bp, Artificial Sequence
  Cys19-F Primer
ctggtggttctctgtgcctgtcttgcggtg

SEQ ID NO: 129 - DNA, 25 bp, Artificial Sequence
  NcoI25-R Primer
ggtatgcgccatggtgatggtgatg

SEQ ID NO: 130 - DNA, 36 bp, Artificial Sequence
  HingeVH- R Primer
cgcagcttttcggcggagaaacggtaacaacggtac

SEQ ID NO: 131 - DNA, 42 bp, Artificial Sequence
  VHhinge-F Primer
ccgtttctccgccgaaaagctgcgataaaacccatacctgcc

SEQ ID NO: 132 - DNA, 41 bp, Artificial Sequence
  HingeTemplate-F Primer
gctgcgataaaacccatacctgcccgccgtgcccgggccag

SEQ ID NO: 133 - DNA, 44 bp, Artificial Sequence
  HingeTemplate-R Primer
gatggtgatggtgctggccggcctggcccgggcacggcgggcag

SEQ ID NO: 134 - DNA, 38 bp, Artificial Sequence
  NcoI38-R Primer
gcggcgccatggtgatggtgatggtgctggccggcctg

SEQ ID NO: 135 - DNA, 45 bp, Artificial Sequence
  HingeVH(E)-R Primer
cgcagcttttcggttccggagaaacggtaacaacggtacccggac

SEQ ID NO: 136 - DNA, 45 bp, Artificial Sequence
  VHhinge(E)-F Primer
ccgtttctccggaaccgaaaagctgcgataaaacccatacctgcc

SEQ ID NO: 137 - DNA, 32 bp, Artificial Sequence
  NdeIVH-F Primer
ggagatatacatatgaaatacctattgcctac

SEQ ID NO: 138 - DNA, 26 bp, Artificial Sequence
  XhoIHA26-R Primer
taccagactcgagctaagaagcgtag

SEQ ID NO: 139 - DNA, 44 bp, Artificial Sequence
  HingeCH1-R Primer
caggtatgggttttatcgcagcttttcggttcaactttcttgtc

SEQ ID NO: 140 - DNA, 39 bp, Artificial Sequence
  CH1Hinge-F Primer
ccgaaaagctgcgataaaacccatacctgcccgccgtgc

SEQ ID NO: 141 - DNA, 45 bp, Artificial Sequence
  HingeHisTemplate-F Primer
cccatacctgcccgccgtgcccgcaccatcaccatcaccatggcg

SEQ ID NO: 142 - DNA, 47 bp, Artificial Sequence
  HingeHisTemplate-R Primer
gtccggaacgtcgtacgggtatgcgccatggtgatggtgatggtgcg

SEQ ID NO: 143 - DNA, 47 bp, Artificial Sequence
  XhoIHA-R Primer
accagactcgagctaagaagcgtagtccggaacgtcgtacgggtatg

SEQ ID NO: 144 - DNA, 26 bp, Artificial Sequence
  XbaIVL-F Primer
ggggaattgtgagcggataacaattc

SEQ ID NO: 145 - DNA, 51 bp, Artificial Sequence
  BamHICK-R Primer
ccgccaccggatccaccaccagattcaccacggttgaaagatttggtaacc

SEQ ID NO: 146 - DNA, 42 bp, Artificial Sequence
  SacIVH-F Primer
gcggtgggagctccggtgaagttcagctggttgaatctggtg

SEQ ID NO: 147 - DNA, 51 bp, Artificial Sequence
  HingeCH1deltaC-R Primer
ctggccggcctggccgctgctgccagatttcggttcaactttcttgtcaac

SEQ ID NO: 148 - DNA, 46 bp, Artificial Sequence
  NcoIHinge-R Primer
gtatgcgccatggtgatggtgatggtgctggccggcctggccgctg

SEQ ID NO: 149 - DNA, 79 bp, Artificial Sequence
  SacIBamHI(-)
cccaccgctaccgccgccttcgctgccgccaccttcgctaccgccaccttcgctgccaccaccttcgctgccgccaccg

SEQ ID NO: 150 - DNA, 33 bp, Artificial Sequence
  L216F Primer
gatccggcagcagcagcagcggcggcgggagct

SEQ ID NO: 151 - DNA, 25 bp, Artificial Sequence
  L216R Primer
cccgccgccgctgctgctgctgccg

SEQ ID NO: 152 - DNA, 36 bp, Artificial Sequence
  L217F Primer
gatccggcagcagcagcagcggcggcggcgggagct

SEQ ID NO: 153 - DNA, 28 bp, Artificial Sequence
  L217R Primer
cccgccgccgccgctgctgctgctgccg

SEQ ID NO: 154 - DNA, 42 bp, Artificial Sequence
  L219F Primer
gatccagcggcagcagcagcagcggcggcggcggcgggagct

SEQ ID NO: 155 - DNA, 34 bp, Artificial Sequence
  L219R Primer
cccgccgccgccgccgctgctgctgctgccgctg

SEQ ID NO: 156 - DNA, 45 bp, Artificial Sequence
  L220F Primer
gatccagcggcggcagcagcagcagcggcggcggcggcgggagct

SEQ ID NO: 157 - DNA, 37 bp, Artificial Sequence
  L220R Primer
cccgccgccgccgccgctgctgctgctgccgccgctg

SEQ ID NO: 158 - protein, 225 aa, Artificial Sequence
  2G12 Fab VH-CH1
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 159 - protein, 214 aa, Artificial Sequence
  2G12 Light Chain
DVVMTQSPSTLSASVGDTITITCRASQSIETWLAWYQQKPGKAPKLLIYKASTLKTGVPSRFSGSGSGTEFTLTISGLQFDDFATYHCQHYAGYSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - RNA, 3 nt, Artificial Sequence
  amber stop RNA
uag

SEQ ID NO: 161 - protein, 123 aa, Artificial Sequence
  3-ALA 2G12 VH domain
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRAADADPFDAWGPGTVVTVSP

SEQ ID NO: 162 - protein, 9 aa, Artificial Sequence
  2G12 CDRL3
QHYAGYSAT

SEQ ID NO: 163 - protein, 9 aa, Artificial Sequence
  3-ALA 2G12 CDRL3
QHAAGAAAT

SEQ ID NO: 164 - DNA, 5882 bp, Artificial Sequence
  2G12 3-ALA pCAL IT* (contains 3Ala CDRH3)
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcggtacccgataaaagcggcttcctgacaggaggccgttttgttttgcagcccacctcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcctagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcctaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtgcggcggacgcggacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 165 - DNA, 41 bp, Artificial Sequence
  2G12LCF primer
gccgctgtgccatcgctcagtaacaattgaattaaggagga

SEQ ID NO: 166 - DNA, 20 bp, Artificial Sequence
  L3R primer
atagtgctggcagtggtagg

SEQ ID NO: 167 - DNA, 55 bp, Artificial Sequence
  2G12 reference sequence
ctacctaccactgccagcactacgctggttactctgctaccttcggtcagggtac

SEQ ID NO: 168 - DNA, 55 bp, Artificial Sequence
  AGYS primer
ctacctaccactgccagcactatnnknnknnknnkgctaccttcggtcagggtac

SEQ ID NO: 169 - DNA, 58 bp, Artificial Sequence
  AGYS+1 primer
ctacctaccactgccagcactatnnknnknnknnknnkgctaccttcggtcagggtac

SEQ ID NO: 170 - DNA, 61 bp, Artificial Sequence
  AGYS+2 primer
ctacctaccactgccagcactatnnknnknnknnknnknnkgctaccttcggtcagggtac

SEQ ID NO: 171 - DNA, 48 bp, Artificial Sequence
  2G12LCR primer
gccgctgtgccatcgctcagtaacttaattaattagcattcaccacgg

SEQ ID NO: 172 - DNA, 6 bp, Artificial Sequence
  MfeI restriction site
caattg

SEQ ID NO: 173 - DNA, 8 bp, Artificial Sequence
  PacI restriction site
ttaattaa

SEQ ID NO: 174 - DNA, 5882 bp, Artificial Sequence
  2G12 3ALA LC pCAL IT* (contains 3-Ala CDRL3)
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcggtacccgataaaagcggcttcctgacaggaggccgttttgttttgcagcccacctcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcctagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcacgccgcgggtgcggccgcgaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcctaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 175 - DNA, 34 bp, Artificial Sequence
  L1R primer
gggcggcgctcttcgtagcgaagtcgtcgaactg

SEQ ID NO: 176 - DNA, 55 bp, Artificial Sequence
  SYGA primer
cgggtaccctgaccgaaggtagcmnnmnnmnnmnnatagtgctggcagtggtagg

SEQ ID NO: 177 - DNA, 58 bp, Artificial Sequence
  SYGA+1 primer
cgggtaccctgaccgaaggtagcmnnmnnmnnmnnmnnatagtgctggcagtggtagg

SEQ ID NO: 178 - DNA, 61 bp, Artificial Sequence
  SYGA+2 primer
cgggtaccctgaccgaaggtagcmnnmnnmnnmnnmnnmnnatagtgctggcagtggtagg

SEQ ID NO: 179 - DNA, 34 bp, Artificial Sequence
  L2F primer
gggcggcgctcttccccgtgttgaaatcaaacgt

SEQ ID NO: 180 - DNA, 7 bp, Artificial Sequence
  SapI restriction site
gctcttc

SEQ ID NO: 181 - protein, 9 aa, Artificial Sequence
  anti-C. albicans 2G12 variant CDRL3
QHYKEWRAS

SEQ ID NO: 182 - protein, 9 aa, Artificial Sequence
  anti-C. albicans 2G12 variant CDRL3
QHYKEWSAT

SEQ ID NO: 183 - protein, 9 aa, Artificial Sequence
  anti-C. albicans 2G12 variant CDRL3
QHYREWSAT

SEQ ID NO: 184 - protein, 9 aa, Artificial Sequence
  anti-C. albicans 2G12 variant CDRL3
QHYLAWSAT

SEQ ID NO: 185 - protein, 9 aa, Artificial Sequence
  anti-C. albicans 2G12 variant CDRL3
QHYKEWWAT

SEQ ID NO: 186 - protein, 9 aa, Artificial Sequence
  anti-C. albicans 2G12 variant CDRL3
QHYREWWAT

SEQ ID NO: 187 - protein, 9 aa, Artificial Sequence
  anti-C. albicans 2G12 variant CDRL3
QHYLSWSAT

SEQ ID NO: 188 - protein, 9 aa, Artificial Sequence
  anti-C. albicans 2G12 variant CDRL3
QHYKPFNAT

SEQ ID NO: 189 - protein, 9 aa, Artificial Sequence
  anti-C. albicans 2G12 variant CDRL3
QHYRPFNAT

SEQ ID NO: 190 - protein, 9 aa, Artificial Sequence
  anti-C. albicans 2G12 variant CDRL3
QHYMPFNAT

SEQ ID NO: 191 - protein, 9 aa, Artificial Sequence
  anti-C. albicans 2G12 variant CDRL3
QHYQPFNAT

SEQ ID NO: 192 - protein, 9 aa, Artificial Sequence
  anti-C. albicans 2G12 variant CDRL3
QHYLPFNAT

SEQ ID NO: 193 - protein, 9 aa, Artificial Sequence
  anti-C. albicans 2G12 variant CDRL3
QHYEPFNAT

SEQ ID NO: 194 - protein, 9 aa, Artificial Sequence
  anti-C. albicans 2G12 variant CDRL3
QHYKPFEAT

SEQ ID NO: 195 - protein, 9 aa, Artificial Sequence
  anti-C. albicans 2G12 variant CDRL3
QHYRPFEAT

SEQ ID NO: 196 - protein, 9 aa, Artificial Sequence
  anti-C. albicans 2G12 variant CDRL3
QHYKPFQAT

SEQ ID NO: 197 - protein, 9 aa, Artificial Sequence
  anti-C. albicans 2G12 variant CDRL3
QHYRPFQAT

SEQ ID NO: 198 - protein, 9 aa, Artificial Sequence
  anti-C. albicans 2G12 variant CDRL3
QHYQPFQAT

SEQ ID NO: 199 - protein, 9 aa, Artificial Sequence
  anti-C. albicans 2G12 variant CDRL3
QHYIPFQAT

SEQ ID NO: 200 - protein, 9 aa, Artificial Sequence
  anti-C. albicans 2G12 variant CDRL3
QHYKPFSAS

SEQ ID NO: 201 - protein, 9 aa, Artificial Sequence
  anti-C. albicans 2G12 variant CDRL3
QHYRPFSAT

SEQ ID NO: 202 - protein, 9 aa, Artificial Sequence
  anti-C. albicans 2G12 variant CDRL3
QHYQPFSAT

SEQ ID NO: 203 - protein, 9 aa, Artificial Sequence
  anti-C. albicans 2G12 variant CDRL3
QHYVPFSAT

SEQ ID NO: 204 - protein, 9 aa, Artificial Sequence
  anti-C. albicans 2G12 variant CDRL3
QHYHPFSAT

SEQ ID NO: 205 - protein, 9 aa, Artificial Sequence
  anti-C. albicans 2G12 variant CDRL3
QHYKPFHAT

SEQ ID NO: 206 - protein, 9 aa, Artificial Sequence
  anti-C. albicans 2G12 variant CDRL3
QHYRPFHAT

SEQ ID NO: 207 - protein, 9 aa, Artificial Sequence
  anti-C. albicans 2G12 variant CDRL3
QHYMPFHAT

SEQ ID NO: 208 - protein, 9 aa, Artificial Sequence
  anti-C. albicans 2G12 variant CDRL3
QHYEPFHAT

SEQ ID NO: 209 - protein, 9 aa, Artificial Sequence
  anti-C. albicans 2G12 variant CDRL3
QHYKPFRAT

SEQ ID NO: 210 - protein, 9 aa, Artificial Sequence
  anti-C. albicans 2G12 variant CDRL3
QHYVPFRAT

SEQ ID NO: 211 - protein, 9 aa, Artificial Sequence
  anti-C. albicans 2G12 variant CDRL3
QHYEPFRAT

SEQ ID NO: 212 - protein, 9 aa, Artificial Sequence
  anti-C. albicans 2G12 variant CDRL3
QHYKPFAAT

SEQ ID NO: 213 - protein, 9 aa, Artificial Sequence
  anti-C. albicans 2G12 variant CDRL3
QHYVPFAAT

SEQ ID NO: 214 - protein, 9 aa, Artificial Sequence
  anti-C. albicans 2G12 variant CDRL3
QHYIPFAAT

SEQ ID NO: 215 - protein, 9 aa, Artificial Sequence
  anti-C. albicans 2G12 variant CDRL3
QHYKPFDAT

SEQ ID NO: 216 - protein, 9 aa, Artificial Sequence
  anti-C. albicans 2G12 variant CDRL3
QHYMPFDAT

SEQ ID NO: 217 - protein, 9 aa, Artificial Sequence
  anti-C. albicans 2G12 variant CDRL3
QHYMPFKAT

SEQ ID NO: 218 - protein, 9 aa, Artificial Sequence
  anti-C. albicans 2G12 variant CDRL3
QHYMPFTAT

SEQ ID NO: 219 - protein, 9 aa, Artificial Sequence
  anti-C. albicans 2G12 variant CDRL3
QHYMPFPAT

SEQ ID NO: 220 - protein, 9 aa, Artificial Sequence
  anti-C. albicans 2G12 variant CDRL3
QHYQPFWAT

SEQ ID NO: 221 - protein, 9 aa, Artificial Sequence
  anti-C. albicans 2G12 variant CDRL3
QHYSPFWAT

SEQ ID NO: 222 - protein, 9 aa, Artificial Sequence
  anti-C. albicans 2G12 variant CDRL3
QHYMPYRAS

SEQ ID NO: 223 - protein, 9 aa, Artificial Sequence
  anti-C. albicans 2G12 variant CDRL3
QHYKPYRAT

SEQ ID NO: 224 - protein, 9 aa, Artificial Sequence
  anti-C. albicans 2G12 variant CDRL3
QHYMPYRAT

SEQ ID NO: 225 - protein, 9 aa, Artificial Sequence
  anti-C. albicans 2G12 variant CDRL3
QHYQPYRAT

SEQ ID NO: 226 - protein, 9 aa, Artificial Sequence
  anti-C. albicans 2G12 variant CDRL3
QHYLPYRAT

SEQ ID NO: 227 - protein, 9 aa, Artificial Sequence
  anti-C. albicans 2G12 variant CDRL3
QHYEPYRAT

SEQ ID NO: 228 - protein, 9 aa, Artificial Sequence
  anti-C. albicans 2G12 variant CDRL3
QHYKPYDAT

SEQ ID NO: 229 - protein, 9 aa, Artificial Sequence
  anti-C. albicans 2G12 variant CDRL3
QHYKPYSAT

SEQ ID NO: 230 - protein, 9 aa, Artificial Sequence
  anti-C. albicans 2G12 variant CDRL3
QHYQPYVAT

SEQ ID NO: 231 - protein, 9 aa, Artificial Sequence
  anti-C. albicans 2G12 variant CDRL3
QHYEPYKAT

SEQ ID NO: 232 - protein, 9 aa, Artificial Sequence
  anti-C. albicans 2G12 variant CDRL3
QHYLPYQAS

SEQ ID NO: 233 - protein, 10 aa, Artificial Sequence
  anti-C. albicans 2G12 variant CDRL3
QHYRPHTGAT

SEQ ID NO: 234 - protein, 10 aa, Artificial Sequence
  anti-C. albicans 2G12 variant CDRL3
QHYTAHDGAT

SEQ ID NO: 235 - protein, 10 aa, Artificial Sequence
  anti-C. albicans 2G12 variant CDRL3
QHYTAHRGAT

SEQ ID NO: 236 - protein, 10 aa, Artificial Sequence
  anti-C. albicans 2G12 variant CDRL3
QHYRAHTGAT

SEQ ID NO: 237 - protein, 10 aa, Artificial Sequence
  anti-C. albicans 2G12 variant CDRL3
QHYTAHTGAT

SEQ ID NO: 238 - protein, 10 aa, Artificial Sequence
  anti-C. albicans 2G12 variant CDRL3
QHYTDHHGAT

SEQ ID NO: 239 - protein, 10 aa, Artificial Sequence
  anti-C. albicans 2G12 variant CDRL3
QHYTDHKGAT

SEQ ID NO: 240 - protein, 10 aa, Artificial Sequence
  anti-C. albicans 2G12 variant CDRL3
QHYTDHRGAT

SEQ ID NO: 241 - protein, 10 aa, Artificial Sequence
  anti-C. albicans 2G12 variant CDRL3
QHYTDHYGAT

SEQ ID NO: 242 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKEWRASFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKEWSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYREWSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYLAWSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKEWWATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYREWWATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYLSWSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFNATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYRPFNATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPFNATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYQPFNATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYLPFNATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYEPFNATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFEATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYRPFEATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFQATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYRPFQATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYQPFQATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYIPFQATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFSASFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYRPFSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYQPFSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 264 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYVPFSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYHPFSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFHATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYRPFHATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPFHATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYEPFHATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYVPFRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYEPFRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFAATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYVPFAATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYIPFAATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPFDATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPFDATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPFKATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPFTATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPFPATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYQPFWATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYSPFWATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPYRASFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPYRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYMPYRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYQPYRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYLPYRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYEPYRATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPYDATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 290 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYKPYSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 291 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYQPYVATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYEPYKATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 293 - protein, 215 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYLPYQASFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 294 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYRPHTGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYTAHDGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYTAHRGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYRAHTGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYTAHTGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYTDHHGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 300 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYTDHKGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 301 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYTDHRGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 302 - protein, 216 aa, Artificial Sequence
  anti-C. albicans 2G12 Fab light chain variant
AGVVMTQSPSTLSASVGDTITITCRASQSIETWLAWYQQKPGKAPKLLIYKASTLKTGVPSRFSGSGSGTEFTLTISGLQFDDFATYHCQHYTDHYGATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 303 - protein, 14 aa, Artificial Sequence
  2G12 CDRH3
KGSDRLSDNDPFDA

SEQ ID NO: 304 - protein, 14 aa, Artificial Sequence
  2G12 3ALA CDRH3
KGSDRAADADPFDA

SEQ ID NO: 305 - protein, 108 aa, Artificial Sequence
  2G12 3ALA CDRL3 VL domain
AGVVMTQSPSTLSASVGDTITITCRASQSIETWLAWYQQKPGKAPKLLIYKASTLKTGVPSRFSGSGSGTEFTLTISGLQFDDFATYHCQHAAGAAATFGQGTRVEIK

SEQ ID NO: 306 - protein, 228 aa, Artificial Sequence
  2G12 3ALA CDRH3 Fab Heavy Chain
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRAADADPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLR

SEQ ID NO: 307 - protein, 215 aa, Artificial Sequence
  2G12 3ALA CDRL3 Fab Light Chain
AGVVMTQSPSTLSASVGDTITITCRASQSIETWLAWYQQKPGKAPKLLIYKASTLKTGVPSRFSGSGSGTEFTLTISGLQFDDFATYHCQHAAGAAATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 454 aa, Artificial Sequence
  2G12 IgG Heavy Chain
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 5 aa, Artificial Sequence
  2G12 CDRH1
AHTMN

SEQ ID NO: 310 - protein, 17 aa, Artificial Sequence
  2G12 CDRH2
SISTSSTYRDYADAVKG

SEQ ID NO: 311 - protein, 11 aa, Artificial Sequence
  2G12 CDRL1
RASQSIETWLA

SEQ ID NO: 312 - protein, 7 aa, Artificial Sequence
  2G12 CDRL2
KASTLKT

SEQ ID NO: 313 - protein, 454 aa, Artificial Sequence
  2G12 mutant V5L, H230S
EVQLLESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSTCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 453 aa, Artificial Sequence
  2G12 mutant D1 V5L (H230 deletion)
EVQLLESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTTCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 452 aa, Artificial Sequence
  2G12 mutant D2 V5L (T229-H230 deletion)
EVQLLESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 451 aa, Artificial Sequence
  2G12 mutant D3 V5L (K228-H230 deletion)
EVQLLESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDTCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 450 aa, Artificial Sequence
  2G12 mutant D4 V5L (D227-H230 deletion)
EVQLLESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCTCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 318 - protein, 448 aa, Artificial Sequence
  2G12 mutant D6 V5L (D227-C232 deletion)
EVQLLESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 448 aa, Artificial Sequence
  2G12 mutant D6GG V5L (D227-C232 deletion)
EVQLLESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 452 aa, Artificial Sequence
  2G12 mutant D2 (T229-H230 deletion)
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTCPPCPPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 107 aa, Artificial Sequence
  2G12 3ALA CDRL3 VL domain
DVVMTQSPSTLSASVGDTITITCRASQSIETWLAWYQQKPGKAPKLLIYKASTLKTGVPSRFSGSGSGTEFTLTISGLQFDDFATYHCQHAAGAAATFGQGTRVEIK

SEQ ID NO: 322 - protein, 214 aa, Artificial Sequence
  2G12 3ALA CDRL3 Fab Light Chain
DVVMTQSPSTLSASVGDTITITCRASQSIETWLAWYQQKPGKAPKLLIYKASTLKTGVPSRFSGSGSGTEFTLTISGLQFDDFATYHCQHAAGAAATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - DNA, 4765 bp, Artificial Sequence
  3-ALA LC 2G12 pCAL G13
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaattgaattaaggaggatataattatgaaatacctgctgccgaccgcagccgctggtctgctgctgctcgcggcccagccggccatggccgccggtgttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcacgcggctggtgcggcggctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaattaattaataaggaggatataattatgaaaaagacagctatcgcgattgcagtggcactggctggtttcgctaccgtagcccaggcggccgcagaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggcgtcgaccaaaggtccgtctgttttcccgctggctccgtcttctaaatctacctctggtggtaccgctgctctgggttgcctggttaaagactacttcccggaaccggttaccgtttcttggaactctggtgctctgacctctggtgttcacaccttcccggctgttctgcagtcttctggtctgtactctctgtcttctgttgttaccgttccgtcttcttctctgggtacccagacctacatctgcaacgttaaccacaaaccgtctaacaccaaagttgacaagaaagttgaaccgaaatcttgcctgcgatcgcggccaggccggccgcaccatcaccatcaccatggcgcatacccgtacgacgttccggactacgcttctactagttaggagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagctagctaacgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 324 - DNA, 42 bp, Artificial Sequence
  2G12LCF1
gccgctgtgccatcgctcagtaaccaattgaattaaggagga

SEQ ID NO: 325 - DNA, 35 bp, Artificial Sequence
  2G12LCR1
ggcggcgctcttctagcgaagtcgtcgaactgcag

SEQ ID NO: 326 - DNA, 54 bp, Artificial Sequence
  2G12ALACF2
gctacctaccactgccagcacgccgcgggtgcggccgcgaccttcggtcagggt

SEQ ID NO: 327 - DNA, 54 bp, Artificial Sequence
  2G12ALACR2
ggtaccctgaccgaaggtcgcggccgcacccgcggcgtgctggcagtggtaggt

SEQ ID NO: 328 - DNA, 35 bp, Artificial Sequence
  2G12LCF3
ggcggcgctcttctacccgtgttgaaatcaaacgt

SEQ ID NO: 329 - DNA, 42 bp, Artificial Sequence
  2G12IgGLC-F
ggtccctggctcgagtgaggttgttatgacccagtctccgtc

SEQ ID NO: 330 - DNA, 44 bp, Artificial Sequence
  2G12IgGLC-R
cctggtaccgaattcttagcattcaccacggttgaaagatttgg

SEQ ID NO: 331 - DNA, 63 bp, Artificial Sequence
  2G12HindIIILC-F1
gtaagcaagcttatggacatgagagtgcctgcacagctgctgggactgctgctgctgtggctg

SEQ ID NO: 332 - DNA, 62 bp, Artificial Sequence
  2G12HindIIILC-F2
ggactgctgctgctgtggctgccaggcgccaagtgcgacgttgttatgacccagtctccgtc

SEQ ID NO: 333 - DNA, 46 bp, Artificial Sequence
  2G12EcoRILC-R
cgctacgaattctcagcattcaccacggttgaaagatttggtaacc

SEQ ID NO: 334 - protein, 453 aa, Artificial Sequence
  pCALM 2G12 IgG Heavy Chain
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 452 aa, Artificial Sequence
  pDR12 2G12 IgG Heavy Chain
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - DNA, 6455 bp, Artificial Sequence
  2G12 pCALM 8His
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgcccaccatggactggacctggcggatcctgttcctggtggccgcggccaccggagcccacagcgaagttcagctggttgaatctggtggtggtctggttaaagctggtggttctctgatcctgtcttgcggtgtttctaacttccgtatctctgctcacaccatgaactgggttcgtcgtgttccgggtggtggtctggaatgggttgcttctatctctacctcttctacctaccgtgactacgctgacgctgttaaaggtcgtttcaccgtttctcgtgacgacctggaagacttcgtttacctgcagatgcataaaatgcgtgttgaagacaccgctatctactactgcgctcgtaaaggttctgaccgtctgtctgacaacgacccgttcgacgcttggggtccgggtaccgttgttaccgtttctccggctagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcgggtctcatcaccaccatcaccatcaccatggcactagtcgggccaagcgggcacccgtgaagcagaccctgaacttcgacctgctgaagctggccggcgacgtggagagcaaccccggccccatgaggctccctgctcagctcctggggctgctaatgctctgggtccctggctcgagtgaggttgttatgacccagtctccgtctaccctgtctgcttctgttggtgacaccatcaccatcacctgccgtgcttctcagtctatcgaaacctggctggcttggtaccagcagaaaccgggtaaagctccgaaactgctgatctacaaggcttctaccctgaaaaccggtgttccgtctcgtttctctggttctggttctggtaccgagttcaccctgaccatctctggtctgcagttcgacgacttcgctacctaccactgccagcactacgctggttactctgctaccttcggtcagggtacccgtgttgaaatcaaacgtaccgttgctgctccgtctgttttcatcttcccgccgtctgacgaacagctgaaatctggtaccgcttctgttgtttgcctgctgaacaacttctacccgcgtgaagctaaagttcagtggaaagttgacaacgctctgcagtctggtaactctcaggaatctgttaccgaacaggactctaaagactctacctactctctgtcttctaccctgaccctgtctaaagctgactacgaaaagcacaaagtttacgcttgcgaagttacccaccagggtctgtcttctccggttaccaaatctttcaaccgtggtgaatgctaagaattcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 337 - DNA, 13309 bp, Artificial Sequence
  2G12 pDR12
attgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagcttcgtgtcaaggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcacagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatcagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagctctagagccgccaccatggaactggggctccgctgggttttccttgttgctattttagaaggtgtccagtgtgaggtgcagctggtggagtctgggggaggcctggtcaaggcgggaggatccctcatactctcctgtggagtctctaattttagaatctctgcccataccatgaattgggtccgccgggttccagggggggggctggagtgggtcgcttccattagtacgagttccacttatagagactatgcagacgctgtgaagggccgattcaccgtttccagagacgacctcgaagactttgtgtatttgcaaatgcacaaaatgagagtcgaagacacggctatttattactgcgccagaaagggatctgacagactaagcgacaacgatccttttgatgcctggggcccagggacagtggtcaccgtctctcccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattcgagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccccctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgcccctgtcctcccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcagacggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggcccgtggccgggggactgttgggcgccatctccttgcatgcaccattccttgcggcggcggtgctcaacggcctcaacctactactgggctgcttcctaatgcaggagtcgcataagggagagcgtcgacctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcatcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagccaagctagatccggctgtggaatgtgtgtcagttagggtgtggaaagtccccaggctccccagcaggcagaagtatgcaaagcatgcatctcaattagtcagcaaccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaacctagcttggggccaccgctcagagcaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaactccaaataggaccctgtgaaggaatccgcatgggagatcatctctgggtggcccgtttcatcttncatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtggtctgactgggttccacgaaacgtccaacatcaacgacttttctgctggtgtcgccaatcgcagtgccagcatccgcattccccggactgtcggccaggagaagaaaggttactttgaagaccgcggcccctctgccaattgtgacccctttgcagtgacagaagccatcgtccgcacatgccttctcaatgagactggccacgagcccttccaatacaaaaactaattagactttgagtgatcttgagcctttcctagttcatcccaccccgccccagagagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagcttcgtgtcaaggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcacagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatcagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttaccatggacatgagggtccccgctcagctcctggggctcctgctgctctggctcccaggtaaggaaggagaacactaggaatttactcagcccagtgtgctcagtactgcctggttattcagggaagtcttcctataatatgatcaatagtatgaatatttgtgtttctatttccaatctcaggtgccaaatgtgatgttgtgatgactcagtctccttccaccctgtctgcatctgtcggagacacaatcaccatcacttgccgggccagtcagagtattgaaacctggttggcctggtatcagcagaagccagggaaagccccaaaactcctaatctacaaggcgtctactttaaaaactggagtcccgtcaagattcagcggcagtggatctggaacagagttcactcttaccatcagtggcctgcagttcgatgactttgcaacttatcactgtcagcactatgctggttattcagccacttttggccagggaaccagggtggagatcaaacgtactgtggctgcaccatctgtcttcatcttcccgccatctgatgagcagttgaaatctgggactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgttgagcaaagcagactacgagaaacacaaagtctacgcctgcgaagtcacccatcagggcctgagttcgcccgtcacaaagagcttcaacaggggagagtgttaatgaattc

(19) United States
(12) Patent Application Publication
(10) Pub. No.: US 2010/0093563 A1
(43) Pub. Date: Apr. 15, 2010

(54) Methods and vectors for display of molecules and displayed molecules and collections

(76) Inventors: Robert Anthony Williamson, La Jolla, CA (US); Jehangir Wadia, San Diego, CA (US); Toshiaki Maruyama, La Jolla, CA (US); Zhifeng Chen, Vista, CA (US); Joshua Nelson, La Jolla, CA (US)

Correspondence Address: K&L Gates LLP, 3580 Carmel Mountain Road, Suite 200, San Diego, CA, 92130, US

(21) Appl. No.: 12/586,307

(22) Filed: Sep. 18, 2009

Related U.S. Application Data
(60) Provisional application No. 61/192,982, filed on Sep. 22, 2008.
(60) Provisional application No. 61/192,960, filed on Sep. 22, 2008.

(51) Int. Cl. C40B 40/08; C07K 19/00; C12N 15/63; C07H 21/04

(52) U.S. Cl. 506/17; 530/391.1; 435/320.1; 536/23.53; 435/69.7

(57) ABSTRACT

Provided herein are methods for generating diverse polypeptide and nucleic acid molecule libraries and collections, and the collections and libraries; methods for selecting variant polypeptides and nucleic acid molecules from the libraries; and molecules selected from the libraries. Exemplary of the polypeptides and nucleic acid molecules are antibodies and nucleic acids encoding the antibodies (including antibody fragments and domain exchanged antibodies). Also provided herein are methods of displaying polypeptides such as antibodies, for example on the surface of genetic packages, such as phage; and libraries and collections of the displayed polypeptides and vectors for producing the displayed polypeptides, libraries and collections. Exemplary of the displayed antibodies are domain exchanged antibodies.

### RELATED APPLICATIONS

Benefit of priority is claimed to U.S. Provisional Application Ser. No. 61/192,982 to Robert Anthony Williamson, Jehangir Wadia, Toshiaki Maruyama, Zhifeng Chen and Joshua Nelson, entitled “METHODS AND VECTORS FOR DISPLAY OF MOLECULES AND DISPLAYED MOLECULES AND COLLECTIONS,” filed on Sep. 22, 2008, and to U.S. Provisional Application Ser. No. 61/192,960 to Robert Anthony Williamson, Jehangir Wadia, Toshiaki Maruyama, Zhifeng Chen and Josh Nelson, entitled “VECTORS FOR EXPRESSION OF DISPLAYED PROTEINS,” filed on Sep. 22, 2008.

This application is related to corresponding International Application No. PCT/US2009/005221 to Robert Anthony Williamson, Jehangir Wadia, Toshiaki Maruyama, Zhifeng Chen and Joshua Nelson, entitled “METHODS AND VECTORS FOR DISPLAY OF MOLECULES AND DISPLAYED MOLECULES AND COLLECTIONS,” filed on the same day herewith, which also claims priority to U.S. Provisional Application Ser. No. 61/192,982 and U.S. Provisional Application Ser. No. 61/192,960.

This application also is related to U.S. application Ser. No. 12/586,273 to Robert Anthony Williamson, Jehangir Wadia, Toshiaki Maruyama, Zhifeng Chen and Joshua Nelson, entitled “METHODS FOR CREATING DIVERSITY IN LIBRARIES AND LIBRARIES, DISPLAY VECTORS AND METHODS, AND DISPLAYED MOLECULES,” filed on the same day herewith, and to International Application No. PCT/US2009/005230 to Robert Anthony Williamson, Jehangir Wadia, Toshiaki Maruyama, Zhifeng Chen and Josh Nelson, entitled “METHODS FOR CREATING DIVERSITY IN LIBRARIES AND LIBRARIES, DISPLAY VECTORS AND METHODS, AND DISPLAYED MOLEULES,” filed on the same day herewith. This application also is related to U.S. Provisional Application No. 61/277,091 to Robert Anthony Williamson, Jehangir Wadia, Michelle Wagner, Joshua Nelson, Toshiaki Maruyama, and Lucy Chammas, entitled “ANTIBODIES AGAINST CANDIDA AND OF USE,” filed on the same day herewith.

The subject matter of each of the above-referenced applications is incorporated by reference in its entirety.

### FIELD OF INVENTION

Provided herein are methods of displaying polypeptides such as antibodies, libraries and collections of the displayed polypeptides and vectors for producing the displayed polypeptides, libraries and collections. Also provided are vectors for expressing polypeptides, wherein the polypeptides are expressed with reduced toxicity to the host cells, and cells and methods of expressing such polypeptides.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy # 1 and Copy # 2), the contents of which are incorporated by reference in their entirety. The computer-readable file on each of the aforementioned compact discs, created on Sep. 18, 2009, is identical, 394 kilobytes in size, and titled 1107SEQ.001.txt.

### BACKGROUND

Domain exchanged antibodies have non-conventional “exchanged” three-dimensional structures, in which the variable heavy chain domain “swings away” from its cognate light chain and interacts instead with the “opposite” light chain, such that the two heavy chains are interlocked. This unusual folding and pairing creates an interface between the two adjacent heavy chain variable regions (V_{H}-V_{H}′ interface). Typically, this interface contributes to a non-conventional antigen binding site containing residues from each V_{H} domain. In one example, mutations in the heavy chain framework contribute to and/or stabilize the domain exchanged configuration. For example, mutation(s) in the joining region between the VH and CH domains can contribute to the domain exchanged configuration. In another example, mutations along the V_{H}-V_{H}′ interface can stabilize the domain-exchange configuration (see, for example, Published U.S. Application, Publication No.: US20050003347).

In one example, the domain exchanged structure, including constrained antibody combining sites, can facilitate antigen binding within densely packed and/or repetitive epitopes, for example, sugar residues on bacterial or viral surfaces, such as, for example, epitopes within high density arrays (e.g. in pathogens and tumor cells) that can be poorly recognized by conventional antibodies.

Methods are needed for display of domain exchanged antibodies and for making display libraries for production and selection of new domain exchange antibodies. Accordingly, it is among the objects herein is to provide methods for producing display libraries for producing and selecting domain exchanged antibodies and new domain exchanged antibodies produced by the methods.

Further, because the expression of domain-exchanged antibodies, like convention antibodies and many other polypeptides, are toxic to the host cell when expressed recombinantly, tools (e.g. nucleic acids, vectors and cells) and methods are needed for expression whereby the toxicity of the antibodies or other protein is reduced. Toxicity of recombinant proteins can hinder both their initial identification and subsequent development and/or modification for research and therapeutic use. For example, effective screening and selection of proteins from libraries, such as, for example, phage display libraries, relies on the stable expression of every protein in the library. Proteins, such as antibodies, that are toxic to host cells typically cannot be recovered using such methods. In some instances, the host cell expressing the protein is non-viable. In other instances, the nucleic acid encoding the protein is modified or deleted to reduce toxicity such that the protein is no longer expressed in its wild-type form. In such examples, the proteins are no longer available in the library for screening and selection, or are present at insufficient levels for recovery. Accordingly, it is among the objects herein is to provide vectors and cells that can be used to express proteins with reduced toxicity to the host cells.

### SUMMARY

Provided herein are methods and vectors for display of polypeptides, and in particular antibodies, typically domain exchanged antibodies (including domain exchanged antibody fragments) and other antibodies (including fragments) that are displayed bivalently (e.g. two separate polypeptide chains interacting via covalent bonds). Also provided are display libraries expressing the antibodies, such as domain exchanged antibodies, methods for selecting polypeptides (e.g. domain exchanged antibodies) from the libraries, and polypeptides (e.g. domain exchanged antibodies) selected from the libraries.

Provided herein are genetic packages on which domain exchanged antibodies are displayed. In one example, the genetic package contains a domain exchanged antibody, wherein the domain exchanged antibody fused to a genetic package display protein, whereby the domain exchanged antibody is displayed on the genetic package; and. As described herein, a domain exchanged antibody typically contains a first variable heavy chain (V_{H}) domain, a second variable heavy chain (V_{H}′) domain, a first variable light chain (V_{L}) domain and a second variable light chain (V_{L}′) domain, or functional domains or regions thereof; and an interface is formed between the V_{H} domain and the V_{H}′ domain. In some instances, the V_{H}′ domain interacts with the V_{L} domain, and the V_{H} domain interacts with the V_{L}′ domain. The domain exchanged antibody can contain one or more of a peptide linker that joins the V_{H} domain and the V_{L}′ domain; a peptide linker that joins the V_{H}′ domain and the V_{L} domain; and a peptide linker that joins the V_{H}′ domain and the V_{H} domain. In some instances, the genetic package display protein is fused to one of the V_{H} domain, V_{H}′ domain, V_{L} domain and the V_{L}′ domain.

The domain exchanged antibodies displayed on the packages also conatin a first constant heavy chain (C_{H}) domain, a second constant heavy chain (C_{H}′) domain, a first constant light chain (C_{L}) domain and a second constant light chain (C_{L}′), or functional regions thereof. In such cases, the V_{H} domain and C_{H} domain can be linked, thereby forming a V_{H}-C_{H} chain; the V_{H}′ domain and C_{H}′ domain can linked, thereby forming a V_{H}′-C_{H}′ chain; the V_{L} domain and C_{L} domain can be linked, thereby forming a V_{L}-C_{L} chain; and the V_{L}′ domain and C_{L}′ domain can be linked, thereby forming a V_{L}′-C_{L}′ chain. Alternatively, these domains can be linked by a peptide linker. In a particular examples, the domain exchanged antibody contains a peptide linker that joins the V_{H} domain and the C_{L}′ domain and a peptide linker that joins the V_{H}′ domain and the C_{L} domain. For display of the domain exchanged antibody, the genetic package display protein can be fused to one or more of the C_{H} domain, C_{H}′ domain C_{L} domain and the C_{L}′ domain.

In some aspects, some of the domains or functional regions thereof have identical amino acid sequences. For example, the V_{H} domain and the V_{H}′ domain or functional regions thereof can have identical amino acid sequences; the V_{L} domain and the V_{L}′ domain or functional regions thereof have identical amino acid sequences; the C_{H} domain and the C_{H}′ domain or functional regions thereof can have identical amino acid sequences; and the C_{L} domain and the C_{L}′ domain or functional regions thereof can have identical amino acid sequences.

In one example, the displayed domain exchanged antibody displayed on the genetic packages contains a fusion protein that contains a domain exchanged antibody domain or functional region thereof fused to a genetic package display protein, and a non-fusion polypeptide that contains a domain exchanged antibody domain or functional region thereof and not a genetic package display protein. Alternatively, or in combination with the above, the displayed domain exchanged antibody contains a single polypeptide chain that contains a fusion protein containing at least two domain exchanged antibody domains or functional regions thereof, fused to a genetic package display protein, and a peptide linker. In some examples, the genetic package a phage, such as a bacteriophage, such as a Ff, M13, fd, or fl bacteriophage.

In some aspects, the domain exchanged antibody displayed on the genetic package is a domain exchanged antibody fragment. Exemplary of the domain exchanged antibody fragments that can be displayed on the genetic packages provided herein include, but are not limited to, domain exchanged Fab fragments, domain exchanged scFv fragment, domain exchanged single chain Fab (scFab) fragments, domain exchanged scFv tandem fragments, domain exchanged scFv hinge fragments and domain exchanged Fab hinge fragments. The domain exchanged antibody fragment typically contains two heavy chain variable region domains (V_{H}) or functional regions thereof, and optionally contains two light chain variable region domains (V_{L}) or functional regions thereof.

In some examples, the domain exchanged antibody fragment contains at least two conventional antibody combining sites, which, in some embodiments, are within less than at or about 100, 90, 80, 70, 60, 50, 40, or 30 angstroms, e.g. less than 100 or less than about 100 angstroms, or within less than 50 or less than about 50 angstroms, or within less than 35 or less than about 35 angstroms of one another. In a particular example, the domain exchanged antibody fragment contains one non-conventional antibody combining site, the non-conventional antibody combining site containing a CDR of each of two heavy chain variable region domains.

The domain exchanged antibodies displayed on the genetic packages provided herein can specifically bind to an antigen, such as a carbohydrate, polysaccharide, proteoglycan, lipid, protein, nucleic acid or glycolipid. In one example, the antigen to which the antibody binds is expressed in or on any cell, tissue, blood, fluid or organism. In a particular embodiment, the domain exchanged antibodies displayed on the genetic packages specifically bind to an antigen expressed on an infectious agent, such as, for example, a microbe, virus, bacteria (including gram negative bacteria and gram positive bacteria), yeast, fungi, and drug-resistant infectious agents. The antigen can be expressed on, for example, a viral surface or a bacterial cell wall, or a cancerous cell or tissue, such as a tumor cell. In one aspect, the domain exchanged antibody displayed on the genetic packages provided herein specifically binds an antigen other than HIV gp120. In one example, the domain exchanged antibody can specifically bind to the antigen other than HIV gp120 with a higher affinity than it binds to HIV gp120, or the domain exchanged antibody does not specifically bind to HIV gp120. In particular examples, the domain exchanged antibody is a 2G12 antibody

Exemplary of the domain exchanged antibodies that can be displayed on the genetic packages provided herein is a modified domain exchanged antibody, wherein the domain exchanged antibody is a modified domain exchanged antibody, containing modification(s) at one or more amino acid residue positions compared to the native unmodified domain exchanged antibody. The domain exchanged antibody can contain modifications in a CDR or framework region, for example, compared to the native antibody. In one example, the modified 2G12 domain exchanged antibody contains modifications at one or more amino acid residue positions in any one or more of: a heavy chain CDR1, a heavy chain CDR2, a heavy chain CDR3, a light chain CDR1, a light chain CDR2 and a light chain CDR3,n particular examples, the domain exchanged antibody is a 2G12 antibody containing modifications at one or more amino acid residue positions compared to a native 2G12 antibody. In some examples, the native 2G12 antibody contains a V_{H} domain containing the sequence of amino acids set forth in SEQ ID NO: 10 and a V_{L} domain containing the sequence of amino acids set forth in SEQ ID NO: 11. Further, the domain exchanged antibody can contain modifications in one or more amino acid residues in a CDR compared to the native antibody. In one example, the modified 2G12 domain exchanged antibody contains modifications at one or more amino acid residue positions in any one or more of: a heavy chain CDR1, a heavy chain CDR2, a heavy chain CDR3, a light chain CDR1, a light chain CDR2 and a light chain CDR3, compared to the 2G12 antibody. In some examples, the domain exchanged antibody contains modifications at one or more amino acid residues selected from among H31, H32, H33, H52, H95, H96, H97, H98, H99, H100, H100a, H100c, H100d, L89, L90, L91, L92, L93, L94 and L95, based on Kabat numbering.

In one aspect, the domain exchanged antibody displayed on the genetic package provided herein contains two V_{H} domains or functional regions thereof, having identical amino acid sequences. Further, the domain exchanged antibody can contain one or more disulfide bonds, such as for example, one or more hinge region disulfide bonds. In a particular aspect, the domain exchanged antibody contains intra-chain disulfide bonds. In some examples, an amino acid position in the heavy chain of the domain exchanged fragment contains an isoleucine (I) to cysteine (C) mutation, compared to the analogous position in a wild-type domain exchanged antibody or a target polypeptide. In further examples, the one or more disulfide bonds in the domain exchanged antibody includes a disulfide bond between amino acids of the two V_{H} domains or functional regions thereof.

The domain exchanged antibodies displayed on the genetic packages provided herein also can contain one or more dimerization domains, such as one or more of a leucine zipper, GCN4 zipper or an antibody hinge region.

In a particular example, the domain exchanged antibody contains a modification in Ile 19 of the V_{H} amino acid sequence of a 2G12 antibody.

In examples where the domain exchanged antibody displayed on a genetic package provided herein contains the fusion protein and the non-fusion polypeptide, the domain exchanged antibody domain or functional region contained in the fusion protein can have an identical amino acid sequence compared to the domain exchanged antibody domain or functional region contained in the non-fusion polypeptide.

Provided herein are compositions containing a plurality of genetic packages described above and provided herein. Also provided are collections of genetic packages, containing genetic packages displaying domain exchanged antibody polypeptides. In some examples, the collection contains the genetic packages described above and provided herein. In one example, the domain exchanged antibody polypeptides displayed on the genetic packages in the collection are variant polypeptides. In one aspect, the collection contains at least 10⁴ or about 10⁴, 10⁵ or about 10⁵, 10⁶ or about 10⁶, 10⁷ or about 10⁷, 10⁸ or about 10⁸, 10⁹ or about 10⁹, 10¹⁰ or about 10¹⁰, 10¹¹ or about 10¹¹, 10¹² or about 10¹², 10¹³ or about 10¹³, or 10¹⁴ or about 10¹⁴ different amino acid sequences among the polypeptide members. In one aspect, the collection contains a diversity ratio that is a high diversity ratio, such as diversity ratios approaching 1, such as, for example, at or about 0.1, 0.2, 0.3, 0.4, 0.5, 0.6, 0.7, 0.8, 0.9 0.91, 0.92, 0.93, 0.94, 0.95. 0.96, 0.97, 0.98, or 0.99.

Provided herein are nucleic acid molecules, such as vectors, for expressing polypeptides. The nucleic acid molecules (e.g. vectors) provided herein contain one or more stop codons that result in limited translation (i.e. translation only some of the time) of an encoded polypeptide. In some examples, the stop codon(s) is located in nucleic acid encoding a leader peptide that is operably linked to nucleic acid encoding a polypeptide of interest. Thus, upon introduction into a partial suppressor cell, in some instances the polypeptide of interest is expressed as a fusion polypeptide with the leader peptide, while in other instances translation is terminated at the stop codon in the nucleic acid encoding the leader peptide, thus limiting the expression of the polypeptide of interest. Limiting the expression of a polypeptide can reduce the toxicity to the host cell that is associated with expression of the polypeptide. Thus, provided herein are nucleic acid molecules for expressing polypeptides, wherein the polypeptides are expressed with reduced toxicity to the host cells compared to in the absence of the stop codon(s).

The nucleic acid molecules, including vectors, provided herein can be used to express polypeptides for display on genetic packages, such as, for example, on bacteriophage. Exemplary of the nucleic acid molecules provided herein are nucleic acid molecules for expressing antibodies or functional fragments thereof, including domain exchanged antibodies or functional fragments thereof, for display on a genetic package. For example, provided herein are nucleic acid molecules, including vectors, for the expression of domain exchanged scFv fragments, domain exchanged scFv tandem fragments; domain exchanged single chain Fab (scFab) fragments, domain exchanged scFv hinge fragments, and domain exchanged Fab hinge fragments. In particular examples, such antibodies and fragments thereof are displayed on genetic packages following expression from the vectors provided herein. Also provided herein are cells and methods of expressing such polypeptides.

Provided herein are nucleic acid molecules containing: a nucleic acid encoding a first leader peptide; a nucleic acid encoding a first polypeptide, wherein the nucleic acid encoding the first leader peptide is operably linked to the nucleic acid encoding the first polypeptide for secretion thereof; a nucleic acid encoding a genetic package display protein, wherein the nucleic acid encoding the genetic package display protein is 3′ of the nucleic acid encoding first polypeptide; and two stop codons. The first stop codon is located in the nucleic acid encoding the first leader peptide or the nucleic acid encoding the first polypeptide, and the second stop codon is located between the nucleic acid encoding the first polypeptide and the nucleic acid encoding the display protein. In some examples, the nucleic acids encoding the first leader peptide, first polypeptide and genetic package display protein are operably linked to a promoter, whereby, upon initiation of transcription from the nucleic acid molecule, a single mRNA transcript that contains nucleic acids encoding the first leader peptide, the first polypeptide and the genetic package display protein is produced.

In some aspects, the nucleic acid encoding the first polypeptide encodes an antibody or functional region thereof, such as a domain exchanged antibody or functional region thereof. In particular examples, the nucleic acid encoding the first polypeptide encodes an antibody domain, such as a heavy chain variable region (V_{H}) domain or functional region thereof, a light chain variable region (V_{L}) domain or functional region thereof, a heavy chain constant region (C_{H}) domain or functional region thereof, or a light chain constant region (C_{L}) domain or functional region thereof. The nucleic acid encoding the first polypeptide can encode two or more antibody domains, such as two or more of a V_{H} domain or functional region thereof, a V_{L} domain or functional region thereof, a C_{H} domain or functional region thereof, and/or a C_{L} domain or functional region thereof. For example, the nucleic acid encoding the first polypeptide can encode a V_{H} domain or functional region thereof and a V_{L} domain or functional region thereof. In other examples, the nucleic acid encoding the first polypeptide encodes a V_{H} domain or functional region thereof, a V_{L} domain or functional region thereof, a C_{H} domain or functional region thereof, and a C_{L} domain or functional region thereof.

The nucleic acid molecules provided herein can contain nucleic acid encoding a first polypeptide, wherein nucleic acid that encodes the first polypeptide encodes a peptide linker. In some examples, the nucleic acid that encodes the first polypeptide encodes a V_{H} domain or functional region thereof, a V_{L} domain or functional region thereof, a C_{H} domain or functional region thereof, and a C_{L} domain or functional region thereof, and a peptide linker, wherein the peptide linker is located between the V_{H} domain and the C_{L} domain in the polypeptide. In other examples, the nucleic acid that encodes the first polypeptide encodes a V_{H} domain or functional region thereof, and a V_{L} domain or functional region thereof, and a peptide linker, wherein the peptide linker is located between the V_{H} domain and the V_{L} domain in the first polypeptide. Such peptide linkers can be, for example, encoded by nucleic acid having a nucleotide sequence set forth in any of SEQ ID NOS: 11, 13, 15, 17, 19, 21 and 23.

The nucleic acid molecules provided herein can further contain: a nucleic acid encoding a second leader peptide; a nucleic acid encoding second polypeptide, wherein the nucleic acid encoding the second leader peptide is operably linked to the nucleic acid encoding the first polypeptide for secretion thereof; and a third stop codon, wherein the third stop codon is located in the nucleic acid encoding the second leader peptide or the nucleic acid encoding the second polypeptide. In some examples, the nucleic acids encoding the second leader peptide, second polypeptide, first leader peptide, first polypeptide, and genetic package display protein are operably linked to a promoter, whereby, upon initiation of transcription from the nucleic acid molecule, a single mRNA transcript that contains nucleic acids encoding the second leader peptide, second polypeptide, first leader peptide, first polypeptide and the genetic package display protein is produced.

In some aspects, the nucleic acid encoding the second polypeptide encodes an antibody or functional region thereof, such as a domain exchanged antibody or functional region thereof. In particular examples, the nucleic acid encoding the second polypeptide encodes an antibody domain selected from among: a V_{H} domain or functional region thereof, a V_{L} domain or functional region thereof, a C_{H} domain or functional region thereof, and a C_{L} domain or functional region thereof. The nucleic acid molecule provided herein can contain nucleic acid encoding a second polypeptide, wherein the nucleic acid encoding the second polypeptide encodes two or more antibody domains, such as, for example, two or more antibody domains are selected from among a V_{H} domain or functional region thereof, a V_{L} domain or functional region thereof, a C_{H} domain or functional region thereof, and/or a C_{L} domain or functional region thereof.

In some aspects, the nucleic acid encoding the first polypeptide encodes a V_{H} domain or functional region and the nucleic acid encoding the second polypeptide encodes a V_{L} domain or functional region thereof. In other aspects, the nucleic acid encoding the first polypeptide encodes a V_{H} domain or functional region thereof and a C_{H} domain or functional domain thereof, and the nucleic acid encoding the second polypeptide encodes a V_{L} domain or functional region thereof and a C_{L} domain or functional domain thereof. In further examples, the nucleic acid encoding the second polypeptide further encodes a peptide linker. Such peptide linkers can be, for example, encoded by nucleic acid having a nucleotide sequence set forth in any of SEQ ID NOS: 11, 13, 15, 17, 19, 21 and 23.

In some examples, one or more additional stop codons are located in one or more of the nucleic acids encoding the first leader peptide, first polypeptide, second leader peptide, second polypeptide. Thus, the nucleic acid molecule can contain an additional 1, 2, 3, 4, 5, 6, 7, 8, 9, 10 or more stop codons. The stop codons in the nucleic acid molecules provided herein can each be selected from among an amber stop codon (UAG or TAG), an ochre stop codon (UAA or TAA) and an opal stop codon (UGA or TGA). In one example, the stop codons are amber stop codons (UAG or TAG).

Also provided herein are nucleic acid molecules containing: a nucleic acid encoding a first leader peptide; a nucleic acid encoding a first polypeptide, wherein the nucleic acid encoding the first leader peptide is operably linked to the nucleic acid encoding the first polypeptide for secretion thereof; a nucleic acid encoding a second leader peptide; a nucleic acid encoding a second polypeptide, wherein the nucleic acid encoding the second leader peptide is operably linked to the nucleic acid encoding the second polypeptide for secretion thereof; a nucleic acid encoding a genetic package display protein, wherein the nucleic acid encoding the genetic package display protein is 3′ of the nucleic acid encoding first polypeptide; and two stop codons, wherein the first stop codon is located in the nucleic acid encoding the first leader peptide and the second stop codon is located in the nucleic acid encoding the second leader peptide. In one example, the nucleic acids encoding the second leader peptide, second polypeptide, first leader peptide, first polypeptide and genetic package display protein are operably linked to a promoter, whereby, upon initiation of transcription from the nucleic acid molecule, a single mRNA transcript that contains nucleic acids encoding the second leader peptide, second polypeptide, first leader peptide, the first polypeptide and the genetic package display protein is produced.

In such nucleic acid molecules, the nucleic acid encoding the first and/or second polypeptide can encode an antibody or functional region thereof, such as a domain exchanged antibody or functional region thereof. In some examples, nucleic acid encoding the first polypeptide and/or the nucleic acid encoding the second polypeptide encodes an antibody domain selected from among a V_{H} domain or functional region thereof, a V_{L} domain or functional region thereof, a C_{H} domain or functional region thereof, and a C_{L} domain or functional region thereof. In one example, the nucleic acid encoding the first polypeptide encodes a V_{H} domain or functional region thereof. In another aspect, the nucleic acid encoding the second polypeptide encodes a V_{L} domain or functional region thereof. In other aspects, the nucleic acid encoding the first polypeptide encodes a V_{H} domain or functional region thereof; and the nucleic acid encoding the second polypeptide encodes a V_{L} domain or functional region thereof. In a particular example, the nucleic acid encoding the first polypeptide and/or the nucleic acid encoding the second polypeptide encodes two or more antibody domains, such as two or more selected from among a V_{H} domain or functional region thereof, a V_{L} domain or functional region thereof, a C_{H} domain or functional region thereof, and a C_{L} domain or functional region thereof. For example, the nucleic acid encoding the first polypeptide can encode a V_{H} domain or functional region thereof and a C_{H} domain or functional domain thereof, and the nucleic acid encoding the second polypeptide can encode a V_{L} domain or functional region thereof and a C_{L} domain or functional domain thereof. Further, the nucleic acid encoding first polypeptide and/or the nucleic acid encoding the second polypeptide also can encodes a peptide linker, such as one encoded by nucleic acid having a nucleotide sequence set forth in any of SEQ ID NOS: 11, 13, 15, 17, 19, 21 and 23. In some examples, the stop codons in the nucleic acid molecules provided herein are each selected from among: an amber stop codon (UAG or TAG), an ochre stop codon (UAA or TAA) and an opal stop codon (UGA or TGA). In one example, the stop codons are amber stop codons (UAG or TAG).

In some aspects, the nucleic acid molecules provided herein contain a nucleic acid encoding the first polypeptide, wherein such nucleic acid encodes a V_{H} domain or a functional region thereof and the V_{H} domain or functional region thereof contains at least one CDR. In some aspects, the V_{H} domain or functional region thereof contains a CDR1, a CDR2, and a CDR3. Further, the nucleic acid encoding the second polypeptide can encode a V_{L} domain or a functional region thereof and the V_{L} domain or functional region thereof contains at least one CDR, such as, for example, a CDR1, a CDR2, and a CDR3.

In particular examples, the nucleic acid encoding the first leader peptide in the nucleic acid molecules provided herein encodes a bacterial leader peptide. In other examples, the nucleic acid encoding the first leader peptide encodes a bacterial leader peptide. For example, the nucleic acid encoding the first leader peptide can encode a Pel B leader peptide or an Omp A leader peptide. Similarly, the nucleic acid encoding the second leader peptide can encode a Pel B leader peptide or an Omp A leader peptide. The Pel B leader peptide can be encoded by, for example, nucleic acid having the sequence of nucleic acids set forth in SEQ ID NO:3. The Omp A leader peptide can be encoded by, for example, nucleic acid having the sequence of nucleic acids set forth in SEQ ID NO:5.

In some aspects, the nucleic acid encoding the genetic package display protein in the nucleic acid molecules provided herein encodes a bacteriophage coat protein, such as, for example, a minor coat protein of filamentous phage or a major coat protein of a filamentous phage. Exemplary of the bacteriophage coat proteins that can be encoded in the nucleic acid molecules provided herein are the gene III protein, gene VIII protein, gene VI protein, gene VII protein and gene IX protein and fragments thereof.

In some examples, the nucleic acid encoding the first polypeptide encodes a domain exchanged antibody or functional region thereof and further encodes a dimerization domain. Similarly, the nucleic acid encoding the second polypeptide can encode a domain exchanged antibody or functional region thereof and can further encode a dimerization domain. In other aspects, the nucleic acid encoding the first polypeptide and/or the nucleic acid encoding the second polypeptide encodes a domain exchanged 2G12 antibody. In particular embodiments, the nucleic acid molecules provided herein encode an antibody fragment selected from among: domain exchanged Fab fragments, domain exchanged scFv fragments, domain exchanged scFv tandem fragments, domain exchanged single chain Fab (scFab) fragments, domain exchanged scFv hinge fragments, and domain exchanged Fab hinge fragments. In one example, the nucleic acid molecule provided herein contains a sequence of nucleotides set forth in SEQ ID NO:28. In some aspects, the nucleic acid molecules provided herein are vectors.

Provided herein are cells containing the nucleic acid molecules described above. In some aspects, the cells are prokaryotic cells, such Escherichia coli cells. In particular examples, the cells are partial suppressor cells, such as, for example, partial amber suppressor cells. Exemplary of such are XL1-Blue, DB3.1, DH5α, DH5αF′, DH5αF′IQ, DH5α-MCR, DH21, EB5α, HB101, RR1, JM101, JM103, JM106, JM107, JM108, JM109, JM110, LE392, Y1088, C600, C600hfl, MM294, NM522, Stb13 and K802 cells. In other aspects, the cells are phage compatible.

Provided herein are methods for producing a first polypeptide and, when a second polypeptide is encoded in the vectors provided herein, also for producing a second polypeptide. In one example, the nucleic acid molecules provided herein are introduced into a cell and the cell is cultured under conditions whereby the first polypeptide is expressed. In some examples, the cell is a partial suppressor cell. In a particular examples, the first and second stop codons in the nucleic acid molecules are amber stop codons, and the cell is a partial amber suppressor cell. Similarly, when the nucleic acid molecule contains the third stop codon, the third stop codon can be an amber stop codon; and the cell can be a partial amber suppressor cell. Exemplary partial amber suppressor cells for use in the methods provided herein include XL1-Blue, DB3.1, DH5α, DH5αF′, DH5αF′IQ, DH5α-MCR, DH21, EB5α, HB101, RR1, JM101, JM103, JM106, JM107, JM108, JM109, JM110, LE392, Y1088, C600, C600hfl, MM294, NM522, Stb13 and K802 cells.

In some examples of the methods provided herein, expression of the encoded first polypeptide results in a fusion polypeptide that contains the first polypeptide fused to the genetic package display protein, and a non-fusion polypeptide that contains the first polypeptide without the genetic package display protein. In some examples, the first polypeptide is an antibody or functional region thereof, such as a domain exchanged antibody or functional region thereof (e.g. a 2G12 domain exchanged antibody or functional region thereof). In a particular example of the methods provided herein, the first polypeptide contains a V_{H} domain from a domain exchanged antibody and a V_{L} domain from a domain exchanged antibody, and expression of the encoded first polypeptide results in a fusion polypeptide that comprises the first polypeptide fused to the genetic package display protein, and a non-fusion polypeptide that comprises the first polypeptide without the genetic package display protein, whereby the V_{H} domain in the fusion polypeptide and the V_{H} domain in the non-fusion polypeptide interact via covalent bond to form a dimer.

In some aspects of the methods provided herein, the nucleic acid molecule provided herein are introduced into the cell and a second polypeptide also is expressed. The second polypeptide can be, for example, an antibody or functional region thereof, such as a domain exchanged antibody or functional region thereof. In one example of the methods provided herein for producing a first and second polypeptide, the first polypeptide contains a V_{H} domain from a domain exchanged antibody and a C_{H} domain from a domain exchanged antibody, the second polypeptide contains a V_{L} domain from a domain exchanged antibody and a C_{L} domain from a domain exchanged antibody, and expression of the encoded first polypeptide results in a fusion polypeptide that comprises the first polypeptide fused to the genetic package display protein, and a non-fusion polypeptide that comprises the first polypeptide without the genetic package display protein, while expression of the encoded second polypeptide results in a non-fusion polypeptide that comprises the second polypeptide without the genetic package display protein, such that one fusion protein containing the first polypeptide, one non-fusion polypeptide containing the first polypeptide, and two non-fusion polypeptides containing the second polypeptide associate to form a domain exchanged Fab fragment.

In some aspects of the methods provided herein, the first polypeptide is expressed at reduced levels compared to in the absence of the stop codon located in the nucleic acid encoding the first leader peptide. Expression of the first polypeptide can be reduced for example, by or by about 10%, 15%, 20%, 25%, 30%, 35%, 40%, 45%, 50%, 55%, 60%, 65%, 70%, 75%, 80% 85% or more compared to in the absence of the stop codon located in the nucleic acid encoding the first leader peptide. Further, in some aspects the first polypeptide is a polypeptide that is toxic to the cell and is expressed with reduced toxicity to the cell compared to in the absence of the stop codon located in the nucleic acid encoding the first leader peptide. For example, toxicity can be reduced by or by about 10%, 15%, 20%, 25%, 30%, 35%, 40%, 45%, 50%, 55%, 60%, 65%, 70%, 75%, 80% 85% or more compared to in the absence of the stop codon located in the nucleic acid encoding the first leader peptide.

In other aspects of the methods provided herein, the second polypeptide is expressed at reduced levels compared to in the absence of the stop codon located in the nucleic acid encoding the second leader peptide. Expression of the second polypeptide can be reduced for example, by or by about 10%, 15%, 20%, 25%, 30%, 35%, 40%, 45%, 50%, 55%, 60%, 65%, 70%, 75%, 80% 85% or more compared to in the absence of the stop codon located in the nucleic acid encoding the second leader peptide. Further, in some examples the second polypeptide is a polypeptide that is toxic to the cell and is expressed with reduced toxicity to the cell compared to in the absence of the stop codon located in the nucleic acid encoding the second leader peptide. For example, toxicity can be reduced by or by about 10%, 15%, 20%, 25%, 30%, 35%, 40%, 45%, 50%, 55%, 60%, 65%, 70%, 75%, 80% 85% or more compared to in the absence of the stop codon located in the nucleic acid encoding the second leader peptide.

In some examples of the methods provided herein for producing a first polypeptide, the first polypeptide is displayed on a genetic package. Similarly, in some examples of the methods provided herein for producing a second polypeptide, the second polypeptide is displayed on a genetic package. In one example, the first polypeptide and the second polypeptide are displayed on a genetic package.

In one aspect of the methods provided herein, when the cell is a phage compatible cell and the genetic package display protein is a phage coat protein, the method also can include a step of infecting the cell with helper phage, such that the first polypeptide is displayed on the surface of the phage produced by the cell.

Also provided herein are nucleic acid libraries, containing the nucleic acid molecules provided herein. Such nucleic acid libraries can be used, for example, to generate phage display libraries.

Provided herein are vectors for display. Exemplary of the vectors include, but are not limited to, a vector containing a nucleic acid encoding a heavy chain variable region (V_{H}) domain of a domain exchanged antibody, or a functional region thereof; a nucleic acid encoding a genetic package display protein, wherein the nucleic acid encoding the genetic package display protein is 3′ of the nucleic acid encoding the V_{H} domain or functional region thereof; and a stop codon, where the stop codon is located between the nucleic acid encoding the V_{H} domain or region thereof and the nucleic acid encoding the display protein. In some examples, the stop codon is an amber stop codon (UAG or TAG), an ochre stop codon (UAA or TAA) or an opal stop codon (UGA or TGA). The vectors provided herein further can contain an additional nucleic acid, such as a nucleic acid encoding a light chain variable region (V_{L}) domain or functional region thereof, a nucleic acid encoding a heavy chain constant region (C_{H}) domain or functional region thereof, and nucleic acid encoding a light chain constant region (C_{L}) domain or functional region thereof. In one aspect, the vectors provided herein contain a nucleic acid encoding a C_{H} domain or functional region thereof, which is located between the nucleic acid encoding the V_{H} domain and the stop codon.

The vectors provided herein also can contain a nucleic acid encoding a peptide linker. In one example, the vector contains a nucleic acid encoding a V_{L} domain or functional region thereof and a nucleic acid encoding a C_{H} domain and a nucleic acid encoding a C_{L} domain or functional region thereof, where the nucleic acid encoding the peptide linker is located between the nucleic acid encoding the V_{H} domain and the nucleic acid encoding the C_{L} domain or functional region thereof. The vector further can contain nucleic acid encoding a V_{L} domain or functional region thereof, where the nucleic acid encoding the peptide linker is located between the nucleic acid encoding the V_{H} domain and the nucleic acid encoding the V_{L} domain or functional region thereof.

In some examples of the vectors provided herein, the nucleic acid encoding the V_{H} domain or functional region thereof, the nucleic acid encoding the genetic package display protein, and the stop codon are operably linked to a promoter, such that upon initiation of transcription from the vector, an mRNA transcript is produced, the mRNA transcript containing nucleic acid encoding the V_{H} domain or functional region thereof, nucleic acid encoding the genetic package display protein, and nucleic acid encoded by the stop codon.

Provided herein are vectors that contain: two nucleic acids encoding heavy chain variable region (V_{H}) domains of a domain exchanged antibody or functional regions thereof; nucleic acid encoding a genetic package display protein, wherein the nucleic acid encoding the genetic package display protein is 3′ of the nucleic acids encoding the V_{H} domains or functional regions thereof; and nucleic acid encoding a peptide linker; wherein the two nucleic acids encoding V_{H} domains or regions thereof encode identical V_{H} domains or regions, and the nucleic acid encoding the peptide linker is between the two nucleic acids encoding V_{H} domains or functional regions thereof. In some examples, such vectors also contain nucleic acid encoding a light chain variable region (V_{L}) domain or functional region thereof. For example, the vector can contain two nucleic acids encoding V_{L} domains, wherein the two encoded V_{L} domains are identical. Further, the vector can contain nucleic acid encoding an additional peptide linker located between the nucleic acids encoding V_{H} and V_{L} domains or regions thereof. In a particular example, the nucleic acids encoding the V_{H} domains or functional regions thereof, the nucleic acid encoding the genetic package display protein, and the nucleic acid encoding the peptide linker, are operably linked to a promoter, such that upon initiation of transcription from the vector, an mRNA transcript is produced, the mRNA transcript containing nucleic acids encoding the V_{H} domains or regions, nucleic acid encoding the genetic package display protein, and nucleic acid encoding the peptide linker.

In some examples, where the vectors provided herein contain nucleic acid(s) encoding a peptide linker(s), the nucleic acid(s) encoding peptide linker(s) contains nucleic acid having the nucleotide sequence set forth in any of SEQ ID NOs: 15, 17, 19, 21, 23, 25 and 27.

Provided herein are vectors for displaying a domain exchanged antibody on a genetic package. These vectors contain: nucleic acid encoding a heavy chain variable region (V_{H}) domain of a domain exchanged antibody or a functional region thereof; nucleic acid encoding a genetic package display protein, wherein the nucleic acid encoding the genetic package display protein is 3′ of the nucleic acid encoding the V_{H} domain or region thereof, and nucleic acid encoding a dimerization domain; wherein the nucleic acid encoding the dimerization domain is located between the nucleic acid encoding the V_{H} domain or region thereof and the sequence encoding the display protein. In some examples, the vectors also contain a stop codon located between the nucleic acid encoding the dimerization domain and the nucleic acid encoding the display protein. This stop codon can be an amber stop codon (UAG or TAG), an ochre stop codon (UAA or TAA) or an opal stop codon (UGA or TGA). In some aspects, the vectors for displaying domain exchanged antibodies on a genetic package also contain one or more additional nucleic acids, such as, for example, nucleic acid encoding a light chain variable region (V_{L}) domain or functional region thereof; nucleic acid encoding a heavy chain constant region (C_{H}) domain or functional region thereof, and nucleic acid encoding a light chain constant region (C_{L}) domain or functional region thereof. In some examples, the functional region of a V_{H} domain contains at least one CDR. For example, the functional region of the V_{H} domain contains a CDR1, a CDR2, and a CDR3. In particular examples of the vectors for displaying a domain exchanged antibodies, the nucleic acid encoding the V_{H} domain or region thereof, the nucleic acid encoding the genetic package display protein, and the nucleic acid encoding the dimerization domain, are operably linked to a promoter, such that upon initiation of transcription from the vector, an mRNA transcript is produced, the mRNA transcript containing nucleic acid encoding the V_{H} domain, nucleic acid encoding the genetic package display protein, and nucleic acid encoding the dimerization domain.

Provided herein are vectors containing: nucleic acid encoding an antibody heavy chain variable region (V_{H}) domain, or a functional region thereof; nucleic acid encoding a genetic package display protein, wherein the nucleic acid encoding the genetic package display protein is 3′ of the nucleic acid encoding the antibody heavy chain variable region (V_{H}) domain or functional region thereof; and a stop codon between the nucleic acid encoding the V_{H} domain or region thereof and the nucleic acid encoding the display protein; wherein the vector does not encode an antibody hinge region or functional region thereof, the vector does not encode a leucine zipper or a GCN4 zipper domain, and upon introduction of the vector into host cell that produces a genetic package and upon expression of the encoded V_{H} protein or region thereof, an antibody containing two copies of the V_{H} domain or region thereof, is displayed on the genetic package. In some examples, such vectors do not contain a dimerization domain other than dimerization domains native to antibody molecules. Further, the vectors also can contain nucleic acid encoding a V_{L} domain or functional region thereof. In some examples, the antibody encoded by the vector is a domain exchanged antibody, including a domain exchanged antibody fragment, such as, for example, a domain exchanged Fab fragment, domain exchanged scFv fragment, domain exchanged scFv tandem fragment, domain exchanged single chain Fab (scFab) fragment, domain exchanged scFv hinge fragment, and domain exchanged Fab hinge fragment.

Provided herein are cells containing the vectors described above and provided herein. The cells can be prokaryotic cells, such as, for example, Escherichia coli cells. In some examples, the cells are partial suppressor cells, such as partial amber suppressor cells. Exemplary of partial amber suppressor cells in which the vectors provided herein can be contained includes XL1-Blue, DB3.1, DH5α, DH5αF′, DH5αF′IQ, DH5α-MCR, DH21, EB5α, HB101, RR1, JM101, JM103, JM106, JM107, JM108, JM109, JM110, LE392, Y1088, C600, C600hfl, MM294, NM522, Stb13 and K802 cells. In some examples, the cells provided herein containing the vectors are phage compatible.

Provided herein are collections of vectors, containing a plurality of the vectors described above and provided herein. In some examples, the vectors in these collections contain variant polynucleotides. In some aspects, the collections of vectors contain at least 10⁴ or about 10⁴, 10⁵ or about 10⁵, 10⁶ or about 10⁶, 10⁷ or about 10⁷, 10⁸ or about 10⁸, 10⁹ or about 10⁹, 10¹⁰ or about 10¹⁰, 10¹¹ or about 10¹¹, 10¹² or about 10¹², 10¹³ or about 10¹³, or 10¹⁴ or about 10¹⁴ different nucleotide sequences among the vector members.

Provided herein are methods for displaying a domain exchanged antibody on the surface of a genetic package. The methods contain the steps of (a) transforming a host cell with a vector, e.g. any of the provided vectors for display of domain exchanged antibodies; and (b) inducing polypeptide expression from the vector, thereby expressing a displayed domain exchanged antibody. In such methods, the displayed domain exchanged antibody contains: a fusion protein, wherein the fusion protein comprises a domain exchanged V_{H} domain or functional region thereof fused to a genetic package display protein, and a non-fusion polypeptide, wherein the non-fusion polypeptide comprises a domain exchanged antibody V_{H} domain or functional region thereof and not a genetic package display protein, wherein the fusion protein and non-fusion polypeptide interact via covalent bond; or a single polypeptide chain, wherein the single polypeptide chain comprises a fusion protein containing at least two domain exchanged V_{H} domains or functional regions thereof, fused to a genetic package display protein, and a peptide linker, whereby the displayed domain exchanged antibody is displayed on the genetic package.

In some examples, the methods for displaying a domain exchanged antibody on the surface of a genetic package also contain a step of inducing expression of a light chain variable region (V_{L}) domain or functional region thereof. The V_{L} domain or functional region thereof can interact with one or more of the V_{H} domain chains via covalent bond.

In some aspects of the methods for displaying a domain exchanged antibody on the surface of a genetic package, the host cell is a partial suppressor cell, such as a partial amber-suppressor cell, including, but not limited to, an XL1-Blue, DB3.1, DH5α, DH5αF′, DH5αF′IQ, DH5α-MCR, DH21, EB5α, HB101, RR1, JM101, JM103, JM106, JM107, JM108, JM109, JM110, LE392, Y1088, C600, C600hfl, MM294, NM522, Stb13 or K802 cell. In other aspects, the domain exchanged antibody is an antibody fragment, such as a domain exchanged Fab fragments, domain exchanged scFv fragments, domain exchanged scFv tandem fragments, domain exchanged single chain Fab (scFab) fragments, domain exchanged scFv hinge fragments, or domain exchanged Fab hinge fragments.

Provided herein are methods for selecting one or more domain exchanged antibodies having a desired binding activity or property. Such methods include the steps of: (a) displaying antibodies from the collection of genetic packages, such as any of the provided genetic packages; (b) exposing the collection to a binding partner, whereby one or more of the antibodies displayed on genetic packages binds to the binding partner; (c) washing, thereby removing unbound genetic packages; and (d) eluting, thereby isolating genetic packages displaying the one or more selected domain exchanged antibodies having the desired binding property or activity. In some aspects of the methods, the binding partner is coupled to a solid support. In other aspects, the solid support is a plate, a bead, a column or a matrix. In further examples of the method, the eluting is carried out with one or more elution buffers; or the washing is carried out with one or more wash buffers

In some examples of the methods for selecting one or more domain exchanged antibodies having a desired binding activity or property, the desired binding property or activity is binding specificity, high affinity binding, high avidity binding, low off-rate or high on-rate. In such examples, high affinity is higher affinity compared a target domain exchanged antibody polypeptide, high avidity is higher avidity compared to a target domain exchanged antibody polypeptide, high on-rate is higher on-rate compared to a target domain exchanged antibody polypeptide, and low off-rate is higher off-rate compared to a target domain exchanged antibody polypeptide. In further examples, more than one genetic packages are isolated in step (d). Steps (b)-(d) can be repeated, such that the collection contains the more than one isolated genetic packages, thereby selecting one or more domain exchanged antibodies from among the selected antibodies.

Also provided herein are domain exchanged antibodies. The domain exchanged antibodies can contain one or more modifications at an amino acid position, based on Kabat number, selected from among H31, H32, H33, H52, H95, H96, H97, H98, H99, H100, H100a, H100c, H100d, L89, L90, L91, L92, L93, L94 and L95, wherein the modification is with reference to the amino acid residue at the corresponding position in domain exchanged antibody 2G12. The modifications can be amino acid replacements with any amino acid. In one example, the modifications is amino acid replacement with an alanine.

In some instances, the domain exchanged antibody is a modified 2G12 domain exchanged antibody. For example, the modified 2G12 domain exchanged antibody can contain modifications compared to an unmodified 2G12 domain exchanged that contains a light chain having a sequence of amino acids set forth in SEQ ID NO:159, and a heavy chain having a sequence of amino acids set forth in SEQ ID NO:308.

Included among the domain exchanged antibodies provided herein are domain exchanged antibody fragments, including, but not limited to, a domain exchanged Fab fragment, a domain exchanged scFv fragment, a domain exchanged single chain Fab (scFab) fragment, a domain exchanged scFv tandem fragment, a domain exchanged scFv hinge fragment and a domain exchanged Fab hinge fragment. The domain exchanged antibodies can contain, for example, any one or more of a heavy chain having a sequence of amino acids set forth in SEQ ID NO: 306, a light chain having a sequence of amino acids set forth in SEQ ID NO: 307 or 322, a V_{H} domain having a sequence of amino acids set forth in SEQ ID NO: 161, or a V_{L} domain having a sequence of amino acids set forth in SEQ ID NO:305 or 321.

Also provided herein are collections, containing a plurality any of the domain exchanged antibodies provided herein, including the 2G12 antibodies. The collections can contain, for example, at least 10⁴ or about 10⁴, 10⁵ or about 10⁵, 10⁶ or about 10⁶, 10⁷ or about 10⁷, 10⁸ or about 10⁸, 10⁹ or about 10⁹, 10¹⁰ or about 10¹⁰, 10¹¹ or about 10¹¹, 10¹² or about 10¹², 10¹³ or about 10¹³, or 10¹⁴ or about 10¹⁴ different amino acid sequences among the modified 2G12 domain exchanged antibody members.

### BRIEF DESCRIPTION OF THE DRAWINGS

FIG. 1: Comparison of Conventional and Domain Exchanged Antibodies

FIG. 1 is an illustrative comparison of a full-length conventional IgG antibody (left) and an exemplary full-length domain exchanged IgG antibody. As shown, the conventional full-length antibody contains two heavy (H and H′) and two light (L and L′) chains, and two antibody combining sites, each formed by residues of one heavy and one light chain. By contrast, the heavy chains in the exemplary domain exchanged antibody are interlocked, resulting in pairing of the heavy chain variable regions (V_{H} and V_{H}′) with the opposite light chain variable regions (V_{L}′ and V_{L}, respectively), forming a pair of conventional antibody combining sites, locked in space. As described herein, the V_{H}-V_{H}′ interface can form a non-conventional antibody combining site, containing residues of the two adjacent heavy chain variable regions (V_{H} and V_{H}′). The number (35 Å (angstroms)) represents the distance between the two conventional antibody combining sites in this exemplary domain exchanged antibody. For each antibody, the two heavy chains, H and H′ are illustrated in grey and black, respectively; the two light chains, L and L′, are illustrated with open and hatched boxes, respectively. The specific domains (e.g. V_{H} C_{H}1, C_{L},) are indicated.

FIG. 2: Domain Exchanged Antibody Fragments

FIG. 2 schematically illustrates examples of a plurality of the provided domain exchanged antibody fragments (domain exchanged Fab fragment (2A); domain exchanged Fab hinge fragment (2B); domain exchanged Fab Cys19 fragment (2C); domain exchanged scFab ΔC² fragment (2D)(i)); domain exchanged scFab ΔC²Cys19 fragment (2D(ii)); domain exchanged scFv tandem fragment (2E); domain exchanged scFv fragment (2F); domain exchanged scFv hinge/scFv hinge (ΔE) fragments (having the same general structure as described herein) (2G); and domain exchanged scFv Cys19 fragment (2H). In the example illustrated in this figure, the fragments are expressed as part of phage coat (cp3) fusion proteins, for display on bacteriophage. “S—S” indicates a disulfide bond; “G3” indicates a cp3 phage coat protein. Specific antibody domains (e.g. V_{H} C_{H}1, C_{L},) are indicated. One heavy (H) and one light (L) chain are illustrated filled in white, while the other heavy (H′) and light (L′) chains are illustrated filled in grey. These fragments are described in detail herein.

FIG. 3: Schematic illustration of fragment Assembly and Ligation/Single Primer Amplification (FAL-SPA) Method for Generating Collections of Assembled Duplexes

FIG. 3 illustrates one example of the provided methods for forming a collection of variant assembled duplexes (to form a nucleic acid library) with Fragment Assembly and Ligation/Single Primer Amplification (FAL-SPA). FIG. 3A: In this illustrated example, pools of randomized duplexes are generated according to the provided methods (open boxes with hatched portions representing randomized portions). Typically, these pools are generated by amplification (not shown) using randomized template oligonucleotides and primers. FIG. 3B: Pools of reference sequence duplexes and pools of scaffold duplexes are generated by amplification, using the target polynucleotide as a template, for example, in a high-fidelity (hi-fi) PCR (the primers are not shown). FIG. 3C: Duplexes from the pools are combined in a Fragment Assembly and Ligation (FAL) step whereby they are denatured and hybridize through complementary regions. As shown, randomized and reference sequence duplex polynucleotides are brought in close proximity as they hybridize to the scaffold duplexes, which contain regions complementary to regions in multiple pools of the other duplexes. Nicks (indicated by arrows) are sealed between the adjacent polynucleotides, forming a pool of assembled polynucleotides. FIG. 3D: The assembled polynucleotides are used as templates in a single primer amplification (SPA) reaction, generating a pool of variant assembled duplexes, each duplex containing sequences from polynucleotides in the randomized and the reference sequence duplex pools. In one example, the assembled duplexes can be cut with restriction enzymes to form assembled duplex cassettes, which can be ligated into vectors. Throughout this figure, two complementary non-gene specific nucleotide sequences (Region X and Region Y) are illustrated as black and grey filled boxes respectively. These non gene-specific regions are contained in the duplexes in two of the reference sequence duplex pools (FIG. 3B), and have complementarity/identity to the single primer pool used in the amplification reaction (FIG. 3D), which contains the nucleotide sequence with identity to Region X, e.g. the nucleotide sequence of Region X.

FIG. 4: Exemplary Phagemid Vector for Display of Domain Exchanged Antibodies

FIG. 4 depicts an exemplary phagemid vector for display of domain exchanged antibodies. The vector contains a lac promoter system, including a truncated lac I gene. The lac I gene encodes the lactose repressor and the lactose promoter and operator. The lac promoter/operator is operably linked to a leader sequence, followed by a nucleic acid encoding a domain exchanged antibody light chain, another leader sequence, and a nucleic acid encoding a domain exchanged antibody heavy chain. Downstream is a tag sequence, followed by a stop codon and nucleic acid encoding a phage coat protein (here gIII encoding cp3). The vector also includes phage and bacterial origin of replications.

FIG. 5: Exemplary Phagemid Vector for Insertion of Nucleic Acid Encoding a Protein for which Reduced Expression is Desired

FIG. 5 depicts an exemplary phagemid vector for insertion of nucleic acid encoding a protein for which reduced expression is desired, such as to reduce toxicity of the protein to the host cell. The vector contains a lac promoter system, including the lac I gene, which encodes the lactose repressor, and the lactose promoter and operator. The lac promoter/operator is operably linked to a leader sequence into which a stop codon has been introduced. One or more restriction enzyme sites are downstream of the leader sequence, allowing for insertion of nucleic acid encoding a protein or domain or fragment thereof. In some examples, the vector contains an additional leader sequence containing a stop codon, followed by one or more restriction enzyme sites, allowing insertion of a second polynucleotide encoding another protein or fragment or domain thereof. Down stream of this is a tag sequence, followed by a stop codon and nucleic acid encoding a phage coat protein. The vector also includes phage and bacterial origin of replications.

FIG. 6: Exemplary Phagemid Vector for Reduced Expression of Antibodies or Antibody Fragments

FIG. 6 depicts an exemplary phagemid vector for expression of antibodies or fragments thereof, including domain exchanged antibodies or fragments thereof. The vector contains a lac promoter system, including the lac I gene, which encodes the lactose repressor, and the lactose promoter and operator. The vector contains nucleic acid encoding an antibody light chain linked at its 5′ end to the 3′ end of a leader sequence into which a stop codon has been introduced, and nucleic acid encoding an antibody heavy chain linked at its 5′ end to the 3′ end of another leader sequence into which a stop codon has been introduced. Downstream of the nucleic acid encoding the heavy chain is a tag sequence, a stop codon and nucleic acid encoding a phage coat protein. The single genetic element containing these leader, antibody chain, tag and phage coat protein is operably linked to the lactose promoter and operator, such that a single mRNA transcript is produced following induction of transcription. When expressed in a partial suppressor cell, soluble (native) antibody light chains, soluble (or native) antibody heavy chains and heavy chain-phage protein fusion proteins are produced.

FIG. 7: pCAL G13 Vector

FIG. 7 is an illustrative map of the pCAL G13 vector, provided and described in detail herein. GIII represents the nucleotide encoding the phage coat protein cp3. “Amber” indicates the position of the amber stop codon (TAG/UAG), adjacent to the cp3 encoding nucleotide.

FIG. 8: 2G12 pCAL Vector

FIG. 8 depicts the 2G12 pCAL vector, provided and described in detail herein. The vector encodes the 2G12 antibody light and heavy chains (2G12 LC and 2G12 HC, respectively) in polynucleotides that are linked to the Pel B and OmpA leader sequences, respectively. The polynucleotides encoding the 2G12 HC are linked to nucleotides encoding a histidine tag, followed by an amber stop codon (*) and a truncated gIII protein. These polynucleotides all are operably linked to the lactose promoter and operator element. Also included in the vector is a truncated lac I gene.

FIG. 9. 2G12 pCAL IT* Vector

FIG. 9 depicts the 2G12 pCAL IT* vector. The 2G12 pCAL IT* vector can be used to express, with reduced toxicity, Fab fragments of the domain exchanged 2G12 antibody, which recognize the HIV gp120 antigen. Expression as both soluble 2G12 Fab fragments and 2G12-gIII coat protein fusion proteins for display on phage particles can be effected in partial amber suppressor cells by virtue of the amber stop codon between the nucleotides encoding the 2G12 heavy chain nucleotides encoding the truncated gIII coat protein. The polynucleotide encoding the 2G12 light chain is linked to the Pel B leader sequence, and the 2G12 heavy chain is linked to the OmpA leader sequence. The inclusion of an amber stop codon in each of the leader sequences results in reduced expression of the 2G12 heavy and light chains in partial amber suppressor strains following induction with, for example IPTG. The reduced expression can lead to reduced toxicity of the 2G12 Fab to the host cells.

FIG. 10: Introduction of Amber Stop Codon in PelB and OmpA Leader Sequences

FIG. 10 depicts the modification of the Pel B and Omp A leader sequences in the 2G12 pCAL ITPO vector to introduce an amber stop codon into each sequence, producing the 2G12 pCAL IT* vector. The stop codons are incorporated by mutation of the CAG triplet encoding a glutamine (Glu, Q) in each of the leader sequences to a TAG amber stop codon. For example, the nucleotide triplet at nucleotides 52-54 of the PelB leader sequence set forth in SEQ ID NO: 1, encoding the glutamine at amino acid position 18 of the PelB leader peptide set forth in SEQ ID NO: 2 was modified to generate a TAG amber stop codon at nucleotides 52-54 (SEQ ID NO:3). Similarly, the nucleotide triplet at nucleotides 58-60 of the OmpA leader sequence set forth in SEQ ID NO: 5, encoding the glutamine at amino acid position 20 of the OmpA leader peptide set forth in SED ID NO: 6) was modified to generate a TAG amber stop codon at nucleotides 58-60 (SEQ ID NO:7).

FIG. 11: Schematic Illustration of Modified Fragment Assembly and Ligation/Single Primer Amplification (mFAL-SPA) Method for Generating Collections of Assembled Duplexes

FIG. 11 one example of the provided methods for forming a collection of variant assembled duplexes using modified Fragment Assembly and Ligation/Single Primer Amplification (mFAL-SPA). FIG. 11A: In this example, pools of randomized duplexes with overhangs are generated (open boxes with hatched portions representing randomized portions). FIG. 11B: Pools of reference sequence duplexes are generated in amplification reactions using the target polynucleotide as a template and primers containing restriction site nucleotide sequences (restriction sites, which are within the portions of the primers and duplexes illustrated as boxes with vertical lines or grey or black fill). FIG. 11C: The reference sequence duplexes are digested with restriction endonucleases (which recognize the site within the vertical line boxes) to form overhangs in the duplexes. FIG. 11D: Reference sequence duplexes with overhangs and randomized duplexes with overhangs are combined in a Fragment Assembly and Ligation (FAL) step, whereby the duplexes hybridize through complementary regions in the overhangs, which are compatible overhangs, forming a pool of intermediate duplexes. A single primer amplification (SPA) reaction then is performed (not shown) using the intermediate duplex polynucleotides as templates. As in FAL-SPA (e.g. FIG. 3) a SPA reaction then is performed with a primer (not shown) having identity to a non gene-specific sequence (Region X; shown in black; contained in the intermediate duplexes, and the pools of reference sequence duplexes) and complementary to another non gene-specific sequence, Region Y, which is illustrated in grey. In one example, the assembled duplexes can be cut with restriction enzymes (recognizing the site within the sequence represented in black) for ligation into vectors.

FIG. 12. 2G12 pCAL ITPO Vector

FIG. 12 depicts the 2G12 pCAL IPTO vector, generated as described in Example 2c(i). The vector was generated by modification of the 2G12 pCAL vector (FIG. 8), wherein the truncated lac I gene of the 2G12 pCAL vector is replaced with a full length lac I gene.

FIG. 13: Randomization of 3-ALA 2G12 Fragment Target Polypeptide Using mFAL-SPA

FIG. 13 illustrates the mFAL-SPA process that was used to randomize the 2G12 domain exchanged Fab fragment target polypeptide, as described in Example 5A, below. FIG. 13A: Four pools of randomized oligonucleotides (H1F, H1R, H3F, and H3R; illustrated as open boxes with hatched portions representing randomized portions) were designed and hybridized to form two pools of randomized duplexes (H1 and H3), containing overhangs. FIG. 13B: Three pools of reference sequence duplexes (1, 2, and 3) were generated using PCR with three pools of forward oligonucleotide primers (F1, F2, F3) and three pools of reverse oligonucleotide primers (R1, R2, R3). Four of the primers, R1, F2, R2 and F3, contained a recognition site for the SAP-I restriction endonuclease (indicated by a portion with vertical lines). FIG. 13C: Reference sequence duplexes were cut with the Sap-I restriction endonuclease, generating reference sequence duplexes with Sap-I overhangs compatible to those in the randomized duplexes. FIG. 13D: The reference sequence and randomized pools of duplexes with overhangs then were combined under conditions whereby they hybridized through complementary overhangs and nicks (indicated with arrows) were sealed with a ligase, forming a pool of intermediate duplexes, which then was used in an SPA reaction (not shown) with a CALX24 single primer pool to generate a collection of variant assembled duplexes. One forward primer pool (F1), and one reverse primer pool (R3) contained a non gene-specific nucleotide sequence (Region X; depicted in black), which was identical to the nucleotide sequence of the CALX24 primer, such that reference sequence duplexes 1 and 3 contained a sequence of nucleotides including Region X, and a complementary Region Y, which served as template sequences for the primers in the SPA. The assembled duplexes can be digested to form assembled duplex cassettes with restriction enzymes recognizing restriction sites within the portion illustrated in black.

FIG. 14: Binding of Domain Exchanged Fragments, Expressed in Bacteria, to gp120 Antigen

FIG. 14 illustrates the results of a binding assay used to evaluate the binding of the indicated exemplary 2G12 domain exchanged antibody fragments (generated as described in Example 8), expressed from BL21 (DE3) host cells, to bind the antigen, gp120 (to which 2G12 antibody specifically binds). Solutions containing secreted and intracellular domain exchanged antibody fragments were obtained from overnight cultures of host cells that had been induced to express the polypeptides. An ELISA was performed as described in Example 8C(ii), below, on 1:5 serial dilutions of the solutions. As described, binding of solutions to plate-bound gp120 was assessed using an HRP-conjugated secondary antibody and a substrate and reading absorbance at 450 nm. Absorbance values are indicated on the Y axis, while dilution factor is indicated on the X axis. Labeled arrows on the graph point to curves representing the domain exchanged Fab hinge, Fab, scFv tandem and scFv hinge fragments (the fragments having strong or moderate binding to the antigen). Error bars represent standard deviation among triplicate samples. The results illustrated in this figure are described in Example 8C(ii) and also are listed in Table 38.

### DETAILED DESCRIPTION

### Outline

### A. Definitions

### B. Overview of the Methods, Vectors and Display Molecules

### C. Antibodies

1. Structural and functional domains of antibodies

2. Antibody fragments

3. Domain exchanged antibodies

4. Antibodies in protein therapeutics
Monoclonal antibodies (MAbs) and antibody libraries

### D. Vectors and Methods

1. Overview of expression and display of polypeptides with reduced toxicity, including domain exchanged antibodies.
a. Expression with reduced toxicity
b. Display of proteins, including domain exchanged antibodies and bivalent antibodies

2. Vectors
a. Introduction of stop codons to reduce expression of proteins
b. Introduction of a stop codon to facilite expression of soluble proteins and fusion proteins
c. Other features
   i. Promoters
      lac promoter
   ii. Leader sequences
   iii. Phage display features
      Expression of soluble proteins and fusion proteins
c. Exemplary polypeptides for expression using the vectors
d. Expression of domain exchanged antibodies from the vectors herein
   i. Peptide linkers
   ii. Dimerization domains
   iii. Mutations promoting dimerization
   iv. Hinge regions
   v. Other dimerization domains
   vi. Exemplary domain exchanged antibodies and fragments
      (1) Domain exchanged Fab Fragment
      (2) Domain exchanged scFv fragment
      (Domain exchanged Fab hinge fragment
      (4) Domain exchanged scFv tandem fragment
      (5) Domain exchanged single chain Fab fragments
      (6) Domain exchanged Fab Cys19
      (7). Domain exchanged scFv hinge
e. Exemplary vectors
   pCAL vectors
   (1). 2G12 pCAL vectors and variants
   (2). 2G12 pCAL IT* and variants
   (3). Vectors for display of other domain exchanged fragments

3. Methods for expression of polypeptides
a. Suppressor tRNAs and partial suppressor cells
   Amber suppressor cells

4. Uses for the vectors and cells for reduced expression of proteins

### E. Methods for Display on Genetic Packages

1. Phage display
a. phagemid and phage vectors
b. Transformation and growth of phage-display compatible cells
c. co-infection with helper phage, packaging and expression
d. Isolation of genetic packages displaying the polypeptides.

2. Other display methods
a. Cell surface display
b. Other display systems
   F. Libraries of Displayed Polypeptides and Selection of Displayed Polypeptides from the Libraries

1. Confirming display of the polypeptides

2. Selection of polypeptides from the collections
a. panning
   i. Incubation of the displayed polypeptides with a binding partner
      2. Washing
      3. Elution of bound polypeptides
c. Amplification and analysis of selected polypeptides
d. Iterative selection

### G. General Host Cell-Vector Systems for Nucleic Acid Amplification and Protein Expression

1. Amplification of nucleic acids

2. expression of encoded polypeptides

3. Host cells
a. Prokaryotic cells
b. Yeast cells
c. Insect cells
d. Mammalian cells
e. Plants

4. Nucleic acid libraries
a. Generating nucleic acid libraries
   i. Selection of target polypeptides
   ii. Design and synthesis of oligonucleotides
   iii. Generation of assembled oligonucleotide duplexes and duplex cassettes
   iv. Ligation of the assembled duplex cassettes into vectors

### EXAMPLES

### A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, macromolecule refers to any molecule having a molecular weight from hundreds to millions of daltons. Macromolecules include peptides, proteins, polypeptides, nucleotides, nucleic acids, and other such molecules that are generally synthesized by biological organisms, but can be prepared synthetically or using recombinant molecular biology methods.

As used herein, “biomolecule” refers to any compound found in nature and any derivatives thereof. Exemplary biomolecules include but are not limited to: oligonucleotides, oligonucleosides, proteins, peptides, amino acids, peptide nucleic acid molecules (PNAs), oligosaccharides and monosaccharides.

As used herein, “polypeptide” refers to two or more amino acids covalently joined. The terms “polypeptide” and “protein” are used interchangeably herein.

As used herein, a native polypeptide or a native nucleic acid molecule is a polypeptide or nucleic acid molecule that can be found in nature. A native polypeptide or nucleic acid molecule can be the wild-type form of a polypeptide or nucleic acid molecule. A native polypeptide or nucleic acid molecule can be the predominant form of the polypeptide, or any allelic or other natural variant thereof. The variant polypeptides and nucleic acid molecules provided herein can have modifications compared to native polypeptides and nucleic acid molecules.

As used herein, the wild-type form of a polypeptide or nucleic acid molecule is a form encoded by a gene or by a coding sequence encoded by the gene. Typically, a wild-type form of a gene, or molecule encoded thereby, does not contain mutations or other modifications that alter function or structure. The term wild-type also encompasses forms with allelic variation as occurs among and between species. As used herein, a predominant form of a polypeptide or nucleic acid molecule refers to a form of the molecule that is the major form produced from a gene. A “predominant form” varies from source to source. For example, different cells or tissue types can produce different forms of polypeptides, for example, by alternative splicing and/or by alternative protein processing. In each cell or tissue type, a different polypeptide can be a “predominant form.”

As used herein, a “polypeptide that is toxic to the cell” refers to a polypeptide whose heterologous expression in a host cell can be detrimental to the viability of the host cell. The toxicity associated with expression of the heterologous polypeptide can manifest, for example, as cell death or a reduced rate of cell growth, which can be assessed using methods well known in art, such as determining the growth curve of the host cell expressing the polypeptide by, for example, spectrophotometric methods, such as the optical density at 600 nm, and comparing it to the growth of the same host cell that does not express the polypeptide. Toxicity associated with expression of the polypeptide also can manifest as vector instability or nucleic acid instability. For example, the vector encoding the polypeptide can be lost from the host cell during replication of the host cell, or the nucleic acid encoding the polypeptide can be lost from the vector or can be otherwise modified to reduce expression of the heterologous polypeptide.

As used herein, a polypeptide domain is a part of a polypeptide (a sequence of three or more, generally 5 or 7 or more amino acids) that is a structurally and/or functionally distinguishable or definable. Exemplary of a polypeptide domain is a part of the polypeptide that can form an independently folded structure within a polypeptide made up of one or more structural motifs (e.g. combinations of alpha helices and/or beta strands connected by loop regions) and/or that is recognized by a particular functional activity, such as enzymatic activity or antigen binding. A polypeptide can have one, typically more than one, distinct domains. For example, the polypeptide can have one or more structural domains and one or more functional domains. A single polypeptide domain can be distinguished based on structure and function. A domain can encompass a contiguous linear sequence of amino acids. Alternatively, a domain can encompass a plurality of non-contiguous amino acid portions, which are non-contiguous along the linear sequence of amino acids of the polypeptide. Typically, a polypeptide contains a plurality of domains. For example, each heavy chain and each light chain of an antibody molecule contains a plurality of immunoglobulin (Ig) domains, each about 110 amino acids in length. Those of skill in the art are familiar with polypeptide domains and can identify them by virtue of structural and/or functional homology with other such domains. For exemplification herein, definitions are provided, but it is understood that it is well within the skill in the art to recognize particular domains by name. If needed, appropriate software can be employed to identify domains.

As used herein, a structural polypeptide domain is a polypeptide domain that can be identified, defined or distinguished by homology of the amino acid sequence therein to amino acid sequences of related family members and/or by similarity of 3-dimensional structure to structure of related family members. Exemplary of related family members are members of the serine protease family. Also exemplary of related family members are members of the immunoglobulin family, for example, antibodies. For example, particular structural amino acid motifs can define an extracellular domain.

As used herein, a functional polypeptide domain is a domain that can be distinguished by a particular function, such as an ability to interact with a biomolecule, for example, through antigen binding, DNA binding, ligand binding, or dimerization, or by enzymatic activity, for example, kinase activity or proteolytic activity. A functional domain independently can exhibit a function or activity such that the domain, independently or fused to another molecule, can perform an activity, such as, for example enzymatic activity or antigen binding. Exemplary of domains are Immunoglobulin domains, variable region domains, including heavy and light chain variable region domains, constant region domains and antibody binding site domains.

As used herein, “extracellular domain” refers to the domain of a cell surface bound receptor or an antibody that is present on the outside surface of the cell and can includes ligand or antigen binding site(s).

As used herein, a transmembrane domain is a domain that spans the plasma membrane of a cell, anchoring the receptor and generally includes hydrophobic residues.

As used herein, a cytoplasmic domain of a cell surface receptor is the domain located within the intracellular space. A cytoplasmic domain can participate in signal transduction.

Those of skill in the art are familiar with these and other domains and can identify them by virtue of structural and/or functional homology with other such domains. For exemplification herein, definitions are provided, but it is understood that it is well within the skill in the art to recognize particular domains by name. If needed, appropriate software can be employed to identify domains.

As used herein, a portion of a polypeptide contains one or more contiguous amino acids within the polypeptide, for example, 1, 2, 3, 4, 5, 6, 8, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50 or more amino acids of the polypeptide, but fewer than all of the amino acids that make up the polypeptide. A portion can be a single amino acid position. A polypeptide domain can contain one, but typically more than one, portion. For example, the amino acid sequence of each CDR is a portion within the antigen binding site domain of an antibody. Each CDR is a portion of a variable region domain. Two or more non-contiguous portions can be part of the same domain.

As used herein, a region of a polypeptide is a portion of the polypeptide containing two or more contiguous amino acids of the polypeptide, for example, 2, 3, 4, 5, 6, 8, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50 or more, typically ten or more, contiguous amino acids, of the polypeptide, for example, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50 or more amino acids of the polypeptide, but not necessarily all of the amino acids that make up the polypeptide.

As used herein, a functional region of a polypeptide is a region of the polypeptide that contains at least one functional domain, which imparts a particular function, such as an ability to interact with a biomolecule, for example, through antigen binding, DNA binding, ligand binding, or dimerization, or by enzymatic activity, for example, kinase activity or proteolytic activity; exemplary of functional regions of polypeptides are antibody domains, such as V_{H}, V_{L}, C_{H}, C_{L}, and portions thereof, such as CDRs, including CDR1, CDR and CDR3, and antigen binding portions, such as antibody combining sites.

As used herein, a functional region of an antibody is a portion of the antibody that contains at least a V_{H}, V_{L}, C_{H} (e.g. C_{H}1, C_{H}2 or C_{H}3), C_{L} or hinge region domain of the antibody, or at least a functional region thereof.

As used herein, a functional region of a domain exchanged antibody is a portion of a domain exchanged antibody that contains at least the domain exchanged antibody's V_{H}, V_{L}, C_{H} (e.g. C_{H}1, C_{H}2 or C_{H}3), C_{L} or hinge region domain, or a functional region of such a domain, such that the functional region of the domain exchanged antibody (either alone or in combination with other domain exchanged antibody domain(s) or region(s) thereof), retains the domain exchanged structure of the domain exchanged antibody, including the V_{H}-V_{H} interface.

As used herein, a functional region of a V_{H} domain is at least a portion of the full V_{H} domain that retains at least a portion of the binding specificity of the full V_{H} domain (e.g. by retaining one or more CDR of the full V_{H} domain), such that the functional region of the V_{H} domain, either alone or in combination with another antibody domain (e.g. V_{L} domain) or region thereof, binds to antigen. Exemplary functional regions of V_{H} domains are regions containing the CDR1, CDR2 and/or CDR3 of the V_{H} domain.

As used herein, a functional region of a V_{L} domain is at least a portion of the full V_{L} domain that retains at least a portion of the binding specificity of the full V_{L} domain (e.g. by retaining one or more CDR of the full V_{L} domain), such that the function region of the V_{L} domain, either alone or in combination with another antibody domain (e.g. V_{H} domain) or region thereof, binds to antigen. Exemplary functional regions of V_{L} domains are regions containing the CDR1, CDR2 and/or CDR3 of the V_{L} domain.

As used herein, a functional region of a domain exchanged V_{H} domain is at least a portion of the full domain exchanged V_{H} domain that retains at least a portion of the binding specificity of the full domain exchanged V_{H} domain (e.g. by retaining one or more CDR domain and residues that promote the V_{H}-V_{H} interface), such that the functional region of a domain exchanged V_{H} domain, either alone or in conjunction with another domain (e.g. a V_{L} domain or another domain exchanged V_{H} domain), or functional region thereof, binds to antigen and retains the domain exchanged configuration, including the V_{H}-V_{H} interface. Exemplary of a functional region of a domain exchanged V_{H} domain is a portion containing the CDR1, CDR2 and/or CDR3 of the full domain exchanged V_{H} domain and any residues necessary to confer the formation of the V_{H}-V_{H} interface.

As used herein, a structural region of a polypeptide is a region of the polypeptide that contains at least one structural domain.

As used herein, a region of a polynucleotide is a portion of the polynucleotide containing two or more, typically at least six or more, typically ten or more, contiguous nucleotides, for example, 2, 2, 3, 4, 5, 6, 8, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50 or more nucleotides of the polynucleotide, but not necessarily all the nucleotides that make up the polynucleotide.

As used herein, a region of a target polynucleotide is a portion of the target polynucleotide that encodes at least a region of the target polypeptide (e.g. encodes a portion of the target polypeptide containing two or more contiguous amino acids, typically ten or more amino acids, of the target polypeptide, for example, 2, 3, 4, 5, 6, 8, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50 or more amino acids of the target polynucleotide).

As used herein, a functional region of a target polynucleotide is a region that encodes at least a functional domain of the polypeptide.

As used herein, a structural region of a target polynucleotide is a region that encodes at least a structural domain of the polypeptide.

As used herein, antibody refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Antibodies include domain exchanged antibodies, including domain exchanged antibody fragments. Hence antibody includes any protein having a binding domain that is homologous or substantially homologous to an immunoglobulin antigen binding domain (antibody combining site). For purposes herein, the term antibody includes antibody fragments, such as, but not limited to, Fab, Fab′, F(ab′)₂, single-chain Fvs (scFv), Fv, dsFv, diabody, Fd and Fd′ fragments Fab fragments, Fd fragments and scFv fragments. Other known fragments include, but are not limited to, scFab fragments (Hust et al., BMC Biotechnology (2007), 7:14), and domain exchanged fragments, such as domain exchanged scFv fragments, domain exchanged scFv tandem fragments, domain exchanged scFv hinge fragments, domain exchanged Fab fragments, domain exchanged single chain Fab fragments (scFab), domain exchanged Fab hinge fragments, and other modified domain exchanged fragments. Antibodies include members of any immunoglobulin class, including IgG, IgM, IgA, IgD and IgE.

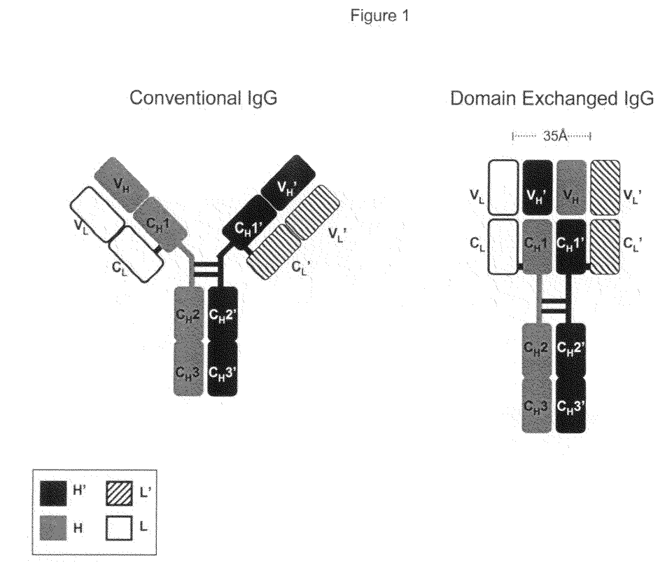

As used herein, a conventional antibody refers to an antibody that contains two heavy chains (which can be denoted H and H′) and two light chains (which can be denoted L and L′) and two antibody combining sites, where each heavy chain can be a full-length immunoglobulin heavy chain or any functional region thereof that retains antigen binding capability (e.g. heavy chains include, but are not limited to, V_{H}, chains V_{H}-C_{H}1 chains and V_{H}-C_{H}1-C_{H}2-C_{H}3 chains), and each light chain can be a full-length light chain or any functional region of (e.g. light chains include, but are not limited to, V_{L} chains and V_{L}-C_{L} chains). Each heavy chain (H and H′) pairs with one light chain (L and L′, respectively). (See e.g., FIG. 1, showing a conventional human full-length IgG antibody compared to a domain exchanged IgG antibody).

As used herein, a domain exchanged antibody refers to any antibody (including any antibody fragment) that has a domain exchanged three-dimensional structural configuration, characterized by the pairing of each heavy chain variable region with the opposite light chain variable region (and optionally the opposite light chain constant region), where the pairing is opposite as compared to heavy-light chain pairing in a conventional antibody, and by the formation of an interface (V_{H}-V_{H}′ interface) between adjacently positioned V_{H} domains (see, e.g. FIG. 1, comparing exemplary conventional and domain exchanged full-length IgG antibodies), including any antibody fragment derived from such an antibody that retains the V_{H}-V_{H}′ interface and at least a portion of the antigen specificity of the antibody. This V_{H}-V_{H}′ interface can contain one or more non-conventional antibody combining sites. In one example, the opposite pairing and V_{H}-V_{H}′ interface are formed by interlocked heavy chains.

As used herein, a full-length antibody is an antibody having two full-length heavy chains (e.g. V_{H}-C_{H}1-C_{H}2-C_{H}3 or V_{H}-C_{H}1-C_{H}2-C_{H}3-C_{H}4) and two full-length light chains (V_{L}-C_{L}) and hinge regions, such as human antibodies produced naturally by antibody secreting B cells and antibodies with the same domains that are synthetically produced.

As used herein, antibody fragment refers to any portion of a full-length antibody that is less than full length but contains at least a portion of the variable region of the antibody that binds antigen (e.g. one or more CDRs and/or one or more antibody combining sites) and thus retains the binding specificity, and at least a portion of the specific binding ability of the full-length antibody; antibody fragments include antibody derivatives produced by enzymatic treatment of full-length antibodies, as well as synthetically, e.g. recombinantly produced derivatives. Examples of antibody fragments include, but are not limited to, Fab, Fab′, F(ab′)₂, single-chain Fvs (scFv), Fv, dsFv, diabody, Fd and Fd′ fragments and domain exchanged fragments, such as domain exchanged scFv fragments, domain exchanged scFv tandem fragments, domain exchanged scFv hinge fragments, domain exchanged Fab fragments, domain exchanged single chain Fab fragments (scFab), domain exchanged Fab hinge fragments, and other modified domain exchanged fragments and other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least about 50 amino acids and typically at least 200 amino acids.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, an Fd fragment is a fragment of an antibody containing a variable domain (V_{H}) and one constant region domain (C_{H}1) of an antibody heavy chain.

As used herein, a conventional Fab fragment (also referred to as simply “Fab fragment”) is an antibody fragment that results from digestion of a full-length immunoglobulin with papain, or a fragment having the same structure that is produced synthetically, e.g. recombinantly. A conventional Fab fragment contains a light chain (containing a V_{L} and C_{L}) and another chain containing a variable domain of a heavy chain (V_{H}) and one constant region domain of the heavy chain (C_{H}1); it can be recombinantly produced.

As used herein, 2G12 refers to the domain exchanged human monoclonal IgG1 antibody produced from the hybridoma cell line CL2 (as described in U.S. Pat. No. 5,911,989; Buchacher et al., AIDS Research and Human Retroviruses, 10(4) 359-369 (1994); and Trkola et al., Journal of Virology, 70(2) 1100-1108 (1996)), and any synthetically, e.g. recombinantly, produced antibody having the identical sequence of amino acids, including any antibody fragment thereof having at least the antigen-binding portions of the heavy and light chain variable region domains to the full-length antibody, such as the 2G12 domain exchanged Fab fragment (see, for example, Published U.S. Application, Publication No.: US20050003347 and Calarese et al., Science, 300, 2065-2071 (2003), including supplemental information). 2G12 antibodies specifically bind HIV gp120 antigen.

As used herein, “gp120” “HIV gp120” and “gp120 antigen” refer to the HIV envelope surface glycoprotein, epitopes of which are specifically recognized and bound by the 2G12 antibody. HIV gp120 (GENBANK gi:28876544) is one of two cleavage products resulting from cleavage of the gp160 precursor glycoprotein (GENBANK g.i. 9629363). Gp120 can refer to the full-length gp120 or a fragment thereof containing epitopes bound by the 2G12 antibody.

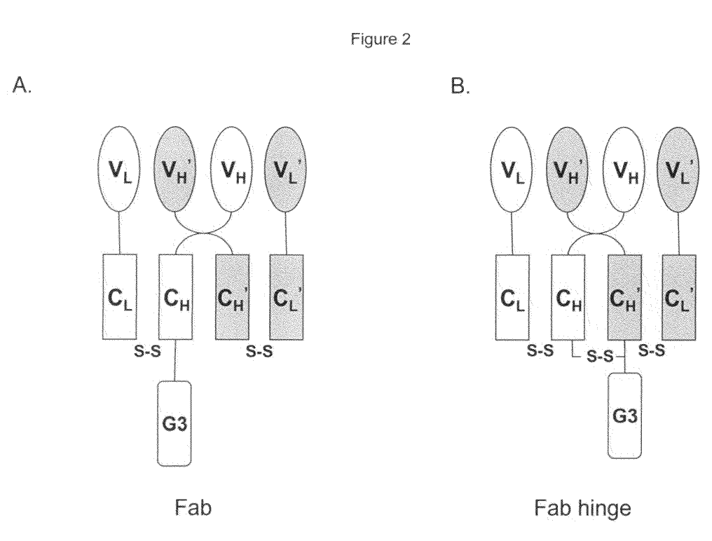

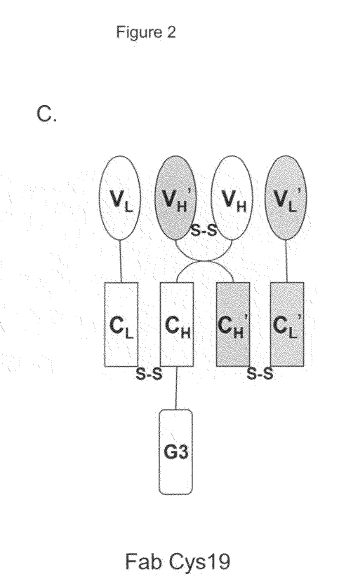

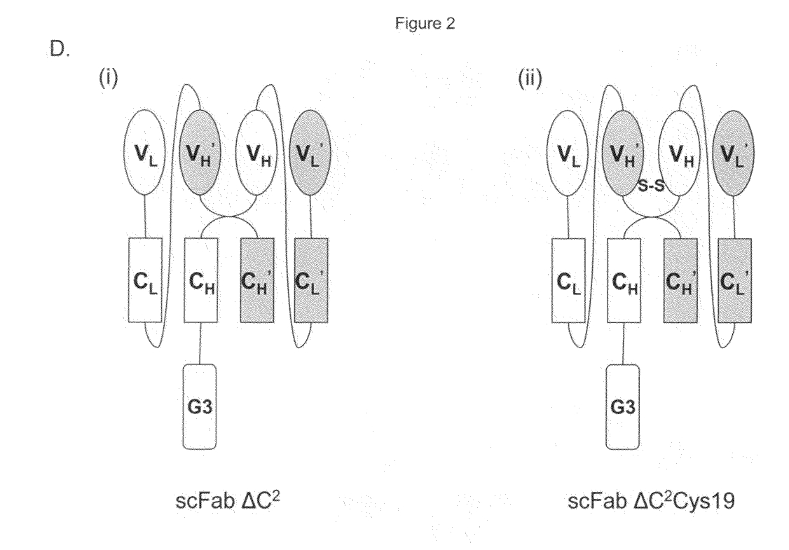

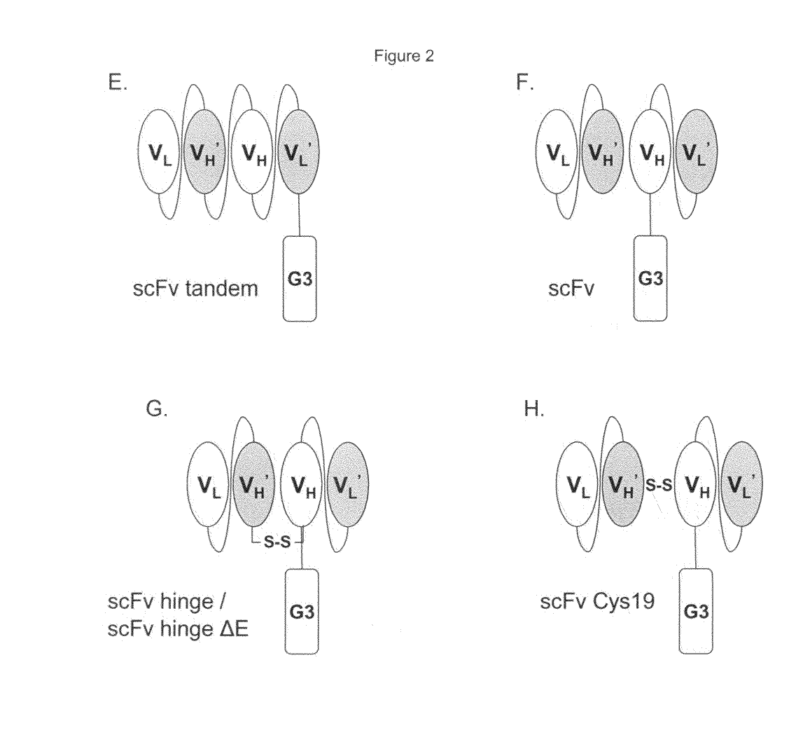

As used herein, a domain exchanged Fab fragment is a domain exchanged antibody fragment that contains two copies each of a light (V_{L}-C_{L}, V_{L}′-C_{L}′) chain and a heavy (V_{H}-C_{H}1, V_{H}′-C_{H}1′) chain, which are folded in the domain exchanged configuration, where each heavy chain variable region pairs with the opposite light chain variable region compared to a conventional antibody, and an interface (V_{H}-V_{H}′) is formed between adjacently positioned V_{H} domains. Typically, the fragment contains two conventional antibody combining sites and at least one non-conventional antibody combining site (contributed to by residues at the V_{H}-V_{H}′ interface). See, for example, FIG. 2A, showing a domain exchanged Fab fragment displayed on phage.

A domain exchanged single chain Fab fragment (scFab) is a domain exchanged Fab fragment, further including peptide linkers between each V_{H} and V_{L}. In some examples of a domain exchanged scFab fragment (e.g. domain exchanged scFabΔC2 fragment), one or more cysteines are mutated compared to the native scFab fragment, to eliminate one or more disulfide bonds between constant regions.

A domain exchanged Fab hinge fragment is a domain exchanged Fab fragment, further containing an antibody hinge region adjacent to each heavy chain constant region.

As used herein, a F(ab′)₂ fragment is an antibody fragment that results from digestion of an immunoglobulin with pepsin at pH 4.0-4.5, or a synthetically, e.g. recombinantly, produced antibody having the same structure. The F(ab′)2 fragment essentially contains two Fab fragments where each heavy chain portion contains an additional few amino acids, including cysteine residues that form disulfide linkages joining the two fragments; it can be recombinantly produced.

A Fab′ fragment is a fragment containing one half (one heavy chain and one light chain) of the F(ab′)2 fragment.

As used herein, an Fd′ fragment is a fragment of an antibody containing one heavy chain portion of a F(ab′)2 fragment.

As used herein, an Fv′ fragment is a fragment containing only the V_{H} and V_{L} domains of an antibody molecule.

As used herein, a conventional scFv fragment (also referred to simply as “scFv” fragment) refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, a domain exchanged scFv fragment is a domain exchanged antibody fragment containing two chains, each of which contains one V_{H} and one V_{L} domain, joined by a peptide linker (V_{H}-linker-V_{L}). The two chains interact through the V_{H} domains, producing the V_{H}-V_{H}′ interface characteristic of the domain exchanged configuration. Typically, the V_{H}-linker-V_{L} sequence of amino acids in each chain is identical. An example is illustrated in FIG. 2F.

In one example, as illustrated in FIG. 2F, when the domain exchanged scFv fragment is displayed on a genetic package, one of the chains is a fusion protein, containing the V_{H}-linker-V_{L} and a coat protein, such as cp3 (coat protein-V_{H}-linker-V_{L}), and the other chain is a soluble chain (V_{H}-linker-V_{L}). Alternatively, both chains can be fusion proteins.

A domain exchanged scFv hinge fragment is a domain exchanged scFv fragment further containing an antibody hinge region adjacent to each V_{H} domain. An example is illustrated in FIG. 2G.

As used herein, a domain exchanged scFv tandem fragment refers to a domain exchanged antibody fragment containing two V_{H} domains and two V_{L} domains, each in a single chain and separated by polypeptide linkers. The linear configuration of these domains is V_{L}-linker-V_{H}-linker-V_{H}-linker-V_{L}. An example is illustrated in FIG. 2E. In one example, for display on genetic packages, the fragment further includes a coat protein, e.g. a phage coat protein, at one or the other end of the molecule, adjacent or in close proximity to one of the V_{L} chains.

As used herein, hsFv refers to antibody fragments in which the constant domains normally present in a Fab fragment have been substituted with a heterodimeric coiled-coil domain (see, e.g., Arndt et al. (2001) J Mol Biol. 7:312:221-228).

As used herein, “antibody hinge region” or “hinge region” refers to a polypeptide region that exists naturally in the heavy chain of the gamma, delta and alpha antibody isotypes, between the C_{H}1 and C_{H}2 domains that has no homology with the other antibody domains. This region is rich in proline residues and gives the IgG, IgD and IgA antibodies flexibility, allowing the two “arms” (each containing one antibody combining site) of the Fab portion to be mobile, assuming various angles with respect to one another as they bind antigen. This flexibility can allow the Fab arms to move in order to align the antibody combining sites to interact with epitopes on cell surfaces or other antigens. Two interchain disulfide bonds within the hinge region stabilize the interaction between the two heavy chains. In some embodiments provided herein, the synthetically produced antibody fragments contain one or more hinge region, for example, to promote stability via interactions between two antibody chains. Hinge regions are exemplary of dimerization domains.

As used herein, “linker” refers to short sequences of amino acids that join two polypeptide sequences (or nucleic acid encoding such an amino acid sequence). “Peptide linker” refers to the short sequence of amino acids joining the two polypeptide sequences. Exemplary of polypeptide linkers are linkers joining two antibody chains in a synthetic antibody fragment such as an scFv fragment. Linkers are well-known and any known linkers can be used in the provided methods. Exemplary of polypeptide linkers are (Gly-Ser)ₙ amino acid sequences, with some Glu or Lys residues dispersed throughout to increase solubility. Other exemplary linkers are described herein; any of these and other known linkers can be used with the provided compositions and methods.

As used herein, dimerization domains are any domains that facilitate interaction between two polypeptide sequences (such as, but not limited to, antibody chains). Dimerization domains include, but are not limited to, an amino acid sequence containing a cysteine residue that facilitates formation of a disulfide bond between two polypeptide sequences, such as all or part of a full-length antibody hinge region, or one or more dimerization sequences, which are sequences of amino acids known to promote interaction between polypeptides, including, but not limited to, leucine zippers, GCN4 zippers, for example, the sequence of amino acids set forth in SEQ ID NO: 9 (GRMKQLEDKVEELLSKNYHLENEVARLKKLVGERG), and mixtures thereof. In some examples of the provided methods and compositions, one or more dimerization domains is included in a domain exchange antibody fragment, in order to promote interaction between chains, and thus stabilize the domain exchange configuration.

As used herein, diabodies are dimeric scFv; diabodies typically have shorter peptide linkers than scFvs, and they preferentially dimerize.

As used herein, humanized antibodies refer to antibodies that are modified to include “human” sequences of amino acids so that administration to a human does not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, idiotype refers to a set of one or more antigenic determinants specific to the variable region of an immunoglobulin molecule.

As used herein, anti-idiotype antibody refers to an antibody directed against the antigen-specific part of the sequence of an antibody or T cell receptor. In principle an anti-idiotype antibody inhibits a specific immune response.

As used herein, “monoclonal antibody” refers to a population of identical antibodies, meaning that each individual antibody molecule in a population of monoclonal antibodies is identical to the others. This property is in contrast to that of a polyclonal population of antibodies, which contains antibodies having a plurality of different sequences. Monoclonal antibodies can be produced by a number of well-known methods (Smith et al., J Clin Pathol (2004) 57, 912-917; and Nelson et al., J Clin Pathol (2000), 53, 111-117). For example, monoclonal antibodies can be produced by immortalization of a B cell, for example through fusion with a myeloma cell to generate a hybridoma cell line or by infection of B cells with virus such as EBV. Recombinant technology also can be used to produce monoclonal antibodies in vitro from clonal populations of host cells by transforming the host cells with plasmids carrying artificial sequences of nucleotides encoding the antibodies.

As used herein, an Ig domain is a domain, recognized as such by those in the art, that is distinguished by a structure, called the Immunoglobulin (Ig) fold, which contains two beta-pleated sheets, each containing anti-parallel beta strands of amino acids connected by loops. The two beta sheets in the Ig fold are sandwiched together by hydrophobic interactions and a conserved intra-chain disulfide bond. Individual immunoglobulin domains within an antibody chain further can be distinguished based on function. For example, a light chain contains one variable region domain (V_{L}) and one constant region domain (C_{L}), while a heavy chain contains one variable region domain (V_{H}) and three or four constant region domains (C_{H}). Each V_{L}, C_{L}, V_{H}, and C_{H} domain is an example of an immunoglobulin domain.

As used herein, a variable region domain is a specific Ig domain of an antibody heavy or light chain that contains a sequence of amino acids that varies among different antibodies. Each light chain and each heavy chain has one variable region domain (V_{L}, and, V_{H}). The variable domains provide antigen specificity, and thus are responsible for antigen recognition. Each variable region contains CDRs that are part of the antigen binding site domain and framework regions (FRs).

As used herein, “antigen binding site,” “antigen combining site” and “antibody combining site” are used synonymously to refer to a domain within an antibody that recognizes and physically interacts with cognate antigen. A native conventional full-length antibody molecule has two conventional antigen combining sites, each containing portions of a heavy chain variable region and portions of a light chain variable region. A conventional antigen binding site contains the loops that connect the anti-parallel beta strands within the variable region domains. The antigen combining sites can contain other portions of the variable region domains. Each conventional antigen binding site contains three hypervariable regions from the heavy chain and three hypervariable regions from the light chain. The hypervariable regions also are called complementarity-determining regions (CDRs).

In one example, a domain-exchanged antibody further contains one or more non-conventional antibody combining site formed by the interface between the two heavy chain variable regions. In this example, the domain exchanged antibody contains two conventional and at least one non-conventional antibody combining site. As used herein, an “antigen binding” portion or region of an antibody is a portion/region that contains at least the antibody combining site (either conventional or non-conventional) or a portion of the antibody combining site that retains the antigen specificity of the corresponding full-length antibody (e.g. a V_{H} portion of the antibody combining site).

As used herein, a non-conventional antibody combining site, antigen binding site, or antigen combining site refers to domain within an antibody that recognizes and physically interacts with cognate antigen but does not contain the conventional portions of one heavy chain variable region and one light chain variable region. Exemplary of non-conventional antibody combining sites is the non-conventional site comprised of regions of the two heavy chain variable regions in a domain exchanged antibody.

As used herein, “hypervariable region,” “HV,” “complementarity-determining region” and “CDR” and “antibody CDR” are used interchangeably to refer to one of a plurality of portions within each variable region that together form an antigen binding site of an antibody. Each variable region domain contains three CDRs, named CDR1, CDR2 and CDR3. The three CDRs are non-contiguous along the linear amino acid sequence, but are proximate in the folded polypeptide. The CDRs are located within the loops that join the parallel strands of the beta sheets of the variable domain.

As used herein, framework regions (FRs) are the domains within the antibody variable region domains that are located within the beta sheets; the FR regions are comparatively more conserved, in terms of their amino acid sequences, than the hypervariable regions.

As used herein, a constant region domain is a domain in an antibody heavy or light chain that contains a sequence of amino acids that is comparatively more conserved than that of the variable region domain. In conventional full-length antibody molecules, each light chain has a single light chain constant region (C_{L}) domain and each heavy chain contains one or more heavy chain constant region (C_{H}) domains, which include, C_{H}1, C_{H}2, C_{H}3 and C_{H}4. Full-length IgA, IgD and IgG isotypes contain C_{H}1, C_{H}2 C_{H}3 and a hinge region, while IgE and IgM contain C_{H}1, C_{H}2 C_{H}3 and C_{H}4. p C_{H}1 and C_{L} domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g. through interactions with various cells, biomolecules and tissues.

As used herein, a target polypeptide is a polypeptide selected for variation, such as by randomization methods for creating nucleic acid and polypeptide libraries, such as those described herein and those known in the art. The target polypeptide can be, for example, a native or wild-type polypeptide, or a polypeptide that contains one or more alterations compared to a native or wild-type polypeptide. In one example, the target polypeptide is a polypeptide selected from a collection of variant polypeptides made according to the methods provided herein. In one example, the sequence of the nucleic acid molecule encoding the target polypeptide is used to design synthetic oligonucleotides for use in the provided methods for creating diversity.

The target polypeptide can be a single chain polypeptide (e.g. a heavy chain of an antibody or a functional region thereof) or can include multiple chains, for example, an entire antibody or antibody fragment. Exemplary of target polypeptides are antibodies, including antibody fragments (for example, a Fab or scFv fragment), antibody chains (e.g. heavy and light chains) and antibody domains (e.g. variable region domains, such as the heavy chain variable region).

As used herein, a target domain is a specific domain within the target polypeptide that is selected for variation using the methods herein. A target polypeptide can have one or more target domains. A target domain can include one, typically more than one, for example 2, 3, 4, 5, 6, 7, 8, 9, 10, 15 or more, target portions.

As used herein, a target portion of a polypeptide is a specific portion within the amino acid sequence of a target polypeptide that is selected for variation using the methods herein. One or more target portions can be selected for variation within a single target polypeptide. The one or more target portions can be within a single target domain or within a plurality of target domains. Each target portion can have one or more target positions.

As used herein, target position of a polypeptide is an individual amino acid position within a target portion that is selected for variation by the methods herein. If the target portion contains only one amino acid in length, the target portion is synonymous with the target position.

As used herein, a target polynucleotide is a polynucleotide including the sequence of nucleotides encoding a target polypeptide or a functional region of the target polypeptide (e.g. a chain of the target polypeptide), and optionally containing additional 5′ and/or 3′ sequence(s) of nucleotides (for example, non-gene-specific nucleotide sequences), for example, restriction endonuclease recognition site sequence(s), sequence(s) complementary to a portion of one or more primers, and/or nucleotide sequence(s) of a bacterial promoter or other bacterial sequence, or any other non gene-specific sequence. The target polynucleotide can be single or double stranded. Target portions within the target polynucleotide encode the target portions of the target polypeptide. Using methods described herein, variant polynucleotides, for example, randomized oligonucleotides, randomized duplex oligonucleotide fragments and randomized oligonucleotide duplex cassettes are synthesized based on the target polynucleotide sequence. Exemplary of target polynucleotides are polynucleotides encoding antibody chains, and polynucleotides encoding antibodies, such as antibody fragments, including domain exchanged antibody fragments (for example, a target polynucleotide encoding a Fab fragment, for example, contained in a vector), antibody chains (e.g. heavy and light chains) and antibody domains (e.g. variable region domains, such as the heavy chain variable region).

As used herein, a variant portion of a polypeptide is a portion that varies in amino acid sequence compared to an analogous portion in a target polypeptide and/or compared to an analogous portion within one or more polypeptides in a collection of variant polypeptides. Typically, each variant portion corresponds to an analogous target portion within the target polypeptide. The amino acid sequence in the variant portion typically is varied by amino acid substitution(s). For example, if an analogous target portion in a target polypeptide contains a valine at a particular amino acid position, a variant portion might have an arginine at the analogous position. The variations alternatively can vary due to additions, deletions or insertions.

As used herein, a variant position of a polypeptide is a single amino acid position of a variant polypeptide that varies compared to an analogous amino acid position in a target polypeptide and/or compared to an analogous position in other members of a collection of variant polypeptides.

As used herein, a variant polypeptide is a polypeptide having one or more, typically at least two, for example, 2, 3, 4, 5, 6, 7, 8, 9, 10, 15 or more, variant portions, compared to a target polypeptide or another polypeptide within a collection (e.g. a pool) of polypeptides. Two or more variant portions within one variant polypeptide typically are non-contiguous in the linear amino acid sequence of the polypeptide. Two or more variant portions can be within the same domain of the variant polypeptide. Two variant portions that are within the same domain can be non-contiguous along the linear amino acid sequence.

For example, a variant antibody variable-region domain polypeptide can contain variant portion(s) within one or more, typically two or three CDRs, where the variant portions vary compared to a native or target antibody variable region polypeptide or compared to other polypeptides in a collection of variant antibody variable domain polypeptides. In one example, the variant antibody polypeptide contains a V_{H} and/or a V_{L} domain, each domain containing three or more variant portions, each within a single CDR. In this example, all the variant portions are within the variant antibody binding site domain. In another example, fewer than each of the three CDRs in a variable region are variant, for example, one or more of CDR1, CDR2 or CDR3 can contain variant portions. In addition to the variant portions, variant polypeptides also contain non-variant portions, which are 100% identical in amino acid sequence to analogous portions of a target polypeptide, a native polypeptide or of the other variant polypeptides in a collection.

As used herein, a collection of variant polypeptides is a collection containing a plurality of analogous polypeptides, each having one or more variant portions compared to a target polypeptide or compared to other polypeptides in the collection. Exemplary of collections of polypeptides are polypeptide libraries, including, but not limited to phage display libraries, such as phage display libraries containing displayed domain exchanged antibodies. It is not necessary that each polypeptide within a variant collection be varied compared to (i.e. contain an amino acid sequence that is different than) the target polypeptide. Nor is it necessary that each polypeptide within the variant collection is varied compared to (i.e. contain an amino acid sequence that is different than) each other polypeptide of the collection. In other words, the amino acid sequence of each individual variant polypeptide is not necessarily different for each member of the collection. Typically, among the variant polypeptides in the collections are at least 10⁴ or about 10⁴, 10⁵ or about 10⁵, 10⁶ or about 10⁶, at least 10⁸ or about 10⁸, at least 10⁹ or about 10⁹, at least 10¹⁰ or about 10¹⁰, or more different polypeptide amino acid sequences. Thus, the collections typically have a diversity of at least 10⁴ or about 10⁴, 10⁵ or about 10⁵, 10⁶ or about 10⁶, at least 10⁸ or about 10⁸, at least 10⁹ or about 10⁹, at least 10¹⁰ or about 10¹⁰, or more.

The variant polypeptides are encoded by variant nucleic acid molecules, typically by variant nucleic acid molecules containing randomized oligonucleotides. The collections of variant polypeptides typically contain at least 10⁶ or about 10⁶ variant polypeptide members, typically at least 10⁷ or about 10⁷ members, typically at least 10⁸ or about 10⁸ members, typically at least 10⁹ or about 10⁹ members, typically at least 10¹⁰ or about 10¹⁰ members or more. More than one variant polypeptide in the collection can contain each individual different amino acid sequence.

As used herein, a modified polypeptide or polynucleotide is a polypeptide or polynucleotide containing one or more amino acid or nucleotide insertions, deletions, additions, substitutions or amino acid or nucleotide modifications, compared to another related molecule, such as a target or native polypeptide or polynucleotide. The modified molecule is said to be modified compared to the other molecule and the modifications typically are described with relation to the particular residues that are modified along the linear amino acid or nucleotide sequence.

As used herein, the term “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine. Nucleic acids can contain nucleotide analogs, including, for example, mass modified nucleotides, which allow for mass differentiation of nucleic acid molecules; nucleotides containing a detectable label such as a fluorescent, radioactive, luminescent or chemiluminescent label, which allow for detection of a nucleic acid molecule; or nucleotides containing a reactive group such as biotin or a thiol group, which facilitates immobilization of a nucleic acid molecule to a solid support. A nucleic acid also can contain one or more backbone bonds that are selectively cleavable, for example, chemically, enzymatically or photolytically cleavable. For example, a nucleic acid can include one or more deoxyribonucleotides, followed by one or more ribonucleotides, which can be followed by one or more deoxyribonucleotides, such a sequence being cleavable at the ribonucleotide sequence by base hydrolysis. A nucleic acid also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)).

As used herein, the terms “polynucleotide” and “nucleic acid molecule” refer to an oligomer or polymer containing at least two linked nucleotides or nucleotide derivatives, including a deoxyribonucleic acid (DNA) and a ribonucleic acid (RNA), joined together, typically by phosphodiester linkages. Polynucleotides also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). Polynucleotides (nucleic acid molecules), include single-stranded and/or double-stranded polynucleotides, such as deoxyribonucleic acid (DNA), and ribonucleic acid (RNA) as well as analogs or derivatives of either RNA or DNA. The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine. Polynucleotides can contain nucleotide analogs, including, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically cleavable. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)). Exemplary of the nucleic acid molecules (polynucleotides) provided herein are oligonucleotides, including synthetic oligonucleotides, oligonucleotide duplexes, primers, including fill-in primers, and oligonucleotide duplex cassettes.

As used herein, a variant nucleic acid molecule (e.g. a variant polynucleotide, such as a variant polynucleotide duplex, for example, a variant assembled polynucleotide duplex) is any nucleic acid molecule (e.g. polynucleotide) having one or more, typically at least two, e.g. 1, 2, 3, 4, 5, 6, 7, 8, 9, 10, 15 or more, variant portions compared to a target nucleic acid sequence, target polynucleotide, or reference sequence, or compared to one or more other variant nucleic acid molecules within a collection of variant nucleic acid molecules. Exemplary of variant nucleic acid molecules are variant polynucleotides, including variant oligonucleotides, for example, randomized oligonucleotides, randomized duplex oligonucleotide fragments and randomized oligonucleotide duplex cassettes. Collections of variant nucleic acid molecules can be used to express a collection of variant polypeptides. A collection of variant nucleic acid molecules, for example, a nucleic acid library, can encode a collection of variant polypeptides.

As used herein, a variant position is a nucleotide position of a variant nucleic acid molecule that varies compared to an analogous nucleotide position in a target polynucleotide or other member of the collection of variant nucleic acids.

As used herein, a collection (or pool) of polypeptides or of nucleic acid molecules refers to a plurality of such molecules, for example, 2 or more, typically 5 or more, and typically 10 or more, such as, for example, at or about 10, 15, 20, 30, 40, 50, 60, 70, 80, 90, 100, 200, 300, 400, 500, 1000, 10⁴, 10⁵, 10⁶, 10⁷, 10⁸, 10⁹, 10¹⁰, 10¹¹, 10¹², 10¹³, 10¹⁴ or more of such molecules. Typically, the members of the pool are analogous to one another. For example, among the provided collections (pools) of polynucleotides are randomized oligonucleotide pools and collections of variant assembled duplexes, where the nucleotide sequences among the members of the pool are analogous.

As used herein, a collection of variant nucleic acid molecules (e.g. collection of variant polynucleotides) is a collection containing a plurality (e.g. 2 or more, and typically 5 or more and typically 10 or more, such as 10, 15, 20, 30, 40, 50, 60, 70, 80, 90, 100, 200, 300, 400, 500, 1000, 10⁴, 10⁵, 10⁶, 10⁷, 10⁸, 10⁹, 10¹⁰, 10¹¹, 10¹², 10¹³, 10¹⁴ or more) of analogous nucleic acid molecules (e.g. variant polynucleotides), each having one or more variant portions compared to a target nucleic acid molecule and/or compared to other nucleic acid molecules in the collection. Exemplary of the collection of variant nucleic acid molecules are nucleic acid libraries, e.g. libraries where the variant nucleic acid molecules are contained in vectors, or where the variant nucleic acid molecules are vectors. It is not necessary that each polynucleotide within a variant collection be varied compared to (i.e. contain a nucleic acid sequence that is different than) the target polynucleotide. Nor is it necessary that each polynucleotide within the variant collection is varied compared to (i.e. contain a nucleic acid sequence that is different than) each other polynucleotide of the collection. In other words, the nucleic acid sequence of each individual variant polynucleotide is not necessarily different for each member of the collection. Typically, among the variant polynucleotide in the collections are at least 10⁴ or about 10⁴, 10⁵ or about 10⁵, 10⁶ or about 10⁶, at least 10⁸ or about 10⁸, at least 10⁹ or about 10⁹, at least 10¹⁰ or about 10¹⁰, or more different polynucleotide nucleic acid sequences. Thus, the collections typically have a diversity of at least 10⁴ or about 10⁴, 10⁵ or about 10⁵, 10⁶ or about 10⁶, at least 10⁸ or about 10⁸, at least 10⁹ or about 10⁹, at least 10¹⁰ or about 10¹⁰, at least 10¹¹ or about 10¹¹, at least 10¹² or about 10¹², at least 10¹³ or about 10¹³, at least 10¹⁴ or about 10¹⁴, or more.

The provided collections of variant polynucleotides typically contain at least 10⁴ or about 10⁴, 10⁵ or about 10⁵, 10⁶ or about 10⁶ variant polynucleotide members, typically at least 10⁷ or about 10⁷ members, typically at least 10⁸ or about 10⁸ members, typically at least 10⁹ or about 10⁹ members, typically at least 10¹⁰ or about 10¹⁰ members or more.

As used herein, the amount of “diversity” in a collection of polypeptides or polynucleotides refers to the number of different amino acid sequences or nucleic acid sequences, respectively, among the analogous polypeptide or polynucleotide members of that collection. For example, a collection of randomized polynucleotides having a diversity of 10⁷ contains 10⁷ different nucleic acid sequences among the analogous polynucleotide members. In one example, the provided collections of polynucleotides and/or polypeptides have diversities of at least at or about 10⁴, 10⁵, 10⁶, 10⁷, 10⁸, 10⁹, 10¹⁰ or more. In another example, the collection of polynucleotides has at least 10⁴ or about 10⁴, 10⁵ or about 10⁵, 10⁶ or about 10⁶, 10⁷ or about 10⁷, 10⁸ or about 10⁸ or 10⁹ or about 10⁹ diversity, each member of the collection contains at least 50 or about 50, at least 100 or about 100, 200 or about 200, 300 or about 300, 500 or about 500, 1000 or about 1000, or 2000 or about 2000 nucleotides in length. In another example, the collection is a collection of randomized polynucleotides, in which, for each randomized position, each member of the collection contains one or the other of two nucleotides (e.g. A and T) at the randomized position and neither of the two nucleotides (e.g. A or T) is present at the position in more than 55% or about 55% of the members. In another example, the collection is a collection of randomized polynucleotides, in which, for each randomized position, each member of the collection contains one of four or more nucleotides (e.g. A, T, G and C or more) at the randomized position, and none of the four or more nucleotides is present at the analogous position in more than 30% of the members.

As used herein, “a diversity ratio” refers to a ratio of the number of different members in the library over the number of total members of the library. Thus, a library with a larger diversity ratio than another library contains more different members per total members, and thus more diversity per total members. The provided libraries include libraries having high diversity ratios, such as diversity ratios approaching 1, such as, for example, at or about 0.1, 0.2, 0.3, 0.4, 0.5, 0.6, 0.7, 0.8, 0.9 0.91, 0.92, 0.93, 0.94, 0.95. 0.96, 0.97, 0.98, or 0.99.

As used herein, a nucleic acid library is a collection of variant nucleic acid molecules. Typically, the nucleic acid library contains vectors containing variant polynucleotides, typically randomized polynucleotides, for example randomized oligonucleotide duplex cassettes. The randomized polynucleotides in the libraries can be generated using any of the methods provided herein. Typically, generation of the libraries includes generation of pools of randomized (or other variant) oligonucleotides. The polynucleotides in the nucleic acid library typically encode variant polypeptides. The libraries provided herein can be used to express collections of variant polypeptides.

As used herein, the terms “oligonucleotide” and “oligo” are used synonymously. Oligonucleotides are polynucleotides that contain a limited number of nucleotides in length. Those in the art recognize that oligonucleotides generally are less than at or about two hundred fifty, typically less than at or about two hundred, typically less than at or about one hundred, nucleotides in length. Typically, the oligonucleotides provided herein are synthetic oligonucleotides. The synthetic oligonucleotides contain fewer than at or about 250 or 200 nucleotides in length, for example, fewer than about 20, 30, 40, 50, 60, 70, 80, 90, 100, 110, 120, 130, 140, 150, 160, 170, 180, 190 or 200 nucleotides in length. Typically, the oligonucleotides are single-stranded oligonucleotides. The ending “mer” can be used to denote the length of an oligonucleotide. For example, “100-mer” can be used to refer to an oligonucleotide containing 100 nucleotides in length. Exemplary of the synthetic oligonucleotides provided herein are positive and negative strand oligonucleotides, randomized oligonucleotides, reference sequence oligonucleotides, template oligonucleotides and fill-in primers are.

As used herein, synthetic oligonucleotides are oligonucleotides produced by chemical synthesis. Chemical oligonucleotide synthesis methods are well known. Any of the known synthesis methods can be used to produce the oligonucleotides designed and used in the provided methods. For example, synthetic oligonucleotides typically are made by chemically joining single nucleotide monomers or nucleotide trimers containing protective groups. Typically, phosphoramidites, single nucleotides containing protective groups are added one at a time. Synthesis typically begins with the 3′ end of the oligonucleotide. The 3′ most phosphoramidite is attached to a solid support and synthesis proceeds by adding each phosphoramidite to the 5′ end of the last. After each addition, the protective group is removed from the 5′ phosphate group on the most recently added base, allowing addition of another phosphoramidite. Automated synthesizers generally can synthesize oligonucleotides up to about 150 to about 200 nucleotides in length. Typically, the oligonucleotides designed and used in the provided methods are synthesized using standard cyanoethyl chemistry from phosphoramidite monomers. Synthetic oligonucleotides produced by this standard method can be purchased from Integrated DNA Technologies (IDT) (Coralville, Iowa) or TriLink Biotechnologies (San Diego, Calif.).

As used herein, a portion of an oligonucleotide contains one or more contiguous nucleotides within the oligonucleotide, for example, 1, 2, 3, 4, 5, 6, 8, 10, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50, 60, 70, 80, 90, 100 or more nucleotides. An oligonucleotide can contain one, but typically more than one, portion.

As used herein, a reference sequence is a contiguous sequence of nucleotides that is used as a design template for synthesizing oligonucleotides according to the methods provided herein. Each reference sequence contains nucleic acid identity to a region of a target polynucleotide, as well as optional additional, deletions, insertions and/or substitutions compared to the region of the target polynucleotide. In one example, the region of the target polynucleotide, to which the reference sequence has identity, includes the entire length of the target polynucleotide. Typically, however, the region of the target polynucleotide, to which the reference sequence contains identity, includes less than the entire length of the target polynucleotide, but at least 2, typically at least 10, contiguous nucleotides of the target polynucleotide. In the provided methods, oligonucleotides in a pool of oligonucleotides are designed based on a reference sequence. In the case of variant oligonucleotides, one or more positions in the oligonucleotides vary compared to the reference sequence. In the case of randomized oligonucleotides, one or more positions (randomized positions) is synthesized using a doping strategy.

In one example, the reference sequence is 100% identical to the region of the target polynucleotide. In another example, the reference sequence is less than 100% identical to the region, such as at or about, or at least at or about, 99, 98, 97, 96, 95, 94, 93, 92, 91, 90%, or less, identical to the region, for example, at least at or about 50%, 55%, 60%, 65%, 70%, 75%, 80%, 85%, 90%, 95%, 96%, 97%, 98%, 99% or any fraction thereof. In one example, the reference sequence contains a region that is identical to the region of the target polynucleotide and an additional region or portion that contains a non gene-specific sequence, or a non-encoding sequence, for example, a regulatory sequence, such as a bacterial leader sequence, promoter sequence, or enhancer sequence; a sequence of nucleotides that is a restriction endonuclease recognition site; and/or a sequence having complementarity to a primer, such as a CALX24 binding sequence. In some cases, the sequence of complementarity to a primer or other additional sequence overlaps with the region of the reference sequence having identity to the target polynucleotide. In one example, the reference sequence contains one or more target portions, each of which corresponds to all or part of a target region within the target polynucleotide to which the reference sequence is identical.

As used herein, when a polypeptide or nucleic acid molecule or region thereof contains or has “identity” or “homology” to another polypeptide or nucleic acid molecule or region, the two molecules and/or regions share greater than or equal to at or about 40% sequence identity, and typically greater than or equal to at or about 50% sequence identity, such as at least at or about 60%, 65%, 70%, 75%, 80%, 85%, 90%, 95%, 96%, 97%, 98%, 99% or 100% sequence identity; the precise percentage of identity can be specified if necessary. A nucleic acid molecule, or region thereof, that is identical or homologous to a second nucleic acid molecule or region can specifically hybridize to a nucleic acid molecule or region that is 100% complementary to the second nucleic acid molecule or region. Identity alternatively can be compared between two theoretical nucleotide or amino acid sequences or between a nucleic acid or polypeptide molecule and a theoretical sequence.

Sequence “identity,” per se, has an art-recognized meaning and the percentage of sequence identity between two nucleic acid or polypeptide molecules or regions can be calculated using published techniques. Sequence identity can be measured along the full length of a polynucleotide or polypeptide or along a region of the molecule. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptides, the term “identity” is well known to skilled artisans (Carrillo, H. & Lipman, D., SIAM J Applied Math 48:1073 (1988)).

Sequence identity compared along the full length of two polynucleotides or polypeptides refers to the percentage of identical nucleotide or amino acid residues along the full-length of the molecule. For example, if a polypeptide A has 100 amino acids and polypeptide B has 95 amino acids, which are identical to amino acids 1-95 of polypeptide A, then polypeptide B has 95% identity when sequence identity is compared along the full length of a polypeptide A compared to full length of polypeptide B. Alternatively, sequence identity between polypeptide A and polypeptide B can be compared along a region, such as a 20 amino acid analogous region, of each polypeptide. In this case, if polypeptide A and B have 20 identical amino acids along that region, the sequence identity for the regions would be 100%. Alternatively, sequence identity can be compared along the length of a molecule, compared to a region of another molecule. As discussed below, and known to those of skill in the art, various programs and methods for assessing identity are known to those of skill in the art. High levels of identity, such as 90% or 95% identity, readily can be determined without software.

Whether any two nucleic acid molecules have nucleotide sequences that are at least 60%, 70%, 80%, 85%, 90%, 95%, 96%, 97%, 98% or 99% “identical” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Altschul, S. F., et al., J Molec Biol 215:403 (1990); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

In general, for determination of the percentage sequence identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). For sequence identity, the number of conserved amino acids is determined by standard alignment algorithms programs, and can be used with default gap penalties established by each supplier. Substantially homologous nucleic acid molecules would specifically hybridize typically at moderate stringency or at high stringency all along the length of the nucleic acid of interest. Also contemplated are nucleic acid molecules that contain degenerate codons in place of codons in the hybridizing nucleic acid molecule.

Therefore, the term “identity,” when associated with a particular number, represents a comparison between the sequences of a first and a second polypeptide or polynucleotide or regions thereof and/or between theoretical nucleotide or amino acid sequences. As used herein, the term at least “90% identical to” refers to percent identities from 90 to 99.99 relative to the first nucleic acid or amino acid sequence of the polypeptide. Identity at a level of 90% or more is indicative of the fact that, assuming for exemplification purposes, a first and second polypeptide length of 100 amino acids are compared, no more than 10% (i.e., 10 out of 100) of the amino acids in the first polypeptide differs from that of the second polypeptide. Similar comparisons can be made between first and second polynucleotides. Such differences among the first and second sequences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleotide or amino acid residue substitutions, insertions, additions or deletions. At the level of homologies or identities above about 85-90%, the result should be independent of the program and gap parameters set; such high levels of identity can be assessed readily, often by manual alignment without relying on software.

As used herein, alignment of a sequence refers to the use of homology to align two or more sequences of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

Related or variant polypeptides or nucleic acid molecules can be aligned by any method known to those of skill in the art. Such methods typically maximize matches, and include methods, such as using manual alignments and by using the numerous alignment programs available (for example, BLASTP) and others known to those of skill in the art. By aligning the sequences of polypeptides or nucleic acids, one skilled in the art can identify analogous portions or positions, using conserved and identical amino acid residues as guides. Further, one skilled in the art also can employ conserved amino acid or nucleotide residues as guides to find corresponding amino acid or nucleotide residues between and among human and non-human sequences. Corresponding positions also can be based on structural alignments, for example by using computer simulated alignments of protein structure. In other instances, corresponding regions can be identified. One skilled in the art also can employ conserved amino acid residues as guides to find corresponding amino acid residues between and among human and non-human sequences.

As used herein, “analogous” and “corresponding” portions, positions or regions are portions, positions or regions that are aligned with one another upon aligning two or more related polypeptide or nucleic acid sequences (including sequences of molecules, regions of molecules and/or theoretical sequences) so that the highest order match is obtained, using an alignment method known to those of skill in the art to maximize matches. In other words, two analogous positions (or portions or regions) align upon best-fit alignment of two or more polypeptide or nucleic acid sequences. The analogous portions/positions/regions are identified based on position along the linear nucleic acid or amino acid sequence when the two or more sequences are aligned. The analogous portions need not share any sequence similarity with one another. For example, alignment (such that maximizing matches) of the sequences of two homologous nucleic acid molecules, each 100 nucleotides in length, can reveal that 70 of the 100 nucleotides are identical. Portions of these nucleic acid molecules containing some or all of the other non-identical 30 amino acids are analogous portions that do not share sequence identity. Alternatively, the analogous portions can contain some percentage of sequence identity to one another, such as at or about 50%, 55%, 60%, 65%, 70%, 75%, 80%, 85%, 90%, 95%, 96%, 97%, 98%, 99%, or fractions thereof. In one example, the analogous portions are 100% identical.

Exemplary of analogous portions, positions and regions are portions, positions and regions that are analogous among members of a provided collection of variant polynucleotides or polypeptides. For example, collections of randomized polynucleotides (e.g. randomized oligonucleotides, assembled duplexes or duplex cassettes) contain randomized portions; the randomized portions contain randomized positions. The randomized portions and positions are analogous among the members of the collection. For example, a single randomized position is analogous among the members. When referring to a collection of randomized nucleic acids, “a randomized position” can be used to describe the randomized position that is analogous among all the members, where the position aligns when two of the members are aligned by best fit. Similarly, reference sequence portions and reference sequence positions are analogous among the members of the collection. In another example, the analogous portions are analogous between a target polypeptide and a variant polypeptide. For example, a variant portion in a variant polynucleotide is analogous to a target portion in a target polypeptide Analogous nucleic acid molecules, sequences and analogous polypeptides are those that share one or more analogous portions or similarity.

As used herein, when it is said that an oligonucleotide or pool of oligonucleotides is synthesized “based on a reference sequence,” this language indicates that that reference sequence was is used as a design template for the oligonucleotide or for each of the oligonucleotides in the pool and that the oligonucleotides in the pool contain portions identical to the reference sequence. Typically, the reference sequence is used to design oligonucleotides, which are synthesized in pools. Each oligonucleotide in a pool of oligonucleotides is designed based on the same reference sequence. In one example, a plurality of oligonucleotide pools can be synthesized to generate a plurality of oligonucleotides for assembling duplex cassettes. In this example, each of the reference sequences that are used as templates for the plurality of pools has sequence identity to a different region of the target polynucleotide. Typically, these different regions overlap along the nucleic acid sequence of the target polynucleotide. It is not necessary that a nucleic acid molecule having the sequence of nucleotides contained in the reference sequence be physically produced. For example, a virtual or theoretical reference sequence can be used as a design template for synthesizing the oligos.

As used herein, a variant portion of a polynucleotide (e.g. an oligonucleotide) is a portion of the polynucleotide having altered nucleic acid sequence compared to an analogous portion of a target polynucleotide, a reference nucleic acid sequence, or compared to an analogous portion in one or more other polynucleotides (e.g. oligonucleotides) within a collection of variant polynucleotides. Typically, each variant portion within each of the polynucleotides is analogous to a target portion within the reference sequence, which is analogous to all or part of a target portion of a target polynucleotide. Typically, the variant portions of the polynucleotides are randomized portions.

As used herein, a randomized portion of a polynucleotide (e.g. oligonucleotide) is a variant portion that varies in nucleic acid sequence compared to analogous portions in a plurality of other members in a collection (e.g. pool) of randomized polynucleotides, e.g. a collection of randomized oligonucleotides. Thus, a plurality of different nucleic acid sequences are represented at a particular randomized portion among the plurality of individual members in the collection. It is not necessary that the randomized portion vary among all the members of the collection, or that the randomized portion in a single polynucleotide vary compared to a target polynucleotide or to a native polynucleotide. Further, a randomized portion does not necessarily vary (compared to analogous portion(s)) at every nucleotide position within the randomized portion, but the nucleotide position at the 5′ end and the nucleotide position at the 3′ end of the randomized portion are randomized positions. In one example, when the randomized portions are part of a synthetic oligonucleotide, they are synthesized using one or more doping strategies during oligonucleotide synthesis. Randomized portions of polynucleotides alternatively can be synthesized by polymerase extension reaction, for example, using a randomized pool of primers and/or using one or more randomized polynucleotides (e.g. oligonucleotides) as a template.

As noted, in some examples, not every nucleotide position in the randomized portion is a randomized position. In one example, one or more positions within the randomized portion is a non-randomized position (e.g. a reference sequence position or variant position). For example, a randomized portion that is ten nucleotides in length can vary at all ten nucleotide positions compared to the reference sequence; alternatively, it can vary at only 5, 6, 7, 8, or 9 of the positions. Typically, at least 50% or at least about 50%, at least 60% or at least about 60%, at least 70% or at least about 70%, at least 80% or at least about 80%, at least 90% or at least about 90%, at least 95% or at least about 95%, at least 99% or at least about 99% or at or about 100% of the positions in the randomized portion are randomized positions. In one example, no more than 2 positions in the randomized portion are non-randomized. In another example, no more than one of the positions in the randomized portion is non-randomized. In another example, each position in the randomized portion is a randomized position. Randomized portions of polynucleotides can encode randomized portions of polypeptides, which are the amino acid portions that are encoded by the randomized portions of the polynucleotide.

The randomized portion can be a single nucleotide, or can be a plurality of contiguous nucleotides, and typically is 1, 2, 3, 4, 5, 6, 7, 8, 9, 10, 11, 12, 13, 14, 15, 20, 25, 30, 35, 40, 45, 50, 75, 80, 90, 100 or more nucleotides, such as, for example, a portion of a nucleic acid molecule that encodes a portion of a polypeptide domain, for example a target domain. Randomization of a randomized portion or position within a randomized portion can be saturating or non-saturating within a collection of randomized oligonucleotides. Along the length of a randomized portion of an oligonucleotide, some positions can be randomized by saturating randomization and others with non-saturating randomization. Similarly, if one randomized portion within an oligonucleotide is saturated, another randomized portion within the same oligonucleotide can be non-saturated.

As used herein, a doping strategy is a method used during chemical oligonucleotide synthesis of randomized portions of oligonucleotides. Doping strategies allow for incorporation of a plurality of different nucleotides at each analogous position within the randomized portion among the members of a pool of randomized oligonucleotides. Typically, positions of the randomized portions within the randomized oligonucleotides are synthesized using a doping strategy, while other portions (e.g. reference sequence portions) are synthesized using conventional synthesis methods. With the doping strategy, the incorporation of a plurality of different nucleotides at analogous positions among the randomized pool members can be carried out in a biased or non-biased fashion.

In one example, when one or more position within the randomized portion is a non-randomized position (e.g. a reference sequence or variant position), not every position within the randomized portion is synthesized using a doping strategy. For example, the randomized portion can contain 1, or more than 1, for example, 2, 3, 4, 5, or more reference sequence or variant positions among the randomized positions, which are not synthesized with a doping strategy.

As used herein, a randomized polynucleotide (e.g. a randomized oligonucleotide, a randomized polynucleotide duplex, e.g. an assembled randomized polynucleotide duplex) is a polynucleotide containing one or more randomized portion, where the randomized portion varies compared to analogous randomized portions among a collection of randomized polynucleotides. Synthetic randomized oligonucleotides are generated in pools of randomized oligonucleotides. Collections of other randomized polynucleotides can be generated from the pools of randomized oligonucleotides using the methods provided herein, for example, using techniques including, but not limited to, polymerase extension, amplification, assembly, hybridization, ligation and other methods.

As used herein, “pool of synthetic oligonucleotides” and “pool of oligonucleotides” refer to a collection of oligonucleotides, where the oligonucleotides are synthesized based on the same reference sequence. The oligonucleotides in the pool typically are synthesized together in the same one or more reaction vessels. It is not necessary that the oligonucleotides in the pool contain 100% identity in nucleotide sequence. For example, in a pool of variant oligonucleotides, the oligonucleotides contain one or more variant portions (e.g. randomized portions) that vary compared to other oligonucleotides in the pool.

As used herein, a pool of duplexes is a collection containing two or more analogous polynucleotide duplexes. Exemplary of the pool of duplexes are pools of reference sequence duplexes, pools of randomized duplexes (where the duplex members of the collection contain one or more randomized portions) and pools of assembled duplexes.

As used herein, a collection of randomized polynucleotides or a pool of randomized oligonucleotides refers to any collection of polynucleotides where each polynucleotide contains one or more randomized portions and the randomized portions are analogous to one another. Exemplary of collections of randomized polynucleotides are pools of randomized oligonucleotides and pools of randomized duplexes. The randomized polynucleotides in the collection, also contain one or more, typically two or more, reference sequence portions, which typically are identical among the members of the collection. Each randomized portion of the individual randomized polynucleotides varies, to some extent, compared to analogous portions within the reference sequence and/or with the analogous portion within the other oligonucleotides in the pool. It is not necessary that each polynucleotide in the collection has a different sequence of nucleotides in the randomized portion. For example, two or more members of the randomized collection can have an identical sequence of nucleotides over the length of the randomized portion. Pools of randomized oligonucleotides are synthesized using one or more doping strategies as described herein.

Typically, among the randomized polynucleotide in the collections are at least 10⁴ or about 10⁴, 10⁵ or about 10⁵, 10⁶ or about 10⁶, at least 10⁷ or about 10⁷, at least 10⁸ or about 10⁸, at least 10⁹ or about 10⁹, at least 10¹⁰ or about 10¹⁰, at least 10¹¹ or about 10¹¹, at least 10¹² or about 10¹², at least 10¹³ or about 10¹³, at least 10¹⁴ or about 10¹⁴, or more different analogous polynucleotide nucleic acid sequences. Thus, the collections typically have a diversity of at least 10⁴ or about 10⁴, 10⁵ or about 10⁵, 10⁶ or about 10⁶, at least 10⁷ or about 10⁷, at least 10⁸ or about 10⁸, at least 10⁹ or about 10⁹ at least 10¹⁰ or about 10¹⁰, at least 10¹¹ or about 10¹¹, at least 10¹² or about 10¹², at least 10¹³ or about 10¹³, at least 10¹⁴ or about 10¹⁴, or more.

In one example, the provided collections of randomized polynucleotides contain at least 10⁴ or about 10⁴, 10⁵ or about 10⁵, 10⁶ or about 10⁶, at least 10⁷ or about 10⁷, at least 10⁸ or about 10⁸, at least 10⁹ or about 10⁹, at least 10¹⁰ or about 10¹⁰, at least 10¹¹ or about 10¹¹, at least 10¹² or about 10¹², at least 10¹³ or about 10¹³, at least 10¹⁴ or about 10¹⁴, or more.

As used herein, a reference sequence portion of a polynucleotide refers generally to a portion of the polynucleotide that contains sequence identity to an analogous portion of a reference sequence or target polynucleotide. In one example, the reference sequence portion contains at or about 100% identity to the reference sequence or target polynucleotide or region thereof. In another example, the reference sequence oligonucleotide contains at or about or at least at or about 50%, 55%, 60%, 65%, 70%, 75%, 80%, 85%, 90%, 95%, 96%, 97%, 98%, 99% or 100% identity to the reference sequence or target polynucleotide or region thereof.

As used herein, a reference sequence portion of a synthetic oligonucleotide is a portion that theoretically contains (i.e. based on oligonucleotide design) at or about 100% identity to the analogous portion in the reference sequence. For example, a reference sequence portion of a randomized oligonucleotide is not randomized and thus is not synthesized using a doping strategy. It is understood, however, that error during synthesis can result in reference sequence portions with less than 100% sequence identity to the reference sequence.

As used herein, a reference sequence oligonucleotide is an oligonucleotide containing nucleic acid sequence identity, and theoretically 100% sequence identity, to the reference sequence used to design the oligonucleotide (e.g. used to design the pool of reference sequence oligonucleotides). In one example, the reference sequence oligonucleotide contains 100% identity to the reference sequence. Alternatively, the reference sequence oligonucleotide can contain less than 100% identity to the reference sequence, such as, for example, at or about or at least at or about 90%, 91%, 92%, 93%, 94%, 95%, 96%, 97%, 98% or 99% sequence identity to the reference sequence. For example, a pool of reference sequence oligonucleotides is designed with the goal that all of the oligonucleotides in the pool are 100% identical to the reference sequence. It is understood, however, that such a pool of oligonucleotides can contain one or more oligonucleotides that, due to error during synthesis, is not 100% identical to the reference sequence, for example, contains one or more deletions, insertions, mutations, substitutions or additions compared to the reference sequence.

As used herein, “reference sequence polynucleotide” is used generally to refer to polynucleotides with identity to one or more reference sequences and/or containing identity to a target polynucleotide or region thereof, and optionally containing one or more additions, deletions, insertions, substitutions or mutations compared to the target polynucleotide or region thereof or reference sequence. In one example, the reference sequence polynucleotide contains at or about 100% identity to the reference sequence or target polynucleotide or region thereof. In another example, the reference sequence oligonucleotide contains at or about or at least at or about 50%, 55%, 60%, 65%, 70%, 75%, 80%, 85%, 90%, 95%, 96%, 97%, 98%, 99% or 100% identity to the reference sequence or target polynucleotide or region thereof.

As used herein, saturating randomization refers to a process by, for each position or tri-nucleotide portion within the randomized portion, each of a plurality of nucleotides or tri-nucleotide combinations is incorporated at least once within a pool of randomized oligonucleotides. Exemplary of a collection of randomized oligonucleotides displaying saturating randomization is one where, within the entire collection, each of the sixty-four possible tri-nucleotide combinations that can be made by the four nucleotide monomers is incorporated at least once at a particular codon position of a particular randomized portion. In another example of a collection of randomized oligonucleotides made by saturating randomization, each of the sixty-four possible tri-nucleotide combinations is incorporated at least once at each tri-nucleotide position over the length of the randomized portion. In another example of a collection of randomized oligonucleotides made by saturating randomization, a tri-nucleotide combination encoding each of the twenty amino acids is incorporated at least once at a particular codon position or at each codon position along the randomized portion. Also exemplary of a collection of oligonucleotides displaying saturating randomization is one where each nucleotide is incorporated at least once at every nucleotide position or at a particular nucleotide position over the length of the randomized portion within the collection of oligonucleotides. Saturation is typically advantageous in that it increases the chances of obtaining a variant protein with a desired property. The desired level of saturation will vary with the type of target polypeptide, the length and number of randomized portion(s) and other factors.

As used herein, non-saturating randomization refers to a process by which fewer than all of a particular number of nucleotide or tri-nucleotide combinations are used at a particular position or tri-nucleotide portion within the randomized portion within the pool of oligonucleotides. For example, non-saturating randomization of a particular tri-nucleotide position might incorporate only 2, 3, 4, 5, 6, 7, 8, 9, 10 or more, but not all the possible, tri-nucleotide combinations at that position within the collection of randomized oligonucleotides. Substitution mutagenesis, where one nucleotide or tri-nucleotide unit is replaced with one other nucleotide or tri-nucleotide unit, is non-saturating and also can be used to create variant oligonucleotides in the methods provided herein.

As used herein, a non-biased doping strategy is a strategy used during random oligonucleotide synthesis, whereby each of a plurality of nucleotides or tri-nucleotides is present at an equal proportion during synthesis of each nucleotide or tri-nucleotide position. Exemplary of a non-biased doping strategy is one whereby each of the four nucleotide monomers (A, G, T and C) is added at an equal proportion during synthesis of each nucleotide position in a randomized portion. Non-biased doping strategies can be referred to as “N” doping strategies or “NNN” doping strategies, where N is A, G, T or C. The strategy can lead to equal frequency of each nucleotide monomer at each randomized position within the collection synthesized using this strategy. Non-biased doping strategies using an equal ratio of each of the nucleotide monomers can be undesirable, as they lead to a relatively high frequency of stop codon incorporation compared to some biased strategies. Because there are sixty-four possible combinations of tri-nucleotide codons, which encode only twenty amino acids, redundancy exists in the nucleotide code. Different amino acids have a more redundant code than others. Thus, non-biased incorporation of nucleotides will not result in an equal frequency of each of the twenty amino acids in the encoded polypeptide. If an equal frequency of amino acids is desired, a non-biased doping strategy using equal ratios of a plurality of tri-nucleotide units, each representing one amino acid, can be employed.

As used herein, a biased doping strategy is a strategy that incorporates particular nucleotides or codons at different frequencies than others, thus biasing the sequence of the randomized portions within a collection towards a particular sequence. For example, the randomized portion, or single nucleotide positions within the randomized portion, can be biased towards a reference nucleic acid sequence or the coding sequence of a target polynucleotide. Biasing positions towards a reference nucleic acid sequence means that, within a collection of randomized oligonucleotides, the nucleotides or codons used in the reference sequence at those nucleotide positions would be more common than other nucleotides or codons. Doping strategies also can be biased to reduce the frequency of stop codons while still maintaining a possibility for saturating randomization.

Exemplary of biased doping strategies used herein are NNK, NNB and NNS, and NNW; NNM, NNH; NND; NNV doping strategies and an NNT, NNA, NNG and NNC doping strategy. In an NNK doping strategy, randomized portions of positive strands are synthesized using an NNK pattern and negative strand portions are synthesized using an MNN pattern, where N is any nucleotide (for example, A, C, G or T), K is T or G and M is A or C. Thus, using this doping strategy, each nucleotide in the randomized portion of the positive strand is a T or G. This strategy typically is used to minimize the frequency of stop codons, while still allowing the possibility of any of the twenty amino acids (listed in table 2) to be encoded by trinucleotide codons at each position of the randomized portion among the randomized oligonucleotides in the pool. Similarly, for the NNB doping strategy, an NNB pattern is used, where N is any nucleotide and B represents C, G or T. For the NNS doping strategy, an NNS pattern is used, where N is any nucleotide and S represents C or G. In an NNW doping strategy, W is A or T; in an NNM doping strategy, M is A or C; in an NNH doping strategy, H is A, C or T; in an NND doping strategy, D is A, G or T; in an NNV doping strategy, G is A, G or C. An NNK doping strategy minimizes the frequency of stop codons and ensures that each amino acid position encoded by a codon in the randomized portion could be occupied by any of the 20 amino acids. With this doping strategy, nucleotides were incorporated using an NKK pattern and a MNN pattern, during synthesis of the positive and negative strand randomized portions respectively, where N represents any nucleotide, K represents T or G and M represents A or C. An NNT strategy eliminates stop codons and the frequency of each amino acid is less biased but omits Q, E, K, M, and W. Other doping strategies include all four nucleotide monomers (A, G, C, T), but at different frequencies. For example, a doping strategy can be designed whereby at each position within the randomized portion, the sequence is biased toward the wild-type sequence or the reference sequence. Other well-known doping strategies can be used with the methods provided herein, including parsimonious mutagenesis (see, for example, Balint et al., Gene (1993) 137(1), 109-118; Chames et al., The Journal of Immunology (1998) 161, 5421-5429), partially biased doping strategies, for example, to bias the randomized portion toward a particular sequence, e.g. a wild-type sequence (see, for example, De Kruif et al., J. Mol. Biol., (1995) 248, 97-105), doping strategies based on an amino acid code with fewer than all possible amino acids, for example, based on a four-amino acid code (see, for example, Fellouse et al., PNAS (2004) 101(34) 12467-12472), and codon-based mutagenesis and modified codon-based mutagenesis (See, for example, Gaytán et al., Nucleic Acids Research, (2002), 30(16), U.S. Pat. Nos. 5,264,563 and 7,175,996).

As used herein, a polynucleotide duplex is any double stranded polynucleotide containing complementary positive and a negative strand polynucleotides. The duplex can contain any number of nucleic acids in length, typically at least at or about 10, 11, 12, 13, 14, 15, 20, 25, 30, 40, 50 nucleotides in length. In some examples, the duplexes contain at least at or about 50, 100, 150, 200, 250, 500, 1000, 1500, 2000 or more nucleotides in length. In other examples, the duplexes contain less than at or about 500 nucleotides in length, for example, less than at or about 250, 200, 150, 100 or 50 nucleotides in length. In another example, the duplex contains the number of nucleotides in length of an entire nucleotide sequence of a gene. Exemplary of a polynucleotide duplex is an oligonucleotide duplex. Duplexes can be formed in a plurality of ways in the provided methods. For example, two or more polynucleotides can be hybridized through complementary regions to form duplexes. In another example, a polymerase reaction, e.g. a single primer extension or an amplification (e.g. PCR) reaction can be used to generate duplexes from single stranded polynucleotides.

As used herein, “assembled polynucleotide duplex” and “assembled duplex” refer synonymously to a polynucleotide duplex made according to the methods herein, having a sequence of nucleotides containing sequences analogous to two or more, typically three or more, for example, 2, 3, 4, 5, 6, 7, 8, 9, 10, 15, 20 or more, synthetic oligonucleotides and/or polynucleotides. Typically, the assembled duplexes are variant duplexes, contained in pools of assembled duplexes. In one example, the assembled duplex is a randomized assembled duplex, which contains one or more randomized portions, for example, 1, 2, 3, 4, 5, 6, 7, 8, 9, 10, 15, 20 or more randomized portions.

Similarly, “Assembled polynucleotide” refers to a polynucleotide made according to the methods herein, having a sequence of nucleotides containing sequences analogous to two or more, typically three or more, for example, 2, 3, 4, 5, 6, 7, 8, 9, 10, 15, 20 or more, synthetic oligonucleotides and/or polynucleotides, such as, but not limited to one strand of an assembled duplex, formed by denaturing the duplex.

As used herein, a collection of assembled polynucleotide duplexes is a collection containing two or more analogous assembled polynucleotide duplexes. Typically, the collection is a collection of variant assembled polynucleotide duplexes, typically randomized assembled polynucleotide duplexes, where the duplexes contain one or more randomized portions that vary compare to the other members of the collection.

As used herein, a large assembled duplex is an assembled duplex containing more than about 50 nucleotides in length, for example, greater than 50, 100, 150, 200, 250, 300, 350, 400, 450, 500, 1000, 1500, 2000 or more nucleotides in length. Typically, a randomized large assembled duplex contains two or more randomized portions, for example 2, 3, 4, 5, 6, 7, 8, 9, 10, 15, 20 or more randomized portions. Typically, at least two of the two or more of the randomized portions within a randomized large assembled duplex cassette are separated by at least about 30 nucleotides, for example, at least about 30, 35, 40, 45, 50, 55, 60, 65, 70, 75, 80, 85, 90, 95, 100, 150, 200, 250 or more nucleotides, along the linear sequence of the duplex cassette.

As used herein, “duplex cassette” refers to any oligonucleotide or polynucleotide duplex (e.g. an assembled duplex) that is capable of being directly inserted into a vector. Typically, the duplex cassette contains two restriction site overhangs that function as “sticky ends” for insertion into a vector cut by restriction endonucleases that cut at those restriction sites. Similarly, “assembled duplex cassette” is used to refer to an assembled duplex that is capable of being directly inserted into a vector. Typically, the duplex cassette contains two restriction site overhangs that function as “sticky ends” for insertion into a vector cut by restriction endonucleases that cut at those restriction sites. Provided herein are collections of assembled duplex cassettes, including randomized assembled duplex cassettes.

As used herein, an intermediate duplex (e.g. intermediate duplex cassette) is any duplex generated in the provided processes for generating collections of variant polynucleotides, such as methods for generating collections of assembled duplexes and duplex cassettes. Further steps are performed using the intermediate duplexes, in order to generate the final products, such as the assembled duplexes or duplex cassettes.

As used herein, a reference sequence duplex is a polynucleotide duplex having identity to a target polynucleotide or region thereof and optionally containing one or more additions, deletions, substitutions and/or insertions. In one example, the reference sequence duplex contains at or about 100% identity to the target polynucleotide or region thereof. In another example, the reference sequence duplex further contains additional portions and/or regions, for example, regions of complementarity/identity to a non gene-specific primer, restriction endonuclease recognition sites, and/or other non gene-specific sequence, including regulatory regions. For example, the reference sequence duplex can contain at or about, or at least at or about 50%, 55%, 60%, 65%, 70%, 75%, 80%, 85%, 90%, 95%, 96%, 97%, 98%, or 99%, or fraction thereof, identity to the target polynucleotide or region thereof. In one example of the provided methods, reference sequence duplexes are combined with randomized oligonucleotide duplexes to assemble intermediate duplexes and assembled duplexes.

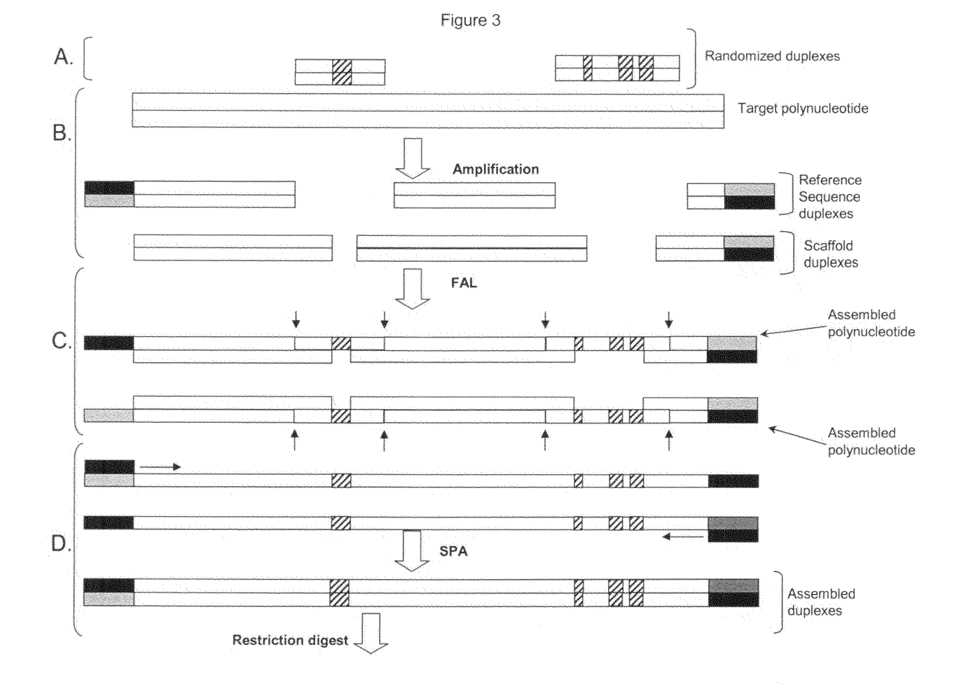

As used herein, a scaffold duplex is a polynucleotide duplex containing regions of complementarity to regions within oligonucleotides or polynucleotides within two different pools of oligonucleotides or polynucleotides or pools of duplexes. Typically, the scaffold duplex is a reference sequence duplex. Exemplary of scaffold duplexes are duplexes that contain a region of complementarity to a region in synthetic oligonucleotides in a pool of randomized oligonucleotides, and a region of complementarity to polynucleotides in another pool of reference sequence duplexes or oligonucleotide duplexes. In one example, the scaffold duplexes is used to assemble intermediate duplexes or assembled polynucleotides by combining the scaffold duplexes and the duplexes with which they share complementarity, which can facilitate ligation of oligonucleotides from the different pools. An example of scaffold duplexes is illustrated in FIG. 3, which depicts the Fragment Assembly and Ligation/Single Primer Amplification (FAL-SPA) method, where intermediate duplexes are formed by hybridizing polynucleotides and oligonucleotides from different pools to strands from scaffold duplexes.

As used herein, a genetic element refers to a gene or nucleic acid, or any region thereof, that encodes a polypeptide or protein or region thereof. In some examples, a genetic element encodes a fusion protein.

As used herein, regulatory region of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g. a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, an “amino acid” is an organic compound containing an amino group and a carboxylic acid group. A polypeptide contains two or more amino acids. For purposes herein, amino acids include the twenty naturally-occurring amino acids, non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see Table 1). The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagine |
| B | Asx | Asn and/or Asp |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence modified, non-natural and unusual amino acids. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions may be made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-isostereomers of amino acids. Exemplary non-natural amino acids are known to those of skill in the art.

As used herein, “similarity” between two proteins or nucleic acids refers to the relatedness between the sequence of amino acids of the proteins or the nucleotide sequences of the nucleic acids. Similarity can be based on the degree of identity of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. Identity refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

As used herein, a positive strand polynucleotide refers to the “sense strand” or a polynucleotide duplex, which is complementary to the negative strand or the “antisense” strand. In the case of polynucleotides which encode genes, the sense strand is the strand that is identical to the mRNA strand that is translated into a polypeptide, while the antisense strand is complementary to that strand. Positive and negative strands of a duplex are complementary to one another.

As used herein, a pair of positive strand and negative strand pools refers to two pools of oligonucleotides, one pool containing positive strand oligonucleotides, and the other pool containing negative strand oligonucleotides, where the oligonucleotides in the positive strand pool are complementary to oligonucleotides in the negative strand pool.

As used herein, “deletion,” when referring to a nucleic acid or polypeptide sequence, refers to the deletion of one or more nucleotides or amino acids compared to a sequence, such as a target polynucleotide or polypeptide or a native or wild-type sequence.

As used herein, “insertion” when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence. As used herein, “additions,” to nucleic acid and amino acid sequences describe addition of nucleotides or amino acids onto either termini compared to another sequence.

As used herein, “substitution” refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Substitution mutations compared to a particular polypeptide can be expressed in terms of the number of the amino acid residue along the length of the polypeptide sequence. For example, a modified polypeptide having a modification in the amino acid at the 19^{th} position of the amino acid sequence that is a substitution of Isoleucine (Ile; I) for cysteine (Cys; C) can be expressed as I19C, Ile19C, or simply C19, to indicate that the amino acid at the modified 19^{th} position is a cysteine. In this example, the molecule having the substitution has a modification at Ile 19 of the unmodified polypeptide.

As used herein, “primary sequence” refers to the sequence of amino acid residues in a polypeptide or the sequence of nucleotides in a nucleic acid molecule.

As used herein, it also is understood that the terms “substantially identical” or “similar” varies with the context as understood by those skilled in the relevant art, but that those of skill can assess such.

As used herein, “primer” refers to a nucleic acid molecule (more typically, to a pool of such molecules sharing sequence identity) that can act as a point of initiation of template-directed nucleic acid synthesis under appropriate conditions (for example, in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers (e.g. two pools of primers) that includes a 5′ (upstream) primer that specifically hybridizes with the 5′ end of a sequence to be amplified (e.g. by PCR) and a 3′ (downstream) primer that specifically hybridizes with the complement of the 3′ end of the sequence to be amplified. Because “primer” can refer to a pool of identical nucleic acid molecules, a primer pair typically is a pair of two pools of primers.

As used herein, “single primer” and “single primer pool” refer synonymously to a pool of primers, where each primer in the pool contains sequence identity with the other primer members, for example, a pool of primers where the members share at least at or about 50, 55, 60, 65, 70, 75, 80, 85, 90, 95, 96, 97, 98, 99 or 100% identity. The primers in the single primer pool (all sharing sequence identity) act both as 5′ (upstream) primers (that specifically hybridize with the 5′ end of a sequence to be amplified (e.g. by PCR)) and as 3′ (downstream) primers (that specifically hybridize with the complement of the 3′ end of the sequence to be amplified). Thus, the single primer can be used, without other primers, to prime synthesis of complementary strands and amplify a nucleic acid in a polymerase amplification reaction. In one example, the single primer is used without other primers to amplify a nucleic acid in an amplification reaction, e.g. by hybridizing to a 5′ sequence in both strands of a polynucleotide duplex. In one such example, a single primer is used to prime complementary strand synthesis (e.g. in a PCR amplification) from the termini (e.g. 5′ termini) of both strands of an oligonucleotide duplex.

As used herein, complementarity, with respect to two nucleotides, refers to the ability of the two nucleotides to base pair with one another upon hybridization of two nucleic acid molecules. Two nucleic acid molecules sharing complementarity are referred to as complementary nucleic acid molecules; exemplary of complementary nucleic acid molecules are the positive and negative strands in a polynucleotide duplex. As used herein, when a nucleic acid molecule or region thereof is complementary to another nucleic acid molecule or region thereof, the two molecules or regions specifically hybridize to each other. Two complementary nucleic acid molecules often are described in terms of percent complementarity. For example, two nucleic acid molecules, each 100 nucleotides in length, that specifically hybridize with one another but contain 5 mismatches with respect to one another, are said to be 95% complementary. For two nucleic acid molecules to hybridize with 100% complementarity, it is not necessary that complementarity exist along the entire length of both of the molecules. For example, a nucleic acid molecule containing 20 contiguous nucleotides in length can specifically hybridize to a contiguous 20 nucleotide portion of a nucleic acid molecule containing 500 contiguous nucleotide in length. If no mismatches occur along this 20 nucleotide portion, the 20 nucleotide molecule hybridizes with 100% complementarity. Typically, complementary nucleic acid molecules align with less than 25%, 20%, 15%, 10%, 5% 4%, 3%, 2% or 1% mismatches between the complementary nucleotides (in other words, at least at or about 75%, 80%, 85%, 90%, 95, 96%, 97%, 98% or 99% complementarity). In another example, the complementary nucleic acid molecules contain at or about or at least at or about 50%, 55%, 60%, 65%, 70%, 75%, 80%, 85%, 90%, 95, 96%, 97%, 98% or 99% complementarity. In one example, complementary nucleic acid molecules contain fewer than 5, 4, 3, 2 or 1 mismatched nucleotides. In one example, the complementary nucleotides are 100% complementary. If necessary, the percentage of complementarity will be specified. Typically the two molecules are selected such that they will specifically hybridize under conditions of high stringency.

As used herein, a complementary strand of a nucleic acid molecule refers to a sequence of nucleotides, e.g. a nucleic acid molecule, that specifically hybridizes to the molecule, such as the opposite strand to the nucleic acid molecule in a polynucleotide duplex. For example, in a polynucleotide duplex, the complementary strand of a positive strand oligonucleotide is a negative strand oligonucleotide that specifically hybridizes to the positive strand oligonucleotide in a duplex. In one example of the provided methods, polymerase reactions are used to synthesize complementary strands of polynucleotides to form duplexes, typically beginning by hybridizing an oligonucleotide primer to the polynucleotide.

As used herein, “region of complementarity” or “portion of complementarity” are used synonymously with “complementary region” or “complementary portion,” respectively, to refer to the region or portion, respectively, of one complementary nucleic acid molecule that specifically hybridizes to a corresponding complementary region or portion on another complementary nucleic acid molecule. For example, the synthetic oligonucleotides produced according to the methods provided herein can contain one or more regions of complementarity to one or more other oligonucleotides, for example, to a fill-in primer. Typically, for specific hybridization of a synthetic oligonucleotide to another polynucleotide, particularly to another oligonucleotide, the synthetic oligonucleotide contains a 5′ and a 3′ region complementary to the other polynucleotide. Typically, each of the 5′ and the 3′ regions of complementarity contains at least about 10 nucleotides in length, for example, at least about 10, 11, 12, 13, 14, 15, 16, 17, 18, 19, 20, 21, 22, 23, 24, 25 or more nucleotides in length.

As used herein, “region of identity” or “portion of identity” are used synonymously with “identical region” or “identical portion,” respectively, to refer to a region or portion, respectively, of one nucleic acid molecule having at least at or about 40% sequence identity, and typically at least at or about 50%, 55%, 60%, 65%, 70%, 75%, 80%, 85%, 90%, 95%, 96%, 97%, 98%, 99% or more, such as 100%, sequence identity to a region or portion in another nucleic acid molecule; specific percent identities can be specified. Typically, the region/portion of identity specifically hybridizes to a sequence of nucleotides that is complementary to the nucleic acid region to which it is identical. For example, the synthetic oligonucleotides produced according to the methods provided herein can contain one or more regions of identity to portions or regions in other polynucleotides, such as other oligonucleotides or target polynucleotides. Typically, the region of identity contains at least about 10 nucleotides in length, for example, at least about 10, 11, 12, 13, 14, 15, 16, 17, 18, 19, 20, 21, 22, 23, 24, 25 or more nucleotides in length.

As used herein, “specifically hybridizes” refers to annealing, by complementary base-pairing, of a nucleic acid molecule (e.g. an oligonucleotide or polynucleotide) to another nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. It is not necessary that two nucleic acid molecules exhibit 100% complementarity in order to specifically hybridize to one another. For example, two complementary nucleic acid molecules sharing sequence complementarity, such as at or about or at least at or about 99%, 98%, 97%, 96%, 95%, 90%, 85%, 80%, 75%, 70%, 65%, 60%, 55% or 50% complementarity, can specifically hybridize to one another. Parameters, for example, buffer components, time and temperature, used in in vitro hybridization methods provided herein, can be adjusted in stringency to vary the percent complementarity required for specific hybridization of two nucleic acid molecules. The skilled person can readily adjust these parameters to achieve specific hybridization of a nucleic acid molecule to a target nucleic acid molecule appropriate for a particular application.

As used herein, “specifically bind” with respect to an antibody refers to the ability of the antibody to form one or more noncovalent bonds with a cognate antigen, by noncovalent interactions between the antibody combining site(s) of the antibody and the antigen.

As used herein, an effective amount of a therapeutic agent is the quantity of the agent necessary for preventing, curing, ameliorating, arresting or partially arresting a symptom of a disease or disorder.

As used herein, unit dose form refers to physically discrete units suitable for human and animal subjects and packaged individually as is known in the art.

As used herein, the singular forms “a,” “an” and “the” include plural referents unless the context clearly dictates otherwise. Thus, for example, reference to compound, comprising “an extracellular domain” includes compounds with one or a plurality of extracellular domains.

As used herein, ranges and amounts can be expressed as “about” a particular value or range. About also includes the exact amount. Hence “about 5 bases” means “about 5 bases” and also “5 bases.’

As used herein, “optional” or “optionally” means that the subsequently described event or circumstance does or does not occur and that the description includes instances where said event or circumstance occurs and instances where it does not. For example, an optionally variant portion means that the portion is variant or non-variant. In another example, an optional ligation step means that the process includes a ligation step or it does not include a ligation step.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

As used herein, a template oligonucleotide or template polynucleotide (also called oligonucleotide template or polynucleotide template) is an oligonucleotide or polynucleotide used as a template in a polymerase extension reaction, for example, in a fill-in reaction, a single-primer amplification reaction, a polymerase chain reaction (PCR) or other polymerase-driven reaction. Any of the synthetic oligonucleotides can be used as template oligonucleotides. The template oligonucleotide contains at least one region that is complementary to primers, such as primers in a primer pool, for example, fill-in primers, non gene-specific primers, primers containing a restriction site sequence, gene-specific primers, single primer pools and primer pairs.

As used herein, a fill-in primer is an oligonucleotide that specifically hybridizes to a template oligonucleotide or polynucleotide and primes a fill-in reaction, whereby a sequence of nucleotides complementary to the template strand is synthesized, thereby generating an oligonucleotide duplex. A single oligonucleotide can both be a template oligonucleotide and a fill-in primer. For example, two oligonucleotides, sharing a region of complementarity, can participate in a mutually primed fill-in reaction, whereby one oligonucleotide primes synthesis of the complementary strand of the other nucleotide, and vice versa. A fill-in reaction is a polymerase reaction carried out using a fill-in primer.

As used herein, a mutually primed fill-in reaction is a fill-in reaction whereby each of two oligonucleotides serves as a fill-in primer to prime synthesis of a strand complementary to the other oligonucleotide. Thus, the two oligonucleotides are both template oligonucleotides and fill-in primers. The two oligonucleotides share at least one region of complementarity. A mutually-primed synthesis reaction can one oligonucleotide serves as a fill-in primer for the other oligonucleotide and vice versa.

As used herein, a non gene-specific sequence is a sequence of nucleotides, for example, in a vector, that does not encode a polypeptide, such as a non-encoding sequence, for example, a regulatory sequence, such as a bacterial leader sequence, promoter sequence, or enhancer sequence; a sequence of nucleotides that is a restriction endonuclease recognition site; and/or a sequence having complementarity to a primer.

As used herein, a non gene-specific primer is a primer that binds to a non gene-specific nucleic acid sequence in a template polynucleotide or oligonucleotide and primes synthesis of the complementary strand of the polynucleotide in an amplification reaction, typically a single-primer extension reaction. Typically, the non gene-specific primer specifically hybridizes to a region of the polynucleotide that corresponds to the non gene-specific region of the polynucleotide, for example, a bacterial promoter sequence or portion thereof.

Alternatively, a gene-specific primer is a primer that binds within a sequence of nucleotides encoding a polypeptide, such as a target or variant polypeptide.

As used herein, a host cell is a cell that is used in to receive, maintain, reproduce and amplify a vector. A host cell also can be used to express the polypeptide encoded by the vector nucleotides, for example, a variant polypeptide. The nucleic acid inserted in the vector, typically a duplex cassette, is replicated when the host cell divides, thereby amplifying the cassette nucleic acids. In one example, the host cell is a genetic package, which can be induced to express the variant polypeptide on its surface. In another example, for example when the genetic package is a virus, for example, a phage, the host cell is infected with the genetic package. For example, the host cells can be phage-display compatible host cells, which can be transformed with phage or phagemid vectors and accommodate the packaging of phage expressing fusion proteins containing the variant polypeptides.

As used herein, a vector is a replicable nucleic acid from which one or more heterologous proteins can be expressed when the vector is transformed into an appropriate host cell and/or introduced into a genetic package. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell and/or genetic package for amplification of the nucleic acid or for expression/display of the polypeptide encoded by the nucleic acid. When the genetic package is a virus, for example, a phage, the genetic package can also be the vector. Alternatively, for example, in the case of phage display, a phagemid vector is used as the vector to introduce the nucleic acids into the genetic package. In this case, the phagemid vector is transformed into a host cell, typically a bacterial host cell. In one example, a helper phage is co-infected to induce packaging of the phage (genetic package), which will express the encoded polypeptide.

As used herein, a genetic package is a vehicle used to display a polypeptide, typically a variant polypeptide produced according to the provided methods. Typically, the genetic package displaying the polypeptide is used for selection of desired variant polypeptides from a collection of variant polypeptides. Genetic packages that can be used with the provided methods include, but are not limited to, bacterial cells, bacterial spores, viruses, including bacterial DNA viruses, for example, bacteriophages, typically filamentous bacteriophages, for example, Ff, M13, fd, and fl. Any of a number of well-known genetic packages can be used in association with the provided methods. A genetic package polypeptide is any polypeptide naturally expressed by the polypeptide, or variant thereof.

As used herein, display refers to the expression of one or more polypeptides on the surface of a genetic package, such as a phage. As used herein, phage display refers to the expression of polypeptides on the surface of filamentous bacteriophage.

As used herein, a phage-display compatible cell or phage-display compatible host cell is a host cell, typically a bacterial host cell, that can be infected by phage and thus can support the production of phage displaying fusion proteins containing polypeptides, e.g. variant polypeptides and can thus be used for phage display. Exemplary of phage display compatible cells include, but are not limited to, XL1-blue cells.

As used herein, panning refers to an affinity-based selection procedure for the isolation of phage displaying a molecule with a specificity for a binding partner, for example, a capture molecule (e.g. an antigen) or sequence of amino acids or nucleotides or epitope, region, portion or locus therein.

As used herein, transformation efficiency refers to the number of bacterial colonies produced per mass of plasmid DNA transformed (colony forming units (cfu) per mass of transformed plasmid DNA).

As used herein, titer with reference to phage refers to the number of colony forming units (cfu) per ml of transformed cells.

As used herein, in silico means performed or contained on a computer or via computer simulation.

As used herein, a stop codon is used to refer to a three-nucleotide sequence that signals a halt in protein synthesis during translation, or any sequence encoding that sequence (e.g. a DNA sequence encoding an RNA stop codon sequence), including the amber stop codon (UAG or TAG)), the ochre stop codon (UAA or TAA)) and the opal stop codon (UGA or TGA)). It is not necessary that the stop codon signal termination of translation in every cell or in every organism. For example, in suppressor strain host cells, such as amber suppressor strains and partial amber suppressor strains, translation proceeds through one or more stop codon (e.g. the amber stop codon for an amber suppressor strain), at least some of the time.

As used herein, the phrase “compared to in the absence of the stop codon” when referring to expression or toxicity of a polypeptide, refers to the expression or toxicity of the polypeptide when expressed from a vector provided herein that contains one or more stop codons that result in limited translation (i.e. translation only some of the time) of the polypeptide, compared the expression or toxicity of the same polypeptide when expressed from a comparable vector, such as the same vector or a vector with comparable characteristics, that does not contain the one or more stop codons that result in limited translation of the polypeptide, when the vectors are introduced into an appropriate partial suppressor cell. For example, the toxicity of the domain exchanged 2G12 Fab fragment when expressed from the 2G12 pCAL IT* vector (that contains amber stop codons in the Pel B and Omp A leader sequences) in an amber suppressor cell is reduced compared to toxicity of the 2G12 Fab fragment when expressed from the 2G12 pCAL G13 vector (that does not contain amber stop codons in the Pel B and Omp A leader sequences) in an amber suppressor cell. Thus, the toxicity of the 2G12 Fab fragment to the host cell expressed from the 2G12 pCAL IT* vector in partial amber suppressor cells is reduced compared to in the absence of the stop codons.

As used herein, a suppressor strain or a suppressor cell refers to organisms or cell (e.g. host cell), in which translation proceeds through a stop codon or termination sequence (read-through) for some percentage of the time. Stop codon suppressor strains contain mutation(s) causing the production of tRNA having altered anti-codons that can read the stop codon sequence, allowing continued protein synthesis. For example, cells of an amber suppressor strain, such as, but not limited to, XL1-Blue cells, contain altered tRNA (e.g. a UAG suppression tRNA gene (having a sup E44 genotype)) allowing them to read through the UAG codon and continue protein synthesis. In suppressor strains containing a sup E44 gene, a glutamine (Gln; Q) is produced from the UAG codon. In one example, the suppressor strains are partial suppressor strains, where translation proceeds through the stop codon less than 100% of the time (thus, effecting less than 100% suppression or read-through), typically no more than 80% suppression, typically no more than 50% suppression, such as no more than at or about 80, 75, 70, 65, 60, 55, 50, 45, 40, 35, 30, 25, 20, or 15% suppression. Efficiency of suppression can depend on several factors, such as the choice of polynucleotide, e.g. vector, containing the amber stop codon. For example, the choice of nucleotide immediately to the 3′ of an amber stop codon can affect the amount of read-through, for example, whether the vector contains a guanine residue or an adenine residue at the position just 3′ of the amber stop codon. Exemplary of partial suppressor strains are amber suppressor strains, e.g. XL1-Blue cells, which carry the E44 genotype. Other suppressor strains are well known (see, e.g. Huang et al., J. Bacteria 174(16) 5436-5441 (1992) and Bullock et al., Biotechniques 5:376-379 (1987)).

As used herein, randomized duplexes are oligonucleotide duplexes containing randomized oligonucleotides and having one or more randomized portions.

As used herein, a ligase is an enzyme capable of creating a covalent bond between a 5′ terminus of one nucleic acid molecule and a 3′ terminus of another nucleic acid molecule, when the 5′ terminus of the first nucleic acid molecule and the 3′ terminus of the second nucleic acid molecule are hybridized to portions on a third nucleic acid molecule, such as a complementary nucleic acid molecule. Thus, a ligase can be used to seal a nick between the 5′ and 3′ termini of two nucleic acid molecules each hybridized to a third nucleic acid molecule, thus forming a duplex. A ligase also can be used to join nucleic acid duplexes with overhangs, for example, restriction site overhangs, such as for insertion into a vector. When the ligase joins the nick between the 5′ and 3′ termini, the 5′ and 3′ nucleic acids of the respective molecules become adjacent nucleotides in the resulting duplex.

The ligase can be any of a number of well-known ligases, such as for example, T4 DNA ligase (from bacteriophage T4) (commercially available, for example, from New England Biolabs, Beverly, Mass.), T7 DNA ligase (from bacteriophage T7), E. coli ligase, tRNA ligase, a ligase from yeast, a ligase from an insect cell, a ligase from a mammal (e.g., murine ligase), and human DNA ligase (e.g., human DNA ligase IV/XRCC4). Exemplary of the ligases used in this step are a DNA ligase, for example, T4 DNA ligase or E. coli DNA ligase, an RNA ligase, for example, T4 RNA ligase, and a thermostable ligase, for example, Ampligase® (EPICENTRE® Biotechnologies, Madison, Wis.). An exemplary ligation reaction is carried out at room temperature, for example at 25° C., for four hours.

As used herein, “nick” describes the break between the 5′ and 3′ termini of two adjacent nucleic acid molecules (both hybridized to a third nucleic acid molecule), which can be joined by formation of a covalent phosphodiester bond by a ligase, producing a duplex. Thus, to “seal” a nick is to cause the formation of the bonds between the adjacent 5′ and 3′ terminal nucleotides in the two molecules, forming a duplex.

As used herein, a restriction enzyme or restriction endonuclease refers to an enzyme that cleaves a polynucleotide duplexes between two or more nucleotides, by recognizing short sequences of nucleotides, called restriction sites or restriction endonuclease recognition sites. Restriction endonucleases, and their recognition sites are well known and any of the known enzymes can be used with the provided methods. Often, cleavage of a duplex by a restriction endonuclease results in “restriction site overhangs,” also called “sticky ends,” which contain a single strand portion on one or both termini of the polynucleotide duplex and can be used in the provided methods to hybridize duplexes containing complementary overhangs, such as for ligation into a vector.

As used herein, “overhang” refers to a 5′ or 3′ portion of a polynucleotide duplex that is single stranded. Thus, while the duplex is a double-stranded nucleic acid molecule, with pairing through complementary nucleotides, the overhangs are single-strand portions that do not pair with complementary nucleotides and “hang over” the end of the duplex. Exemplary of overhangs are restriction site overhangs, which are generated by cutting with restriction enzymes; each restriction enzyme produces characteristic overhangs by cutting at particular sites in double stranded nucleic acid molecules.

As used herein, a single primer extension reaction is a method whereby a complementary strand of a polynucleotide is synthesized using a single primer (e.g. a single primer pool) and a polymerase. Typically, the single primer extension is not an amplification reaction, and thus does not include multiple rounds or cycles. Thus, one complementary strand is synthesized and multiple copies are not produced.

As used herein “amplification” refers to a method for increasing the number of copies of a sequence of a polynucleotide using a polymerase and typically, a primer. An amplification reaction results in the incorporation of nucleotides to elongate a polynucleotide molecule, such as a primer, thereby forming a polynucleotide molecule, e.g. a complementary strand, which is complementary to a template polynucleotide. In one example, the formed new polynucleotide strand can then be used as a template for synthesis of an additional complementary polynucleotide in a subsequent cycle. Typically, one amplification reaction includes many rounds (“cycles”) of this process, whereby polynucleotides in the first round or cycle are denatured and used as template polynucleotides in a subsequent cycle. Each cycle includes one extension reaction, whereby a complementary strand is synthesized. Amplification reactions include, but are not limited to, polymerase chain reactions (PCR), reverse-transcriptase (RT)-PCR, RNA PCR, LCR, multiplex PCR, panhandle PCR, capture PCR, expression PCR, 3′ and 5′ RACE, in situ PCR and ligation-mediated PCR.

As used herein, “binding partner” refers to a molecule (such as a polypeptide, lipid, glycolipid, nucleic acid molecule, carbohydrate or other molecule), with which another molecule specifically interacts, for example, through covalent or noncovalent interactions, such as the interaction of an antibody with cognate antigen. The binding partner can be naturally or synthetically produced. In one example, desired variant polypeptides are selected using one or more binding partners, for example, using in vitro or in vivo methods. Exemplary of the in vitro methods include selection using a binding partner coupled to a solid support, such as a bead, plate, column, matrix or other solid support; or a binding partner coupled to another selectable molecule, such as a biotin molecule, followed by subsequent selection by coupling the other selectable molecule to a solid support. Typically, the in vitro methods include wash steps to remove unbound polypeptides, followed by elution of the selected variant polypeptide(s). The process can be repeated one or more times in an iterative process to select variant polypeptides from among the selected polypeptides.

As used herein, a binding activity is a characteristic of a molecule, e.g. a polypeptide, relating to whether or not, and how, it binds one or more binding partners. Binding activities include ability to bind the binding partner(s), the affinity with which it binds to the binding partner (e.g. high affinity), the avidity with which it binds to the binding partner, the strength of the bond with the binding partner and specificity for binding with the binding partner.

As used herein, affinity describes the strength of the interaction between two or more molecules, such as binding partners, typically the strength of the noncovalent interactions between two binding partners. The affinity of an antibody for an antigen epitope is the measure of the strength of the total noncovalent interactions between a single antibody combining site and the epitope. Low-affinity antibody-antigen interaction is weak, and the molecules tend to dissociate rapidly, while high affinity antibody-antigen binding is strong and the molecules remain bound for a longer amount of time. Methods for calculating affinity are well known, such as methods for determining dissociation constants. Affinity can be estimated empirically or affinities can be determined comparatively, e.g. by comparing the affinity of one antibody and another antibody for a particular antigen. Affinity can be compared to another antibody, for example, “high affinity” of a variant antibody polypeptide or modified antibody polypeptide can refer to affinity that is greater than the affinity of the target or unmodified antibody.

As used herein, “off-rate” when referring to an antibody, refers to the dissociation rate constant (k_{ff}), or rate at which the antibody dissociates from bound antigen. Off-rate can be compared to another antibody, for example, “low off rate” of a variant antibody polypeptide or modified antibody polypeptide can refer to an off-rate that is lower than the off-rate of the target or unmodified antibody.

As used herein, “on-rate,” when referring to an antibody, refers to the dissociation rate constant (kₒₙ), or rate at which the antibody associates (binds) to its antigen. On-rate can be compared to another antibody, for example, “high on-rate” of a variant antibody polypeptide or modified antibody polypeptide can refer to an on-rate that is greater than the on-rate of the target or unmodified antibody.

As used herein, antibody avidity refers to the strength of multiple interactions between a multivalent antibody and its cognate antigen, such as with antibodies containing multiple binding sites associated with an antigen with repeating epitopes or an epitope array. A high avidity antibody has a higher strength of such interactions compared with a low avidity antibody.

As used herein, a high-fidelity polymerase is a polymerase that can be used to perform polymerase reactions with an error frequency rate that is not more than at or about 4×10⁻⁶ mutations per base pair per amplification cycle (e.g. PCR cycle), such as, for example, not more than at or about 2×10⁻⁶, and not more than at or about 1.3×10⁻⁶ mutations per base pair per cycle, or fewer. In one example, the high-fidelity polymerase is an error-free polymerase. A particular error rate can be specified. Exemplary of high fidelity polymerases is the Advantage® HF 2 polymerase (Clonetech), which produces at or about 30-fold higher fidelity than Taq polymerase.

As used herein, “coupled” means attached via a covalent or noncovalent interaction. For example, in the provided methods, one or more binding partners can be coupled to a solid support for selection of variant polypeptides.

As used herein, “bind” refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs. Exemplary of bonds are antibody-antigen interactions and receptor-ligand interactions. When an antibody “binds” a particular antigen, bind refers to the specific recognition of the antigen by the antibody, through cognate antibody-antigen interaction, at antibody combining sites. Binding can also include association of multiple chains of a polypeptide, such as antibody chains which interact through disulfide bonds.

As used herein, a disulfide bond (also called an S—S bond or a disulfide bridge) is a single covalent bond derived from the coupling of thiol groups. Disulfide bonds in proteins are formed between the thiol groups of cysteine residues, and stabilize interactions between polypeptide domains, such as antibody domains.

As used herein, “display protein” and “genetic package display protein” refer synonymously to any genetic package polypeptide for display of a polypeptide on the genetic package, such that when the display protein is fused to (e.g. included as part of a fusion protein with) a polypeptide of interest (e.g. target or variant polypeptide provided herein), the polypeptide is displayed on the outer surface of the genetic package. The display protein typically is present on or within the outer surface or outer compartment of a genetic package (e.g. membrane, cell wall, coat or other outer surface or compartment) of a genetic package, e.g. a viral genetic package, such as a phage, such that upon fusion to a polypeptide of interest, the polypeptide is displayed on the genetic package.

As used herein, a coat protein is a display protein, at least a portion of which is present on the outer surface of the genetic package, such that when it is fused to the polypeptide of interest, the polypeptide is displayed on the outer surface of the genetic package. Typically, the coat proteins are viral coat proteins, such as phage coat proteins. A viral coat protein, such as a phage coat protein associates with the virus particle during assembly in a host cell. In one example, coat proteins are used herein for display of polypeptides on genetic packages; the coat proteins are expressed as portions of fusion proteins, which contain the coat protein sequence of amino acids and a sequence of amino acids of the displayed polypeptide, such as a variant polypeptide provided herein. In the provided methods, nucleic acid encoding the coat protein is inserted in a vector adjacent or in close proximity to the nucleic acid encoding the polypeptide, e.g. the variant polypeptide. The coat protein can be a full-length coat protein or any portion thereof capable of effecting display of the polypeptide on the surface of the genetic package.

Exemplary of coat proteins are phage coat proteins, such as, but not limited to, (i) minor coat proteins of filamentous phage, such as gene III protein (gIIIp, cp3), and (ii) major coat proteins (which are present in the viral coat at 10 copies or more, for example, tens, hundreds or thousands of copies) of filamentous phage such as gene VIII protein (gVIIIp, cp8); fusions to other phage coat proteins such as gene VI protein, gene VII protein, or gene IX protein (see, e.g., WO 00/71694); and portions (e.g., domains or fragments) of these proteins, such as, but not limited to domains that are stably incorporated into the phage particle, e.g. such as the anchor domain of gIIIp, or gVIIIp. Additionally, mutants of gVIIIp can be used which are optimized for expression of larger peptides, such as mutants having improved surface display properties, such as mutant gVIIp (see, for example, Sidhu et al. (2000) J. Mol. Biol. 296:487-495).

As used herein, a fusion protein is a polypeptide engineered to contain sequences of amino acids corresponding to two distinct polypeptides, which are joined together, such as by expressing the fusion protein from a vector containing two nucleic acids, encoding the two polypeptides, in close proximity, e.g. adjacent, to one another along the length of the vector. Exemplary of a fusion protein is a coat protein-polypeptide fusion, for example, a coat protein fused to a variant polypeptide, which are displayed on the surfaces of genetic packages. A non-fusion polypeptide is a polypeptide that is not part of a fusion protein containing a coat protein, such as a soluble polypeptide.

As used herein, “adjacent” nucleotides, nucleotide sequences, nucleic acids, amino acids, amino acid residues, or amino acids, are nucleotides, nucleotide sequences, nucleic acids, amino acids, amino acid residues, or amino acids that are immediately next to one another along the length of the linear nucleic acid or amino acid sequence. When it is said that a particular nucleotide, nucleotide sequence, nucleic acid, amino acid, amino acid residue, or amino acid is “between” or “located between” two other such molecules, this description refers to the location of the sequences or residues along the linear length of the amino acid or nucleic acid sequence, unless otherwise indicated.

Exemplary of coat proteins are phage coat proteins, such as, but not limited to, (i) minor coat proteins of filamentous phage, such as gene III protein (gIIIp, cp3), and (ii) major coat proteins (which are present in the viral coat at 10 copies or more, for example, tens, hundreds or thousands of copies) of filamentous phage such as gene VIII protein (gVIIIp, cp8); fusions to other phage coat proteins such as gene VI protein, gene VII protein, or gene IX protein (see, e.g., WO 00/71694); and portions (e.g., domains or fragments) of these proteins, such as, but not limited to domains that are stably incorporated into the phage particle, e.g. such as the anchor domain of gIIIp, or gVIIIp. Additionally, mutants of gVIIIp can be used which are optimized for expression of larger peptides, such as mutants having improved surface display properties, such as mutant gVIIp (see, for example, Sidhu et al. (2000) J. Mol. Biol. 296:487-495).

As used herein, “drug-resistant” refers to the inability of an infectious agent or other microbe to be treated by drug that typically is used to treat similar types of infectious agents. It is not necessary that the drug-resistant agent be resistant to treatment with every drug.

As used herein, equimolar concentrations refers to the presence of two or more molecules at the same or about the same number of molecules within a sample, e.g. within a pool of polynucleotides.

As used herein, a “property” of a polypeptide, such as an antibody or other therapeutic polypeptide, refers to any property exhibited by a polypeptide, including, but not limited to, binding specificity, structural configuration or conformation, protein stability, resistance to proteolysis, conformational stability, thermal tolerance, and tolerance to pH conditions. Changes in properties can alter an “activity” of the polypeptide. For example, a change in the binding specificity of the antibody polypeptide can alter the ability to bind an antigen, and/or various binding activities, such as affinity or avidity, or in vivo activities of the therapeutic polypeptide.

As used herein, an “activity” or a “functional activity” of a polypeptide, such as an antibody or other therapeutic polypeptide, refers to any activity exhibited by the polypeptide. Such activities can be empirically determined. Exemplary activities include, but are not limited to, ability to interact with a biomolecule, for example, through antigen binding, DNA binding, ligand binding, or dimerization, enzymatic activity, for example, kinase activity or proteolytic activity. For an antibody (including fragments), activities include, but are not limited to, the ability to specifically bind a particular antigen, affinity of antigen binding (e.g. high or low affinity), avidity of antigen binding (e.g. high or low avidity), on-rate, off-rate, effector functions, such as the ability to promote antigen neutralization or clearance, and in vivo activities, such as the ability to prevent infection or invasion of a pathogen, or to promote clearance, or to penetrate a particular tissue or fluid or cell in the body. Activity can be assessed in vitro or in vivo using recognized assays, such as ELISA, flow cytometry, BIAcore or equivalent assays to measure on- or off-rate, immunohistochemistry and immunofluorescence histology and microscopy, cell-based assays, flow cytometry, binding assays, such as the panning assays described herein. For example, for an antibody polypeptide, activities can be assessed by measuring binding affinities, avidities, and/or binding coefficients (e.g. for on-/off-rates), and other activities in vitro or by measuring various effects in vivo, such as immune effects, e.g. antigen clearance, penetration or localization of the antibody into tissues, protection from disease, e.g. infection, serum or other fluid antibody titers, or other assays that are well know in the art. The results of such assays that indicate that a polypeptide exhibits an activity can be correlated to activity of the polypeptide in vivo, in which in vivo activity can be referred to as therapeutic activity, or biological activity. Activity of a modified polypeptide can be any level of percentage of activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of activity compared to the unmodified polypeptide. Assays to determine functionality or activity of modified (e.g. variant) antibodies are well known in the art.

As used herein. “therapeutic activity” refers to the in vivo activity of a therapeutic polypeptide. Generally, the therapeutic activity is the activity that is used to treat a disease or condition. Therapeutic activity of a modified polypeptide can be any level of percentage of therapeutic activity of the unmodified polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more of therapeutic activity compared to the unmodified polypeptide.

As used herein, “exhibits at least one activity” or “retains at least one activity” refers to the activity exhibited by a modified polypeptide, such as a variant polypeptide produced according to the provided methods, such as a modified, e.g. variant antibody or other therapeutic polypeptide (e.g. a modified 2G12 antibody), compared to the target or unmodified polypeptide, that does not contain the modification. A modified (e.g. variant) polypeptide that retains an activity of a target polypeptide can exhibit improved activity or maintain the activity of the unmodified polypeptide. In some instances, a modified (e.g. variant) polypeptide can retain an activity that is increased compared to an target or unmodified polypeptide. In some cases, a modified (e.g. variant) polypeptide can retain an activity that is decreased compared to an unmodified or target polypeptide. Activity of a modified (e.g. variant) polypeptide can be any level of percentage of activity of the unmodified or target polypeptide, including but not limited to, 1% of the activity, 2%, 3%, 4%, 5%, 10%, 20%, 30%, 40%, 50%, 60%, 70%, 80%, 90%, 91%, 92%, 93%, 94%, 95%, 96%, 97%, 98%, 99%, 100%, 200%, 300%, 400%, 500%, or more activity compared to the unmodified or target polypeptide. In other embodiments, the change in activity is at least about 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, or more times greater than unmodified or target polypeptide. Assays for retention of an activity depend on the activity to be retained. Such assays can be performed in vitro or in vivo. Activity can be measured, for example, using assays known in the art and described in the Examples below for activities such as but not limited to ELISA and panning assays. Activities of a modified (e.g. variant) polypeptide compared to an unmodified or target polypeptide also can be assessed in terms of an in vivo therapeutic or biological activity or result following administration of the polypeptide.

As used herein, a “polypeptide that is toxic to the cell” refers to a polypeptide whose heterologous expression in a host cell can be detrimental to the viability of the host cell. The toxicity associated with expression of the heterologous polypeptide can manifest, for example, as cell death or a reduced rate of cell growth, which can be assessed using methods well known in art, such as determining the growth curve of the host cell expressing the polypeptide by, for example, spectrophotometric methods, such as the optical density at 600 nm, and comparing it to the growth of the same host cell that does not express the polypeptide. Toxicity associated with expression of the polypeptide also can manifest as vector instability or nucleic acid instability. For example, the vector encoding the polypeptide can be lost from the host cell during replication of the host cell, or the nucleic acid encoding the polypeptide can be lost from the vector or can be otherwise modified to reduce expression of the heterologous polypeptide.

As used herein, a “leader peptide” or a “signal peptide” refers to a peptide that can mediate transport of a linked, such as a fused, polypeptide to the cell surface or exterior of intracellular membranes, such as to the periplasm of bacterial cells. Leader peptides typically are at least 10, 20, 30, 40, 50, 60, 70, 80 or more amino acids long. Typically, the leader peptide is linked to the N-terminus of the polypeptide to facilitate translocation of that polypeptide across an intracellular membrane Leader peptides include any of eukaryotic, prokaryotic or viral origin. Exemplary of bacterial leader peptides include, but are not limited to, the leader peptide from Pectate lyase B protein from Erwinia carotovora (PelB) and the E. coli leader peptides from the outer membrane protein (OmpA; U.S. Pat. No. 4,757,013); heat-stable enterotoxin II (StII); alkaline phosphatase (PhoA), outer membrane porin (PhoE), and outer membrane lambda receptor (LamB). Non-limiting examples of viral leader peptides include the N-terminal signal peptide from the bacteriophage proteins pIII and pVIII, pVII, and pIX. Leader peptides are encoded by leader sequences.

As used herein, “expression” refers to the process by which polypeptides are produced by transcription and translation of polynucleotides. Thus, expression of a protein requires both transcription and translation. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and the Bradford protein assay. For the purposes herein, the level of expression of a protein is measured as the amount of protein produced per cell. Thus, in instances where the expression of a protein is reduced compared to expression of the same protein in a different setting, the amount of protein produced per cell is reduced compared to the amount of protein produced from a cell in the different setting to which it is being compared. For example, if the expression of a 2G12 domain exchanged antibody from the 2G12 pCAL IT* vector in a partial suppressor cell is reduced compared to expression of a 2G12 domain exchanged antibody from the 2G12 pCAL vector in a partial suppressor cell is reduced, it means that the amount of 2G12 antibody produced from the 2G12 pCAL IT* vector in a single cell is less, on average, than the amount of 2G12 antibody produced from the 2G12 pCAL vector in a single cell.

As used herein, “located in the nucleic acid encoding” when referring to the position of a stop codon located in the nucleic acid encoding a polypeptide, means that the stop codon can be at any position in the coding sequence of the polypeptide, including in the middle of the coding sequence or at the 5′ or 3′ ends of the coding sequence.

### B. Overview of the Methods, Vectors and Display Molecules

Provided are display methods and displayed molecules, vectors for display, and collections of the displayed molecules. The displayed molecules include polypeptides, such as antibodies, and typically are domain exchanged antibodies, such as domain exchanged antibody fragments. The molecules are displayed on genetic packages, such as phage.

In general, display of polypeptides on genetic packages, e.g. in a phage display library, can be used to produce and select polypeptides from a collection, e.g. a collection of variant polypeptides; selection can be based on a desired property of the polypeptides, such as binding to a binding partner, e.g. an antigen, such as with a particular affinity. Display methods, tools and collections can be used to produce and select variant polypeptides with desired properties. Such methods and libraries can be used, for example, to generate new antibodies, such as antibodies that bind to a desired target, e.g. with a particular affinity or avidity.

Domain exchanged antibodies are characterized by a non-conventional three-dimensional configuration containing an interface between two heavy chain variable regions. The display of antibodies having this configuration on genetic packages by conventional methods, e.g. in conventional phage display, is not straightforward. Further, the expression of domain exchanged antibodies, like other antibodies, can be toxic to host cells. Thus, provided herein are methods and vectors for display of domain exchanged antibodies, wherein the toxicity associated with expression of the antibodies is reduced, and the antibodies are expressed and/or displayed on the genetic packages in the correct configuration. The provided methods and vectors also can be used to display polypeptides other than domain exchanged fragments, such as antibodies that are displayed in bivalent form, e.g. antibodies having two heavy and two light chain portions.

To facilitate display of the domain exchanged antibodies on the genetic packages, the vectors provided herein can contain stop codons, such as amber stop codons (UAG or TAG)), ochre stop codons (UAA or TAA) and opal stop codons (UGA or TGA), between a nucleic acid encoding all or part of the domain exchanged antibody and a display protein (e.g. coat protein). To reduce toxicity of the domain exchanged antibodies to the host cell, the vectors also can contain one or more stop codons, such as amber stop codons (UAG or TAG)), ochre stop codons (UAA or TAA) and opal stop codons (UGA or TGA), in the nucleic acid encoding the antibody, or in the nucleic acid encoding a leader peptide at the N-terminus of the antibody. Incorporation of such stop codons effectively reduces the level of expression of the antibody in an appropriate host cell, such as a partial suppressor cell, thereby reducing toxicity. The vectors provided herein can be used to express and/or display polypeptides other than domain exchanged antibodies. In particular, the vectors provided herein can be used to express and/or display, with reduced toxicity, other polypeptides whose expression typically is toxic to the host cells.

Thus, provided are methods, compositions and tools (e.g. vectors) for display of polypeptides including, but not limited to, domain exchanged antibodies (including domain exchanged antibody fragments) on genetic packages, such as phage; genetic packages displaying the domain exchanged antibodies, including collections of the genetic packages (e.g. phage display libraries); methods for using the genetic packages to select domain exchanged antibodies; and domain exchanged antibodies selected from the collections. Exemplary of the tools for display are vectors for displaying the polypeptides, e.g. vectors for display of domain exchanged antibodies, such as phage display vectors containing nucleic acids encoding domain exchanged antibodies, antibody domains, and/or functional portions thereof, and coat protein(s), for example, phage coat proteins, such as cp3 (encoded by gene III) and cp8 (encoded by gene VIII).

The provided display methods and tools (e.g. vectors) can be used to display the polypeptides in a display library, e.g. a library displaying variant polypeptides. The library polypeptides can be encoded by nucleic acids in vectors within a nucleic acid library containing variant polynucleotides. In one example, the variant polynucleotides and polypeptides are varied compared to a target polypeptide, e.g. a target domain exchanged antibody. For example, the display library can be used to generate and select new variant domain exchanged antibodies, for example, antibodies having binding specificity for desired antigens, and/or antibodies having improved binding affinity or avidity or other properties. The display library can be generated by variation of nucleic acid encoding the domain exchanged antibody 2G12 or a fragment thereof, or can be generated by variation of nucleic acid encoding other domain exchanged antibodies. Thus, also provided are displayed polypeptides and polypeptides selected from the collections, e.g. displayed domain exchanged antibodies and antibodies selected from the collections.

### C. Antibodies

Antibodies are produced naturally by B cells in membrane-bound and secreted forms and specifically recognize and bind antigen epitopes through cognate interactions. Antibody-antigen binding can initiate multiple effector functions, which cause neutralization and clearance of toxins, pathogens and other infectious agents.

Diversity in antibody specificity arises naturally due to recombination events during B cell development. Through these events, various combinations of multiple antibody V, D and J gene segments, which encode variable regions of antibody molecules, are joined with constant region genes to generate a natural antibody repertoire with large numbers of diverse antibodies. A human antibody repertoire contains more than 10¹⁰ different antigen specificities and thus theoretically can specifically recognize any foreign antigen. Antibodies include such naturally produced antibodies, as well as synthetically, i.e. recombinantly, produced antibodies, such as antibody fragments, including domain exchanged antibodies.

In folded antibody polypeptides, binding specificity is conferred by antigen binding site domains, which contain portions of heavy and/or light chain variable region domains. Other domains on the antibody molecule serve effector functions by participating in events such as signal transduction and interaction with other cells, polypeptides and biomolecules. These effector functions cause neutralization and/or clearance of the infecting agent recognized by the antibody. Domains of antibody polypeptides can be varied according to the methods herein to alter specific properties.

1. Structural and Functional Domains of Antibodies

Full-length antibodies contain multiple chains, domains and regions. A full length conventional antibody contains two heavy chains and two light chains, each of which contains a plurality of immunoglobulin (Ig) domains. An Ig domain is characterized by a structure called the Ig fold, which contains two beta-pleated sheets, each containing anti-parallel beta strands connected by loops. The two beta sheets in the Ig fold are sandwiched together by hydrophobic interactions and a conserved intra-chain disulfide bond. The Ig domains in the antibody chains are variable (V) and constant (C) region domains.

Each full-length conventional antibody light chain contains one variable region domain (V_{L}) and one constant region domain (C_{L}). Each full-length conventional heavy chain contains one variable region domain (V_{H}) and three or four constant region domains (C_{H}) and, in some cases, hinge region. Owing to recombination events discussed above, nucleic acid sequences encoding the variable region domains differ among antibodies and confer antigen-specificity to a particular antibody. The constant regions, on the other hand, are encoded by sequences that are more conserved among antibodies. These domains confer functional properties to antibodies, for example, the ability to interact with cells of the immune system and serum proteins in order to cause clearance of infectious agents. Different classes of antibodies, for example IgM, IgD, IgG, IgE and IgA, have different constant regions, allowing them to serve distinct effector functions.

Each variable region domain contains three portions called complementarity determining regions (CDRs) or hypervariable (HV) regions, which are encoded by highly variable nucleic acid sequences. The CDRs are located within the loops connecting the beta sheets of the variable region Ig domain. Together, the three heavy chain CDRs (CDR1, CDR2 and CDR3) and three light chain CDRs (CDR1, CDR2 and CDR3) make up a conventional antigen binding site (antibody combining site) of the antibody, which physically interacts with cognate antigen and provides the specificity of the antibody. A whole antibody contains two identical antibody combining sites, each made up of CDRs from one heavy and one light chain. Because they are contained within the loops connecting the beta strands, the three CDRs are non-contiguous along the linear amino acid sequence of the variable region. Upon folding of the antibody polypeptide, the CDR loops are in close proximity, making up the antigen combining site. The beta sheets of the variable region domains form the framework regions (FRs), which contain more conserved sequences that are important for other properties of the antibody, for example, stability. As described herein, non-conventional antibody combining site(s) in domain exchanged antibodies are made up of residues from adjacent V_{H} domains.

The methods provided herein can be used to vary any domain(s) and/or portion(s) in target antibody polypeptides to generate collections of variant antibody polypeptides having varied structural and/or functional properties.

2. Antibody Fragments

The antibodies include antibody fragments, which are derivatives of full-length antibody that contain less than the full sequence of the full-length antibodies but retain at least a portion of the full-length antibodys' specific binding abilities. Examples of antibody fragments include, but are not limited to, Fab, Fab′, F(ab′)₂, single-chain Fvs (scFv), Fv, dsFv, diabody, Fd and Fd′ fragments, and domain exchanged fragments such as domain exchanged Fab, scFv and other domain exchanged fragments, and other fragments, including modified fragments (see, for example, Methods in Molecular Biology, Vol 207: Recombinant Antibodies for Cancer Therapy Methods and Protocols (2003); Chapter 1; p 3-25, Kipriyanov). Antibody fragments can include multiple chains linked together, such as by disulfide bridges and can be produced recombinantly. Antibody fragments also can contain synthetic linkers, such as peptide linkers, to link two or more domains.

3. Domain Exchanged Antibodies

a. Structure of Domain Exchanged Antibodies

Domain exchanged antibodies are antibodies, including antibody fragments, having the domain exchanged structure, which in general is characterized by a configuration having two interlocked V_{H} domains, with an interface forming between the interlocked V_{H} domains (V_{H}-V_{H}′ interface). Typically, the V_{H} domains interact with opposite V_{L} domains compared to the interaction in a conventional antibody (see, for example, Published U.S. Application, Publication No.: US20050003347). FIG. 1 shows a schematic comparison of exemplary conventional and domain exchanged IgG antibody structures. In this example, the full-length folded domain exchanged antibody adopts an unusual structure, in which the two heavy chain variable regions swing away from their cognate light chains and pair instead with the “opposite” light chain variable regions. A full-length (e.g. intact IgG) domain exchange antibody can exist as monomers or substantially as dimers (see e.g., West et al. (2009) J Virol., 83:98-104). Domain-exchanged antibody fragments, for example Fab fragments, exist as dimers due to the interface formed by two interlocking V_{H} domains.

The adoption of the domain exchanged configuration can occur due to mutation(s) in the heavy chains, such as within the joining region between the V_{H} and C_{H} regions. In the exemplary domain exchanged full-length antibody illustrated in FIG. 1, the variable region of each heavy chain (V_{H} and V_{H}′, respectively) interacts with the variable region on the opposite light chain compared with the interactions between the constant regions of the molecule (C_{H}-C_{L}). Additional framework mutations along the V_{H}-V_{H}′ interface can act to stabilize this domain-exchange configuration (see, for example, Published U.S. Application, Publication No.: US20050003347). In one example, the interaction between the V_{H} domains is promoted/stabilized by differences in amino acid residues in the V_{H} domains compared to conventional antibodies, such as, but not limited to, mutations at positions 19, 57, 77, 84 and 113, using Kabat numbering, such as Ile at position 19, Arg at position 57, Val at position 84 and/or Pro at position 113.

Because of the unique interaction of the V_{H} and V_{L} domains of a domain exchanged antibody, resulting in two interlocked V_{H} domains, and the V_{H} domains interacting with opposite V_{L} domains compared to the interaction in a conventional antibody, fragments of domain exchanged antibodies contain twice the number of domains as fragments of conventional antibodies. Typically, the fragments are dimeric. For example, a domain exchanged Fab fragment contains one light chain (V_{L} and C_{L}) and a heavy chain fragment, containing a variable domain of a heavy chain (V_{H}) and one constant region domain of the heavy chain (C_{H}), like a conventional fragment, but because the V_{H} domain swings away from its cognate V_{L} domain, it can interact with another, opposite, V_{L} domain. Thus, a dimer is formed, containing a pair of interlocked Fabs where each V_{H} domain interacts with the V_{L} domain that is “opposite” to the interaction that occurs through the constant regions (see e.g. FIG. 2A-D), depicting a domain exchanged Fab fragment as part of a bacteriophage coat protein 3 (cp3) fusion protein. Similarly, other fragments of domain exchanged antibodies have twice the number of V_{H} and/or V_{L} domains as the corresponding conventional antibody fragment. For example, domain exchanged scFv antibody fragments have two V_{L} domains and two V_{H} domains (see e.g. FIG. 2E-H), in contrast to conventional scFv antibody fragments, which have only one V_{L} domain and one V_{H} domain.

In conventionally structured IgG, IgD and IgA antibodies, the hinge regions between the C_{H}1 and C_{H}2 domains can provide flexibility, resulting in mobile antibody combining sites that can move relative to one another to interact with epitopes, for example, on cell surfaces. In domain exchanged antibodies, by contrast, this flexible arrangement is not adopted. In one example, domain exchanged antibodies can contain two conventional antibody combining sites and a non-conventional antibody combining site, which is formed by the interface between the two adjacently positioned heavy chain variable regions, all of which are in close proximity with one another and constrained in space, as illustrated in the exemplary IgG in FIG. 1. Typically, where a domain exchanged antibody contains two conventional antibody combining sites, the sites are within less than or about 100, 90, 80, 70, 60, 50, 40, or 30 angstroms of one another. For example, exemplary domain exchanged antibodies can have two conventional antibody combining sites that are less than 100 or less than about 100 angstroms from one another; less than 50 or less than about 50 angstroms from one another, or less than 35 or less than about 35 angstroms from one another. In contrast, the distance between conventional binding sites of conventional IgG antibodies typically is greater than 120 angstroms (West et al., (2009) J. Virol. 83:98-104). For example, an IgG antibody specific for gp120 was found to have a distance between the conventional binding sites of 171 angstroms (Saphire et al., (2001) Science 293:1155-1159).

Exemplary of domain exchanged antibodies are those that specifically bind epitopes within densely packed and/or repetitive epitope arrays, such as sugar residues on bacterial or viral surfaces. The unusual domain exchanged configuration can promote binding to such epitopes. In some examples, domain exchanged antibodies can recognize and bind epitopes within high density arrays, which evolve, for example, in pathogens and tumor cells as means for immune evasion. Examples of such high density/repetitive epitope arrays include, but are not limited to, epitopes contained within bacterial cell wall carbohydrates and carbohydrates and glycolipids displayed on the surfaces of tumor cells or viruses. Such epitopes are not optimally recognized by conventional (non-domain exchanged) antibodies. In one example, the high density and/or repetitiveness of epitopes can render simultaneous binding of both antibody-combining sites of a conventional antibody energetically disfavored.

Thus, in one example, domain exchanged antibodies specifically bind to, and can be used to target (e.g. therapeutically; e.g. by high affinity binding), epitopes that conventional antibodies typically cannot specifically bind or, can bind only with low affinity. Exemplary of such epitopes include, but are not limited to, epitopes on antigens expressed in or on cells, tissues, blood, fluids and organisms, including infectious agents, such as microbes, viruses, bacteria (gram negative and gram positive bacteria), yeast, and fungi, including drug-resistant and poorly immunogenic infectious agents. Exemplary antigens are poorly immunogenic polysaccharide antigens of bacteria, fungi, viruses and other infectious agents, such as drug-resistant agents (e.g. drug resistant microbes) and tumor cells, including antigens expressed on viral surfaces and bacterial surfaces, such as cell walls.

Exemplary domain exchanged antibody fragments are illustrated in FIG. 2 and described in Example 8. These fragments and methods for their generation are described in further detail below. FIG. 2 depicts the antibody fragments as part of bacteriophage coat protein 3 (cp3) fusion proteins, for display on filamentous bacteriophage. Alternatively, any of the fragments depicted in FIG. 2 and described herein can be adapted for display on other genetic packages, for example, using different genetic package vectors and coat proteins. Alternatively, the fragments can be produced as non-fusion protein fragments for purposes other than display on genetic packages. The fragments described below are exemplary and the methods for vector design can be used in various combinations to generate other related domain exchanged fragments for display on genetic packages.

b. 2G12 and Variants Thereof.

Exemplary of a domain exchanged antibody that can be displayed with the provided methods and vectors, and used in the collections and libraries herein, is the 2G12 antibody, which is a broadly neutralizing anti-HIV antibody. With its domain exchanged structure 2G12 binds with high affinity to oligomannose residues on the surface of HIV. 2G12 binds to α1→2 mannose epitope on the outer face of HIV gp120 antigen. 2G12 antibodies include the domain exchanged human monoclonal IgG1 antibody produced from the hybridoma cell line CL2 (as described in U.S. Pat. No. 5,911,989; Buchacher et al., AIDS Research and Human Retroviruses, 10(4) 359-369 (1994); and Trkola et al., Journal of Virology, 70(2) 1100-1108 (1996)), as well as any synthetically, e.g. recombinantly, produced antibody having the identical sequence of amino acids, and any antibody fragment thereof having identical heavy and light chain variable region domains to the full-length antibody, such as the 2G12 domain exchanged Fab fragment (see, for example, Published U.S. Application, Publication No.: US20050003347 and Calarese et al., Science, 300, 2065-2071 (2003), which contains a heavy chain (V_{H}-C_{H}1) having the sequence of amino acids set forth in SEQ ID NO: 158 (EVQLVESGGGLVKAGGSLILSCGVSNFRISAHTMNWVRRVPGGGLEWVASIS TSSTYRDYADAVKGRFTVSRDDLEDFVYLQMHKMRVEDTAIYYCARKGSDR LSDNDPFDAWGPGTVVTVSPASTKGPSVFPLAPSSKSTSGGTAALGCLVKDYF PEPVTVSWNSGALTSGVHTFPAVLQSSGLYSLSSVVTVPSSSLGTQTYICNVN HKPSNTKVDKKVEPKs); and a light chain (V_{L}) having the sequence of amino acids set forth in SEQ ID NO: 159 (VVMTQSPSTLSASVGDTITITCRASQSIETWLAWYQQKPGKAPKWYKASTL KTGVPSRFSGSGSGTEFTLTISGLQFDDFATYHCQHYAGYSATFGQGTRVEIK RTVAAPSVFIFPPSDEQLKSGTASVVCLLNNFYPREAKVQWKVDNALQSGNS QESVTEQDSKDSTYSLSSTLTLSKADYEKHKVYACEVTHQGLSSPVTKSFNRG E).

With respect to SEQ ID NO:308, the FR1 corresponds to amino acids 1-30; the CDR1 corresponds to amino acids 31-35 the FR2 corresponds to amino acids 36-49; the CDR2 corresponds to amino acids 50-66; the FR3 corresponds to amino acids 67-98; the CDR3 corresponds to amino acids 99-112, the FR4 corresponds to amino acids 113-123; the C_{H}1 corresponds to amino acids 124-225; the hinge amino acids correspond to amino acids 226-236; and the C_{H}2-C_{H}3 amino acids correspond to amino acids 237-454. With respect to SEQ ID NO:159, the FR1 corresponds to amino acids 1-22; the CDR1 corresponds to amino acids 23-33; the FR2 corresponds to amino acids 34-48; the CDR2 corresponds to amino acids 49-55; the FR3 corresponds to amino acids 56-87; the CDR3 corresponds to amino acids 88-96; the FR4 corresponds to amino acids 97-106; the C_{L} corresponds to amino acids 107-213.

Also included are 2G12 antibody fragments having at least the antigen-binding portions of the 2G12 V_{H} domain (SEQ ID NO: 10; EVQLVESGGGLVKAGGSLILSCGVSNFRISAHTMNWVRRVPGGGLEWVASIS TSSTYRDYADAVKGRFTVSRDDLEDFVYLQMHKMRVEDTAIYYCARKGSDR LSDNDPFDAWGPGTVVTVSP), and typically of the 2G12 V_{L} domain (SEQ ID NO: 11: (DVVMTQSPSTLSASVGDTITITCRASQSIETWLAWYQQKPGKAPKWYKAST LKTGVPSRFSGSGSGTEFTLTISGLQFDDFATYHCQHYAGYSATFGQGTRVEI K) or SEQ ID NO: 12 (AGVVMTQSPSTLSASVGDTITITCRASQSIETWLAWYQQKPGKAPKWYKA STLKTGVPSRFSGSGSGTEFTLTISGLQFDDFATYHCQHYAGYSATFGQGTRV EIK)) of the full-length human antibody and retaining specific binding to the epitope(s) of the HIV gp120 antigen (e.g. as described in U.S. Pat. No. 5,911,989 and in Published U.S. Application, Publication No.: US20050003347).

Amino acid residues in the V_{H} domains of 2G12 (e.g. amino acids at positions 19 (Ile), 57 (Arg), 77 (Phe), 84 (Val) and 113 (Pro), based on Kabat numbering), which vary compared to analogous residues in conventional antibodies, promote and/or stabilize the domain exchanged structure and stabilize the interface between the two V_{H} domains (U.S. Publication No.: US20050003347). With its domain exchanged structure, 2G12 binds with high affinity to oligomannose residues on the surface of HIV. 2G12 antibodies with differing sequences also are known and can be used in the methods, vectors, nucleic acids and libraries herein. These include, for example, a 2G12 having a replacement of V5L and H237S in the heavy chain sequence (SEQ ID NO:313; see e.g. West et al. (2009) J. Virol., 83:98-104)

Also exemplary of the domain exchanged antibodies are modified 2G12 antibodies, containing one or more modifications compared to a 2G12 antibody, such as modifications in CDR(s). Exemplary of a modified 2G12 domain exchanged antibody that can be used in the provided methods, vectors and collections is the 3-Ala 2G12 antibody, and fragments or intact IgG molecules thereof, and the 3-Ala LC 2G12 antibody or intact IgG molecules, and fragments thereof. 3-Ala 2G12 is a modified 2G12 antibody having three mutations to alanine in the amino acid sequence of the heavy chain antigen binding domain, rendering it non-specific for the antigen (gp120; GenBank g.i. no.: 28876544) that is recognized by the native 2G12 antibody. The 3-Ala 2G12 V_{H} domain contains the sequence of amino acids set forth in SEQ ID NO: 161 (EVQLVESGGGLVKAGGSLILSCGVSNFRISAHTMNWVRRVPGGGLEWVASIS TSSTYRDYADAVKGRFTVSRDDLEDFVYLQMHKMRVEDTAIYYCARKGSDR AADADPFDAWGPGTVVTVSP), and has alanine substitutions at positions 9H100, H100a, H100c by Kabat numbering (corresponding to positions 104, 105 and 107 in SEQ ID NO:161). Thus, the 3-ALA 2G12 antibody does not specifically bind gp120. Also exemplary of the domain exchanged antibodies are modified 3-ALA 2G12 antibodies, having modification(s) compared to a 3-ALA 2G12 antibody, such as modifications in one or more CDRs, such as those described herein.

3-Ala LC 2G12 is a modified 2G12 antibody having three mutations to alanine in the amino acid sequence of the light chain antigen binding domain, rendering it non-specific for the both gp120 and Candida albicans. These mutations are at positions L91, L94 and L95 by Kabat numbering. Thus, exemplary 3-Ala LC 2G12 V_{L} domains include those having a sequence of amino acids set forth in SEQ ID NO:305 and 321. Also exemplary of the domain exchanged antibodies are modified 3-Ala LC 2G12 antibodies, having modification(s) compared to a 3-Ala LC 2G12 antibody, such as modifications in one or more CDRs, such as those described herein, including those with a CDRL3 having a sequence set forth in any of SEQ ID NOS:181-241; and those with a light chain having a sequence set forth in any of SEQ ID NOS:242-302. In one example, the modified 3-Ala LC 2G12 antibodies bind specifically to Candida species, including C. albicans.

Also included among the modified 2G12 domain exchanged antibodies that can be used with the methods, vectors, nucleic acids and libraries provided herein, such as for expression, display and further modification of the antibodies, are any described in the art. As a full-length antibody 2G12 exists in both monomeric and dimeric form. Mutations can be made in 2G12 that increases the 2G12 dimer/monomer ratio; dimers can be separately purified therefrom (see e.g. West et al. (2009) J. Virol., 83:98-104). Such dimers can exhibit increased potency and antigen-binding affinity. Exemplary of such mutations include hinge deletion mutants, including but not limited to, mutations corresponding to mutations in 2G12 heavy chain sequence set forth in SEQ ID NO:313 that include deletion of residue 237; deletion of residues 236 to 237; deletion of residues 235 to 237; deletion of residues 232 to 237; deletion of residues 232 to 239; and deletion of residues 232 to 239 and two proline to glycine substitutions at amino acid positions P240G and P241G. Such exemplary 2G12 mutants are set forth in SEQ ID NO:314-320. It is understood that any of the antibodies provided herein can further contain such mutations in the antibody to increase dimer formation of a full-length 2G12 antibody.

Other variant 2G12 antibodies or fragments thereof can be generated using 2G12 nucleic acid libraries into which diversity has been introduced. Any method for creating diversity can be used, including the methods described herein and elsewhere (including related U.S. patent application No. [Attorney Docket No. 3800013-00031/1106] and related International Patent Application No. [Attorney Docket No. 3800013-00032/1106PC]). The variant polynucleotides can be expressed using the vectors and cells provided herein, and displayed on genetic packages, such as phage, which can then be screened for a desired specificity. This process is exemplified in Examples 9-15, in which variant 2G12 antibodies with specificity for Candida were generated using the methods, vectors and cells provided herein. Such a process can be used to generate 2G12 domain exchanged antibodies with any desired specificity.

c. Other Domain Exchanged Antibodies

Any domain exchanged antibody can be used with the methods, genetic packages, vectors and libraries provided herein. As discussed above, domain-exchanged antibodies have a particular structure containing an interface formed by two interlocking V_{H} domains (VH-VH′ interface); as a result, unlike conventional antibodies, domain-exchanged antibodies are able to specifically bind epitopes that are densely packed or repetitive. As discussed further below, one of skill in the art can use any screening method that permits identification of a domain-exchanged antibody or a fragment thereof. In some examples, other natural domain exchanged antibodies are identified. In other examples, domain exchanged antibodies are created from conventional antibodies (see e.g. U.S. Patent Publication No. 20050003347). U.S. Patent Publication No. 20050003347 describes the structure and properties of an exemplary domain exchanged antibodies. Using such teachings, one of skill in the art can generate other domain exchanged antibodies from the germline sequences of conventional antibodies by incorporating these structural attributes into the conventional antibody. For example, mutations can be introduced into the conventional antibody t positions corresponding to amino acid positions 19, 57, 77 and 113 (based on kabat numbering) of the heavy chain, to formation and stabilization of the V_{H}-V_{H} interface. Further, position 38 of the light chain and position 39 of the heavy chain, which typically are conserved glutamine residues in conventional antibodies, can be modified to weaken the V_{H} and V_{L} interface. This can be desirable for the formation of domain exchanged antibodies. Other amino acid positions that can be modified, such as by amino acid replacement, in a conventional antibody to generate a domain-exchanged antibody include, but are not limited to, amino acid positions 70, 72, 79, 81 and 84 of the heavy chain. Thus, domain exchanged antibodies other than 2G12 can be generated and used in the methods, vectors and collections herein. In some examples, the nucleic acid encoding theses domain exchanged antibodies are fragments thereof are used to nucleic generate libraries, which are then introduced into vectors and/or cells to express and display the antibodies on phage, as described herein, and selected and screened for desired specificity.

One of skill in the art is familiar with the structure of a domain-exchanged binding molecule and methods to confirm the identification thereof (see, for example, Published U.S. Application, Publication No.: US20050003347). Conventional full-length antibodies, such as conventional full length IgG antibodies, generally contain two antigen-binding sites separated by distances that are greater than 120 Å, generally 150-170 Å. In contrast, domain-exchanged antibodies have at least two antigen-binding sites separated by a distance that is less than 120 Å, such as less than 100 Å, 90 Å, 80 Å, 70 Å, 60 Å, 50 Å, 40 Å or 30 Å. For example, the antigen-binding sites in 2G12 are separated by about 35 Å (see e.g., West et al. (2009) J Virol., 83:98-104). In some instances, as described herein, a domain exchange antibody that is a full-length intact IgG can exist as monomers or substantially as dimers (see e.g., West et al. (2009) J Virol., 83:98-104). Hence, as intact IgG molecules, domain-exchanged antibodies form a compact structure, monomeric or dimeric, that can be identified by various methods known to one of skill in the art, including, but not limited to, size exclusion chromatography with in-line static light scattering and refractive index monitoring, electron microscopy, sedimentation equilibrium analytical ultracentrifugation, gel filtration, native gel electrophoresis, sedimentation coefficients and/or negative-stain electron microscopy (West et al. (2009) J Virol., 83:98-104; Roux et al. (2004) Mol. Immunol., 41:1001-1011; Calarese et al. (2005) Science, 300:2065-2071; Published U.S. Application, Publication No.: US20050003347).

In other antibody forms, such as antibody fragments of a full-length IgG, domain-exchanged antibodies exist as dimers due to the interface formed by two interlocking V_{H} domains. For example, in their Fab form, domain-exchanged binding molecules exist as Fab dimers. Those of skill in the art are familiar with assays to assess the oligomeric state of proteins, such as antibodies, for example assays to assess the presence of a Fab dimer of a domain-exchanged binding molecule. Such assays include, for example, sedimentation equilibrium analytical ultracentrifugation, gel filtration, native gel electrophoresis, sedimentation coefficients and/or negative-stain electron microscopy (Roux et al. (2004) Mol. Immunol., 41:1001-1011; Calarese et al. (2005) Science, 300:2065-2071; Published U.S. Application, Publication No.: US20050003347).

4. Antibodies in Protein Therapeutics

Antibodies have various characteristics, e.g. diversity, specificity and effector functions, that render them attractive candidates for protein-based therapeutics. Numerous therapeutic and diagnostic monoclonal antibodies (MAbs) are used to treat and diagnose human diseases, for example, cancer and autoimmune diseases. In designing antibody therapeutics, it is desirable to create improved antibodies, for example, antibodies with higher specificity and/or affinity and antibodies that are more bioavailable, or stable or soluble in particular cellular or tissue environments. Available techniques for generating improved antibody therapeutics are limited.

Monoclonal Antibodies (MAbs) and Antibody Libraries

MAb production first was accomplished in 1975 by fusion of B cells to tumor cells to make clonal hybridoma cells line secreting MAbs. MAbs since have been produced using other immortalization techniques. Immortalization of B cells to produce a MAb with desired specificity typically requires isolation of B cells from an immunized non-human animal or from blood of an immunized or infected human donor. Non-human therapeutic antibodies are problematic due to immunogenicity of non-human sequences. In attempts to overcome this difficulty, various genetic techniques have been used to engineer chimeric or humanized antibodies in which the non-antigen-binding portions of the antibodies are encoded by human sequences. Transgenic animals also can be used to produce fully human antibodies.

Recombinant DNA technology has allowed production of antibodies and antibody fragments by cloning of human antibody sequences and expression in host cells. Using recombinant techniques, antibody coding sequences can be manipulated to vary specificity and other properties. These techniques have been used to create collections of antibodies (antibody libraries), particularly phage display libraries, with diverse arrays of antigen specificities for selection of antibodies having desired properties. For example, synthetic and semi-synthetic antibody libraries are made by techniques that synthetically mutate or randomize particular portions of antibody variable region genes, for example by PCR using degenerate primers and cassette mutagenesis.

### D. Vectors and Methods

Expression and display of domain exchanged antibodies using conventional methods and vectors can be difficult. In the first instance, like many other antibodies and other proteins, recombinant expression of domain exchanged antibodies can be toxic to the host cells. Toxicity of domain exchanged antibodies and other recombinant proteins to the host cell can hinder both their initial identification and subsequent development and/or modification for research and therapeutic use. For example, effective screening and selection of domain exchanged antibodies or other proteins from libraries, such as, for example, phage display libraries, relies on the stable expression of every antibody or protein in the library. Proteins, such as antibodies, that are toxic to host cells typically cannot be recovered using such methods. In some instances, the host cell expressing the protein is non-viable. In other instances, the nucleic acid encoding the protein is modified or deleted to reduce toxicity such that the protein is no longer expressed in its original form. In such examples, the proteins are no longer available in the library for screening and selection, or are present at insufficient levels for recovery.

In the second instance, the unique configuration of domain exchanged antibodies, which in general is characterized by a configuration having two interlocked VH domains, with an interface forming between the interlocked VH domains (VH-VH′ interface), makes it difficult to express and display on genetic packages, such as phage, thus limiting conventional methods for screening and selection of domain exchanged antibodies, including variants thereof. Thus, provided herein are nucleic acids (such as vectors), cells and methods for expression and/or display of domain exchanged antibodies and other polypeptides.

The advantages of the vectors provided herein are two-fold. In the first instance, the vectors are designed to reduced the toxicity associated with expression of a particular polypeptides, such as an antibody or other polypeptide whose expression can be toxic to the host cell. The vectors provided herein contain one or more stope codons that effectively down regulate expression of the encoded protein(s) when the vectors are introduced into a suitable partial suppressor strain. Thus, the vectors can be used to more efficiently express any polypeptide that typically exhibits toxicity to a host cell. Exemplary of toxic polypeptides that can be expressed from the vectors provided herein are antibodies and fragments thereof, including domain exchanged antibodies and fragments thereof.

In the second instance, the vectors are designed to express and display domain exchanged antibodies and Fab fragments in the correct configuration. Exemplary domain exchanged antibody fragments that can be expressed and displayed using the vectors and methods provided herein include, but are not limited to, domain exchanged Fab fragments, domain exchanged single chain Fab fragments, domain exchanged scFv fragments and variations of these fragments. Thus, the vectors provided herein include those that are designed to reduce toxicity of a polypeptide to the host cell, and those designed to express and display antibodies, in particular, domain exchanged antibodies.

Provided herein are nucleic acids, including vectors, that can be used to express and display domain exchanged antibodies in the correct configuration. Also provided are nucleic acids, including vectors, that can be used to express polypeptides, such as antibodies, including domain exchanged antibodies, with reduced toxicity to the host cells compared to when the polypeptides are expressed using other nucleic acids, including vectors, and methods. In some instances, nucleic acids, including vectors, provided herein can be used to express and display domain exchanged antibodies in the correct configuration with reduced toxicity to the host cell.

1. Overview of Expression and Display of Polypeptides with Reduced Toxicity, Including Domain Exchanged Antibodies.

a. Expression with Reduced Toxicity

The expression of recombinant proteins in systems, such as bacterial expression systems, has lead to increased understanding of the function of various proteins and allowed for the identification and development of proteins for research and therapeutic use. Many proteins, however, are toxic to host cells. This can hinder both their initial identification and subsequent development and/or modification for research and therapeutic use. For example, effective screening and selection of proteins from libraries, such as, for example, phage display libraries, relies on the stable expression of every protein in the library. Proteins that are toxic to host cells typically cannot be recovered using such methods. In some instances, the host cell expressing the protein is non-viable. In other instances, the nucleic acid encoding the protein is modified or deleted to reduce toxicity such that the protein is no longer expressed in its wild-type form. In such examples, the proteins are no longer available in the library for screening and selection, or are present at such low levels that they are not sufficiently recovered.

Several strategies have been developed to reduce the toxicity of recombinant proteins to host cells, with varying degrees of success. For example, tight control of toxic gene transcription and translation, such as by the use of non-leaky and/or inducible promoters, can be used to control the timing and extent of protein production. Other strategies include, but are not limited to, using antisense technology to bind to the mRNA encoding the toxic protein; phage-mediated delivery of the highly selective T7 RNA polymerase to facilitate expression in T7 gene 1-deficient cells; using invertible, competitive and/or hybrid promoters; using the full length lac Promoter/Operator region to regulate expression; and controlling the vector copy number (see e.g., Saida et al (2006) Cur. Port. Pept. Sci. 7; 47-56).

Provided herein are vectors for the expression of proteins with reduced toxicity, in which strategic incorporation of one or more stop codons into the vector results in reduced translation of the protein encoded by the vector, compared to translation of the same protein from a comparable vector without the stop codon(s) (i.e. compared to in the absence of the stop codon(s)), when the vectors are introduced into an appropriate partial suppressor cell. Thus, the vectors provided herein effectively “down regulate” the expression of the protein, reducing toxicity of the proteins to the host cell. The stop codon(s) is introduced into the genetic element encoding the protein for which reduced expression is desired. In some examples, the stop codon is incorporated into the coding sequence of this protein. In other examples, the stop codon is introduced into nucleic acid encoding a polypeptide that is fused to the N-terminus of protein for which reduced expression is desired. For example, in some aspects, the vectors provided herein contain genetic element that contains nucleic acid encoding a leader peptide linked to the nucleic acid encoding the protein for which reduced expression is desired, and the stop codon is introduced into the leader sequence.

Using the vectors provided herein, the level of expression of the protein of interest can be modulated depending upon the host cell in which it is being expressed. If the vectors is introduced into a host cell containing wild-type tRNA molecules (i.e. non suppressor cells) the presence of the stop codon in the mRNA transcribed from the genetic element encoding the protein of interest terminates translation. Thus, no protein is expressed. If the vector is introduced into a cell containing suppressor tRNAs (i.e. a suppressor cell), instead of terminating translation of the polypeptide at the stop codon, the suppressor tRNA incorporates an amino acid into the growing polypeptide, thereby allowing “read through” and continued synthesis of the protein. Suppressor tRNAs can arise by mutations in the gene encoding the tRNA. For example, a mutation in the tyrT gene changes the anticodon in the tRNA so that it recognizes the stop codon 5′ UAG 3′ in the mRNA and, instead of terminating, inserts a tryrosine at that position in the polypeptide chain. Typically however, suppressor tRNAs facilitate read through only part of the time (i.e. with low efficiency, resulting in “partial suppressor cells”), while some of the time translation is terminated at the stop codon. Thus, expression of the protein in partial suppressor cells is effectively down-regulated, as only some of the transcripts are translated through the stop codon by the suppressor tRNAs. This reduced expression results in reduced toxicity to the cell, while still maintaining sufficient expression levels for isolation and/or functional analysis of the protein.

The vectors provide herein can, therefore, be used to express any protein at reduced levels to reduce toxicity to the host cell. In some examples, the protein is an antibody. The vectors provided herein can be used to express full length antibodies or fragments thereof, such as Fab, Fab′, F(ab)₂, single-chain Fvs (scFv), Fv, dsFv, diabody, Fd and Fd′ fragments. As discussed below, in a particular example, the vectors are used to express domain exchanged antibodies and fragments thereof.

b. Display of Proteins, Including Domain Exchanged Antibodies and Fragments Thereof.

Provided herein are vectors that can be used to express a protein of interest, such as an antibody or fragment thereof, by itself, or as a fusion protein. In particular, provided herein are vectors that can be used to express a protein, such as the antibody or fragment thereof, by itself, or as a fusion protein with a genetic package display protein, such as a phage coat protein. Such vectors facilitate the display of domain exchanged antibodies on a genetic package. This can be achieved by introducing a stop codon, such as an amber stop codon (UAG or TAG)), the ochre stop codon (UAA or TAA)) and the opal stop codon (UGA or TGA)), between the nucleic acid encoding the protein of interest (such as an antibody) and the nucleic acid encoding the phage coat protein. When expressed in an appropriate partial suppressor cell, there is partial read through of the stop codon, resulting in a mixed collection of polypeptides. When there is read through of the stop codon, the protein of interest, such as the antibody or fragment thereof, is expressed as a fusion with the phage coat protein. When there is no read through (i.e. translation is terminated), the protein is produced without fusion to the coat protein, and thus is secreted as a soluble polypeptide. In one example, the mixed population contains between or about 50% and or about 75% soluble protein, and between or 25% and or about 50% protein-coat protein fusion protein. Thus, the vectors provided herein can be used to express proteins for phage display libraries and other display libraries, and also can be used to express soluble polypeptides that are not fused to the phage coat protein.

In one example, the soluble protein expressed from the vector interacts with the fusion protein expressed from the same vector, for example, through hydrophobic interactions and/or disulfide bonds, so that both polypeptides are expressed on the surface of the phage. Such a process can be of particular use in the expression of domain exchanged antibodies.

Display of domain exchanged antibodies on genetic packages (such as, for example, phage display) using conventional methods and vectors is not straightforward. With conventional phage display methods, antibodies typically are displayed as conventional Fab fragments or conventional scFv fragments. For Fab fragments, each fragment contains one heavy chain (containing one heavy chain variable region (V_{H}) and first constant region domain (C_{H}1)) and one light chain (containing one light chain variable region (V_{L}) and constant region (C_{L})). These two chains are expressed as separate polypeptides that pair through heavy-light chain interactions to form the conventional antibody fragment molecule. For phage display of the conventional Fab fragment, the heavy chain portion typically is fused to a phage coat protein as described herein below, such as gene III protein, to form a fusion protein. For scFv fragments, each fragment contains one heavy chain variable region (V_{H}) and one light chain variable region (V_{L}), which are connected by a peptide linker and expressed as a single chain. For phage display of the conventional scFv fragment, the single V_{H}-linker-V_{L} chain is fused to a phage coat protein to form a fusion protein.

Thus, with the conventional phage display methods, the displayed antibody fragment typically contains a single antibody combining site. By contrast, domain exchanged antibodies contain an interface between the two interlocked V_{H} domains (V_{H}-V_{H}′ interface), which can be promoted, for example, by mutations in the V_{H} domains that cause them to interact with one another and to pair with opposite V_{L} chains compared with conventional antibodies, as illustrated in FIG. 1. Such antibodies are not easily expressed and displayed using conventional methods. Generally, bivalent antibody molecules (having two antibody combining sites), such as F(ab′)2 fragments are not easily expressed in bacterial cells. One report describes phage display constructs for expression of F(ab′)2-like molecules containing two heavy chains (V_{H}-C_{H}1—each part of a coat fusion protein) and light chains (V_{L}-C_{L}); each construct contained all or part of a dimerization domain having a leucine zipper and an antibody hinge region. (Lee et al., Journal of Immunological Methods, 284 (2004) 119-132; see also U.S. publication No. US 2005/0119455). In this report, when an amber stop codon sequence was included between the V_{H}-C_{H}1- and phage coat protein-coding sequences, hinge region cysteines and at least part of the leucine zipper domain were required for the bivalent display.

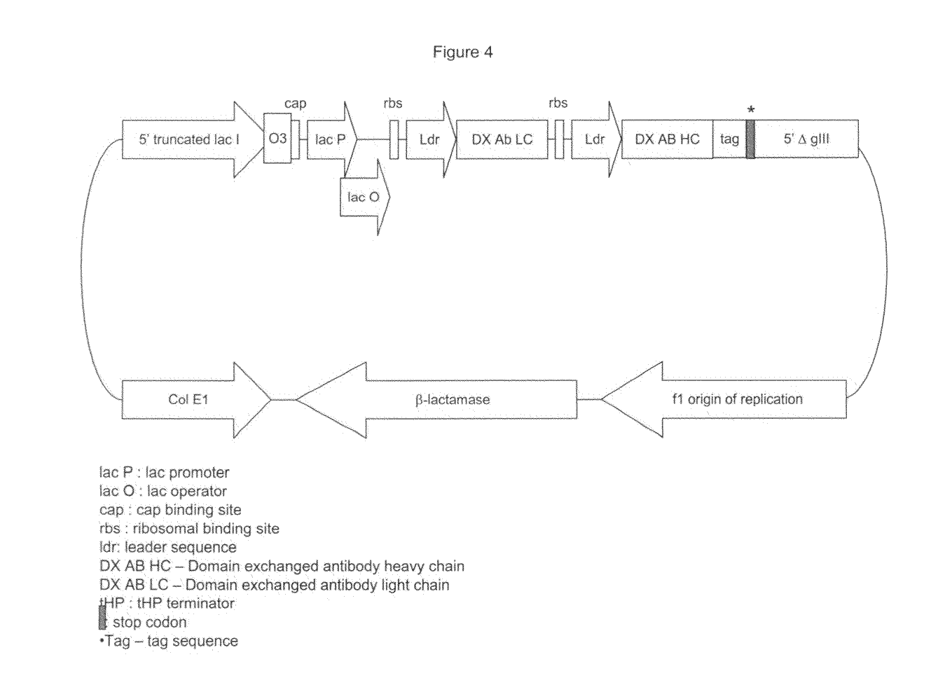

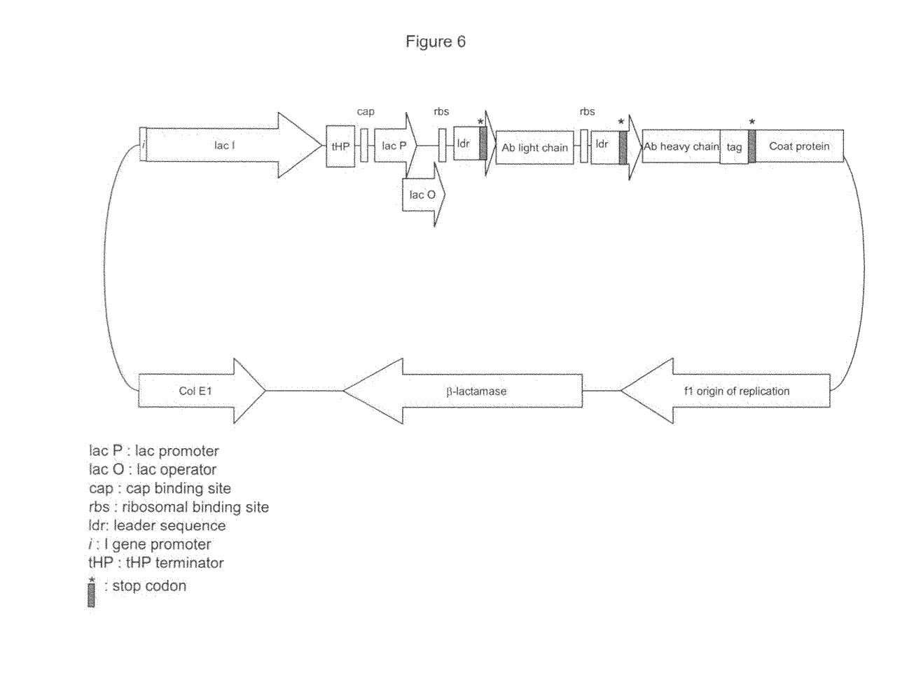

By incorporation of a stop codon, such as an amber stop codon (UAG or TAG)), the ochre stop codon (UAA or TAA)) and the opal stop codon (UGA or TGA)), between the nucleic acid encoding the antibody heavy chain and the phage coat protein, the vectors provided herein facilitate the formation of the unique configuration of domain exchanged antibodies and fragments thereof and their display on phage. For example, a Fab fragment of a domain exchanged antibody can be expressed from the vectors provided herein in partial suppressor cells. The Fab fragment is produced by expressing from the same vector, such as one illustrated in FIG. 4 or 6, a soluble light chain, a soluble heavy chain and a heavy chain fused to the phage coat protein. The domain exchanged Fab fragment can then be formed by association of soluble two light chains with the soluble heavy chain and heavy chain-phage coat protein fusion protein, as shown in FIG. 2A.

Thus, provided herein are vectors and methods for display of domain exchanged antibodies, including domain exchanged antibody fragments, and other bivalent antibodies. Provided also are various domain exchanged antibody fragments, including displayed domain exchanged antibody fragments, expressed and or displayed using the vectors provided herein. Exemplary domain exchanged antibody fragments are illustrated in FIG. 2, which illustrates the fragments displayed on phage. These fragments alternatively can be expressed as soluble proteins and can be displayed using other display systems. The fragments and methods for their generation are described in further detail below. FIG. 2 depicts the displayed antibody fragments as part of bacteriophage coat protein 3 (cp3) fusion proteins, for display on filamentous bacteriophage. Alternatively, any of the fragments depicted in the figure and described herein can be adapted for display on other genetic packages, for example, using different genetic package vectors and coat proteins. Alternatively, the fragments can be produced as non-fusion protein fragments for purposes other than display on genetic packages. The fragments described below are exemplary and the methods for vector design can be used in various combinations to generate other related domain exchanged fragments for display on genetic packages.

Thus, the provided domain exchanged fragments can be displayed on genetic packages in the appropriate domain exchanged configuration. The provided methods and genetic packages can be used to select new domain exchanged antibodies, for example, domain exchanged antibodies having particular antigen-specificity, for example, by using one or more of the provided methods for introducing diversity in proteins. In one example, domain exchanged antibodies have specificity for Candida albicans are generated using the methods providing herein.

The phagemid vectors provided herein can be used to generate diverse phage display libraries in which otherwise toxic antibodies (including conventional antibodies or fragments thereof and domain exchanged antibodies or fragments thereof, can be expressed on the surface of phage and enriched by selection. For example, the vectors can be used to generate nucleic acid libraries encoding variant antibodies or fragments thereof, including variant domain exchanged antibodies or fragments thereof. The nucleic acid libraries can be introduced into the appropriate partial suppressor cells, that are phage-display compatible, to generate a phage display library in which the variant antibodies or fragments thereof are displayed on the surface of the phage. Because the antibodies are expressed at reduced levels, toxicity is reduced. This results in a diverse library in which each variant antibody is stably expressed and can be screened and selected. For example, recovery and enrichment of the Fab fragment of domain exchanged human monoclonal antibody 2G12 (U.S. Pat. No. 5,911,989; Buchacher et al., (1994) AIDS Res. Hum Retroviruses, 10(4) 359-369; and Trkola et al., (1996) J. Virol, 70(2) 1100-1108) is enhanced using a vector in which expression of the Fab is reduced by incorporation of a stop codon in the leader sequence upstream of the nucleic acid encoding the 2G12 Fab (see Example 2, below). Selection of 2G12 domain-exchanged antibodies, or other domain exchanged antibodies, with specificity for any other antigens also is facilitated using the vectors and methods provided herein. For example, variant 2G12 domain exchanged antibodies specific for Candid albicans can be identified using the methods and vectors provided herein (see Examples 9-15).

In a particular example, the vectors also contain one or more stop codons that result in reduced toxicity to the host cell upon the expression of the protein, such as the antibody, as described above. Thus, provided herein are phagemid vectors that can be used to express a protein, such as an antibody or fragment thereof, on the surface of phage, such as in a phage display library, with reduced toxicity to the host cell. Because of the reduced toxicity of the expressed and displayed antibodies (or other proteins) using the vectors provided herein, these antibodies can be recovered and enriched following selection using, for example, phage display methods.

2. Vectors

The vectors an nucleic acids provided herein contain one or more stop codons, such as an amber stop codon (UAG or TAG)), ochre stop codon (UAA or TAA)) or opal stop codon (UGA or TGA)), that either a) effectively down regulate the expression of the encoded protein(s) when the vectors are introduced into a suitable partial suppressor strain, thus reducing toxicity of the protein, or b) facilitate expression of both soluble proteins and fusion proteins. In some examples, the vectors and nucleic acids provided herein contain two more stop codons that together result in reduced expression of the encoded protein(s) (resulting in reduced toxicity) and result in expression of both soluble proteins and fusion proteins, when the vectors are introduced into a suitable partial suppressor strain. Typically, the fusion proteins are fusions containing a genetic package display protein, such as a phage coat protein.

For reduced toxicity, the stop codon(s) are introduced into a leader sequence that is operably linked to the nucleic acid encoding the protein for which reduced expression is desired, and/or introduced into the coding sequence of the protein for which reduced expression is desired. The vectors can contain 1, 2, 3, 4, 5, 6, 7, 8, 9, 10 or more stop codons in the leader sequence and/or encoding nucleic acid of the protein of interest. For expression of both soluble proteins and fusion proteins, such as soluble antibodies and antibody-display protein fusion proteins, the stop codon is introduced between, for example, the nucleic acid encoding the antibody and the nucleic acid encoding the display protein.

When the vectors are introduced into a suitable partial suppressor strain that contains suppressor tRNAs that recognize the stop codon, in some instances read through of the stop codon can occur, while in other instances translation is terminated at the stop codon and the full length protein can be expressed. Thus, in vectors containing a stop codon between, for example, the nucleic acid encoding the antibody and the nucleic acid encoding the display protein, both soluble and fusion proteins are generated. With vectors containing one or more stop codons in the leader sequence and/or encoding nucleic acid of the protein of interest, reduced expression of the protein is observed compared to the expression of the same protein from a comparable vector that does not contain the introduced stop codon in the leader sequence or in the nucleic acid encoding the protein. Thus, provided herein are vectors that contain nucleic acid encoding one or more proteins for which reduced expression is desired. Also provided herein are vectors into which nucleic acid encoding a protein for which reduced expression is desired can be inserted, such that the encoded protein is expressed at reduced levels when the vector is introduced into a partial suppressor cell.

The vectors provided herein contain all of the necessary transcription, translation and regulatory elements for expression and/or display of one or more proteins of interest, such as one or more antibodies or antibody fragments. In some instances, the expression of the protein of interest is reduced when the vectors are transformed into an appropriate partial suppressor cell, compared to if the protein was expressed from a vector that does not contain the one or more introduced stop codons described above. Optionally, nucleic acid encoding other recombinant proteins or fragments thereof also are included in the vectors, such as selectable markers, repressors, inducers, tags and genetic package display proteins, such as phage coat proteins. Any suitable vector that can be modified by introduction of one or more stop codons to reduce the expression of one or more proteins of interest, as described below, can be used to generate the vectors provided herein. Such vectors include those for eukaryotic, such as mammalian, expression or prokaryotic expression, such as bacterial expression. Included amongst the vectors provided herein are plasmids, cosmids and phagemid vectors.

In one example, the vectors exhibits the ability to confer display of the polypeptide on the surface of a genetic package. When the genetic package is a virus, for example, a bacteriophage, the vector can be the genetic package. Alternatively, the vector can be separate from the genetic package, but encode a polypeptide displayed by the genetic package. Exemplary of such a vector is a phagemid vector, which encodes a polypeptide to be expressed on a bacteriophage, for example, a filamentous bacteriophage. Thus, in a particular example, the vectors are phagemid vectors that can be used to display proteins as fusion proteins with the phage coat protein on the surface of phage. Other cell surface display systems are known in the art and include, but are not limited to ice nucleation protein (Inp)-based bacterial surface display system (Lebeault J M (1998) Nat Biotechnol. 16: 576 80), yeast display (e.g. fusions with the yeast Aga2p cell wall protein; see U.S. Pat. No. 6,423,538), insect cell display (e.g. baculovirus display; see Ernst et al. (1998) Nucleic Acids Research, Vol 26, Issue 7 1718-1723), mammalian cell display, and other eukaryotic display systems (see e.g. 5,789,208 and WO 03/029456). The vectors provided herein can be used in any of these systems to display a protein of interest (provided that the host cells contain an appropriate functional suppressor tRNA and that the vectors contain the appropriate elements for replication, amplification, transcription and translation in that host cell), wherein the protein is expressed at reduced levels to reduce toxicity compared to the expression and toxicity of the protein when translated from a vector that does not contain the above-described stop codons (i.e. compared to in the absence of the stop codons).

The vectors provided herein contain an origin of replication and, typically, one or more selectable markers. Selectable markers include, but are not limited to, antibiotic resistance gene(s), where the corresponding antibiotic(s) is added to the cell culture medium to select for cells containing the vector, or any other type of selectable marker gene known in the art, such as a prototrophy-restoring gene wherein the vector is introduced into a host cell that is auxotrophic for the corresponding trait, e.g., a biocatalytic trait such as an amino acid biosynthesis or a nucleotide biosynthesis trait, or a carbon source utilization trait. Other regulatory elements can be included in the vector to enhance protein expression and regulation. Such elements include, but are not limited to, transcriptional enhancer sequences, translational enhancer sequences, promoters, activators, translational start and stop signals, transcription terminators, cistronic regulators, polycistronic regulators, tag sequences, such as nucleotide sequence “tags” and “tag” polypeptide coding sequences, which can facilitate identification, separation, purification, and/or isolation of an expressed polypeptide. For example, the vectors provided herein can contain a tag sequence, such as adjacent to the coding sequence of the protein. In one embodiment, the tag sequence allows for purification of the protein for which reduced expression is desired. For example, the tag sequence can be an affinity tag, such as a hexa-histidine affinity tag or a glutathione-S-transferase tag. The tag can also be a fluorescent molecule, such as yellow green fluorescent protein (GFP), or analogs of such fluorescent proteins. The tag can also be a portion of an antibody molecule, or a known antigen or ligand for a known binding partner useful for purification.

The nucleic acid encoding the protein(s) of interest typically is operably linked to, or contains, one or more of the following regulatory elements: a promoter, a ribosome binding site (RBS), a transcription terminator and translational start and stop signals. Many specific and consensus RBSs are known and can be used in the vectors provided herein (see e.g., Frishman et al., (1999) Gene 234(2):257-65; Suzek et al., (2001) Bioinformatics 17(12): 1123-30, and Shultzaberger et al., (2001) J. Mol. Biol. 313:215-228). In some examples, the vector contains a series of regulatory regions from a particular source. For example, the vectors provided herein can contain the repressor, promoter, operator, cap binding site, and RBS from the lactose operon from E. coli. In some examples, to promote secretion of the expressed proteins from the cytoplasm of the host cell into the periplasm or cell culture medium, the nucleic acid encoding the protein(s) of interest also is operably linked to nucleic acid encoding a leader peptide (i.e. a leader sequence). For example, the vector can contain a genetic element encoding a leader sequence and the coding sequence of a protein for which reduced expression is desired. This genetic element can be transcribed and translated as a single mRNA transcript and polypeptide, respectively. The translated leader peptide-protein fusion protein is translocated, for example, through the cytoplasmic membrane at which point the leader peptide is cleaved to release the soluble protein.

The vectors provided herein can contain nucleic acid encoding one or more proteins or fragments or domains thereof, for reduced expression to reduce toxicity compared to in the absence of the stop codons. For example, the vectors can contain nucleic acid encoding 1, 2, 3, 4, 5, 6 or more proteins or fragments thereof. For example, the vector can contain nucleic acid encoding two separate subunits of a protein, such as the A and B subunit of a toxin. In another particular example, the vectors contain nucleic acid encoding an antibody or fragments thereof. For example, the vector can contain nucleic acid encoding for a heavy chain and nucleic acid encoding for a light chain. In instances where two or more proteins or fragments thereof are expressed from the vector, the proteins can be produced from one mRNA transcript. For example, the nucleic acid encoding the two or more proteins can be under the control of a single set of transcriptional regulatory elements. Further, the mRNA can contain one or more RBSs, resulting in the translation of a single polypeptide or two or more polypeptides. In another example, the nucleic acid encoding the two or more proteins or fragments thereof can be under the control of two or more sets of transcriptional elements, thereby producing two or more mRNA transcripts.

In one embodiment, the vectors encode genetic package display proteins and can be used to display one or more proteins of interest on the a genetic package. In a particular example, the vectors are phagemid vectors and can be used to display the protein of interest as a fusion protein on the surface of phage particles. Phagemid vectors typically contain less than 6000 nucleotides and do not contain a sufficient set of phage genes for production of stable phage particles after transformation of host cells. The necessary phage genes typically are provided by co-infection of the host cell with helper phage, for example M13K01 or M13VCS. Typically, the helper phage provides an intact copy of the gene III coat protein and other phage genes required for phage replication and assembly. Because the helper phage has a defective origin of replication, the helper phage genome is not efficiently incorporated into phage particles relative to the plasmid that has a wild type origin. Thus, the phagemid vector includes a phage origin of replication for incorporation of the vector can be packaged into bacteriophage particles when host cells transformed with the phagemid are infected with helper phage, e.g. M13K01 or M13VCS. See, e.g., U.S. Pat. No. 5,821,047. The phagemid genome typically contains a selectable marker gene, e.g. Amp^{R} or Kan^{R} (for ampicillin or kanamycin resistance, respectively) for the selection of cells that are infected by the phage.

The vectors provided herein can be generated by standard cloning and recombinant techniques well known to those of ordinary skill in the art. To produce the vectors provided herein, for example, one or more features of an existing expression vector can be modified, removed or replaced, and one or more additional features can be incorporated. Exemplary vectors that can be modified, such as by recombinant techniques, to produce the vectors provided herein include, but are not limited to, the pET expression vectors (see, U.S. Pat. No. 4,952,496; available from NOVAGEN®, Madison, Wis., through EMD Biosciences; see, also literature published by Novagen describing the system), with which target genes are expressed under control of strong bacteriophage T7 transcription and translation signals, induced by providing a source of T7 RNA polymerase in the host cell. pET expression vectors include the pET-28 a-c vectors, pET 15b, pET19b and the pETDuet coexpression vectors. Other exemplary vectors that can be modified to produce the vectors provided herein include, for example, pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance.

In some instances, the vectors provided herein are phagemid vectors. Phagemid vectors are well known in the art (see, e.g., Andris-Widhopf et al. (2000) J Immunol Methods, 28: 159-81; Armstrong et al. (1996) Academic Press, Kay et al., Ed. pp. 35-53; Corey et al. (1993) Gene 128(1):129-34; Cwirla et al. (1990) Proc Natl Acad Sci USA 87(16):6378-82; Fowlkes et al. (1992) Biotechniques 13(3):422-8; Hoogenboom et al. (1991) Nuc Acid Res 19(15):4133-7; McCafferty et al. (1990) Nature 348(6301):552-4; McConnell et al. (1994) Gene 151(1-2):115-8; Scott and Smith (1990) Science 249(4967):386-90). Phagemid vectors contain a bacterial origin of replication and a phage origin of replication so that the plasmid is incorporated into bacteriophage particles when bacterial cells bearing the plasmid are infected with helper phage. In some examples, existing phagemid vectors are modified as described herein to produce phagemid vectors that facilitate reduced expression of one or more encoded proteins. Exemplary phagemid vectors that can be modified as described herein include, but are not limited to, pBluescript, pBK-CMV® (Stratagene) and pCAL vectors, which contain a sequence of nucleotides encoding the C-terminal domain of filamentous phage M13 Gene III coat protein.

In one example, the vectors provided herein are pCAL phagemid vectors. In a particular example, the vectors provided herein are produced by modification of pCAL phagemid vectors. Exemplary of pCAL vectors for modification as described herein are pCAL G13 and pCAL A1, having the sequences of nucleotides set forth in SEQ ID NOS.: 9 and 10, respectively. pCAL G13 and pCAL A1 contain the gIII gene encoding the M13 gene III (gIII) coat protein, preceded by a multiple cloning site, into which a polynucleotide can be inserted. Each of these vectors further contains an amber stop codon DNA sequence (TAG) encoding the RNA amber stop codon (UAG), just upstream of the gene III coding sequence. Thus, the vectors are designed such that polynucleotides encoding a protein of interest can be inserted just upstream of the amber stop codon and operably linked to the nucleic acid encoding the gIII coat protein. When introduced into partial amber suppressor cells, the protein of interest is expressed as a fusion protein with the gIII coat protein when read through of the stop codon occurs, and also can be expressed as a soluble protein alone when translation is terminated at the stop codon.

The pCAL G13 vector contains a guanine residue at the position just 3′ of the amber stop codon, while the pCAL A1 vector contains an adenine at this position. These differing amino acids confer different properties to the vector, such that different amounts of readthrough at the amber-stop codon occurs. Thus, the choice of vector will determine how much read-through occurs at the amber stop codon when using a partial suppressor strain, thus controlling the relative amount of fusion versus non-fusion target/variant polypeptide translated from the vector.

The vectors provided herein can be generated using standard recombinant techniques well known to those of skill in the art. It is understood that any one or more elements of the vector described herein can be substituted or replaced with a comparable element that retains essentially the same function. In other instances, any one or more elements can be removed or added, provided the vector retains the ability to introduce the nucleic acid encoding the protein of interest into a partial suppressor host cell and replicate the nucleic acid, and that, when expressed from the vector, the protein of interest is expressed at reduced levels.

a. Introduction of Stop Codons to Reduce Expression of Proteins

Provided herein are vectors for the expression of proteins, wherein toxicity of the protein is reduced by effectively down regulating expression of the protein. This is effected by introducing one or more stop codons, such as amber, ochre or opal stop codons, into the genetic element encoding the protein such that when the vector is introduced into an appropriate partial suppressor host cell, translation of the full length protein is effected only part of the time. For example, one or more amber stop codons can be introduced into the genetic element encoding the protein for which reduced expression is desired. When the vector is transformed into a partial amber suppressor strain that contains an amber suppressor tRNA, partial read through of the stop codon results and there is reduced expression of the protein compared to the expression of the same protein from a vector that does not contain the amber stop codon.

There are three different types of stop codons, each containing a different trinucleotide; amber (UAG; encoded by TAG), ochre (UAA; encoded by TAA) and opal (UGA; encoded by TGA). These stop codons can be recognized by specific suppressor tRNAs that incorporate a specific amino acid into the elongating polypeptide. Thus, instead translation terminating at the stop codon translation continues and the full length protein is produced. For example, some amber suppressor tRNAs can recognize the amber stop codon and insert a glutamine residue. In other examples, the amber suppressor tRNA inserts a serine, tyrosine, lysine or leucine. In other examples, an ochre suppressor tRNA can recognize the ochre stop codon and insert a glutamine, while other ochre suppressor tRNAs insert a lysine, and still others insert a tyrosine. Similarly, there exists opal suppressor tRNAs that recognize the opal stop codon and insert, for example, a glycine residue, or a tryptophan residue.

The stop codon(s) can be introduced into the coding sequence of the protein of interest, i.e. into the coding sequence of the protein for which reduced expression is desired to reduce toxicity, such as the domain exchanged antibody. Thus, upon translation in a partial suppressor cell, both a full length polypeptide (if there is read through of the stop codon) and a truncated polypeptide (if there is no read through and translation terminates at the stop codon) is produced. In instances where the stop codon(s) is introduced into the coding sequence of the protein of interest, the stop codon(s) typically is introduced such that termination occurs at an earlier stage of translation rather than at a later stage. For example, the stop codon(s) can be introduced in the first 10, 20, 30, 40, 50 or more nucleotides of the sequence encoding the protein for which expression will be reduced.

In a particular example, the polynucleotide encoding the protein of interest is operably linked at the 5′ end to the 3′ end of a leader sequence in the vector, and the stop codon(s) is introduced into the leader sequence. This single genetic element encoding both the leader peptide and the protein of interest is operably linked to a promoter, thus resulting in a single mRNA transcript. Translation of the resulting transcript in a partial suppressor strain, therefore, produces a full length leader peptide-protein fusion protein when there is read through of the stop codon(s), and also a truncated leader peptide, without the protein of interest, is produced if there is no read through and translation terminates at the stop codon in the leader sequence. Thus, the protein of interest is translated and expressed only part of the time. In further examples, the vector contains two or more nucleic acid regions, each encoding a protein for which reduced expression is desired, wherein each nucleic acid region is linked to a separate leader sequence and a stop codon is introduced into each leader sequence. For example, the vectors provided herein can contain nucleic acid encoding for an antibody light chain that is operably linked to a leader sequence (e.g. the PelB leader sequence) and nucleic acid encoding for an antibody heavy chain that is operably linked to another leader sequence (e.g. the OmpA leader sequence), wherein each leader sequence contains an amber stop codon. Thus, when introduced into a partial amber suppressor cell, expression of both the leader peptide-heavy chain fusion protein and leader peptide-light chain fusion protein is reduced compared to expression when the leader sequences do not contain the amber stop codons. The leader sequences are then cleaved from the light and heavy chains by bacterial peptidases following translocation across the cytoplasmic membrane.

Any number of stop codons, such as amber, ochre and/or opal stop codons, can be introduced into any regions of the genetic element encoding the polypeptide of interest, such as a domain exchanged antibody. For example, 1, 2, 3, 4, 5, 6, 7, 8, 9, 10 or more stop codons can introduced. Typically, a higher number of stop codons will result in greater reduction of expression. The stop codons can be incorporated into the nucleic acid encoding the leader peptide, or can be incorporated into the nucleic acid encoding the polypeptide of interest. In instances where antibodies, such as domain exchanged antibodies, are encoded by the vector, one or more stop codons, such as 1, 2, 3, 4, 5, 6, 7, 8, 9, 10 or more stop codons, can be incorporated into the leader sequence, and/or nucleic acid encoding the light chains, and/or nucleic acid encoding the heavy chain.

The vectors provided herein can be designed such that the amino acid that is incorporated into the growing polypeptide at the site of the introduced stop codon is that which normally would be found at that position in the polypeptide. This can be achieved by replacing a codon that encodes an amino acid that is carried by a suppressor tRNA with the stop codon that is recognized by that suppressor tRNA. For example, if the seventh amino acid of a polypeptide is glutamine then the seventh codon can be replaced by an amber stop codon, and the vector can be introduced into a partial amber suppressor cell that contains an amber suppressor tRNA (i.e. a suppressor tRNA that recognizes the amber stop codon) that carries a glutamine residue at its aminoacyl site (i.e. an amber suppressor tRNA^{Gln} molecule). Thus, when read through occurs, a glutamine residue is incorporated at the seventh amino acid position of the polypeptide, thus preserving the wild-type amino acid sequence of the protein. In another example, if the partial suppressor cell that is used as the host cell contains an amber suppressor tRNA that introduces a tyrosine residue into the growing polypeptide (i.e. an amber suppressor tRNA^{Tyr} molecule), then the amber stop codon can be incorporated into the vector, such as in the leader sequence operably linked to the protein of interest, in place of a codon encoding a tyrosine residue. Thus, when read through occurs in a partial amber suppressor cell, the polypeptide is produced with a tyrosine at the position encoded by the amber stop codon, thus preserving the wild type amino acid sequence of the polypeptide. In other instances, the amino acid that is incorporated at the site of the introduced stop codon is different to the amino acid that is normally present at that position in the polypeptide. Typically, the amino acid that is introduced, however, is one that does not alter the conformation and/or function of the translated protein. As noted above and below in section D, a range of natural and synthetic suppressor tRNAs exist that incorporate various amino acid residues at the different stop codons. Further, additional suppressor tRNA molecules can be generated by mutation of the tRNA anticodon using recombinant techniques well known in the art. Thus, a variety of wild type codons can be selected as the site for introduction of the stop codon, resulting in incorporation of the wild-type amino acid residue by a suitable suppressor tRNA when the vector is introduced into an appropriate partial suppressor strain.

The efficiency of suppression can be affected by the amino acids adjacent to the introduced stop codon (see e.g. Urban et al., (1996) Nucl. Acids. Res. 24(17): 3424-3430). In some examples, single nucleotide changes can be made 3′ or 5′ of the stop codon to increase or decrease suppression efficiency. In other examples, multiple nucleotide changes can be made immediately 3′ or 5′ of the stop codon to increase or decrease suppression efficiency. One of skill in the art can modify the sequence adjacent to the introduced stop codon to increase or decrease the suppression efficiency observed when the vector is introduced into an appropriate partial suppressor cell.

b. Introduction of a Stop Codon to Facilite Expression of Soluble Proteins and Fusion Proteins

Provided herein are vectors for the expression of both soluble proteins and fusion proteins. In particular, provided herein are phagemid vectors for the expression of both soluble proteins and protein-display protein fusion proteins, and the display thereof. This is effected by incorporation of a stop codon between the nucleic acid encoding the protein of interest and the nucleic acid encoding the display protein. Such termination or stop codons include, for example, the amber stop codon (UAG; encoded by TAG)), the ochre stop codon (UAA; encoded by TAA) and the opal stop codon (UGA; encoded by TCA). When expressed in an appropriate partial suppressor strain (e.g. an amber partial suppressor strain if an amber stop codon is introduced), translation can continue through the stop codon, thus generating detectable quantities of a fusion protein containing the protein of interest and the coat protein, or can be terminated at the stop codon, thus producing the protein of interest alone.

Thus, in one example, the presence of a stop codon, such as an amber stop codon, in the vectors provided herein between the sequence encoding the polypeptide of interest and the coat protein is used to regulate expression of the polypeptide-coat protein fusion protein versus the polypeptide alone, in an suppressor strain of host cell (e.g. an amber suppressor strain). For example, an amber stop codon can be included between the 3′ end of a polynucleotide encoding an antibody heavy chain and the 5′ end of a nucleic acid encoding a phage coat protein, for example, gene III coat protein. When the vector is introduced into a partial amber suppressor strain, a mixed collection of polypeptides is produced. The mixed population contains some fusion proteins containing the antibody heavy chain and coat protein, and some heavy chain polypeptides that are not part of fusion proteins with phage coat proteins, and thus, are soluble. In one example, the mixed population contains between 50% or about 50% and 75% or about 75% soluble polypeptide, for example, soluble heavy chain polypeptide, and between 25% or about 25% and 50% or about 50% fusion protein.

In some instances, the soluble polypeptide interacts with the fusion protein, for example, through hydrophobic interactions and/or disulfide bonds, so that both polypeptides are expressed on the surface of the phage. For example, the vectors provided herein can encode a domain exchanged Fab, wherein a single genetic element encodes a leader peptide linked to a light chain (V_{L}C_{L}), and another leader peptide linked to a heavy chain (V_{H}C_{H}) that is linked to a phage coat protein. Stop codons are present in the nucleic acid encoding the leader peptides, so that expression of the domain exchanged Fab is reduced in partial suppressor cells. A stop codon also is present between the nucleic acid encoding the antibody heavy chain and the nucleic acid encoding the phage coat protein. Thus, in a partial suppressor cell, soluble light chains, soluble heavy chains and heavy chain-coat protein fusion proteins are produced. Two soluble light chains can associate with a soluble heavy chain and a heavy chain-phage coat protein fusion and form the “interlocked” configuration that is characteristic of domain exchanged antibodies (described below), in which the domain exchanged Fab actually contains a pair of interlocked Fabs whereby each V_{H} domain interacts with the V_{L} domain that is “opposite” to the interaction that occurs through the constant regions (see FIG. 2a).

b. Other Features

As discussed above, the vectors provided herein typically contain other elements and/or genes that facilitate regulated and efficient expression of proteins and fragments or domains thereof. In particular, regulatory elements such as promoters can be selected for additional control of expression, while leader sequences that encode peptide leaders can be operably linked to the nucleic acid encoding the protein of interest to ensure efficient transport from the cytoplasm to the periplasm of the host cell or the cell culture medium. Additionally, the vectors provided herein, such as the phagemid vectors provided herein, can contain other elements to facilitate display of the protein of interest on the surface of phage. Thus, such phagemid vectors can be used to generate phage display libraries in which proteins, such as antibodies, including domain exchanged antibodies, are stably expressed at reduced levels, allowing for subsequent selection and enrichment.

i. Promoters

The vectors provided herein contain one or more promoters operably linked to the genetic element or nucleotides encoding the protein for which reduced expression is desired. In some embodiments, non-regulatable promoters are used. Regulatable or non regulatable (e.g. constitutive) promoters can be used. An example of a non-regulatable promoter is the gIII promoter. In other examples, regulatable promoters are used in the vectors provided herein. The use of regulatable promoters can provide another level of protein expression control, whereby expression of the protein, even in a suppressor or partial suppressor strain, is initiated only when the appropriate conditions are provided.

Many regulatable (e.g., inducible and/or repressible) promoter sequences are known and can be used in the vectors provided herein. Such sequences include regulatable promoters whose activity can be altered or regulated by the intervention of the user, e.g., by manipulation of an environmental parameter, such as, for example, temperature or by addition of stimulatory molecule or removal of a repressor molecule. For example, an exogenous chemical compound can be added to regulate transcription of some promoters. Regulatable promoters can contain binding sites for one or more transcriptional activator or repressor protein. Synthetic promoters that include transcription factor binding sites can be constructed and also can be used as regulatable promoters. Exemplary regulatable promoters include promoters responsive to an environmental parameter, e.g., thermal changes, hormones, metals, metabolites, antibiotics, or chemical agents. In some examples, regulatable promoters are induced and/or repressed by one or more molecules. In other examples, inducible promoters are induced by a process of derepression, e.g., inactivation of a repressor molecule.

Regulatable promoters appropriate for use in E. coli include promoters that contain transcription factor binding sites from the lac, tac, trp, trc, and tet operator sequences, or operons, the alkaline phosphatase promoter (pho), an arabinose promoter such as an araBAD promoter, the rhamnose promoter, the promoters themselves, or functional fragments thereof (see, e.g., Elvin et al. (1990) Gene 37: 123-126; Tabor and Richardson, (1998) Proc. Natl. Acad. Sci. U.S.A. 1074-1078; Chang et al. (1986) Gene 44: 121-125; Lutz and Bujard, (1997) Nucl. Acids. Res. 25: 1203-1210; D. V Goeddel et al. (1979) Proc. Nat. Acad. Sci. U.S.A., 76:106-110; J. D. Windass et al. (1982) Nucl. Acids. Res., 10:6639-57; R. Crowl et al. (1985) Gene, 38:31-38; Brosius (1984) Gene 27: 161-172; Amanna and Brosius, (1985) Gene 40: 183-190; Guzman et al. (1992) J. Bacteriol., 174: 7716-7728; Haldimann et al. (1998) J. Bacteriol., 180: 1277-1286).

A regulatable promoter sequence also can be indirectly regulated. Examples of promoters that can be engineered for indirect regulation include, but are not limited to, the phage lambda PR, PL, phage T7, SP6, and T5 promoters. For example, the regulatory sequence is repressed or activated by a factor whose expression is regulated, e.g., by an environmental parameter. One example of such a promoter is a T7 promoter. The expression of the T7 RNA polymerase can be regulated by an environmentally-responsive promoter such as the lac promoter. For example, the cell can include a heterologous nucleic acid that includes a sequence encoding the T7 RNA polymerase and a regulatory sequence (e.g., the lac promoter) that is regulated by an environmental parameter. The activity of the T7 RNA polymerase also can be regulated by the presence of a natural inhibitor of RNA polymerase, such as T7 lysozyme.

In another configuration, the lambda PL can be engineered to be regulated by an environmental parameter. For example, the cell can include a nucleic acid that encodes a temperature sensitive variant of the lambda repressor. Raising cells to the non-permissive temperature releases the PL promoter from repression.

The regulatory properties of a promoter or transcriptional regulatory sequence can be easily tested by operably linking the promoter or sequence to a sequence encoding a reporter protein (or any detectable protein). This promoter-report fusion sequence is introduced into a bacterial cell, typically in a plasmid or vector, and the abundance of the reporter protein is evaluated under a variety of environmental conditions. A useful promoter or sequence is one that is selectively activated or repressed in certain conditions.

lac Promoter

Exemplary of regulatable promoters is the lac promoter, which can be induced by lactose or structurally related molecules such as isopropyl-beta-D-thiogalactoside (IPTG) and also can be repressed by glucose. In one example, the vectors provided herein contain the full length lac I gene (encoding the lac repressor), which is driven by the I gene promoter, followed by the tHP transcription terminator, a cap binding site, and the lac promoter (lacP) and lac operator (lacO). The regulatory response to lactose requires the constitutively-expressed lac repressor, which binds very tightly to the lac operator in the absence of lactose and interferes with binding of RNA polymerase to the promoter, inhibiting transcription of the operably linked protein. In the presence of lactose or a suitable equivalent, such as IPTG, however, the lactose metabolite allolactose binds to the repressor, causing a conformational change that renders the repressor unable to bind to the operator, thereby allowing binding of the RNA polymerase and transcription of the protein.

ii. Leader Sequences

For efficient isolation of the expressed protein, elements can be include in the vectors provided herein to secrete the protein into the culture medium or, in the case of gram-negative bacteria (e.g. E. coli), into the periplasmic space (or periplasm) between the inner and outer cell membranes. Secreted proteins typically are soluble and can readily be separated from contaminating host proteins and other cellular components. Further, secretion of the protein is required for efficient display on genetic packages, such as bacteriophage. The entry of almost all secreted proteins to the secretory pathway, in both prokaryotes and eukaryotes, is directed by specific N-terminal signal peptides, or leader peptides (encoded by leader sequences). These leader peptides are cleaved from the protein by membrane bound peptidases following translocation of the protein through the membrane. Thus, in some examples, the vectors provided herein contain a leader sequence operably linked to the 5′ end of the nucleic acid encoding the protein for which reduced expression is desired, such that upon expression, the protein is directed through the secretory pathway by the leader peptide and secreted into the periplasm or cell culture medium. In examples where more than one protein of interest is encoded by the vector, a leader sequence can be operably linked to each nucleic acid sequence encoding each protein. For example, the vectors provided herein can contain a genetic element operably linked to a promoter, wherein the genetic element encodes a leader peptide and a protein for which reduced expression is desired. Thus, upon transcription and translation, a polypeptide containing the leader peptide fused to the protein of interest if produced and transported across the membrane, where the leader peptide is cleaved to release the soluble protein. Typically, the leader sequence in the genetic element contains a stop codon, such as an amber stop codon, to reduce expression of the linked protein in partial suppressor cells, as described above. In another example, the vector contains a genetic element operably linked to a promoter, wherein the genetic element encodes a leader peptide linked to a protein, and another leader peptide linked to another protein. Typically, each of the leader sequences contains a stop codon to facilitate reduced expression of both proteins in partial suppressor cells.

Any suitable leader sequence known in the art can be included in the vectors provided herein to direct secretion of the proteins to the periplasm or cell culture medium. For expression in E. coli, for example, a suitable prokaryotic leader sequence encoding a prokaryotic leader peptide is used. Most prokaryotic leader peptides are 20-30 amino acids in length, with the hydrophobic region (12-14 amino acid residues in length) in the middle, and a positively charged region close to the N-terminus (Pugsley (1993) Microbiol. Rev. 57:50-108). A number of leader peptides from prokaryotic proteins and from phage proteins are known in the art (see, for example, Gennity et al. (1990) J. Bioeng. Biomemb. 22:233-269) and can be used in the vectors herein. Examples of suitable leader peptides for the secretion of proteins from E. coli include, but are not limited to, the leader peptide from Pectate lyase B protein from Erwinia carotovora (PelB) and the E. coli leader peptides from the outer membrane protein (OmpA; U.S. Pat. No. 4,757,013); heat-stable enterotoxin II (StII); alkaline phosphatase (PhoA), outer membrane porin (PhoE), and outer membrane lambda receptor (LamB). Non-limiting examples of viral leader peptides include the N-terminal signal peptide from the bacteriophage proteins pIII and pVIII, pVII, and pIX. Also included in the leader peptides that can be used in the vectors herein are modified and/or synthetic leader peptides, such as those described in U.S. Pat. Nos. 5,470,719 and 6,875,590, and International Patent Publication No. WO2003040335.

iii. Phage Display Features

In some embodiments, the vectors provided herein are phagemid vectors for use in generating phage display libraries in which a protein, such as an antibody or fragment thereof, including domain exchanged antibodies or fragments thereof, are displayed on the surface of phage. Phage display systems typically utilize filamentous phage, such as M13, fd, and fl. In some examples using filamentous phage, the protein for which reduced expression is desired is fused to a phage coat protein anchor domain. In order to generate phage display libraries containing fusion proteins using the vectors provided herein, the nucleic acid encoding the protein(s) for which reduced expression is desired is near, typically adjacent or nearly adjacent to (along the linear nucleic acid sequence) the nucleic acid encoding a phage coat protein. In one example, the polynucleotide encoding the protein of interest is fused to nucleic acids encoding the C-terminal domain of filamentous phase M13 Gene III (gIIIp; g3p; cp3, gene 3 protein)

Phage coat proteins that can be used for display of polypeptides and that, therefore, can be encoded in the vectors provided herein, include (i) minor coat proteins of filamentous phage, such as gene III protein (gIIIp), and (ii) major coat proteins of filamentous phage such as gene VIII protein (gVIIIp). Fusions to other phage coat proteins such as gene VI protein, gene VII protein, or gene IX protein also can be used (see, e.g., International Patent Publication No. WO 00/71694). Alternatively, nucleic acids encoding portions (e.g., domains or fragments) of these proteins can be included the vectors. Useful portions include domains that are stably incorporated into the phage particle so that the fusion protein remains in the particle throughout a screening and/or selection procedure, such as, for example, a selection procedure as described below. In one example, the anchor domain of gIIIp is used (see, e.g., U.S. Pat. No. 5,658,727). In another example, gVIIIp is used (see, e.g., U.S. Pat. No. 5,223,409). In one example, the gVIIIp is a mature, full-length gVIIIp fused to the protein for which reduced expression is desired. Filamentous phage display systems typically use protein fusions to attach the heterologous amino acid sequence to a phage coat protein or anchor domain. For example, the phage can include a gene that encodes a signal sequence, the heterologous amino acid sequence, and the anchor domain, e.g., a gIIIp anchor domain.

Valency of the fusion protein displayed on the genetic package can be controlled by choice of phage coat protein and the nucleic acids encoding the coat protein. For example, gIIIp proteins typically are incorporated into the phage coat at three to five copies per virion. Fusion of gIIIp to variant proteases thus produces a low-valency. In comparison, gVIII proteins typically are incorporated into the phage coat at 2700 copies per virion (Marvin (1998) Curr. Opin. Struct. Biol. 8:150-158). Due to the high-valency of gVIIIp, peptides greater than ten residues are generally not well tolerated by the phage. Phagemid systems can be used to increase the tolerance of the phage to larger peptides, by providing wild-type copies of the coat proteins to decrease the valency of the fusion protein. Additionally, mutants of gVIIIp can be used which are optimized for expression of larger peptides. In one such example, a mutant gVIIp was obtained in a mutagenesis screen for gVIIIp with improved surface display properties (Sidhu et al. (2000) J. Mol. Biol. 296:487-495).

In one example, the vectors provided herein are designed so that the fusion protein further includes a flexible peptide linker or spacer, a tag or detectable polypeptide, a protease site, or additional amino acid modifications to improve the expression and/or utility of the fusion protein containing the protein of interest and coat protein. For example, addition of a nucleic acid encoding a protease site can allow for efficient recovery of desired bacteriophages following a selection procedure. Exemplary tags and detectable proteins are known in the art and include for example, but not limited to, a histidine tag, a hemagglutinin tag, a myc tag or a fluorescent protein. In another example, the nucleic acid encoding the protease-coat protein fusion can be fused to a leader sequence in order to improve the expression of the polypeptide. Exemplary of leader sequences include, but are not limited to, PelB and OmpA.

d. Exemplary Polypeptides for Expression Using the Vectors

The vectors provided herein can be used to express any protein. In some examples, the vectors can be used to express polypeptides for which reduced expression is desired. In other examples, the vectors are used to produce soluble proteins and fusion proteins. In particular examples, the vectors are phagemid vectors and are used in, for example, the generation of phage display libraries in which a protein, such as an antibody, is displayed on the surface of a phage. In a particular example, the vectors contain polynucleotides from a nucleic acid library, such as variant polynucleotides from a nucleic acid library, such as those generated using the methods described in related U.S. application No. [Attorney Docket No. 3800013-00031/1106] and International Application No. [Attorney Docket No. 3800013-00032/1106PC] and summarized below and exemplified in Example 5, below. Thus, in one example, a collection of the phagemid vectors provided herein containing variant polynucleotides encoding variant polypeptides can function as a nucleic acid library and can be used to generate a phage display library. In one example, the polynucleotides, including variant polynucleotides, contained in the vectors encode an antibody, such as a domain exchanged antibody, or domain or fragment thereof, that is expressed as a fusion protein with the phage coat protein and displayed on the surface of phage. As discussed, in some instances, the vectors can be used to reduce the toxicity of the expressed protein. By reducing the toxicity of the expressed polypeptide, such as a domain exchanged antibody, to the host cell using the vectors and methods provided herein, a more diverse and stable library can be generated. Thus, using the vectors and methods provided herein, proteins that typically are toxic to the host cell and which may otherwise have been undetected in phage display libraries due to their instability, can be identified, selected, and/or enriched.

Although any polypeptide can be expressed using the vectors provided herein, in some instances, the vectors are of particular use in the expression of proteins that exhibit toxicity. Exemplary proteins that exhibit toxicity and that can be expressed from the vectors provided herein include eukaryotic and prokaryotic proteins, such as proteins from humans and other mammals, non-mammalian animals, plants, insects, yeast, bacteria and viruses. Further, the proteins can be, for example, membrane proteins, cytoplasmic proteins, structural proteins, soluble proteins, glycoproteins or nucleases. Non-limiting examples of proteins that can be encoded by nucleic acid contained in the vectors herein for reduced expression include, include, but are not limited to, viral proteins such as the HIV-1 env protein, rabies virus glycoprotein and vesicular stomatitis virus G protein; bacterial proteins such as Pseudomonas exotoxin A, cholera toxin, diphtheria toxin, E. coli toxins, botulinum toxin, anthrax toxin, pertussis toxin, shiga toxin, ricin, tetanus toxin, and Staphylococcal toxins; and human proteins such as TNF-α, TNF-β, IFN-γ, IL-2, Fas ligand and antibodies, fragments and domains thereof.

In some examples, the proteins encoded in, and expressed from, the vectors provided herein are antibody polypeptides, including antibody fragments. Thus, in some instances, the vectors provided herein can contain nucleic acid encoding any antibody, domain or fragment thereof, such that when the vector is introduced into a suitable partial suppressor cell, expression of the antibody is reduced compared to expression of the same antibody from a vector that does not contain the introduced stop codon(s), as described above. In some examples, the vectors provided herein are phagemid vectors and the antibody that is encoded by the vector is expressed as a fusion protein with the phage coat protein for display on phage.

The vectors provided herein can be used to express any antibody or fragment thereof, or domain thereof, at reduced levels. One of skill in the art can readily identify the nucleic acid encoding an antibody of interest and introduce it, such as by standard cloning techniques, into a vector provided herein so that, when the vector is introduced into an appropriate partial suppressor cell, expression of the antibody is reduced compared to when the same antibody is expressed from a similar vector that does not contain the introduced stop codons. The nucleic acid encoding an antibody or fragment thereof can be introduced, for example, down stream of a leader sequence that contains a stop codon, such as an amber stop codon. Thus, when a partial amber suppressor strain is transformed with the vector, translation of the complete leader peptide-antibody fusion protein occurs only part of the time, while at other times, translation terminates at the stop codon in the leader sequence. In some instances, two or more domains of an antibody are expressed as two or more polypeptides. For example, a Fab fragment can be expressed from the vectors provided herein from one transcript that encodes two leader peptides, each fused to a heavy chain or a light chain. Thus the vector can contain a promoter operably linked to a leader sequence, polynucleotides encoding a light chain, another leader sequence and polynucleotides encoding a heavy chain. Ribosome binding sites are positioned before each leader sequence. Thus, a single transcript is produced from which two polypeptides are expressed (leader peptide-light chain and leader peptide-heavy chain). In further examples, one of the antibody chains, such as the heavy chain, also can be fused to a phage coat protein by operably linking the polynucleotides encoding the heavy chain to polynucleotides encoding a coat protein, such as the gIII (or G3) coat protein. In a particular embodiment, a stop codon separated the nucleic acid encoding the heavy chain and the nucleic acid encoding the gIII coat protein, such that upon expression in a suitable partial suppressor cell, both soluble Fab fragments and Fab-gIII fusion protein are produced. Using similar strategies, one of skill in the art can express any antibody or fragment thereof, including Fab, Fab′, F(ab′)₂, single-chain Fvs (scFv), Fv, dsFv, diabody, Fd and Fd′ fragments, from the vectors provided herein for reduced expression in a partial suppressor strain. In one example, the vectors provided herein encode a domain exchanged antibody.

d. Expression of Domain Exchanged Antibodies from the Vectors Herein

The provided vectors can be used to display domain exchanged antibodies (which are bivalent antibodies with two interlocked heavy chains), and other bivalent antibodies, on the surface of genetic packages. Due to the unusual configuration of domain exchanged antibodies and fragments thereof, their display on phage can be problematic using conventional phage display methods. For example, a conventional Fab fragment contains one light chain (V_{L} and C_{L}) and a heavy chain fragment, containing a variable domain of a heavy chain (V_{H}) and one constant region domain of the heavy chain (C_{H}1). Conventional phage display methods used to generate phage displayed Fab fragments include, for example, generating a vector for expression of a heavy chain-coat protein fusion polypeptide and a native light chain polypeptide, which then interact to form the Fab fragment.

In contrast, because of the mutation within the joining region between the V_{H} and C_{H}, the variable heavy chain domain of a domain-exchange antibody “swings away” from its cognate light chain, and instead interacts with the “opposite” light chain (the light chain other than the light chain with which the variable constant region interacts). Additional framework mutations along the V_{H}-V_{H}′ interface act to stabilize this domain-exchange configuration. Because of this altered configuration, a domain-exchange Fab fragment contains not the typical heavy chain/light chain pair, but a pair of interlocked Fabs where each V_{H} domain interacts with the V_{L} domain that is “opposite” to the interaction that occurs through the constant regions. Due to this unusual configuration, conventional means of expressing a heavy chain-coat protein fusion and a native light chain cannot be used to display domain exchanged antibody Fab fragments. Display of other domain exchanged fragments, for example, scFv domain exchanged fragments, presents similar limitations.

Thus, to display domain exchanged antibodies and fragments on phage using the vectors provided herein, the vectors are designed such that two distinct heavy chains can be expressed: one (V_{H}) expressed as part of a fusion protein with a phage coat protein, and the other (V_{H}′) expressed as a native (or soluble) heavy chain. The vector also encodes light chain polypeptides. Following expression, two soluble light chains can associate with a soluble heavy chain and a heavy chain-phage coat protein fusion and form the “interlocked” configuration that is characteristic of domain exchanged Fab to display domain exchanged Fab fragments on phage. In one example, the two distinct heavy chains are encoded by and expressed from a single genetic element, e.g. a single nucleic acid (sequence of nucleotides) in a vector. Thus, in this example, because they are encoded by a single genetic element, the amino acid sequences of the two heavy chains (V_{H} and V_{H}′) within the two polypeptides are 100% identical. This can be achieved by generating a vector that contains a polynucleotide encoding the heavy chain linked to a polynucleotide encoding the phage coat protein, whereby the polynucleotides are separated by a stop codon, such as an amber stop codon. Thus, when the vector is incorporated into an appropriate partial suppressor cell, such as an amber partial suppressor cell if the stop codon is an amber stop codon, both the native heavy chain and the heavy chain-phage coat protein fusion protein are expressed.

Domain exchanged antibody fragments that can be expressed using the vectors provided herein are illustrated in FIGS. 2a-h, which depicts the antibody fragments as part of bacteriophage coat protein 3 (G3) fusion proteins for display on filamentous bacteriophage. Alternatively, any of the fragments depicted in the figure and described herein can be adapted for display on other genetic packages, for example, using different genetic package vectors and coat proteins. Further, the fragments can be produced as non-fusion protein fragments for purposes other than display on genetic packages. The fragments described below are exemplary and the methods for vector design can be used in various combinations to generate other related domain exchanged fragments for display on genetic packages.

In one example, the vectors provided herein are phagemid vectors and the domain exchanged antibodies or fragment thereof are expressed for display on phage. Display of domain exchanged Fab fragments, domain exchanged scFv fragments, and related fragments can be achieved by inserting into the vector a nucleotide sequence encoding a stop codon, for example, an amber stop codon (UAG or TAG)), an ochre stop codon (UAA or TAA) or an opal stop codon (UGA or TGA), between the nucleic acid encoding all or part of the antibody fragment and the nucleic acid encoding the phage coat protein. For example, the polynucleotides encoding all or part of the domain exchanged antibody fragments are linked at the 5′ end to a leader sequence into which a stop codon has been introduced, thus facilitating reduced expression in an suitable partial suppressor cell. Thus, upon expression in a suitable partial suppressor cell, the domain exchanged fragment is expressed as a fusion protein with the phage coat protein when there is readthrough of the stop codon between the nucleic acid sequence encoding the antibody chain and the gene encoding the phage coat protein, and also is expressed as a soluble antibody when translation is terminated at the stop codon between the nucleic acid sequence encoding the antibody chain and the gene encoding the phage coat protein. Thus, this partial read-through of the stop codon between the nucleic acid encoding all or part of the antibody fragment and the nucleic acid encoding the phage coat protein results in a mixed collection of polypeptides. The mixed collection contains some polypeptide fusion proteins and some soluble polypeptides, which are not part of coat protein fusions. In one example, the mixed population contains between 50% or about 50% and 75% or about 75% soluble polypeptide and between 25% or about 25% and 50% or about 50% polypeptide-coat protein fusion protein.

In addition to inserting a stop codon between the polynucleotide encoding the antibody chain and the polynucleotide encoding a phage coat protein, other modifications also can be made to the domain exchanged antibody to optimize expression and structure of the protein. For example, nucleic acid encoding the domain exchanged antibody can be modified to encode a peptide linker(s) between antibody domains; be modified, such as by mutation to facilitate amino acid substitutions, to promote covalent intra-chain interactions, for example, by promoting formation of disulfide bonds; and be modified to encode additional domains, such as dimerization domains and/or hinge regions and combinations thereof.

Exemplary of the domain exchanged fragments that can be encoded by the vectors provided herein are fragments in which two chains (e.g. two V_{H}-C_{H}1 heavy chains or two V_{H}-linker-V_{L} single chains), encoded by the same genetic element (e.g. nucleotide sequence), are expressed on one phage as part of the domain exchanged antibody fragment. Typically, in this example, one of the chains is expressed as a soluble, non-fusion protein (e.g. V_{H}-C_{H}1 or V_{H}-V_{L}) and the other is expressed as a phage coat protein fusion protein (e.g. V_{H}-C_{H}1-cp3 or V_{L}-V_{H}-cp3). In this example, however, the antibody chain portion of the polypeptides is identical because they are encoded by the same genetic element. Also exemplary of the provided fragments are those (e.g. scFv tandem), containing multiple domains (e.g. V_{H}, V_{L}, C_{H}1, C_{L}) that are connected with peptide linkers to form the two heavy chain and two light chain domains of the domain exchanged configuration. Thus, using the vectors provided herein for display of domain exchanged fragments, two copies of a chain of the fragment, for example, two copies of the V_{H}-C_{H}1 heavy chain or the V_{H}-linker-V_{L} chain, can be expressed, one as a fusion protein and one as a soluble protein. These two chains interact on the surface of the phage through conventional and/or artificial interactions (e.g. hydrophobic interactions, disulfide bonds and/or dimerization domains), to display domain exchanged antibodies with two conventional antigen combining sites.

Exemplary of domain exchanged fragments that can be displayed on phage using the phagemid vectors provided herein are the domain exchanged Fab fragment (illustrated in FIG. 2a), the domain exchanged scFv fragment (illustrated in FIG. 2f), and variations thereof. Thus, in one example, the vector contains nucleic acid encoding the V_{H}-C_{H}1 chain, followed by nucleic acid encoding a stop codon (e.g. the amber stop codon (TAG)), followed by a nucleic acid encoding a coat protein. A leader sequence containing a stop codon is linked to the 5′ end of the nucleic acid encoding the V_{H}-C_{H}1 chain. The vector also includes a leader sequence containing a stop codon linked to nucleic acid encoding a light chain (V_{L}-C_{L}). When expressed in an appropriate partial suppressor host cell, two separate heavy chain elements (V_{H}-C_{H}1 and V_{H}-C_{H}1-coat protein fusion) are produced from a single copy of the encoding nucleic acid. These two copies of the heavy chain assemble, along with two soluble light chains (V_{L}-C_{L}), to form the domain exchanged “Fab” antibody on the surface of the genetic package, having two conventional antibody combining sites. Due to the stop codons in the leader sequences, the light and heavy chains are expressed at reduced levels in a partial suppressor cell compared to the expression levels of the same protein using a vector that does not contain the stop codons in the leader sequence.

In another example, the vectors provided herein encode one V_{H} and one V_{L} domain, joined by a peptide linker (V_{H}-linker-V_{L}), and can be used to express and display a domain exchanged scFv fragment. For example, the vector can contain a leader sequence into which a stop codon has been introduced. This leader sequence is linked to the polynucleotide encoding the V_{H}-linker-V_{L}, which is linked to a polynucleotide encoding a phage coat protein. A stop codon also separates the coding sequences of the V_{H}-linker-V_{L} and phage coat protein. Thus, upon expression in a partial suppressor cell, both the V_{H}-linker-V_{L}-phage coat protein fusion protein and the V_{H}-linker-V_{L} soluble protein are expressed at reduced levels. These two chains can then interact through the V_{H} domains, providing the interlocked domain exchanged scFv configuration (FIG. 2f).

Also exemplary of displayed (e.g. phage-displayed) domain exchanged antibody fragments that are generated using the provided stop codon methods are the domain exchanged Fab hinge fragment (example illustrated in FIG. 2b), the domain exchanged Fab Cys19 fragment (example illustrated in FIG. 2c), the domain exchanged scFab ΔC2 and scFab ΔC2 Cys19 fragments (example illustrated in FIG. 2d), scFv hinge fragment (example illustrated in FIG. 2g) and scFv Cys19 fragments (example illustrated in FIG. 2h).

i. Peptide Linkers

In some examples, the domain exchange structure of displayed antibody fragments is promoted by including nucleotide sequences encoding peptide linkers, between sequences encoding the antibody fragment. This technique can be used to promote and/or stabilize the domain exchanged configuration. In some examples, the peptide linkers bring two antibody variable domains (encoded by separate genetic elements within the vector) into proximity, allowing formation of the domain exchanged three-dimensional structure with two heavy chain and two light chain variable regions. In another example, the domain exchanged structure is stabilized by the use of peptide linkers between two or more chains.

Exemplary of domain exchanged fragments containing peptide linkers to promote domain exchanged configuration is the domain exchanged scFv tandem fragment. An example of this fragment displayed on phage, as part of a cp3 fusion protein, is illustrated in FIG. 2e. In the nucleic acid molecule encoding this fragment, three polynucleotides encoding peptide linkers are inserted between the nucleic acids encoding a first V_{L} and first V_{H} chain, between the nucleic acids encoding the first V_{H} and a second V_{H} chain, and between nucleic acids encoding the second V_{H} and a second V_{L} chain. Thus, while for display of a domain exchanged Fab fragment, two heavy chains (soluble and fusion protein) are encoded by a single genetic element, as described above, the scFv tandem vector, by contrast, carries two copies each of identical nucleic acid molecules encoding the light chain and heavy chain variable region domains, all four of which are joined by nucleic acids encoding peptide linkers. Thus, in the fragment, two heavy and two light chain variable region domains are joined by peptide linkers. In the case of a displayed domain exchanged scFv tandem fragment (as illustrated in FIG. 2e), the four chains are expressed as a single chain coat protein fusion molecule, on the genetic package surface, to form the domain exchanged structure.

In another example, peptide linkers are used to promote stability of a domain exchanged scFv fragment, an example of which is illustrated in FIG. 2f. As described above, this fragment contains two chains, each containing one V_{H} and one V_{L} domain, joined by a peptide linker. The two chains interact through the V_{H} domains, providing the domain exchanged configuration. For display of the domain exchanged scFv fragment, one chain is expressed as a soluble V_{H}-linker-V_{L} and the other chain is expressed as a V_{H}-linker-V_{L}-coat protein fusion protein, as described above. In a further example, the domain exchanged Fab fragment encoded by the vectors provided herein contains nucleic acid sequences encoding peptide linkers between the V_{L}-C_{L} coding sequence and the V_{H}-C_{H}1-coat protein coding sequence, thereby generating, upon expression in a partial suppressor strain, one V_{L}-C_{L}-linker-V_{H}-C_{H}1-coat protein fusion chain and one soluble V_{L}-C_{L}-linker-V_{H}-C_{H}1 chain, which pair on the phage surface to form a single chain Fab (scFab) fragment, such as the scFabΔC² fragment (FIG. 2d(i)). As illustrated in FIG. 2d(i), in the scFab ΔC² fragment, two cysteines can be mutated to ablate formation of the disulfide bonds between the constant regions, as the presence of the linkers makes these disulfide bonds unnecessary for stabilizing the folded antibody fragment. A modified scFab ΔC² fragment, the scFab ΔC²Cys19 fragment, which contains an Ile19 to Cys19 mutation to promoter a disulfide bridge between VH-VH′ interface, also can be encoded in the vectors provided herein.

Linkers for use in antibody fragments are well known in the art. Exemplary linkers that can be inserted between chains in the provided methods are listed in Table 3. Methods for preparation of these linkers and their insertion into vectors for expression of domain exchanged antibody fragments are well known in the art and described elsewhere (see e.g. related U.S. application No. [Attorney Docket No. 3800013-00031/1106] and International Application No. [Attorney Docket No. 3800013-00032/1106PC].

TABLE 3

Linkers for generating domain exchanged antibody
fragments for phage display

| | | | SEQ ID | Amino |
|---|---|---|---|---|
| | | | NO | acid |
| Linker | Nucleotide sequence encoding | SEQ ID NO | (amino | length of |
| Name | linker | (nucleotide) | acid) | linker |
| Linker 1 | GGTGGTTCGTCTGGATCTTCCTCCT | 11 | 12 | 18 |
| | CTGGTGGCGGTGGCTCGGGCGGTG | | | |
| | GTGGC | | | |
| Linker 2 | GGAGGATCCGGCAGCAGCAGCAGC | 13 | 14 | 18 |
| | GGCGGCGGCGGCGGGAGCTCCGGC | | | |
| | GGCGGA | | | |
| L216 | GGAGGATCCGGCAGCAGCAGCAGC | 15 | 16 | 16 |
| | GGCGGCGGGAGCTCCGGCGGCGGA | | | |
| L217 | GGAGGATCCGGCAGCAGCAGCAGC | 17 | 18 | 17 |
| | GGCGGCGGCGGGAGCTCCGGCGGC | | | |
| | GGA | | | |
| L219 | GGAGGATCCAGCGGCAGCAGCAGC | 19 | 20 | 19 |
| | AGCGGCGGCGGCGGCGGGAGCTCC | | | |
| | GGCGGCGGA | | | |
| L220 | GGAGGATCCAGCGGCGGCAGCAGC | 21 | 22 | 20 |
| | AGCAGCGGCGGCGGCGGCGGGAGC | | | |
| | TCCGGCGGCGGA | | | |
| BamHISacI | GATCCGGTGGCGGCAGCGAAGGTG | 23 | 24 | 29 |
| | GTGGCAGCGAAGGTGGCGGTAGCG | | | |
| | AAGGTGGCGGCAGCGAAGGCGGCG | | | |
| | GTAGCGGTGGGAGCT | | | |

ii. Dimerization Domains

In some examples, one or more dimerization domains are included in the displayed domain exchange antibody fragment, in order to promote interaction between chains, and stabilize the domain exchange configuration. Thus, in some examples, the provided vectors include nucleic acids encoding one or more dimerization domains which can promote interaction between polypeptide chains and can stabilize the domain exchange configuration. Dimerization domains include any domain that facilitates interaction between two polypeptide sequences (e.g. antibody chains). Dimerization domains can include, for example, an amino acid sequence containing a cysteine residue that facilitates formation of a disulfide bond between two polypeptide sequences. In one example, the dimerization domain includes all or part of a full-length antibody hinge region. Dimerization domains can include one or more dimerization sequences, which are sequences of amino acids known to promote interaction between polypeptides. Such dimerization domains are well known, and for example, leucine zippers, GCN4 zippers, for example, the sequence of amino acids set forth in SEQ ID NO: 9 (GRMKQLEDKVEELLSKNYHLENEVARLKKLVGERG), and mixtures thereof.

In one example, the dimerization domains are generated by mutation of the antibody chains, for example, the heavy chain variable regions, to promote their interaction. In another example, the dimerization domains are generated by insertion of additional nucleotide sequence encoding a dimerization sequence or sequence encoding one or more cysteine residues, for example, at the C- or N-terminal end of one or more antibody chain. Exemplary of such sequences are sequences encoding leucine zippers, CCN4 zippers or antibody hinge regions. Such additional sequences can be inserted so that the dimerization domains occur between the antibody chains or at the C-terminal end of an antibody chain, for example, between the heavy chain and the phage coat protein. In one example, the dimerization domain is located at the C-terminal end of the heavy chain variable or constant domain sequence and/or between the heavy chain variable or constant domain sequence and any viral coat protein component sequence.

iii. Mutations Promoting Dimerization

In one example, one or more mutations is made to the nucleotide sequence encoding the domain exchange antibody fragment in order to facilitate and/or stabilize display of the fragment with the appropriate configuration. Exemplary of such mutations are mutations that result in amino acid substitution(s) that introduce one or more additional cysteine residues into the antibody, to promote formation of disulfide bridges, e.g. between different heavy and/or light chain domains, in order to stabilize the domain exchanged structure.

Exemplary of such mutations is one made by mutating the nucleotide sequence encoding the 19^{th} amino acid in the 2G12 antibody heavy chain, such that this amino acid is changed from an isoleucine (Ile) to a cysteine (Cys) residue. In one example, this mutation or other similar mutation is made to other domain exchanged antibodies. This substitution promotes formation of a disulfide bridge between the two heavy chain variable regions, stabilizing the domain exchanged configuration. Exemplary of the antibody fragments having this mutation are the domain exchanged Fab Cys19 (illustrated in FIG. 2c), which is identical to the domain exchanged Fab fragment, but carries this Ile-Cys mutation; the domain exchanged scFab ΔC²Cys19 (illustrated in FIG. 2d(ii)), which is identical to the domain exchanged scFab ΔC² fragment but further carries this mutation; and the scFv Cys19 (illustrated in FIG. 2h), which is identical to the domain exchanged ScFv fragment, but carries this additional mutation.

Other mutations that stabilize intra-chain interactions are known in the art. Any known method for stabilizing interactions can be used with the provided methods to generate constructs for phage display of domain exchanged antibody fragments.

iv. Hinge Regions

In some examples, the hinge region of the antibody molecule is included in the domain exchanged antibody fragment for display on genetic packages. The hinge region of IgG, IgD and IgA antibody molecules, located between the C_{H}1 and C_{H}2 regions, contains cysteine residues that promote formation of disulfide bonds between heavy chains. Nucleotide sequences encoding the hinge region can be included in the nucleic acid encoding the domain exchanged antibodies for expression of domain exchanged antibody fragments (e.g. Fab, scFv) from the vectors provided herein to promote interaction between the two heavy chains, thus stabilizing the domain exchanged configuration.

Exemplary of displayed domain exchanged antibody fragments that contain hinge regions are illustrated in FIGS. 2b (domain exchanged Fab hinge) and 2g (domain exchanged scFv hinge). Thus, included amongst the vectors provided herein are phagemid vectors that contain a nucleic acid encoding a hinge region between the nucleic acid encoding the C_{H}1 domain (e.g. Fab hinge) or a variable region (e.g. scFv hinge) of a domain exchanged antibody fragment and the nucleic acid encoding the coat protein (for example, gene III as illustrated in FIG. 2b). Thus, the domain exchanged Fab hinge fragment is identical to the domain exchanged Fab fragment, except that each heavy chain further includes a hinge region in each heavy chain following the C_{H}1 region, which promotes interaction between the two heavy chains. Similarly, a phagemid vector encoding a domain exchanged scFv hinge fragment can contain nucleic acid encoding a hinge region between the nucleic acids encoding the V_{H} domain and the coat protein. Thus, the domain exchanged scFv hinge fragment is identical to the domain exchanged scFv fragment, with the exception that a hinge region is included in each chain, promoting formation of a disulfide bridge, which can stabilize the configuration of the domain exchanged fragment.

v. Other Dimerization Domains

Other domains that can be used to promote interaction between molecules (e.g. antibody chains) are well known (see, for example, U.S. Published Application No.: US20050119455, describing use of a leucine zipper dimerization domain to promote interaction between antibody chains to increase avidity in a phage displayed divalent Fab fragment). Dimerization domains can include, for example, an amino acid sequence comprising a cysteine residue that facilitates formation of a disulfide bond between two polypeptide sequences. Dimerization domains can include one or more dimerization sequences, which are sequences of amino acids known to promote interaction between polypeptides. Such dimerization domains are well known, and include, for example, leucine zippers, GCN4 zippers, for example, the sequence of amino acids set forth in SEQ ID NO: 9 (GRMKQLEDKVEELLSKNYHLENEVARLKKLVGERG), and mixtures thereof.

vi. Exemplary Domain Exchanged Antibodies and Fragments

Exemplary of domain exchanged antibodies for expression by the vectors provided herein is the 2G12 antibody, which includes the domain exchanged human monoclonal IgG1 antibody produced from the hybridoma cell line CL2 (as described in U.S. Pat. No. 5,911,989; Buchacher et al., AIDS Research and Human Retroviruses, 10(4) 359-369 (1994); and Trkola et al., Journal of Virology, 70(2) 1100-1108 (1996)), as well as any synthetically, e.g. recombinantly, produced antibody having the identical sequence of amino acids, and any antibody fragment thereof having identical heavy and light chain variable region domains to the full-length antibody, such as the 2G12 domain exchanged Fab fragment (see, for example, Published U.S. Application, Publication No.: US20050003347 and Calarese et al., Science, 300, 2065-2071 (2003). 2G12 includes antibodies (such as fragments) having at least the antigen binding portions of the heavy chains of the monoclonal IgG1 (e.g. the sequence of amino acids set forth in SEQ ID NO: 25) and typically at least the antigen binding portion(s) of the light chain (e.g. the light chain having the sequence of amino acids set forth in SEQ ID NO: 26 or SEQ ID NO: 27) of nucleic acids set forth in 2G12 antibody specifically binds HIV gp120 antigen (the HIV envelope surface glycoprotein, gp120, GENBANK gi:28876544, which is generated by cleavage of the precursor, gp160, GENBANK g.i. 9629363). Also exemplary of the domain exchanged antibodies are 3-Ala 2G12 antibodies, including fragments thereof, which are modified 2G12 antibodies having three mutations to alanine in the amino acid sequence encoding the heavy chain antigen binding domain, rendering it non-specific for the cognate antigen (gp120) of the native 2G12 antibody. These and other domain exchanged antibodies or fragments thereof can be encoded by the vectors provided herein and expressed at reduced levels in partial suppressor cells. In some examples, the domain exchanged antibodies or fragments thereof are expressed from the phagemid vectors provided herein and displayed on the surface of phage, such as in a phage display library.

FIG. 2 illustrates exemplary displayed domain exchanged fragments that can be made using the provided methods and vectors. The examples illustrated in FIG. 2 are displayed on bacteriophage, as fusion proteins containing part of the cp3 coat protein. These fragments, and variations thereof, can also be displayed using other coat proteins and/or in other display systems.

(1) Domain Exchanged Fab Fragment

As illustrated in FIG. 2A, the domain exchanged Fab fragment contains two heavy chains (one soluble and one fusion protein) and two light chains. The displayed domain exchanged Fab fragment can be generated using a vector containing a nucleic acid encoding the V_{H}-C_{H}1 chain, followed by a nucleic acid encoding a stop codon (e.g. the amber stop codon (TAG)), followed by a nucleic acid encoding a coat protein (such as a phage coat protein, e.g. cp3, encoded by gene III, as depicted in the example in FIG. 2A). In one example, the vector also includes the nucleic acid encoding a light chain (V_{L}-C_{L}). Alternatively, the light chain can be expressed from another vector, which is used to transform the same host cell. The vectors for display of the domain exchanged Fab antibody are designed such that, when expressed in a partial suppressor host cell (e.g. XL1-Blue or ER2738 cells), two separate heavy chain elements (V_{H}-C_{H}1 and V_{H}-C_{H}1-coat protein fusion) are produced from a single copy of the encoding nucleic acid. These two copies of the heavy chain assemble, along with two soluble light chains produced by the same vector or a different vector, to form the domain exchanged “Fab” antibody on the surface of the genetic package, having two conventional antibody combining sites.

(2). Domain Exchanged scFv Fragment

As illustrated in FIG. 2F, the displayed domain exchanged scFv fragment contains two chains, each of which contains one V_{H} and one V_{L} domain, joined by a peptide linker (V_{H}-linker-V_{L}). One of these chains is a fusion protein and further contains the sequence of a coat protein (the example in FIG. 2F illustrates a fusion with phage coat protein cp3). Thus, one of the chains is a fusion protein, containing the V_{H}-linker-V_{L} and a coat protein, such as cp3 (coat protein-V_{H}-linker-V_{L}). The other chain is a soluble chain (V_{H}-linker-V_{L}). In the folded domain exchanged scFv fragment, the two chains interact through the V_{H} domains, providing the interlocked domain exchanged configuration.

The domain exchanged scFv fragment can be generated with a vector containing a nucleic acid encoding the V_{H}-linker-V_{L} single chain, followed by a sequence encoding a stop codon (e.g the amber stop codon (TAG)), followed by a sequence encoding a coat protein (e.g. a phage coat protein such as gene III, as depicted in FIG. 2F). Such a vector is designed so that, when expressed in a partial suppressor host cell (e.g. XL1-Blue or ER2738 cells), a soluble single chain (V_{H}-linker-V_{L}) and a fusion protein single chain (coat protein-V_{H}-linker-V_{L}) are produced, and assemble on the phage surface to form the domain exchanged “scFv” antibody on the surface of phage, having two chains (one soluble, one fusion protein) and two conventional antibody combining sites. The two chains are encoded by a single copy of the genetic element in the vector.

For display of the domain exchanged scFv fragment, one of the chains contains a coat protein, in proximity to a coat protein (cp3/GeneIII, as shown in FIG. 2F). In this example, the polynucleotide encoding the domain exchanged scFv fragment contains one nucleic acid encoding the V_{H} domain, one nucleic acid encoding the V_{L} domain and one nucleic acid encoding the coat protein. The polynucleotide further contains a nucleic acid encoding a polypeptide linker between the V_{H} and V_{L} domains and a nucleic acid encoding a stop codon between the V_{H} and coat protein encoding sequences. Thus, when the construct is expressed in partial suppressor strains, the two chains (one soluble, one fusion protein) are expressed and displayed on the genetic package surface as a domain exchanged antibody complex.

(3). Domain Exchanged Fab Hinge Fragment

Also exemplary of displayed (e.g. phage-displayed) domain exchanged antibody fragments that are generated using the provided stop codon methods are domain exchanged Fab hinge fragments.

As illustrated in FIG. 2B, the display vector encoding the domain exchanged Fab hinge fragment is generated by inserting a nucleic acid encoding a hinge region into the domain exchanged Fab fragment vector, between the nucleic acid encoding the C_{H}1 domain and the nucleic acid encoding the coat protein (for example, gene III as illustrated in FIG. 2B). Thus, the domain exchanged Fab hinge fragment is identical to the domain exchanged Fab fragment, except that each heavy chain further includes a hinge region in each heavy chain following the C_{H}1 region, which promotes interaction between the two heavy chains.

(4). Domain Exchanged scFv Tandem Fragment

An example of this fragment displayed on phage, as part of a cp3 fusion protein, is illustrated in FIG. 2E. In the nucleic acid molecule encoding this fragment, three nucleic acids encoding peptide linkers are inserted between the nucleic acids encoding a first V_{L} and first V_{H} chain, between the nucleic acids encoding the first V_{H} and a second V_{H} chain, and between nucleic acids encoding the second V_{H} and a second V_{L} chain. Thus, while for display of a domain exchanged Fab fragment, two heavy chains (soluble and fusion protein) are encoded by a single genetic element, the scFv tandem vector, by contrast, carries two copies each of identical nucleic acid molecules encoding the light chain and heavy chain variable region domains, all four of which are joined by nucleic acids encoding peptide linkers. Thus, in the fragment, two heavy and two light chain variable region domains are joined by peptide linkers. In the case of a displayed domain exchanged scFv tandem fragment (as illustrated in FIG. 2E), the four chains are and expressed as a single chain coat protein fusion molecule, on the genetic package surface, to form the domain exchanged structure. Thus, in this fragment, the peptide linkers are used instead of the stop codon to provide multiple heavy and light chains in the same domain exchanged fragment.

(5). Domain Exchanged Single Chain Fab Fragments

In another example, illustrated in FIG. 2D(i), the displayed domain exchanged Fab fragment is modified by inserting sequences encoding peptide linkers between the V_{L}-C_{L} sequence and the V_{H}-C_{H}1-coat protein (e.g. geneIII) sequence, thereby generating (upon expression in a partial suppressor strain) one V_{L}-C_{L}-linker-V_{H}-C_{H}1-coat protein fusion chain and one soluble V_{L}-C_{L}-linker-V_{H}-C_{H}1 chain, which pair on the genetic package surface to form a single chain Fab (scFab) fragment, such as the scFab ΔC², having the domain exchanged configuration. As illustrated in FIG. 2D(i), in the scFab ΔC² fragment, two cysteines are mutated to ablate formation of the disulfide bonds between the constant regions, as the presence of the linkers makes these disulfide bonds unnecessary for stabilizing the folded antibody fragment. A modified scFab ΔC² fragment, the scFab ΔC²Cys19 fragment, is described below.

(6). Domain Exchanged Fab Cys19

The domain exchanged Fab Cys 19 fragment is illustrated in FIG. 2C. It is identical to the domain exchanged Fab fragment, but carries this Ile-Cys mutation; the domain exchanged scFab ΔC²Cys19 (illustrated in FIG. 2D(ii)), which is identical to the domain exchanged scFab ΔC² fragment but further carries this mutation; and the scFv Cys 19 (illustrated in FIG. 2H), which is identical to the domain exchanged ScFv fragment, but carries this additional mutation. Nucleic acid sequences of exemplary vectors encoding domain exchanged 2G12 Fab Cys19, scFab ΔC²Cys19, and scFv Cys19 fragments are set forth in SEQ ID NOs: 29, 30 and 31, respectively.

(7). Domain Exchanged scFv Hinge

Similarly, the display vector encoding the domain exchanged scFv hinge fragment (illustrated in FIG. 2G) is generated by inserting into the vector encoding the domain exchanged scFv fragment a nucleic acid encoding a hinge region between the nucleic acids encoding the V_{H} and the coat protein. Thus, the domain exchanged scFv hinge fragment is identical to the domain exchanged Fab fragment, with the exception that a hinge region is included in each chain, promoting formation of a disulfide bridge, which can stabilize the configuration of the domain exchanged fragment.

e. Exemplary Vectors

Exemplary of the vectors provided herein are phagemid vectors for use in the display of a protein of interest, such as an antibody or fragment thereof. In some instances, the vectors are designed for reduced expression of the protein, to effect reduced toxicity to the host cell. In other instances, the vector is designed for expression of both soluble proteins and fusion proteins that can be displayed on the surface of phage. In some examples, the vectors have properties for both purposes. In a particular example, the vectors provided herein are phagemid vectors that contain nucleic acid encoding an antibody, such as domain exchanged antibody, or fragments or domains thereof, including Fab, Fab′, F(ab′)₂, single-chain Fvs (scFv), Fv, dsFv, diabody, Fd or Fd′ fragments. When expressed in partial suppressor cells, the antibodies or fragments thereof are expressed both as soluble proteins and as fusion proteins with a phage coat protein. In a particular example, the vectors provided herein encode a Fab fragment, such as a domain exchanged Fab fragment.

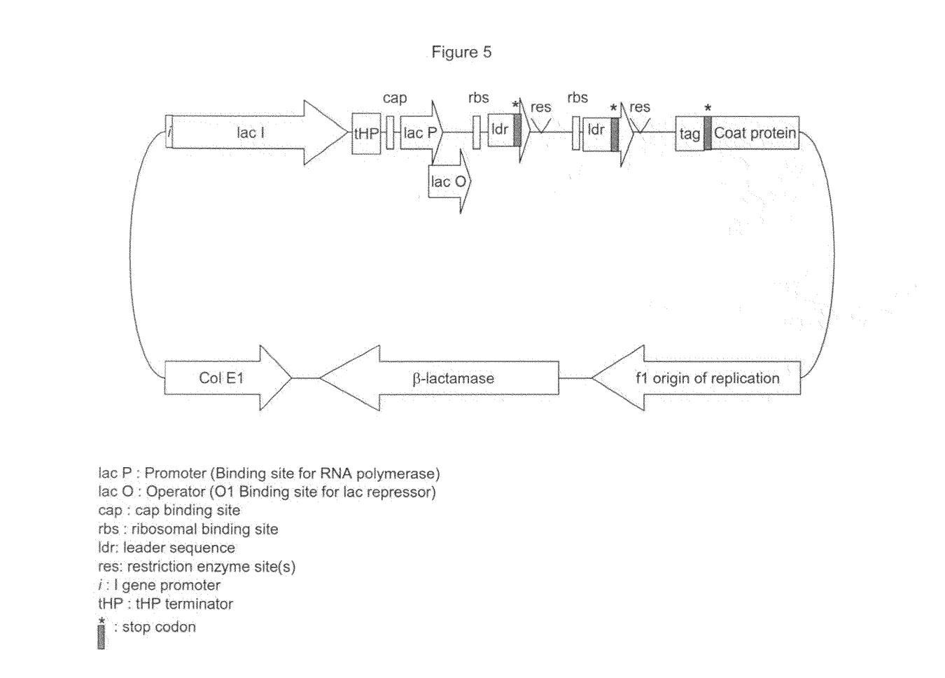

FIG. 5 illustrates an exemplary phagemid vector that can be used to insert nucleic acid encoding a protein for which reduced expression is desired. Such a vector includes a lac promoter system operably linked to a leader sequence into which a stop codon has been introduced. One or more restriction enzyme recognition sequences (e.g. a multiple cloning site) are downstream of the leader sequence, allowing for insertion of nucleic acid encoding a protein or domain or fragment thereof. Down stream of this is a tag sequence, followed by a stop codon and nucleic acid encoding a phage coat protein. In a further example, the vector contains an additional leader sequence containing a stop codon, followed by one or more restriction enzyme recognition sequences, allowing insertion of a second polynucleotide encoding another protein or fragment or domain thereof. As will be appreciated by one of skill in the art, additional elements and features can be included in the vector or substituted for those illustrated, while still maintaining the function of the vector, i.e. the ability to express a protein at reduced levels by the incorporation of one or more stop codons, such as the incorporation of one or more stop codon in a leader sequence. For example, different promoters can be used to replace the lac promoter system. In other instances, various elements can be excluded, such as the tag sequence.

In a particular embodiment, the phagemid vectors provided herein can be used to express an antibody, such as a domain exchanged antibody, or fragments or domains thereof, at reduced levels to reduce toxicity. For example, the vector can be used to express a Fab fragment at reduced levels. Thus, a phagemid vector provided herein can contain nucleic acid encoding an antibody light chain operably linked at its 5′ end to the 3′ end of a leader sequence into which a stop codon has been introduced, and nucleic acid encoding an antibody heavy chain operably linked at its 5′ end to the 3′ end of a leader sequence into which a stop codon has been introduced (FIG. 6). The single genetic element containing these leader and antibody chain sequences is operably linked to the lactose promoter and operator, such that their expression is regulated by lactose or an appropriate lactose substitute, such as IPTG. Further, the vector contains nucleic acid encoding a tag and a phage coat protein downstream of the nucleic acid encoding the heavy chain. The nucleic acid encoding the tag is followed by a stop codon. Thus, when introduced into an appropriate partial suppressor cell, the heavy chain is expressed as a soluble protein (with a tag) and as a fusion protein with the phage coat protein, and the light chain is expressed as a soluble protein. Inclusion of the stop codon in the leader sequences linked to the nucleic acid encoding the heavy and light chains facilitates reduced expression of the these proteins in corresponding partial suppressor cells (i.e. amber partial suppressor cells if amber stop codons is introduced), thus reducing the toxicity of these proteins to the host cell.

pCAL Vectors

Provided are for display of polypeptides, such as domain exchanged antibodies include vectors for display of bivalent antibodies, and vectors for display with reduced toxicity compared to vectors not containing stop codons, e.g. by providing reduced expression. Exemplary of the provided vectors include, but are not limited to, pCAL vectors, such as vectors having the sequence of nucleic acids set forth in any of SEQ ID NOs: 13 (pCAL G13), 14 (pCAL A1), 32 (2G12 pCAL G13), 33 (3-ALA 2G12 pCAL G13), 34 (2G12 pCAL A1), 35 (2G12 pCAL IT*) and 36 (2G12 pCAL ITPO), which are described herein. The pCAL vectors contain nucleic acids encoding part (e.g. C-terminus) of the filamentous phase M13 Gene III coat proteins.

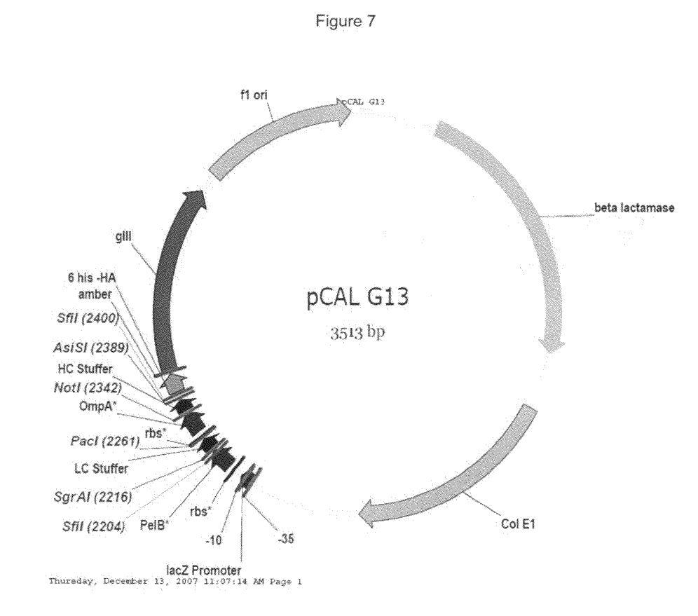

Exemplary of the pCAL vectors are, pCAL G13 and pCAL A1, having the sequences of nucleotides set forth in SEQ ID NOs.: 13 and 14, respectively. pCAL G13 and pCAL A1 contain a truncated gIII gene, encoding a truncated M13 gene III coat protein, preceded by a multiple cloning site, into which a polynucleotide, for example, a polynucleotide containing a target polynucleotide, can be inserted. Example 2A, below describes methods for generating the pCAL G13 and pCAL A1 vectors. A map of pCAL G13 is shown in FIG. 7.

The pCAL vectors further contain amber stop codon DNA sequences (TAG, SEQ ID NO: 37), which encode the RNA amber stop codon (UAG; SEQ ID NO: 160), just upstream of the nucleic acid encoding the portion of geneIII. Thus, the vectors are designed such that polynucleotides, e.g. domain exchanged antibody-encoding polynucleotides, can be inserted just upstream of the amber stop codon. The presence of the amber stop codon allows regulation of polypeptide expression, for example, by expression in a partial amber suppressor host cell as described in section (f), below. For example, expression in a partial amber suppressor host cell can be carried out to regulate the frequency at which fusion protein and soluble polypeptides, respectively, are produced.

Different pCAL vectors provided herein can result in different amounts of readthrough through the amber-stop codon. For example, the pCAL G13 vector contains a guanine residue at the position just 3′ of the amber stop codon, while the pCAL A1 vector contains an adenine at this position. Choice of vector can determine how the relative amount of read-through that occurs through the stop codon, e.g. when using a partial suppressor strain, and thus can regulate the relative amount of fusion versus non-fusion target/variant polypeptide translated from the vector.

The provided vectors include vectors, e.g. pCAL vectors, containing nucleic acids encoding domain exchanged Fab fragments, such as, but not limited to, domain exchanged Fab fragment of the 2G12 antibody and domain exchanged Fab fragment of the 3-Ala 2G12 antibody, which contains 3 mutations in the antibody combining site compared to the 2G12 antibody as described herein.

(1). 2G12 pCAL Vectors and Variants

The provided vectors include pCAL vectors for expression and display of the domain exchanged antibody, 2G12, 2G12 variants (3-ALA 2G12 and 3-ALA LC 2G12), domain exchanged Fab fragments of 2G12, 3-ALA 2G12 and 3-ALA LC 2G12, and other fragments and variants, and fragments of variant domain exchanged antibodies that contain modifications compared to 2G12.

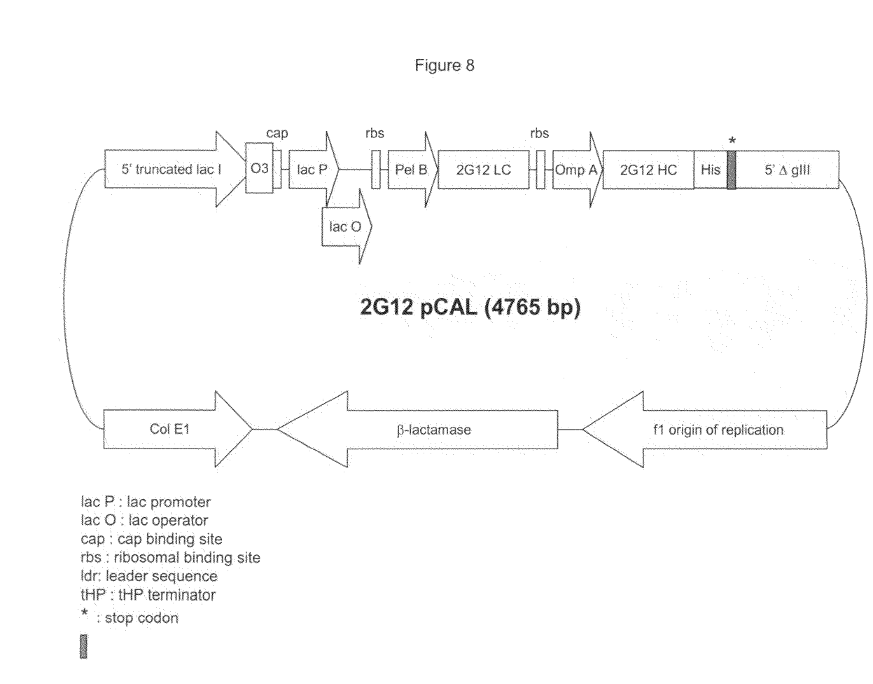

An exemplary vector, the 2G12 pCAL G13 vector (also called the 2G12 pCAL vector) contains the nucleotide sequence set forth in SEQ ID NO: 32, is produced as described in Example 2B(i). This vector, which is set forth schematically in FIG. 8, contains a nucleic acid encoding heavy and light chain domains of the 2G12 antibody. Expression as both soluble 2G12 Fab fragments and 2G12-gIII coat protein fusion proteins for display on phage particles can be effected from this vector in partial amber suppressor cells by virtue of the amber stop codon between the nucleotides encoding the 2G12 heavy chain nucleotides encoding the truncated gIII coat protein, using the provided methods. In this vector, the polynucleotide encoding the 2G12 light chain is operably linked to the Pel B leader sequence (the nucleic acid sequences encoding the leader peptides from the pectate lyase B protein from Erwinia carotovora), while the 2G12 heavy chain is operably linked to the OmpA leader sequence (the nucleic acid sequence encoding the leader peptide from the E. coli outer membrane protein. The 2G12 pCAL vector further contains a truncated lac I gene; the lac I gene encodes the lactose repressor molecule. Ribosome binding sites upstream of both the PelB and OmpA leader sequences facilitate translation. The 2G12 pCAL G13 vector (SEQ ID NO: 32) can be used to display a 2G12 domain exchanged Fab antibody fragment on phage.

Another exemplary vector, the 3-Ala pCAL G13 vector, contains the nucleotide sequence set forth in SEQ ID NO: 33 and is produced as described in Example 2B(iii), below. This vector contains nucleic acid encoding heavy and light chain domains of 3-ALA 2G12 and is otherwise identical to the 2G12 pCAL G13 vector. The 3-Ala pCAL G13 vector can be used to display the 3-Ala 2G12 Fab fragment on phage. Example 4, below, describes display of 2G12 domain exchanged Fab fragment on phage using this vector. Examples 6 and 7 describe studies demonstrating antigen-specific selection by panning using the displayed 2G12 domain exchanged Fab fragment, expressed from this vector. Another exemplary vector is the 3-Ala LC pCAL G13 vector (SEQ ID NO:323), which contains the 3-Ala LC light chain.

(2). 2G12 pCAL IT* and Variants

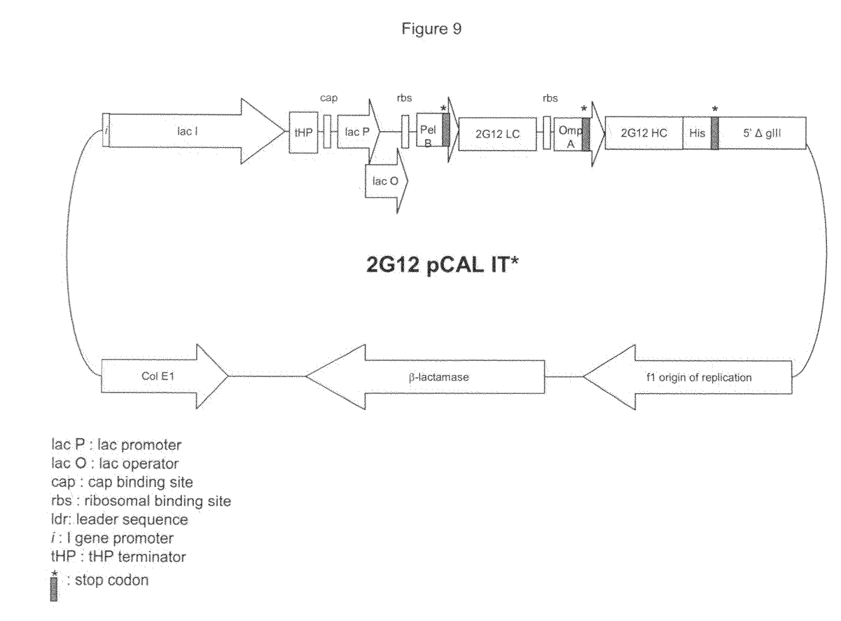

Exemplary of phagemid vectors provided herein is the 2G12 pCAL IT* vector. This vector, which is schematically depicted in FIG. 9 and has a sequence of nucleotides set forth in SEQ ID NO:35, was generated as described in Example 2C, below. The 2G12 pCAL IT* vector can be used to express, with reduced toxicity (compared to the absence of stop codons in leader sequences), Fab fragments of the domain exchanged 2G12 antibody, which recognize the HIV gp120 antigen. Expression as both soluble 2G12 Fab fragments and 2G12-gIII coat protein fusion proteins for display on phage particles can be effected in partial amber suppressor cells by virtue of the amber stop codon between the nucleotides encoding the 2G12 heavy chain nucleotides encoding the truncated gIII coat protein.

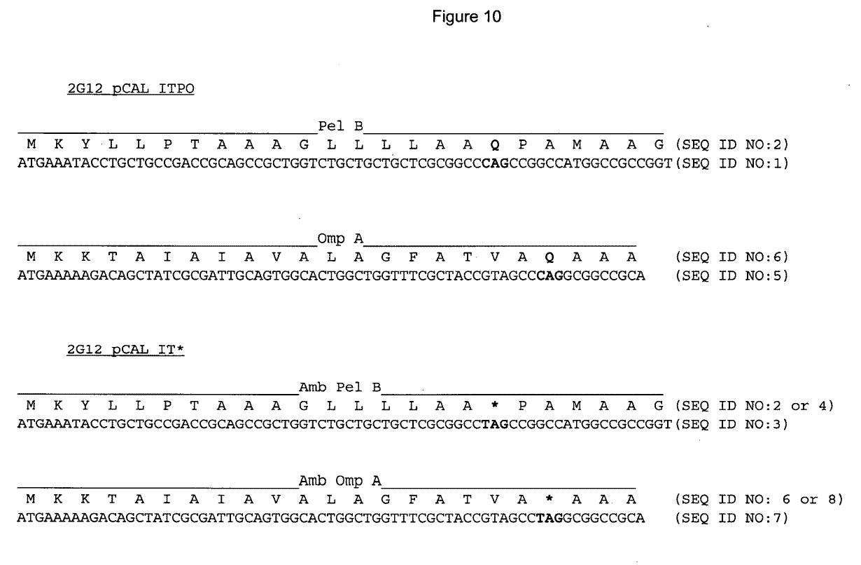

The polynucleotide encoding the 2G12 light chain is operably linked to the Pel B leader sequence (the nucleic acid sequences encoding the leader peptides from the pectate lyase B protein from Erwinia carotovora), while the 2G12 heavy chain is operably linked to the OmpA leader sequence (the nucleic acid sequence encoding the leader peptide from the E. coli outer membrane protein. The inclusion of an amber stop codon in each of the leader sequences results in reduced expression of the 2G12 heavy and light chains in partial amber suppressor strains, and, therefore, reduced toxicity. The stop codons are incorporated by mutation of the CAG triplet encoding a glutamine (Glu, Q) in each of the leader sequences to a TAG amber stop codon (see, FIG. 10). For example, the nucleotide triplet at nucleotides 52-54 of the PelB leader sequence set forth in SEQ ID NO:1, encoding the glutamine at amino acid position 18 of the PelB leader peptide set forth in SEQ ID NO:2, was modified to generate a TAG amber stop codon at nucleotides 52-54 (SEQ ID NO:3). Thus, upon expression in a partial amber suppressor cell, in some instances read though occurs to produce a polypeptide encoding the PelB leader peptide linked to the 2G12 light chain, while in other instances, translation is terminated at the stop codon and a truncated 17 amino acid PelB leader peptide is produced, with no expression of the 2G12 light chain. Similarly, the nucleotide triplet at nucleotides 58-60 of the OmpA leader sequence set forth in SEQ ID NO: 5, encoding the glutamine at amino acid position 20 of the OmpA leader peptide set forth in SEQ ID NO: 6) was modified to generate a TAG amber stop codon at nucleotides 58-60 (SEQ ID NO: 7). Thus, upon expression in a partial amber suppressor cell, in some instances read though occurs to produce a polypeptide encoding the OmpA leader peptide linked to the 2G12 heavy chain, while in other instances, translation is terminated at the stop codon and a truncated 19 amino acid OmpA leader peptide is produced, with no expression of the 2G12 heavy chain.

To further regulate expression of the 2G12 heavy and light chains, the transcription of both is under the control of the lac promoter/operator system. The 2G12 pCAL IT* vector contains the full length lac I gene, which encodes the lactose repressor molecule. In the absence of lactose or another suitable inducer, such as IPTG, the repressor binds to the operator and interferes with binding of the RNA polymerase to the promoter, inhibiting transcription of the operably linked heavy and light chain genes. In the presence of lactose or a suitable equivalent, such as IPTG, the lactose metabolite allolactose binds to the repressor, causing a conformational change that renders the repressor unable to bind to the operator, thereby allowing binding of the RNA polymerase and transcription of a single transcript encoding the 2G12 light and heavy chains. Ribosome binding sites upstream of both the PelB and OmpA leader sequences facilitate translation.

Also provided are variations of the 2G12 pCAL IT* vector. In one example, the 2G12 pCAL IT* vector was further modified by the introduction of three alanine amino acid substitutions in the light chain CDR3 of 2G12. The modification of the 2G12 pCAL IT* vector was carried out using overlapping PCR mutagenesis and cloning at the SgrAI and PacI sites of the 2G12 pCAL IT* vector (as described in Example 9) to produce the 2G12 3Ala LC pCAL IT* vector (SEQ ID NO:174). This vector can be used, therefore, for expression of the 2G12 3Ala LC Fab fragment, which contains mutations at positions L91, L94 and L95 by Kabat numbering, and can have V_{L} domain with a sequence set forth in SEQ ID NO: 305.

(3). Vectors for Display of Other Domain Exchanged Fragments

The provided vectors further include vectors for display of other domain exchanged antibody fragments (e.g. other 2G12 fragments), such as fragments containing dimerization domains, such as hinge regions, cysteines forming disulfide bridges, and single chain fragments, such as domain exchanged single chain Fab fragments and domain exchanged scFv fragments, and combinations thereof (see, for example, FIG. 2). Example 8 describes the generation of constructs for the display of various other 2G12 fragments, in addition to the 2G12 domain exchanged Fab fragment on phage. Such additional fragments include the domain exchanged Fab hinge fragment (expressed from the vector containing the nucleotide sequence set forth in SEQ ID NO: 38, which contains an additional sequence in the Fab-encoding sequence, that encodes a hinge region between the heavy chain constant region and the gene III coat protein encoding sequence); the 2G12 domain exchanged Fab Cys19 fragment (expressed from the vector containing the nucleotide sequence set forth in SEQ ID NO: 29, which contains a mutation in the heavy chain of the Fab fragment, resulting in an Ile-Cys mutation to promote interaction of the two heavy chain variable regions of the Fab fragment); the 2G12 domain exchanged scFab ΔC²Cys19 (expressed from the vector containing the nucleotide sequence set forth in SEQ ID NO: 30, which contains the same mutation in the heavy chain of the Fab fragment, resulting in an Ile-Cys mutation, and contains a sequence encoding a linker between the heavy and light chains); the 2G12 domain exchanged scFv fragment (expressed from the vector containing the nucleotide sequence set forth in SEQ ID NO: 39, which contains one V_{H} encoding sequence and one V_{L} encoding sequence, followed by an amber stop codon, promoting formation of a domain exchanged scFv fragment with two conventional antibody combining sites); the 2G12 domain exchanged scFv tandem fragment (expressed from the vector containing the nucleotide sequence set forth in SEQ ID NO: 40, which includes the sequence for an additional V_{H} and an additional V_{L} region, separated by a linker sequence, for expression of two heavy chain variable domains and two light chain variable region domains from the single vector); the 2G12 domain exchanged scFv hinge and scFv hinge (ΔE) fragments (expressed from the vector containing the nucleotide sequence set forth in SEQ ID NO: 41, and SEQ ID NO: 42, respectively, each of which contains the sequence of the scFv encoding vector, with an additional hinge-region encoding sequence, to promote interaction between the two single chains in the fragment); and the 2G12 domain exchanged scFv Cys 19 fragment (expressed from the vector containing the nucleotide sequence set forth in SEQ ID NO: 31, which contains the sequence of the scFv fragment with the mutation in the heavy chain variable region, resulting in an Ile-Cys mutation to promote interaction of the two heavy chain variable regions of the scFv fragment). Example 8, below, describes a study demonstrating expression and display of some of these fragments.

3. Methods for Expression of Polypeptides

To express the protein(s) from the provided vectors that contain stop codon nucleic acids, the vectors are transformed into an appropriate partial suppressor host cell strain. Thus, provided herein are cells for the expression and display of proteins, including domain exchanged antibodies. In some instances, the suppression efficiency (i.e. the efficiency with which the suppressor tRNA effects read through) of the partial suppressor cell into which the vector has been transformed is less than or about 90%, such as no more than or about 85%, 80%, 75%, 70%, 65%, 60%, 55%, 50%, 45%, 40%, 35%, 30%, 25%, 20%, or 15%. Thus, by introducing the vectors provided herein into partial suppressor cells, the expression of proteins encoded by the vectors can be reduced by or about 10%, 15%, 20%, 25%, 30%, 35%, 40%, 45%, 50%, 55%, 60%, 65%, 70%, 75%, 80% 85% or more compared to expression of the proteins from a comparable vector that does not contain the introduced stop codons.

The type of host cell used to express the protein of interest from the vectors provided herein will depend upon the type of stop codon incorporated into the vector, such as between the polypeptide (e.g. antibody chain) and the coat protein, or into the leader sequence that is linked to nucleic acid encoding the protein of interest. For example, if one or more amber stop codons are introduced into the vector, then the vector is transformed into a partial amber suppressor strain that harbors an amber suppressor tRNA molecule. If one or more ochre stop codons are introduced into vector, the vector is transformed into a partial ochre suppressor strain that harbors an ochre suppressor tRNA molecule. Further, a host cell typically is chosen in which the suppressor tRNA molecule will incorporate the desired amino acid residue when read through of the stop codon occurs (such as the wild-type amino acid or another desired amino acid). For example, if the vector contains an amber stop codon that was introduced in place of a glutamine codon (or where a glutamine is desired), then the vector can be introduced into a partial amber suppressor strain that expresses an amber suppressor tRNA that incorporates a glutamine residue at the TAG codon.

The vector can be introduced into the partial amber suppressor cell using any method known in the art, including, but not limited to, electroporation and chemical transformation. Following transformation into an appropriate partial suppressor strain, in some instances, expression of the polypeptides can be induced in the host cells. For example, if transcription is under control of a regulatable promoter, then the appropriate conditions can be generated to induce transcription. Further, in some examples, the host cells are phage-display compatible host cells, and are used to display the protein(s) of interest on the surface of a bacteriophage, for example, in a phage display library. By generating phage display libraries, the proteins displayed on the phage can be screened, analyzed and selected for based on various properties, such as binding activities. such as described in more detail below.

i. Suppressor tRNAs and Partial Suppressor Cells

The vectors provided herein are transformed into a suitable partial suppressor cell. When the vectors are harbored in such cells, two possible events can occur when a ribosome encounters the stop codon that was introduced into the vector, in a host cell containing an appropriate suppressor tRNA: (1) termination of polypeptide elongation can occur if the appropriate release factors associate with the ribosome, or (2) an amino acid can be inserted into the growing polypeptide chain if a suppressor tRNA associates with the ribosome. The efficiency of suppression (read-through) depends upon how well the suppressor tRNA is charged with the appropriate amino acid, the concentration of the suppressor tRNA in the cell, and the “context” of the stop codon in the mRNA. For example, as noted above, the nucleotide on the 3′ side of the codon can affect how much read through translation occurs. In some instances, the suppression efficiency (i.e. the efficiency with which the suppressor tRNA effects read through) is less than or about 90%, such as no more than or about 85%, 80%, 75%, 70%, 65%, 60%, 55%, 50%, 45%, 40%, 35%, 30%, 25%, 20%, or 15%.

The selection of the appropriate partial suppressor host cell strain for transformation with the vectors provided herein is based upon the type of suppressor tRNA molecule that is contained in the host cell. In addition to selection based on whether the cells suppressor tRNA molecule is an amber, ochre or opal suppressor tRNA, selection also can be based on what amino acid residue is incorporated by the suppressor tRNA when read through of the introduced stop codon occurs. For example, if an opal stop codon has been introduced into the vector, and this opal stop codon is introduced such that it replaces a wild type tyrosine codon, then the vector can be introduced into a partial opal suppressor cell that has an opal suppressor tyrosine tRNA molecule (tRNA^{Tyr}) that introduces a tyrosine residue at the opal stop codon.

In one example, the 2G12 pCAL IT* vector, in which amber stop codons have been introduced into the PelB and Omp leader sequences (by replacement of the glutamine codon (GAG) with the amber stop codon (TAG)) that are linked to the nucleic acid encoding the 2G12 light and heavy chains, respectively, and also introduced between the polynucleotides encoding the heavy chain and the phage coat protein, can be transformed into a phage display compatible partial amber suppressor strain that harbors an amber suppressor glutamine tRNA (tRNA^{Gln}) and that introduces a glutamine residue at the amber stop during translation. Thus, the translated leader-antibody chain fusion polypeptides maintain the wild-type amino acid sequence. Following cleavage of the leader peptides, the 2G12 light chains, 2G12 heavy chains, and 2G12 heavy chain-gIIIp fusion proteins are secreted and can associate with one another to form 2G12 domain exchanged Fab fragments on the surface of phage.

The suppressor tRNAs in the partial suppressor cells can be natural or synthetic. In some instances, the suppressor tRNA is encoded in the genome of the suppressor cell. In other examples, the suppressor tRNA is encoded in a plasmid or bacteriophage or other vector carried by the suppressor cell. Thus, partial suppressor cells can be produced by introducing a modified gene encoding a suppressor tRNA molecule, such as one contained on a plasmid, into a non suppressor cell. Many suppressor tRNA molecules are known in the art and can be utilized in the methods herein to express proteins at reduced levels from the vectors provided herein (see e.g., Miller et al., (1989) Genome 21:905-908, Kleina et al., (1990) J. Mol. Biol. 212:295-318, Huang et al., (1992) J. Bacteriol. 174:5436-5441, Taira et al (2006) Nuc. Acids Symp. Series 50:233-234, Kleina et al., (1990) J. Mol. Biol. 213:705-717, Normanly et al., (1990) J. Mol. Biol. 213:719-726; Kohrer et al., (2004) Nucl. Acids Res. 32:6200-6211, Normanly et al., (1986) Proc. Nat. Acad. Sci. USA 83:6548-6552. The suppressor tRNAs can be naturally found in the partial suppressor cell strains, or can be introduced into a non suppressor cell to generate a partial suppressor cell. For example, a plasmid or bacteriophage encoding the suppressor tRNA can be introduced into a non suppressor strain to generate the desired partial suppressor strain. Table 4 provides non-limiting examples of E. coli suppressor tRNAs that recognize the amber, ochre or opal stop codon. The table sets forth the suppressor name, the type of suppressor (amber, opal or ochre), the amino acid that is inserted during read through, and the reported observed suppression efficiency.

TABLE 4
E. coli suppressor tRNAs

| | | | |
|---|---|---|---|
| | | Amino acid | Supression |
| Suppressor | Type | inserted | efficiency |

Natural suppressors

| | | | |
|---|---|---|---|
| supE | Amber | Gln | 1-61% |
| supP | Amber | Leu | 30-100% |
| supD | Amber | Ser | 6-54% |
| supU | Amber | Trp | |
| supF | Amber | Tyr | 11-100% |
| supZ | Amber | Tyr | |
| supB | Ochre | Gln | |
| supL (supG) | Ochre | Lys | |
| supN | Ochre | Lys | |
| supC | Ochre | Tyr | |
| supM | Ochre | Tyr | |
| glyT | Opal | Gly | |
| trpT | Opal | Trp | 0.1-30% |

Synthetic suppressors

| | | | |
|---|---|---|---|
| pGIFB:Ala | Amber | Ala | 8-83% |
| pGIFB:Cys | Amber | Cys | 17-51% |
| pGIFB:Glu | Amber | Glu (85%) | 8-100% |
| | | Gln (15%) | |
| pGIFB:Gly | Amber | Gly | 39-67% |
| pGIFB:His | Amber | His | 16-100% |
| pGIFB:Phe | Amber | Phe | 48-100% |
| pGIFB:Pro | Amber | Pro | 9-60% |
| tRNA(CUAAla2) | Amber | Ala | |
| tRNA(CUAGly1) | Amber | Gly | |
| tRNA(CUAHisA) | Amber | His | |
| tRNA(CUALys) | Amber | Lys | |
| tRNA(CUAProH) | Amber | Pro | |
| tRNAPheCUA | Amber | Phe | 54-100% |
| tRNACysCUA | Amber | Cys | 17-50% |

Amber Suppressor Cells

In one example, the vectors provided herein contain one or more introduced amber stop codons, such as between a nucleic acid encoding an antibody chain and nucleic acid encoding a coat protein, or in the nucleic acid encoding a leader peptide that is linked to the nucleic acid encoding the protein for which reduced expression is desired. Thus, to express the proteins (such as two proteins, one fusion protein and one soluble protein, from a single genetic element), the vectors are introduced into a partial amber suppressor cell. These cells contain amber suppressor tRNA molecules that recognize the UAG codon on the mRNA transcript and insert an amino acid into the polypeptide. As noted above, the efficiency with which the amber stop codon is suppressed (i.e. the efficiency with which read through occurs) depends on several factors. For the purposes herein, however, the vectors provided herein are introduced into partial amber suppressor cells in which suppression efficiency is less than or about 90%, such as no more than at or about 85%, 80%, 75%, 70%, 65%, 60%, 55%, 50%, 45%, 40%, 35%, 30%, 25%, 20%, or 15%.

Exemplary of partial amber suppressor cells are those that carry the supE amber suppressor tRNA. The supE tRNA molecule is a mutant form of a wild-type tRNA^{Gln} molecule, which recognizes a 5′ CAG 3′ codon in the mRNA and inserts glutamine (Gln, Q) into the growing polypeptide chain. In contrast, the supE tRNA contains a mutation in the anticodon (relative to the wild-type tRNA) such that it recognizes the amber stop codon (5′ UAG 3′) in the mRNA inserts a glutamine residue (Gln, Q). E. coli cells that contain the supE tRNA suppressor (sometimes denoted as being positive for the supE44 genotype), and are thus amber suppressor cells (including partial amber suppressor cells) include, but are not limited to, XL1-Blue, DB3.1, DH5α, DH5αF′, DH5αF′IQ, DH5α-MCR, DH21, EB5α, HB101, RR1, JM101, JM103, JM106, JM107, JM108, JM109, JM110, LE392, Y1088, C600, C600hfl, MM294, NM522, Stb13 and K802 cells. Typically, amber suppressor cells containing the supE suppressor tRNA are partial suppressor cells with a suppression efficiency of approximately 1-60% (see, e.g. Kleina et al., (1990) J. Mol. Biol. 212:295-318). In some examples, the partial amber suppressor strains also are phage display compatible. Thus, when phagemid vectors are introduced into these cells, the protein can be displayed on the surface of a phage, as described below.

4. Uses for the Vectors and Cells for Reduced Expression of Proteins

In some instances, the vectors and cells provided herein can be used to express proteins, such as antibodies, in particular domain exchanged antibodies, at reduced levels, thereby reducing toxicity to the host cells. The level of expression is still sufficient, however, for purification, isolation and/or functional analysis of the protein. Typically, proteins that are toxic to cells are not stably expressed and their isolation is problematic. This can be due, for example, to the host cells dying before the protein has accumulated at sufficient levels, or can be due to instability of the nucleic acid encoding the protein, resulting in, for example, truncated forms of the protein. Thus, use of the vectors and cells provided herein to stably express the protein of interest, such as a domain exchanged antibody, at reduced levels can facilitate isolation, purification and recovery of the protein.

In some examples, the vector can be used to display the polypeptide of interest on a genetic package, such as by fusion of the polypeptide with a genetic package display protein. For example, the vector can be a phagemid vector and the protein for which reduced expression is desired is expressed as a fusion protein with a phage coat protein and displayed on the surface of a phage particle. In a particular example, the phagemid vectors provided herein can be used to produce nucleic acid libraries that can then be used to generate phage display libraries. Similarly, polynucleotides in existing nucleic acid libraries can be inserted into the phagemid vectors provided herein. The polynucleotides encode polypeptides, such as, for example, antibodies or fragments thereof, for which reduced expression is desired for reduced toxicity. Typically, diverse nucleic acid libraries are generated that contain variant polynucleotides that encode variant polypeptides. Methods for creating diversity in a nucleic acid libraries are well known in the art can be employed with the vectors provided herein. In some examples, the phagemid vectors contain variant polynucleotides that encode variant antibodies or domains or fragments thereof, including domain exchanged antibodies or domains or fragment thereof. Thus, the vectors provided herein can be used to generate phage display libraries in which variant polynucleotides, such as variant antibodies, are displayed and selected (see e.g., Examples 9-15).

Use of the vectors provided herein to generate diverse nucleic acid libraries for the production of diverse phage libraries can enhance the recovery and enrichment of proteins from such libraries. Effective screening and selection of proteins from libraries such as phage display libraries relies on the stable expression of every protein in the library. Proteins that are toxic to host cells typically cannot be recovered using such methods. In some instances, the host cell expressing the protein is non-viable. In other instances, the nucleic acid encoding the protein is modified or deleted to reduce toxicity such that the protein is no longer expressed in its wild-type form. In such examples, the proteins typically are not present in the library at sufficient levels for screening and selection. Because of the reduced toxicity of the proteins using the vectors provided herein, such proteins can be recovered and enriched following selection compared to if other vectors are used.

### E. Methods for Display on Genetic Packages

Methods for displaying polypeptides on the surface of genetic packages, e.g. in libraries, are well known and include, for example, phage display (see, e.g., Barbas, C. F., 3rd et al., 2001. Phage Display: A Laboratory Manual. Cold Spring Harbor Laboratory Press, Cold Spring Harbor, N.Y.; Clackson et 25 al. (1991) Making Antibody Fragments Using Phage Display Libraries, Nature, 352:624-628) and methods for display on other genetic packages. The provided methods and vectors for display of polypeptides, such as domain exchanged antibodies, can be used to display polypeptides on the surface of any genetic package.

Exemplary genetic packages include, but are not limited to, bacterial cells, bacterial spores, viruses, including bacterial DNA viruses, for example, bacteriophages, typically filamentous bacteriophages, for example, Ff, M13, fd, and fl (see, e.g., Barbas, C. F., 3rd et al., 2001. Phage Display: A Laboratory Manual. Cold Spring Harbor Laboratory Press, Cold Spring Harbor, N.Y.; Clackson et 25 a/. (1991) Making Antibody Fragments Using Phage Display Libraries, Nature, 352:624-628; Glaser et al. (1992) Antibody Engineering by Condon-Based Mutagenesis in a Filamentous Phage Vector System, J. Immunol., 149:3903 3913; Hoogenboom et al. (1991) Multi-Subunit Proteins on the Surface of Filamentous Phage: Methodologies for Displaying Antibody (Fate) Heavy and 30 Light Chains, Nucleic Acids Res., 19:4133-41370; Clackson and Lowman, Phage Display: A Practical Approach; (2004) Oxford University Press (Chapter 1, Russel et al., An introduction to Phage Biology and Phage Display, p. 1-26; Chapter 2, Sidhu and Weiss Constructing Phage display libraries by oligonucleotide-directed mutagenesis, p 27-41)), baculoviruses (see, e.g., Boublik et a/., (1995) Eukaryotic Virus Display: Engineering the Major Surface Glycoproteins of the Autographa California Nuclear Polyhedrosis Virus (ACNPV) for the Presentation of Foreign Proteins on the Virus Surface, Bio/Technology, 13:1079-1084). Typically, polypeptides are displayed on genetic packages in collections of genetic packages, such as phage display libraries, which can be used to select particular polypeptides from the collections using the provided methods. Display of the polypeptides on genetic packages allows selection of polypeptides having desired properties, for example, the ability to bind with a particular binding partner.

1. Phage Display

Typically, the genetic packages are phage, and the polypeptides are expressed with phage display. Methods for generating phage display libraries are well known (see Barbas, C. F., 3rd et al., 2001. Phage Display: A Laboratory Manual. Cold Spring Harbor Laboratory Press, Cold Spring Harbor, N.Y.; Clackson and Lowman, Phage Display: A Practical Approach; (2004) Oxford University Press (Clackson and Lowman, Phage Display: A Practical Approach; (2004) Oxford University Press (Chapter 1, Russel et al., An introduction to Phage Biology and Phage Display, p. 1-26; Chapter 2, Sidhu and Weiss Constructing Phage display libraries by oligonucleotide-directed mutagenesis, p 27-41)). The provided vectors and display methods, e.g. for display of domain exchanged antibodies, can be used in combination with any known general methods for phage display, with modifications according to the provided methods.

For phage display, libraries of polypeptides, such as the domain exchanged antibodies (e.g. domain exchanged antibody fragments) can be expressed on the surfaces of bacteriophages, such as, but not limited to, M13, fd, fl, T7, and λ phages (see, e.g., Santini (1998) J. Mol. Biol. 282:125-135; Rosenberg et al. (1996) Innovations 6:1-6; Houshmand et al. (1999) Anal Biochem 268:363-370, Zanghi et al. (2005) Nuc. Acid Res. 33(18)e160:1-8). Phage display is described, for example, in Ladner et al., U.S. Pat. No. 5,223,409; Rodi et al. (2002) Curr. Opin. Chem. Biol. 6:92-96; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

For display of polypeptides on phage, host cells capable of phage infection and packaging are transformed with phage vectors, typically phagemid vectors, containing polynucleotides encoding the polypeptides. In one example, the host cells are partial suppressor cells, such as any of the cells described in section D(2)(f), above, provided the cells are compatible with phage display. Following amplification, phage packaging and protein expression is induced, typically by co-infection with a helper phage. Generally, the polypeptides are exported to the periplasm (e.g. as part of a fusion protein) for assembly into phage during phage packaging. Following phage packaging, the polypeptides are expressed on the surface of phage, typically as part of fusion proteins, each containing a polypeptide of interest and a portion of a phage coat protein. The phage displaying the fusion proteins can be isolated and analyzed, and used to select desired polynucleotides.

Generally, to produce the fusion protein, polypeptides are fused to bacteriophage coat proteins with covalent, non-covalent, or non-peptide bonds. (See, e.g., U.S. Pat. No. 5,223,409, Crameri et al. (1993) Gene 137:69 and WO 01/05950). For example, nucleic acids encoding the variant polypeptides can be fused to nucleic acids encoding the coat proteins (e.g. by introduction into a vector encoding the coat protein) to produce a polypeptide-coat protein fusion protein, where the polypeptide is displayed on the surface of the bacteriophage. Additionally, the fusion protein can include a flexible peptide linker or spacer, a tag or detectable polypeptide, a protease site, or additional amino acid modifications to improve the expression and/or utility of the fusion protein. For example, addition of a protease site can allow for efficient recovery of desired bacteriophages following a selection procedure. Exemplary tags and detectable proteins are known in the art and include for example, but not limited to, a histidine tag, a hemagglutinin tag, a myc tag or a fluorescent protein.

Phage display systems typically utilize filamentous phage, such as M13, fd, and fl. In some examples using filamentous phage, the display protein is fused to a phage coat protein anchor domain. The fusion protein can be co-expressed with another polypeptide having the same anchor domain, e.g., a wild-type or endogenous copy of the coat protein. Phage coat proteins that can be used for protein display include (i) minor coat proteins of filamentous phage, such as the bacteriophage M13 gene III protein (also called gIIIp, cp3, g3p; GENBANK g.i. 59799327, having the amino acid sequence set forth in SEQ ID NO: 43: MKKLLFAIPLVVPFYSHSAETVESCLAKPHTENSFTNVWKDDKTLDRYANYE GCLWNATGVVVCTGDETQCYGTWVPIGLAIPENEGGGSEGGGSEGGGSEGG GTKPPEYGDTPIPGYTYINPLDGTYPPGTEQNPANPNPSLEESQPLNTFMFQNN RFRNRQGALTVYTGTVTQGTDPVKTYYQYTPVSSKAMYDAYWNGKFRDCA FHSGFNEDPFVCEYQGQSSDLPQPPVNAGGGSGGGSGGGSEGGGSEGGGSEG GGSEGGGSGGGSGSGDFDYEKMANANKGAMTENADENALQSDAKGKLDSV ATDYGAAIDGFIGDVSGLANGNGATGDFAGSNSQMAQVGDGDNSPLMNNFR QYLPSLPQSVECRPFVFSAGKPYEFSIDCDKINLFRGVFAFLLYVATFMYVFST FANILRNKES), and (ii) major coat proteins of filamentous phage such as gene VIII protein (gVIIIp, cp8). Fusions to other phage coat proteins such as gene VI protein, gene VII protein, or gene IX protein can also be used (see, e.g., WO 00/71694).

Portions (e.g., domains or fragments) of these phage proteins may also be used. Useful portions include domains that are stably incorporated into the phage particle, e.g., so that the fusion protein remains in the particle throughout a selection procedure. In one example, the anchor domain of gIIIp is used (see, e.g., U.S. Pat. No. 5,658,727). In another example, gVIIIp is used (see, e.g., U.S. Pat. No. 5,223,409), which can be a mature, full-length gVIIIp fused to the display protein. The filamentous phage display systems typically use protein fusions to attach the heterologous amino acid sequence to a phage coat protein or anchor domain. For example, the phage can include a gene that encodes a signal sequence, the heterologous amino acid sequence, and the anchor domain, e.g., a gIIIp anchor domain.

Valency of the expressed fusion protein can be controlled by choice of phage coat protein. For example, gIIIp proteins typically are incorporated into the phage coat at three to five copies per virion. Fusion of gIIIp to variant proteases thus produces a low-valency. In comparison, gVIII proteins typically are incorporated into the phage coat at 2700 copies per virion (Marvin (1998) Curr. Opin. Struct. Biol. 8:150-158). Due to the high-valency of gVIIIp, peptides greater than ten residues are generally not well tolerated by the phage. Phagemid systems can be used to increase the tolerance of the phage to larger peptides, by providing wild-type copies of the coat proteins to decrease the valency of the fusion protein. Additionally, mutants of gVIIIp can be used which are optimized for expression of larger peptides. In one such example, a mutant gVIIp was obtained in a mutagenesis screen for gVIIIp with improved surface display properties (Sidhu et al. (2000) J. Mol. Biol. 296:487-495).

a. Phagemid and Phage Vectors

Nucleic acids suitable for phage display, e.g., phage vectors, are known in the art (see, e.g., Andris-Widhopf et al. (2000) J Immunol Methods, 28: 159-81, Armstrong et al. (1996) Academic Press, Kay et al., Ed. pp. 35-53; Corey et al. (1993) Gene 128(1):129-34; Cwirla et al. (1990) Proc Natl Acad Sci USA 87(16):6378-82; Fowlkes et al. (1992) Biotechniques 13(3):422-8; Hoogenboom et al. (1991) Nuc Acid Res 19(15):4133-7; McCafferty et al. (1990) Nature 348(6301):552-4; McConnell et al. (1994) Gene 151(1-2):115-8; Scott and Smith (1990) Science 249(4967):386-90).

A library of nucleic acids encoding the polypeptide-coat protein fusion proteins can be incorporated into the genome of the bacteriophage, or alternatively inserted into in a phagemid vector. In a phagemid system, the nucleic acid encoding the display protein is provided on a phagemid vector, typically of length less than 6000 nucleotides. The phagemid vector includes a phage origin of replication so that the plasmid is incorporated into bacteriophage particles when bacterial cells bearing the plasmid are infected with helper phage, e.g. M13K01 or M13VCS. Phagemids, however, lack a sufficient set of phage genes in order to produce stable phage particles after infection. These phage genes can be provided by a helper phage. Typically, the helper phage provides an intact copy of the gene III coat protein and other phage genes required for phage replication and assembly. In one example, because the helper phage has a defective origin of replication, the helper phage genome is not efficiently incorporated into phage particles relative to the plasmid that has a wild type origin. See, e.g., U.S. Pat. No. 5,821,047. The phagemid genome contains a selectable marker gene, e.g. Amp.sup.R or Kan.sup.R (for ampicillin or kanamycin resistance, respectively) for the selection of cells that are infected by a member of the library.

In another example of phage display, vectors can be used that carry nucleic acids encoding a set of phage genes sufficient to produce an infectious phage particle when expressed, a phage packaging signal, and an autonomous replication sequence. For example, the vector can be a phage genome that has been modified to include a sequence encoding the display protein. Phage display vectors can further include a site into which a foreign nucleic acid sequence can be inserted, such as a multiple cloning site containing restriction enzyme digestion sites. Foreign nucleic acid sequences, e.g., that encode display proteins in phage vectors, can be linked to a ribosomal binding site, a signal sequence (e.g., a M13 signal sequence), and a transcriptional terminator sequence.

Vectors can be constructed by standard cloning techniques to contain sequence encoding a polypeptide that includes a polypeptide of interest and a portion of a phage coat protein, and which is operably linked to a regulatable promoter. In some examples, a phage display vector includes two nucleic acids that encode the same region of a phage coat protein. For example, the vector includes one sequence that encodes such a region in a position operably linked to the sequence encoding the display protein, and another sequence which encodes such a region in the context of the functional phage gene (e.g., a wild-type phage gene) that encodes the coat protein. Expression of the wild-type and fusion coat proteins can aid in the production of mature phage by lowering the amount of fusion protein made per phage particle. Such methods are particularly useful in situations where the fusion protein is less tolerated by the phage.

Regulatable promoters can also be used to control the valency of the display protein. Regulated expression can be used to produce phage that have a low valency of the display protein. Many regulatable (e.g., inducible and/or repressible) promoter sequences are known. Such sequences include regulatable promoters whose activity can be altered or regulated by the intervention of user, e.g., by manipulation of an environmental parameter, such as, for example, temperature or by addition of stimulatory molecule or removal of a repressor molecule. For example, an exogenous chemical compound can be added to regulate transcription of some promoters. Regulatable promoters can contain binding sites for one or more transcriptional activator or repressor protein. Synthetic promoters that include transcription factor binding sites can be constructed and can also be used as regulatable promoters. Exemplary regulatable promoters include promoters responsive to an environmental parameter, e.g., thermal changes, hormones, metals, metabolites, antibiotics, or chemical agents. Regulatable promoters appropriate for use in E. coli include promoters which contain transcription factor binding sites from the lac, tac, trp, trc, and tet operator sequences, or operons, the alkaline phosphatase promoter (pho), an arabinose promoter such as an araBAD promoter, the rhamnose promoter, the promoters themselves, or functional fragments thereof (see, e.g., Elvin et al. (1990) Gene 37: 123-126; Tabor and Richardson, (1998) Proc. Natl. Acad. Sci. U.S.A. 1074-1078; Chang et al. (1986) Gene 44: 121-125; Lutz and Bujard, (1997) Nucl. Acids. Res. 25: 1203-1210; D. V Goeddel et al. (1979) Proc. Nat. Acad. Sci. U.S.A., 76:106-110; J. D. Windass et al. (1982) Nucl. Acids. Res., 10:6639-57; R. Crowl et al. (1985) Gene, 38:31-38; Brosius (1984) Gene 27: 161-172; Amanna and Brosius, (1985) Gene 40: 183-190; Guzman et al. (1992) J. Bacteriol., 174: 7716-7728; Haldimann et al. (1998) J. Bacteriol., 180: 1277-1286).

The lac promoter, for example, can be induced by lactose or structurally related molecules such as isopropyl-beta-D-thiogalactoside (IPTG) and is repressed by glucose. Some inducible promoters are induced by a process of derepression, e.g., inactivation of a repressor molecule.

A regulatable promoter sequence can also be indirectly regulated. Examples of promoters that can be engineered for indirect regulation include: the phage lambda P_{R}, P_{L}, phage T7, SP6, and T5 promoters. For example, the regulatory sequence is repressed or activated by a factor whose expression is regulated, e.g., by an environmental parameter. One example of such a promoter is a T7 promoter. The expression of the T7 RNA polymerase can be regulated by an environmentally-responsive promoter such as the lac promoter. For example, the cell can include a heterologous nucleic acid that includes a sequence encoding the T7 RNA polymerase and a regulatory sequence (e.g., the lac promoter) that is regulated by an environmental parameter. The activity of the T7 RNA polymerase can also be regulated by the presence of a natural inhibitor of RNA polymerase, such as T7 lysozyme.

In another configuration, the lambda P_{L} can be engineered to be regulated by an environmental parameter. For example, the cell can include a nucleic acid that encodes a temperature sensitive variant of the lambda repressor. Raising cells to the non-permissive temperature releases the P_{L} promoter from repression.

The regulatory properties of a promoter or transcriptional regulatory sequence can be easily tested by operably linking the promoter or sequence to a sequence encoding a reporter protein (or any detectable protein). This promoter-report fusion sequence is introduced into a bacterial cell, typically in a plasmid or vector, and the abundance of the reporter protein is evaluated under a variety of environmental conditions. A useful promoter or sequence is one that is selectively activated or repressed in certain conditions.

In some embodiments, non-regulatable promoters are used. For example, a promoter can be selected that produces an appropriate amount of transcription under the relevant conditions. An example of a non-regulatable promoter is the gIII promoter.

b. Transformation and Growth of Phage-Display Compatible Cells

For phage display using a phagemid vector, host cells compatible with phage display (typically partial suppressor cells, such as cells described in section D(2)(f) above), for example, XL1-Blue cells, are transformed, e.g. by electroporation or other known transformation methods with vectors containing polynucleotides encoding the proteins for display. The transformed cells can be grown for amplification of the vector nucleic acids, for example, for subsequent sequence analysis or pooling for re-transformation. In one example, transformed cells are grown in suitable medium, for example, SB medium supplemented with antibiotics, and incubated for use in phage display to express the variant polypeptides.

c. Co-Infection with Helper Phage, Packaging and Expression

When a phagemid vector is used, phage packaging and display of the polypeptides is induced by co-infection with helper phage, for example, with VCS M13 helper phage. Methods for transformation, growth and phage packaging and propagation are well-known (see Clackson and Lowman, Phage Display: A Practical Approach; (2004) Oxford University Press (Chapter 2, Constructing Phage display libraries by oligonucleotide-directed mutagenesis, Sidhu and Weiss, p. 27-41). Any phage display method can be used. In general, host cells transformed with the vector nucleic acids are incubated in medium. Helper phage is added and the cells are incubated. Typically, polypeptide expression is induced, for example, by IPTG. Exemplary protocols are described in Examples 4, 6, 7 and 8E, below. Generally, the expressed polypeptide (e.g. the polypeptide contained as part of a phage coat protein fusion) is directed to the periplasm of the bacterial host cell (e.g. using methods described above) so it can be assembled into phage.

d. Isolation of Genetic Packages Displaying the Polypeptides.

Following induction, phage displaying the polypeptides are produced from, typically secreted by, the host cells. The phage can be isolated, for example, by precipitation, and then assayed and/or used for selection of desired variant polypeptides.

For example, following phage propagation, the phage (genetic packages) displaying the polypeptides can be isolated from the host cells or from the media containing the host cells. For example, phage secreted in the culture medium can be precipitated using well-known methods. Typically, phage is precipitated and the precipitate collected by centrifugation. The precipitate typically is resuspended in a buffer and the solution centrifuged to remove debris (clearing).

In an exemplary protocol, cultures containing propagated phage are centrifuged, for example, at 8000 rpm for 10 minutes with the break on, and the supernatant retained. In this example, the pelleted cells optionally can be retained for assays, for example, sequencing of the nucleic acids in the vectors, or for iterative processes, and the supernatant can be transferred, and the phage precipitated from the supernatant. In one example, polyethylene glycol (for example, 20% PEG-8000 in 2.5 M NaCl, added at an amount to produce a final concentration of 4% PEG-8000, 0.5 M NaCl) is added to the supernatant and incubated on ice for approximately 30 minutes, to precipitate the phage. In this example, the phage then is centrifuged at 13,000 rpm, for 20 minutes are 4° C. The supernatant then is discarded (e.g. poured off) and the precipitated phage is dried, for example by inverting the tube, for 5-10 minutes. The precipitated phage then can be resuspended, for example in 1 mL 1% BSA and 1×PBS, and transferred to a microcentrifuge tube, which then is centrifuged (to clear the precipitate), for example, at 13,500 rpm, at 25° C., for 5 minutes. The supernatant then contains the phage, which can be used, for example, in screening and/or selection steps, for example, to isolate one or more desired variant polypeptides.

The selected polypeptides and/or phage displaying the polypeptides can be used in an iterative process, by repeating one or more aspects of the provided methods.

2. Other Display Methods

Other known display methods can be used. Display systems include, for example, prokaryotic or eukaryotic cells. Exemplary of systems for cell surface expression include, but are not limited to, bacteria, yeast, insect cells, avian cells, plant cells, and mammalian cells (Chen and Georgiou (2002) Biotechnol Bioeng 79: 496-503). In one example, the bacterial cells for expression are Escherichia coli.

a. Cell Surface Display

Polypeptides can be displayed as part of a fusion protein with a protein that is expressed on the surface of the cell, such as a membrane protein or cell surface-associated protein. For example, a polypeptide can be expressed in E. coli as a fusion protein with an E. coli outer membrane protein (e.g. OmpA), a genetically engineered hybrid molecule of the major E. coli lipoprotein (Lpp) and the outer membrane protein OmpA or a cell surface-associated protein (e.g. pili and flagellar subunits). Generally, when bacterial outer membrane proteins are used for display of heterologous peptides or proteins, expression is achieved through genetic insertion into permissive sites of the carrier proteins. Expression of a heterologous peptide or protein is dependent on the structural properties of the inserted protein domain, since the peptide or protein is more constrained when inserted into a permissive site as compared to fusion at the N- or C-terminus of a protein. Modifications to the fusion protein can be done to improve the expression of the fusion protein, such as the insertion of flexible peptide linker or spacer sequences or modification of the bacterial protein (e.g by mutation, insertion, or deletion, in the amino acid sequence). Enzymes, such as β-lacatamase and the Cex exoglucanase of Cellulomonas fimi, have been successfully expressed as Lpp-OmpA fusion proteins on the surface of E. coli (Francisco J. A. and Georgiou G. Ann N Y Acad. Sci. 745:372-382 (1994) and Georgiou G. et al. Protein Eng. 9:239-247 (1996)). Other peptides of 15-514 amino acids have been displayed in the second, third, and fourth outer loops on the surface of OmpA (Samuelson et al. J. Biotechnol. 96: 129-154 (2002)). Thus, outer membrane proteins can carry and display heterologous gene products on the outer surface of bacteria.

In another example, polypeptides are fused to autotransporter domains of proteins such as the N. gonorrhoeae IgA1 protease, Serratia marcescens serine protease, the Shigella flexneri VirG protein, and the E. coli adhesin AIDA-I (Klauser et al. EMBO J. 1991-1999 (1990); Shikata S, et al. J Biochem. 114:723-731 (1993); Suzuki T et al. J Biol Chem. 270:30874-30880 (1995); and Maurer J et al. J Bacteriol. 179:794-804 (1997)). Other autotransporter proteins include those present gram-negative species (e.g. E. coli, Salmonella serovar Typhimurium, and S. flexneri). Enzymes, such as β-lactamase, have been successful expressed on the surface of E. coli using this system (Lattemann C T et al. J Bacteriol. 182(13): 3726-3733 (2000)).

Bacteria can be recombinantly engineered to express a fusion protein, such a membrane fusion protein. Polynucleotides encoding the polypeptides for display can be fused to nucleic acids encoding a cell surface protein, such as, but not limited to, a bacterial OmpA protein. The nucleic acids encoding the polypeptides can be inserted into a permissible site in the membrane protein, such as an extracellular loop of the membrane protein. Additionally, a nucleic acid encoding the fusion protein can be fused to a nucleic acid encoding a tag or detectable protein. Such tags and detectable proteins are known in the art and include for example, but not limited to, a histidine tag, a hemagglutinin tag, a myc tag or a fluorescent protein. The nucleic acids encoding the fusion proteins can be operably linked to a promoter for expression in the bacteria, For example nucleic acid can be inserted in a vectors or plasmid, which can carry a promoter for expression of the fusion protein and optionally, additional genes for selection, such as for antibiotic resistance. The bacteria can be transformed with such plasmids, such as by electroporation or chemical transformation. Such techniques are known to one of ordinary skill in the art.

Proteins in the outer membrane or periplasmic space usually are synthesized in the cytoplasm as premature proteins, which are cleaved at a signal sequence to produce the mature protein that is exported outside the cytoplasm. Exemplary signal sequences used for secretory production of recombinant proteins for E. coli are known. The N-terminal amino acid sequence, without the Met extension, can be obtained after cleavage by the signal peptidase when a gene of interest is correctly fused to a signal sequence. Thus, a mature protein can be produced without changing the amino acid sequence of the protein of interest (Choi and Lee. Appl. Microbiol. Biotechnol. 64: 625-635 (2004)).

Other known cell surface display methods can be used, including, but not limited to, ice nucleation protein (Inp)-based bacterial surface display system (Lebeault J M (1998) Nat Biotechnol. 16: 576 80), yeast display (e.g. fusions with the yeast Aga2p cell wall protein; see U.S. Pat. No. 6,423,538), insect cell display (e.g. baculovirus display; see Ernst et al. (1998) Nucleic Acids Research, Vol 26, Issue 7 1718-1723), mammalian cell display, and other eukaryotic display systems (see e.g. 5,789,208 and WO 03/029456). The vectors provided herein can be used in any of these systems to display a protein of interest, such as a domain exchanged antibody, provided that the host cells contain an appropriate functional suppressor tRNA and that the vectors contain the appropriate elements for replication, amplification, transcription and translation in the host cell.

b. Other Display Systems

Other display formats also can be used. Exemplary other display formats include nucleic acid-protein fusions, ribozyme display (see e.g. Hanes and Pluckthun (1997) Proc. Natl. Acad. Sci. U.S.A. 13:4937-4942), bead display (Lam, K. S. et al. Nature (1991) 354, 82-84; K. S. et al. (1991) Nature, 354, 82-84; Houghten, R. A. et al. (1991) Nature, 354, 84-86; Furka, A. et al. (1991) Int. J. Peptide Protein Res. 37, 487-493; Lam, K. S., et al. (1997) Chem. Rev., 97, 411-448; U.S. Published Patent Application 2004-0235054) and protein arrays (see e.g. Cahill (2001) J. Immunol. Meth. 250:81-91, WO 01/40803, WO 99/51773, and US2002-0192673-A1).

In specific other cases, it can be advantageous to instead attach the polypeptides, or phage libraries or cells expressing variant polypeptides, to a solid support. For example, in some examples, cells expressing polypeptides can be naturally adsorbed to a bead, such that a population of beads contains a single cell per bead (Freeman et al. Biotechnol. Bioeng. (2004) 86:196-200). Following immobilization to a glass support, microcolonies can be grown and screened with a chromogenic or fluorogenic substrate. In another example, variant polypeptides or phage libraries or cells expressing variant polypeptides can be arrayed into titer plates and immobilized.

### F. Libraries of Polypeptides, Including Displayed Polypeptides and Selection of Displayed Polypeptides from the Libraries

Also provided herein are collections, including libraries and display libraries (e.g. phage display libraries) containing the polypeptides, such as domain exchanged antibodies, methods for making the libraries, and methods for selecting polypeptides, e.g. domain exchanged antibodies, from the libraries. In particular, provided herein are antibody libraries (e.g. domain exchanged antibody libraries). Any known methods for generating libraries containing variant polynucleotides and/or polypeptides (e.g. methods described herein and methods described in U.S. application No. [Attorney Docket No. 3800013-00031/1106] and International Application No. [Attorney Docket No. 3800013-00032/1106PC] can be used with the provided methods and vectors to generate display libraries, e.g. phage display libraries, of domain exchanged antibodies, and to select variant domain exchanged antibodies from the libraries. The libraries can be used in screening assays to select variant domain-exchanged antibodies from the library for any antigen, including, for example, any Candida antigen as exemplified in Examples 9-16. To facilitate screening, antibody libraries typically are screened using a display technique, such that there is a physical link between the individual molecules of the library (phenotype) and the genetic information encoding them (genotype). These methods include, but are not limited to, cell display, including bacterial display, yeast display and mammalian display, phage display (Smith, G. P. (1985) Science 228:1315-1317), mRNA display, ribosome display and DNA display.

Provided herein are domain exchange libraries. Like other libraries, these contain members having mutations compared to a target polypeptide, such as a domain exchanged antibody. Such libraries can be used to select new domain exchanged antibodies, for example, based on their ability to bind particular antigens with a desired affinity. Domain-exchanged antibody libraries are generated from nucleic acid molecule(s) encoding two VH chains and two VL chains, whereby the VH domains interact producing a V_{H}-V_{H}′ interface characteristic of the domain exchanged configuration. The nucleic acid molecules can be generated separately, such that upon expression of the antibody a domain-exchanged antibody is formed. For example, variant nucleic molecules can be generated encoding a VH chain of a domain-exchanged antibody and/or variant nucleic acid molecules can be generated encoding a VL chain of a domain-exchanged antibody. Upon co-expression of the nucleic acid molecules in a cell, a variant-domain exchanged-antibody is generated. Alternatively, a single nucleic acid molecule can be generated that encodes both the variant VH and VL chains of a domain-exchanged antibody. This is exemplified herein, for example, using a pCAL vector or variant or mutant thereof. In such a vector, a single nucleic acid molecule encodes both the heavy and light chain domains of a domain-exchanged antibody, for example, 2G12. In any of the libraries herein, the nucleic acid molecules also can further contain nucleotides for the hinge region and/or constant regions (e.g. CL or CH1, CH2 and/or CH3) of the domain-exchanged antibody. Further, the nucleic acid molecules optionally can include nucleotides encoding peptide linkers and/or dimerization domains. Methods to generate and express antibodies are described herein, and can be adapted for use in generating any domain-exchanged antibody library. Hence, the domain-exchanged antibody libraries can include members that are full-length antibodies, or that are antibody fragments thereof. Generally, domain-exchanged antibody libraries are Fab libraries.

A domain-exchanged antibody library includes light chain libraries, whereby each member contains variant residues only in the light chain. In another example, a domain-exchanged antibody includes heavy chain libraries, whereby each member contains variant residues only in the heavy chain of the domain-exchanged antibody. In a further example, domain exchanged antibody libraries include libraries where members include variant residues in both the heavy and light chain of the library. In all examples, the libraries of domain-exchanged antibodies are diverse, and contain least at or about 10⁴, 10⁵, 10⁶, 10⁷, 10⁸, 10⁹, 10¹⁰ 10¹¹, 10¹², 10¹³ 10¹⁴, or more, different polynucleotide sequences.

In generating the libraries, any domain-exchanged antibody can serve as the template for generating variant members of the libraries. Exemplary of a domain-exchanged antibody is 2G12 or an antigen fragment thereof. A domain-exchanged antibody also includes any antibody containing one or more mutations at isoleucine (Ile) at position 19, arginine (Arg) at position 57, phenylalanine (Phe) at position 77 and proline (Pro) at position 113, where numbering is based on kabat numbering. Further residues for amino acid mutation include amino acid residues 39, 70, 72, 79, 81 and 84 based on kabat numbering. In particular, the mutations are arginine (Arg) at position 39, serine (Ser) at position 70, Asparagine (Asn) at position 72 and Tyrosine (Tyr) at position 79, Glutamine (Gln) at position 81, Valine (Val) at position 84, based on kabat numbering. As discussed elsewhere herein, one of skill in the art able to identify a domain-exchanged binding molecule based on structural and other properties, for example, oligomerization state.

Exemplary template antibodies for use in the libraries herein do not bind to the target antigen. This ensures that when the libraries are created, the members of the library include minimal carryover of the backbone template vector. Where such carryover does exist, the template backbone vector is non-binding and will not be selected in screening or selection methods herein. For example, for use in identifying variants that bind to gp120 or Candida, exemplary templates include the 2G12 antibody or fragment thereof containing alanine mutations in the CDR H3 of the variable heavy chain (designated 3-ALA) at amino acid residues 104, 105 and 107 corresponding to amino acid residues in the V_{H} domain set forth in SEQ ID NO:. Also exemplary of a non-binding backbone domain exchanged antibody binding molecule is a 2G12 antibody or fragment thereof containing alanine mutations in the CDR L3 of the variable light chain (designated 3-ALA LC) at amino acid residues 91, 94 and 95 (amino acid residues 91, 94 and 95 by Kabat numbering) corresponding to amino acid residues in the V_{L} domain set forth in SEQ ID NO:305. Additionally, amino acid residues 91, 94 and 95 of SEQ ID NO:321 correspond to amino acid residues 92, 95 and 96 of SEQ ID NO:305. The 3-ALA and 3-ALA LC 2G12 molecules do not bind gp120 or Candida antigen.

Libraries can be generated by diversification of any one or more up to all residues in the CDR L1, L2, L3, H1, H2 and/or H3 of a template domain-exchanged antibodies. Diversification also can be effected in amino acid residues in the framework regions or hinge regions. One of skill in the art knows and can identify the CDRs and FR based on kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). For example, diversification of any one or more up to all residues in 2G12 can be effected, for example, amino acid residues in the CDR H11 (amino acid residues 31-35 of SEQ ID NO:154); CDR H2 (amino acid residues 50-66 of SEQ ID NO:154); CDR H3 (amino acid residues 99-112 of SEQ ID NO:154); CDRL1 (amino acid residues 24-34 of SEQ ID NO:155); CDR L2 (amino acid residues 50-56 of SEQ ID NO:155) and/or CDR L3 (amino acid residues 89-97 of SEQ ID NO:155).

Exemplary of residues selected for diversification are those that are directly involved in antigen-binding. In one example, residues involved in antigen-binding can be identified empirically, for example, by mutagenesis experiments directly assessing binding to an antigen. In another example, residues involved in antigen-binding can be elucidated by analysis of crystal structures of the domain-exchanged binding molecule with the antigen or a related antigen or other antigen. For example, crystal structures of 2G12 complexed with various antigens can be used to elucidate and identify potential antigen-binding residues. It is contemplated that such residues may be involved in binding to diverse antigens.

For example, based on crystal structure analysis of 2G12 binding to various antigens, exemplary antigen binding residues include, but are not limited to, L93 to L94 in CDR L3; H31, H32 and H33 in CDRH1; H52a in CDRH2; and H95, H96, H97, H98, H99, H100 in CDR H3, where residues are based on kabat numbering (Clarese et al. (2005) 300:2065). Other residues for diversification include L89, L90, L91, L92 and L95 in CDR L3; and H96, H100, H100a, H100c and H100d of CDRH3. For examples, exemplary of residues in the heavy chain for diversification include residues in the CDR H1 and CDR H3. For example, any one of amino acid residues H32, H33, H96, H100, H100a, H100c and H100d (corresponding to residues H32, H33, H100, H104, H105, H107 and H108 in SEQ ID NO:154) can be selected for diversification in generating a 2G12 heavy chain antibody library. In another example, exemplary of residues in the light chain for diversification include residues in the CDR3. For example, any one of amino acid residues L89 to L95 (corresponding to residues L89 to L95 in SEQ ID NO:155) can be selected for diversification in generating a 2G12 light chain antibody library.

Various well-known methods can be used in combination with the provided display methods to select desired polypeptides from the collections of displayed polypeptides (e.g. domain exchanged antibodies). For example, methods for selecting desired polypeptides from phage display libraries include panning methods, where phage displaying the polypeptides are selected for binding to a desired binding partner (see, for example, Clackson and Lowman, Phage Display: A Practical Approach; (2004) Oxford University Press (Chapter 1, Russel et al., An introduction to Phage Biology and Phage Display, pp. 1-26; Chapter 4, Dennis and Lowman, Phage selection strategies for improved affinity and specificity of proteins and peptided pp. 61-83)). Polypeptides selected from the collections optionally can be amplified, and analyzed, for example, by sequencing nucleic acids or in a screening assay (see, for example, Phage Display: A Practical Approach; (2004) Oxford University Press (Chapter 5, De Lano and Cunningham, Rapid screening of phage displayed protein binding affinities by phage ELISA pp 85-94)) to determine whether the selected polypeptide(s) has a desired property. In one example, iterative selection steps are performed in order to enrich for a particular property of the variant polypeptide.

1. Confirming Display of the Polypeptides

Typically, prior to selection of polypeptides from a collection, e.g. a phage display library, one or more methods is used to determine successful expression and/or display of the variant polypeptides. Such methods are well-known and include phage enzyme-linked immunosorbent assays (ELISAs), as described hereinbelow, for detection of binding to a binding partner, and/or detection of an epitope tag on the expressed polypeptides, such as a His6 tag, which can be detected by binding to metal-chelating matrices or anti-His antibodies bound to solid supports.

2. Selection of Polypeptides from the Collections

Also provided herein are methods for selecting polypeptides, e.g. domain exchanged antibodies, from the collections of displayed polypeptides, and displayed polypeptides selected from the collections. Typically, or more selection steps is carried out to select one or more variant polypeptides from the provided collections, e.g. phage display libraries ((see, for example, Clackson and Lowman, Phage Display: A Practical Approach; (2004) Oxford University Press (Chapter 1, Russel et al., An introduction to Phage Biology and Phage Display, pp. 1-26; Chapter 4, Dennis and Lowman, Phage selection strategies for improved affinity and specificity of proteins and peptided pp. 61-83)). Typically, the selection step is a panning step, whereby phage displaying the polypeptide are selected for their ability to bind to a desired binding partner (e.g. an antigen).

a. Panning

Panning methods for selection of phage-displayed polypeptides are well-known, and can be used with the provided methods and collections. Generally, a binding partner (an antigen or epitope in the case of a variant antibody polypeptide collection) is presented to the collection of phage and the collection enriched for members that bind, for example, with high affinity, to the binding partner.

In an exemplary panning process for selecting polypeptides from the libraries, the binding partner (e.g. antigen) is be coated on to microtiter wells and incubated with the collections of variant polypeptides expressed on the surface of phage. After washing non-specific binders from the wells using buffers known to those skilled in the art (e.g 1× phosphate buffered saline pH 7.4 with 0.01% Tween 20), the remaining variants are eluted with an elution buffer (e.g. 0.1 M HCl pH 2.2 with Glycine and Bovine Serum Albumin 1 mg/mL) and bacteria are infected with the eluted phage for the expansion of specific variants. This procedure can be repeated (e.g. 2-6 times) in an iterative screening process as described below, for the enrichment of specific variants with higher affinity.

i. Incubation of the Displayed Polypeptides with a Binding Partner

For panning, a binding partner is presented to the collection of phage displaying the polypeptides (e.g. domain exchanged antibody fragments). A number of means for presenting the binding partner to the phage are well-known and all can be used with the provided methods. In one example, the binding partner is immobilized on a solid support (e.g. a bead, column or well). Alternatively, the phage and a soluble binding partner can be incubated in solution, followed by capture of the binding partner. Alternatively, whole cells expressing the binding partner can be used to select phage. In vivo methods for selection also are known and can be used with the provided methods.

For immobilization of the binding partner, a number of solid supports can be used. Exemplary supports include resins and beads (e.g. sepharose, controlled-pore glass), plates (e.g. microtiter (96 and 384 well) plates, and chips (e.g. dextran-coated chips (BIAcore, Inc.)). In one example, the binding partner is immobilized by coupling to an affinity tag (e.g. biotin, His6) and immobilization on a solid support coated with a molecule having affinity for the tag (e.g. avidin, Ni²+). For binding of the phage to binding partners in solution, the phage can be selected by a second capture step using an appropriate matrix.

Prior to incubation of the phage with the binding partner, a blocking step can be carried out to prevent non-specific selection of phage. Binding reagents are well known and include bovine serum albumin (BSA), ovalbumin, casein and nonfat milk. An exemplary blocking step includes incubation of the blocking buffer (e.g. 4% nonfat dry milk in PBS) for one hour at 37° C. The blocking buffer can be discarded prior to incubation of the phage collection with the binding partner.

Typically, for incubation of the phage with the binding partner, a number of dilutions of the precipitated phage (e.g. prepared using a two- four- six- or ten-fold dilution curve) are prepared and incubated with the binding partner. In one example, where the binding partner is immobilized in wells of a microtiter plate, the phage dilutions are incubated in buffer (e.g. blocking buffer, optionally containing polysorbate 20), for example, for one to two hours, at room temperature or at 37° C., with optional rocking. Choice of buffer for the binding of the phage to the binding partner is based on several parameters, including the affinity of the target polypeptide or desired polypeptide for the binding partner and for the nature of the binding. For example, more or less protein can be included depending on the affinity. In some cases, it is necessary to include cations or cofactors to facilitate binding.

In one example, a competing decoy binding partner is included during the incubation step, for example, to reduce the possibility of selecting non-specific binders and/or to select polypeptides having high affinity for the binding partner. In another example, a non-specific polypeptide, having none or low affinity for the binding partner, is included in the panning step.

Typically, a first panning step, for example, using phage displaying only the target polypeptide, is conducted to verify the accuracy of the panning procedure.

ii. Washing

Following incubation with the binding partner, non-binding phage and/or polypeptides are washed away using one or more wash buffers. Typical wash buffers include PBS, and PBS supplemented with polysorbate 20 (Tween 20), for example, at 0.05%. Depending on the desired stringency, the wash buffer and/or length/number of washes can be varied, according to methods well known to the skilled artisan. Conditions of the binding and washing steps can be varied to adjust stringency, according to various parameters, for example, affinity of the target or desired polypeptide for the binding partner.

In one example, after washing, some of the samples can be used to analyze the polypeptides, for example, by performing an ELISA-based assay as described hereinbelow, to determine whether any of the polypeptides have bound to the binding partner. For example, when the panning is carried out in a well of a microtiter plate, duplicate wells for each dilution can be used. In this example, one of the wells from each sample is used to elute bound phage, while the phage bound to the other duplicate well is retained for analysis, e.g. by ELISA-based assay. Alternatively, the panning procedure can be continued, by eluting bound phage, which potentially display polypeptides having desired properties.

iii. Elution of Bound Polypeptides

After washing to remove non-bound phage, the phage expressing polypeptides that have bound to the binding partner are eluted using one of several well known elution methods, typically by reduction of the pH of the solution, recovery of phage, and neutralization, or addition of a competing polypeptide which can compete for binding to the binding partner. Exemplary of the elution step is reduction of the pH to approximately 2 (e.g. 2.2) by incubation of the bound phage with 10-100 mM hydrochloric acid (HCL), pH 2.2, or with 0.2 M glycine, (e.g. for 10 minutes at room temperature (e.g. 25° C.)), followed by removal of the eluate and addition of 1-2 M Tris-base (pH 8.0-9.0) to neutralize the pH. In some examples, multiple elution steps are carried out and the eluates pooled for subsequent steps.

Efficient elution can be assessed by analysis of the eluate, or alternatively, by performing an analysis on the solid support from which the phage have been eluted, e.g. by performing an ELISA-based assay as described hereinbelow.

c. Amplification and Analysis of Selected Polypeptides

In one example, displayed polypeptides (e.g. displayed domain exchanged antibodies) selected in the panning step are amplified for analysis and/or use in subsequent panning steps. The amplification step amplifies the genome of the genetic package, e.g. phage. This amplification can be useful for expressing the polypeptide encoded by the selected phage, for example, for use in analysis steps or subsequent panning steps in iterative selection processes as described hereinbelow, and for identification of the variant polypeptide and polynucleotide encoding the polypeptide, such as by subsequent nucleic acid sequencing.

In this example, following elution, the phage nucleic acids are amplified in an appropriate host cell. In one example, the selected phage is incubated with an appropriate host cell (e.g. XL1-Blue cells) to allow phage adsorption (for example, by incubation of eluted phage with cells having an O.D. between 0.3 and 0.6 for 20 minutes at room temperature). After this incubation to allow phage adsorption, a small volume of nutrient broth is added and the culture agitated to facilitate phage DNA replication in the multiplying host cell. After this incubation, the culture typically is supplemented with an antibiotic and/or inducer and the cells grown until a desired optical density is reached. The phage genome can contain a gene encoding resistance to an antibiotic to allow for selective growth of the cells that maintain the phage vector DNA. The amplification of the display source, such as in a bacterial host cell, can be optimized in a variety of ways. For example, the host cells can be added in vast excess to the genetic packages recovered by elution, thereby ensuring quantitative transduction of the genetic package genome. The efficiency of transduction optionally can be measured when phage are selected.

In another example, after selection of one or more displayed polypeptides, for example, by panning using a phage display library as described above, the polypeptide(s) are purified and analyzed. Exemplary analysis methods include general recombinant DNA techniques, routine to those of skill in the art. The vector containing the polynucleotide encoding the selected variant polypeptide (e.g. the phagemid vector), can be isolated to enable purification of the selected protein. For example, following infection of E. coli host cells with selected phage as set forth above, the individual clones can be picked and grown up for plasmid purification using any method known to one of skill in the art, and if necessary can be prepared in large quantities, such as for example, using the Midi Plasmid Purification Kit (Qiagen). The purified plasmid can used for nucleic acid sequencing to identify the sequence of the variant polynucleotide and, by extrapolation, the sequence of the variant polypeptide, or can be used to transfect into any cell for expression, such as by not limited to, a mammalian expression system. If necessary, one or two-step PCR can be performed to amplify the selected sequence, which can be subcloned into an expression vector of choice. The PCR primers can be designed to facilitate subcloning, such as by including the addition of restriction enzyme sites. Following transfection into the appropriate cells for expression, such as is described in detail hereinabove, the selected polypeptides can be tested in a number of assays.

In one example, the polypeptides are analyzed for the ability to bind one or more binding partners. For example, if the polypeptide is an antibody, the polypeptide can be analyzed for ability to interact with a particular antigen, and for affinity for the antigen. In this example the binding partner is attached to a support, such as a solid support, and the polypeptides (e.g. precipitated phage) incubated with the support, followed by a wash to remove unbound polypeptides, and detection, for example, using a labeled antibody. Exemplary of supports to which the binding partner can be attached are wells, for example, microtiter wells, beads, e.g. sepharose beads, and/or beads for use in flow cytometry.

In one example, an ELISA-based assay is used, whereby the desired binding partner is coated onto wells of a microtiter plate, the plate is blocked with protein (e.g. bovine serum albumin) and the polypeptides, e.g. precipitated phage, are incubated with the coated wells. Following incubation, the unbound polypeptides are washed away in one or more wash steps and the bound polypeptides are detected, for example, using a detection antibody, for example, an antibody labeled with a fluorescent or enzyme marker. In the case of an enzyme marker, detection is carried out by incubation with a substrate, followed by reading of absorbance at an appropriate wavelength. Such binding assays can be used to evaluate polypeptides expressed from host cells, including polypeptides expressed on precipitated phage, including polypeptides selected using the panning methods provided herein, in order to verify their desired properties.

d. Iterative Selection

In one example, the screening of collections of displayed polypeptides is performed using an iterative process (e.g. multiple rounds of panning), for example, to optimize variation of the polypeptides, to enrich the selected polypeptides for one or more desired characteristics, and to increase one or more desired properties. Thus, in methods of iterative screening, a polypeptide can be evolved by performing the panning steps, described hereinabove, a plurality of times. In one example, the same parameters are used in each successive round. Typically, the successive rounds are performed using varying parameters, such as for example, by using different binding partners and/or decoys, or by increasing stringency of washes and/or binding steps.

In one example of iterative screening, selected polypeptides (optionally first amplified and analyzed) are used in multiple additional rounds of screening, by pooling the selected polypeptides (e.g. eluted phage), propagation of nucleic acids encoding the polypeptides in host cells, expression (e.g. phage display) of the selected polypeptides, and a subsequent round of panning. Multiple rounds, e.g. 2, 3, 4, 5, 6, 7, 8, or more rounds, of screening can be performed. In this example of iterative screening, the variant polypeptide collection used in the successive round of screening includes the polypeptides selected in the previous round. Alternatively, the multiple rounds of screening can be performed using the initial collection of polypeptides.

In an alternative example of iterative screening, a new polypeptide collection can be generated, that has been further varied. In one such example, one or more selected variant polypeptides is/are used as target polypeptides for variation using the methods provided herein.

In one example, a first round panning of the collection of polypeptides library can identify variant polypeptides containing one or more particular mutations (e.g. mutations in the CDR region(s) compared to an antibody target polypeptide), which alter one or more properties (e.g. antigen specificity) of the target polypeptide. In this example, a second round of variation and selection then can be performed, where the selected polypeptide(s) are used as target polypeptides for further variation, but the sequences of one or more of the particular mutations (e.g. the CDR sequences), are held constant, and new variant and/or randomized positions are selected for variation outside of these regions. After an additional round of screening, the selected polypeptides further can be subjected to additional rounds of variation and screening. For example, 2, 3, 4, 5, or more rounds of polypeptide variation and screening can be performed. In some examples, a property of the polypeptides (for example, the affinity of an antibody polypeptide for a specific antigen) is further optimized with each round of selection.

### G. General Host Cell-Vector Systems for Nucleic Acid Amplification and Protein Expression

Various combinations of host cells and vectors can be used to receive, maintain, reproduce and amplify nucleic acids (e.g. nucleic acid libraries encoding antibodies such as domain exchanged antibodies), and to express polypeptides encoded by the nucleic acids, such as the displayed polypeptides (e.g. domain exchanged antibodies) provided herein. In general, the choice of host cell and vector depends on whether amplification, polypeptide expression, and/or display on a genetic package, is desired. In one example, the same host cell and/or vector is used to amplify the nucleic acids, express the polypeptide and for display on a genetic package. In another example, different host cells and/or vectors are used. Methods for transforming host cells are well known. Any known transformation method, for example, electroporation, can be used to transform the host cell with nucleic acids.

In some examples, domain-exchanged antibodies are expressed in host cells and produced therefrom. The domain-exchanged antibodies can be expressed as full-length domain-exchanged antibodies, or as antibodies that are less then full length, for example, as domain-exchanged antibody fragments, including, but not limited to Fabs, Fab hinge fragment, scFv fragment, scFv tandem fragment and scFv hinge and scFv hinge (ΔE) fragments. Thus, for example, it is understood that any of the antibodies provided herein can be produced in any form so long as the resulting antibodies are domain-exchanged antibodies, which have a particular structure containing an interface formed by two interlocking V_{H} domains (VH-VH′ interface). For example, domain-exchanged antibodies provided herein generally contain at least two VH chains and two VL chains, whereby the VH domains interact producing a V_{H}-V_{H}′ interface characteristic of the domain exchanged configuration. The antibodies can further be produced to contain a hinge region, constant region or linkers.

1. Amplification of Nucleic Acids

In one example, vectors, such as the provided display vectors and other vectors, are used to transform host cells for amplification of nucleic acids encoding the provided polypeptides. When the vectors are used to transform host cells, the nucleic acids are replicated as the host cell divides, amplifying the nucleic acids.

Nucleic acids are amplified, for example, to isolate the nucleic acids encoding polypeptides such as displayed polypeptides, e.g. to determine the nucleic acid sequence or for use in transformation of other host cells. In one example, after transforming the host cells with the vectors, the host cells are incubated in medium, for example, SOC (Super Optimal Catabolite) medium (Invitrogen™; for 1 liter: 20 grams (g) Bacto Tryptone; 5 g Yeast Extract; 0.58 g Sodium Chloride (NaCl); 0.186 g Potassium Chloride (KCl) in distilled water); SB (Super Broth) medium (for 1 liter: 30 g tryptone, 20 g yeast extract, 10 g MOPS in distilled water); or LB (Luria broth) medium (for 1 L: 10 g Bacto Tryptone; 5 g yeast extract; 10 g NaCl, in distilled water) in the presence of one or more antibiotics, for selection of cells successfully transformed with vector nucleic acids containing insert, typically at 37° C. In one example, the incubated host cells are grown overnight at 37° C. on agar plates supplemented with one or more antibiotics and/or glucose, for generation of clonal colonies, each containing host cells transformed with a single vector nucleic acid.

One or more colonies can be picked for isolation of nucleic acids for use in subsequent steps, for example, in nucleic acid sequencing. Alternatively, picked colonies can be pooled and used to re-transform additional host cells, for example, phage-compatible host cells. In another example, the colonies can be picked and grown, and then the cultures used to induce protein expression from the host cells, for example, to assay expression of the variant polypeptides in the host cells, prior to phage display.

The colonies can be used to determine transformation efficiency, for example, by calculating the number of transformants generated from a library, by multiplying the number of colonies by the culture volume and dividing by the plating volume (same units), using the following equation: [# colonies/plating volume×[culture volume)/microgram DNA]×dilution factor.

Nucleic acids encoding domain exchanged antibodies can be introduced into vectors for expression thereof. For example, after insertion of the nucleic acid, the vectors typically are used to transform host cells, for example, to amplify the recombined antibody genes for replication and/or expression thereof. In such examples, a vector suitable for high level expression is used.

In one example, nucleic acid encoding the heavy chain of a domain-exchanged antibody is ligated into a first expression vector and nucleic acid encoding the light chain of a domain-exchanged antibody is ligated into a second expression vector. The expression vectors can be the same or different, although generally they are sufficiently compatible to allow comparable expression of proteins (heavy and light chain) therefrom. For example, to generate a domain-exchanged Fab, sequences encoding the V_{H}-C_{H}1 can be cloned into a first expression vector and sequences encoding the V_{L}-C_{L} domains can be cloned into a second expression vector. An exemplary expression vector includes pTT5 (NRC Biotechnology Research) for expression in HEK293-6E cells. Other expression vectors and host cells are described below. The first and second expression vectors are co-transfected into host cells, typically at a 1:1 ratio. Upon expression of two copies of an antibody fragment chain (e.g., two copies of the V_{H}-C_{H}1 chain and V_{L}-C_{L}), two heavy chain variable regions (V_{H}) interlock and further pair with a light chain variable region (V_{L}) to generate domain-exchanged Fab dimers. If desired, the vectors also can contain further sequences encoding additional constant region(s) or hinge regions to generate other antibody forms. For example, a full-length domain exchanged antibody can be generated including in a first expression vector, encoding the heavy gene, sequences for the hinge and Fc regions. Upon co-expression with the second expression vector encoding the V_{L}-C_{L} domains a full-length domain-exchanged antibody is expressed. Using these exemplified methods, it is within the level of one of skill in the art to generate other antibody forms, including other antibody fragment forms of domain-exchanged antibodies.

In an another example, nucleic acid molecules encoding both the heavy and light chain of a domain-exchanged antibodies are expressed from the same vector. This is exemplified above with respect to display vectors. It is understood that any of the display vectors, for example, any pCAL vector, described above can be used to produce soluble protein. For example, such vectors can be modified to not include the display protein (e.g. coat protein). Alternatively, vectors that do not contain a stop codon in the leader sequence but that do contain a stop codon between the nucleic acid encoding the antibody and the coat protein, can be introduced into a non-suppressor host cell strain. Upon expression, there is no readthrough of the stop codon, so that only soluble antibody chains are expressed without fusion to a coat protein.

Using either of the above methods, one of skill in the art can generate a full-length domain-exchanged antibody, or an domain-exchanged antibody fragment such as any described herein below.

2. Expression of Encoded Polypeptides

In another example, expression of polynucleotides encoded by the vectors is induced in host cells. Induction of polypeptide expression can be used to isolate and analyze polypeptides encoded by nucleic acids, such as nucleic acid libraries, encoding the polypeptides. Host cells for expression include display-compatible host cells (e.g. phage display compatible), which can be used to display the polypeptides on the surface of a genetic package (e.g. a bacteriophage), for example, in a phage display library.

In one example, polypeptide expression is induced from the host cells for isolation and analysis of the polypeptides, for example, to determine if polypeptides in a collection bind a particular binding partner, e.g. an antigen. Methods for inducing polypeptide expression from host cells are well known and vary depending on choice of vector and host cell. In one example, one or more colonies is picked and grown in medium supplemented with antibiotic and grown until a desired Optical Density (O.D.) is reached. Protein expression then can be induced by well-known methods, for example, by addition of isopropyl-beta-D-thiogalactopyranoside (IPTG) and continued growth.

Methods for purification of polypeptides, including domain exchanged antibodies, from host cells will depend on the chosen host cells and expression systems. For secreted molecules, proteins generally are purified from the culture media after removing the cells. For intracellular expression, cells can be lysed and the proteins purified from the extract. In one example, polypeptides are isolated from the host cells by centrifugation and cell lysis (e.g. by repeated freeze-thaw in a dry ice/ethanol bath), followed by centrifugation and retention of the supernatant containing the polypeptides. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary further the proteins can be extracted and further purified using standard methods in the art.

Proteins, such as the provided domain exchanged antibodies, can be purified, for example, from lysed cell extracts, using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind proteases can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

The isolated polypeptides then can be analyzed, for example, by separation on a gel (e.g. SDS-Page gel), size fractionation (e.g. separation on a Sephacryl™ S-200 HiPrep™ 16×60 size exclusion column (Amersham from GE Healthcare Life Sciences, Piscataway, N.J.). Isolated polypeptides can also be analyzed in binding assays, typically binding assays using a binding partner bound to a solid support, for example, to a plate (e.g. ELISA-based binding assays) or a bead, to determine their ability to bind desired binding partners. The binding assays described in the sections below, which are used to assess binding of precipitated phage displaying the polypeptides, also can be used to assess polypeptides isolated directly from host cell lysates. For example, binding assays can be carried out to determine whether antibody polypeptides bind to one or more antigens, for example, by coating the antigen on a solid support, such as a well of an assay plate and incubating the isolated polypeptides on the solid support, followed by washing and detection with secondary reagents, e.g. enzyme-labeled antibodies and substrates.

Polypeptides, such as any set forth herein, including antibodies or fragments thereof, can be produced by any method known to those of skill in the art including in vivo and in vitro methods. Desired polypeptides can be expressed in any organism suitable to produce the required amounts and forms of the proteins, such as for example, needed for analysis, administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of polypeptides. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

3. Host Cells

A variety of host cells can be used. These include but are not limited to mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

For display of the polypeptides on genetic packages, a host cell is selected that is compatible with such display. Typically, the genetic package is a virus, for example, a bacteriophage, and a host cell is chosen that can be infected with bacteriophage, and accommodate the packaging of phage particles, for example XL1-Blue cells. In another example, the host cell is the genetic package, for example, a bacterial cell genetic package, that expresses the variant polypeptide on the surface of the host cell.

a. Prokaryotic Cells

Prokaryotes, especially E. coli, provide a system for producing large amounts of proteins. Typically, E. coli host cells are used for amplification and expression of the provided variant polypeptides. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Proteins, such as any provided herein, can be expressed in the cytoplasmic environment of E. coli. For some polypeptides, the cytoplasmic environment, can result in the formation of insoluble inclusion bodies containing aggregates of the proteins. Reducing agents such as dithiothreotol and β-mercaptoethanol and denaturants, such as guanidine-HCl and urea can be used to resolubilize the proteins, followed by subsequent refolding of the soluble proteins. An alternative approach is the expression of proteins in the periplasmic space of bacteria which provides an oxidizing environment and chaperonin-like and disulfide isomerases and can lead to the production of soluble protein. For example, for phage display of the proteins, the proteins are exported to the periplasm so that they can be assembled into the phage. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used for expression and production of polypeptides, such as any described herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as variant polypeptides provided herein. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including the variant polypeptides provided herein. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be to express polypeptides such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce proteases or modified proteases (see for example, Mayfield et al. (2003) PNAS 100:438-442). Because plants have different glycosylation patterns than mammalian cells, this can influence the choice of protein produced in these hosts.

4. Nucleic Acid Libraries

In one example, the provided vectors and methods for display can be used to generate nucleic acid libraries and polypeptide libraries encoded by the nucleic acid libraries, such as display libraries, e.g. phage display libraries, which contain diversity among the members of the library. Thus, provided are collections of vectors (nucleic acid libraries), such as collections for expressing diverse domain exchanged antibodies, and libraries displaying the encoded diverse polypeptides, e.g. domain exchanged antibodies, and antibodies selected from the libraries. Methods for generating libraries (collections) of variant nucleic acid molecules (nucleic acid libraries) are well known in the art and can be used to generate collections of variant polypeptides, such as display libraries, in combination with the provided methods.

a. Generating Nucleic Acid Libraries

The vectors provided herein can be used to generate nucleic acid libraries. In some instances, polynucleotides in existing nucleic acid libraries are inserted into the phagemid vectors provided herein. For example, nucleic acid libraries containing polynucleotides encoding proteins, such as, for example, antibodies, such as domain exchanged antibodies, can be inserted into the vectors herein. Typically, the nucleic acid libraries contain a diverse collection of polynucleotides. Methods for generating nucleic acid libraries and for creating diversity in the nucleic acid library are well know in the art and can be employed to generate nucleic acid libraries for use with the vector provided herein. Approaches for generating diversity include targeted and non-targeted approaches well known in the art.

Known approaches for generating diverse nucleic acid and polypeptide libraries include, but are not limited to:

non-targeted approaches (whereby diversity is introduced at random) such as recombination approaches (e.g. chain shuffling, (Marks et al., J. Mol. Biol. (1991) 222, 581-597; Barbas et al., Proc. Natl. Acad. Sci. USA (1991) 88, 7978-7982; Lu et al., Journal of Biological Chemistry (2003) 278(44), 43496-43507; Clackson et al., Nature (1991) 352, 624-628; Barbas et al., Proc. Natl. Acad. Sci. USA (1992) 89, 10164; U.S. Pat. Nos. 6,291,161, 6,291,160, 6,291,159, 6,680,192, 6,291,158, and 6,969,586); and “sexual PCR” (Stemmer, Nature (1994) 340, 389-391; Stemmer, Proc. Natl. Acad. Sci. USA (1994) 10747-10751; and U.S. Pat. No. 6,576,467; Boder et al., PNAS (2000) 97(20), 10701-10705)); and error-prone PCR (Zhou et al., Nucleic Acids Research (1991) 19(21), 6052; Gram et al. Proc. Natl. Acad. Sci. USA 89, 3567-3580; Rice et al., Proc. Natl. Acad. Sci. USA (1992) 89 5467-5471; Fromant et al., Analytical Biochemistry (1995) 224(1) 347-353; Mondon et al., Biotechnol. J. (2007) 2, 76-82 U.S. Application Publication No. 2004/0110294; Low et al., J. Mol Biol. (1996) 260(3) 359-368; Orencia et al., Nature Structural Biology (2001) 8(3) 238-242; and Coia et al., J Immunol Methods (2001) 251(1-2) 187-193);

targeted approaches (for mutating particular positions or portions), such as cassette mutagenesis (Wells et al., Gene (1985) 34, 315-323; Oliphant et al., Gene (1986) 44, 177-183; Borrego et al., Nucleic Acids Research (1995) 23, 1834-1835; Baca et al., The Journal of Biological Chemistry (1997) 272(16) 10678-10684; Breyer and Sauer Journal of Biological Chemistry (1989) 264(22) 13355-13360; Oliphant and Strul Proc. Natl. Acad. Sci. USA (1989) 86, 9094-9098; U.S. Pat. No. 7,175,996; Borrego et al., Nucleic Acids Research (1995) 23, 1834-1835; and Wells et al., Gene (1985) 34, 315-323); mutual primer extension (Oliphant et al., Gene (1986) 44, 177-183; Bryer and Sauer Journal of Biological Chemistry (1989) 264(22) 13355-13360; Oliphant and Strul Proc. Natl. Acad. Sci. USA (1989) 86, 9094-9098) template-assisted ligation and extension (Baca et al., The Journal of Biological Chemistry (1997) 272(16) 10678-10684); codon cassette mutagenesis (Kegler-Ebo et al., Nucleic Acids Research, (1994) 22(9), 1593-1599; Kegler-Ebo et al., Methods Mol Biol., (1996), 57, 297-310); oligonucleotide-directed mutagenesis (Brady and Lo, Methods Mol Biol. (2004), 248, 319-26; Rosok et al., The Journal of Immunology, (1998) 160, 2353-2359) and amplification using degenerate oligonucleotide primers (U.S. Pat. Nos. 5,545,142, 6,248,516, and 7,189,841; Barbas et al., Proc. Natl. Acad. Sci. USA (1992) 89, 4557-4461; Pini et al., The Journal of Biological Chemistry (1998) 273(34), 21769-21776; Ho et al., The Journal of Biological Chemistry (2005), 280(1), 607-617), including overlap and two-step PCR (Higuchi et al., Nucleic Acids Research (1988); 16(15), 7351-7367; Jang et al., Molecular Immunology (1998), 35, 1207-1217; Brady and Lo, Methods Mol Biol. (2004), 248, 319-26; Burks et al., Proc. Natl. Acad. Sci. USA (1997) 94, 412-417; Dubreuil et al., The Journal of Biological Chemistry (2005) 280(26), 24880-24887); and

combined approaches, such as combinatorial multiple cassette mutagenesis (CMCM) and related techniques (Crameri and Stemmer, Biotechniques, (1995), 18(2), 194-6; and US2007/0077572; De Kruif et al., J. Mol. Biol. (1995) 248, 97-105; Knappik et al., J. Mol. Biol. (2000), 296(1), 57-86; and U.S. Pat. No. 6,096,551).

Exemplary of the methods for generating diverse nucleic acid libraries, such as with the provided vectors, are those described in related U.S. application No. [Attorney Docket No. 3800013-00031/1106] and International Application No. [Attorney Docket No. 3800013-00032/1106PC], and those exemplified in Example 5, below. The collections of variant polynucleotides produced using such methods contain diversity, typically at least at or about 10⁴, 10⁵, 10⁶, 10⁷, 10⁸, 10⁹, 10¹⁰ 10¹¹, 10¹², 10¹³ 10¹⁴, or more, different polynucleotide sequences, and each member of the collection contains at least 100 or about 100, 200 or about 200, 300 or about 300, 500 or about 500, 1000 or about 1000, or 2000 or about 2000 nucleotides in length. A brief summary of these methods is provided in the following sections, and one method is exemplified in Example 5.

i. Selection of Target Polypeptides

In a first step of an exemplary method for making collections of variant polynucleotides (i.e. a nucleic acid library) that encode variant polypeptides (such as in a phage display library), a target polypeptide is selected for variation. For the purposes herein, the target polypeptide is typically an antibody, particularly a domain exchanged antibody. In one example, the target polypeptide is a native polypeptide. In another example, the target polypeptide is a variant polypeptide, for example a variant polypeptide generated by the methods herein (e.g. a variant antibody or antibody fragment from an antibody library generated using the provided methods). Exemplary of target polypeptides are antibodies, antibody domains, antibody fragments and antibody chains, as well as regions within the antibody fragments, domains and chains. The target polypeptide is encoded by a target polynucleotide. One or more target domains, target portions and/or target positions can be specifically selected for variation within the target polypeptide.

The target domains, portions and/or positions typically are selected based on a desire to generate a collection of polypeptides that vary in a particular structural or functional property compared to the target polypeptide. For example, for alteration of a polypeptide function, a functional domain that contributes to or affects that function can be selected as the target domain. In one example, when it is desired to generate a collection of variant antibody polypeptides with varying antigen specificities or binding affinities, an antigen binding site domain is selected as a target domain within a target antibody polypeptide. One or more target portions can be selected within the target domain. For example, each target portion of an antigen binding site domain can include part or all of an amino acid sequence of a CDR. In one example, each CDR within an antibody variable region or within an entire antibody binding site is selected as a target portion. Alternatively, the target portions can be selected at random along the amino acid sequence of the target polypeptide.

ii. Design and Synthesis of Oligonucleotides

Oligonucleotides are designed and synthesized for use in nucleic acid libraries that encode the variant polypeptides. Oligonucleotide design is based on a target polynucleotide encoding the target polypeptide or, typically, a region and/or domain of the target polynucleotide. A reference sequence (a sequence of nucleotides containing sequence identity to a region of the target polynucleotide) is used as a design template for synthesizing the oligonucleotides. The oligonucleotides can be variant oligonucleotides, for example, randomized oligonucleotides. Alternatively, the oligonucleotides can be reference sequence oligonucleotides, which have identity, such as at or about 100% sequence identity, to the reference sequence that is used in designing the oligonucleotides. Typically, variant (e.g. randomized) and reference sequence oligonucleotides are synthesized and then assembled by one of the provided methods, to make a collection of variant nucleic acids (e.g. collection of variant assembled duplexes or duplex cassettes).

Typically, the oligonucleotides are synthetic oligonucleotides, which are synthesized in pools of oligonucleotides. Each synthetic oligonucleotide in a pool is designed based on the same reference sequence. Each randomized oligonucleotide in a pool of randomized oligonucleotides has at least one, typically at least two, reference sequence portions and at least one, for example, 1, 2, 3, 4, 5, 6, 7, 8, 9, 10 or more, randomized portions. Randomized positions within the randomized portion(s) are synthesized using one or more of a plurality of doping strategies.

In one example, a plurality of pools of oligonucleotides, typically more than two, for example 2, 3, 4, 5, 6, 7, 8, 9, 10, 15, 20 or more pools of oligonucleotides, is synthesized. In one example, oligonucleotides are designed so that oligonucleotides from each of the plurality of pools can be assembled in subsequent steps to form assembled duplex cassettes. In some such examples, assembled duplexes are generated by hybridization of positive and negative strand oligonucleotides within the plurality of pools and/or by polymerase reactions, such as amplification reactions, including, but not limited to, polymerase chain reaction (PCR), followed by formation of assembled duplex cassettes, for example, by restriction digest. In some examples, intermediate duplexes are formed before forming the assembled duplexes. Typically, in these examples, the reference sequences used to design the individual pools of oligonucleotides have sequence identity to different regions along the target polynucleotide. In one example, two or more of these different regions are overlapping along the sequence of the target polynucleotide.

Biased and non-biased doping strategies can be used during synthesis of randomized portions in pools of randomized oligonucleotides. In non-biased doping strategies, each of a plurality of nucleotides or tri-nucleotides is present at an equal proportion during synthesis of each nucleotide or tri-nucleotide position. In biased doping strategies, particular nucleotide monomers or codons are included at different frequencies than others, thus biasing the sequence of the randomized portions within a collection towards a particular sequence within the randomized portions.

Non-biased randomization is carried out using a non-biased doping strategy where each of a plurality of nucleotide monomers or trimers are added at equal percentages during synthesis of the randomized position. Exemplary of a non-biased doping strategy is “NNN,” one whereby each of the four nucleotide monomers (A, G, T and C) is added at an equal proportion during synthesis of each nucleotide position in a randomized portion. The strategy can lead to equal frequency of each nucleotide monomer at each randomized position within the collection synthesized using this strategy. Non-biased doping strategies using an equal ratio of each of the nucleotide monomers can be undesirable, as they lead to a relatively high frequency of stop codon incorporation compared to some biased strategies. Because there are sixty-four possible combinations of tri-nucleotide codons, which encode only twenty amino acids, redundancy exists in the nucleotide code. Different amino acids have a more redundant code than others. Thus, non-biased incorporation of nucleotides will not result in an equal frequency of each of the twenty amino acids in the encoded polypeptide. If an equal frequency of amino acids is desired, a non-biased doping strategy using equal ratios of a plurality of tri-nucleotide units, each representing one amino acid, can be employed.

In biased randomization, a doping strategy is used in synthesis of the randomized positions to incorporate particular nucleotides or codons at different frequencies than others, biasing the sequence of the randomized portions towards a particular sequence. For example, the randomized portion, or single nucleotide positions within the randomized portion, can be biased towards a reference nucleotide sequence or the coding sequence of a target polynucleotide. Biasing positions towards a reference nucleotide sequence means that, within a collection of randomized oligonucleotides, the nucleotides or codons used in the reference sequence at those nucleotide positions would be more common than other nucleotides or codons. Doping strategies also can be biased to reduce the frequency of stop codons while still maintaining a possibility for saturating randomization. Alternatively, the doping strategy can be non-biased, whereby each nucleotide is inserted at an equal frequency.

Exemplary of biased doping strategies used herein are NNK, NNB and NNS, and NNW; NNM, NNH; NND; NNV doping strategies and an NNT, NNA, NNG and NNC doping strategy. In an NNK doping strategy, randomized portions of positive strands are synthesized using an NNK pattern and negative strand portions are synthesized using an MNN pattern, where N is any nucleotide (for example, A, C, G or T), K is T or G and M is A or C. Thus, using this doping strategy, each nucleotide in the randomized portion of the positive strand is a T or G. This strategy typically is used to minimize the frequency of stop codons, while still allowing the possibility of any of the twenty amino acids (listed in table 2) to be encoded by trinucleotide codons at each position of the randomized portion among the randomized oligonucleotides in the pool. Similarly, for the NNB doping strategy, an NNB pattern is used, where N is any nucleotide and B represents C, G or T. For the NNS doping strategy, an NNS pattern is used, where N is any nucleotide and S represents C or G. In an NNW doping strategy, W is A or T; in an NNM doping strategy, M is A or C; in an NNH doping strategy, H is A, C or T; in an NND doping strategy, D is A, G or T; in an NNV doping strategy, G is A, G or C. An NNK doping strategy minimizes the frequency of stop codons and ensures that each amino acid position encoded by a codon in the randomized portion could be occupied by any of the 20 amino acids. With this doping strategy, nucleotides were incorporated using an NKK pattern and a MNN pattern, during synthesis of the positive and negative strand randomized portions respectively, where N represents any nucleotide, K represents T or G and M represents A or C. An NNT strategy eliminates stop codons and the frequency of each amino acid is less biased but omits Q, E, K, M, and W. Other doping strategies include all four nucleotide monomers (A, G, C, T), but at different frequencies. For example, a doping strategy can be designed whereby at each position within the randomized portion, the sequence is biased toward the wild-type sequence or the reference sequence. Other well-known doping strategies can be used with the methods provided herein, including parsimonious mutagenesis (see, for example, Balint et al., Gene (1993) 137(1), 109-118; Chames et al., The Journal of Immunology (1998) 161, 5421-5429), partially biased doping strategies, for example, to bias the randomized portion toward a particular sequence, e.g. a wild-type sequence (see, for example, De Kruif et al., J. Mol. Biol., (1995) 248, 97-105), doping strategies based on an amino acid code with fewer than all possible amino acids, for example, based on a four-amino acid code (see, for example, Fellouse et al., PNAS (2004) 101(34) 12467-12472), and codon-based mutagenesis and modified codon-based mutagenesis (See, for example, Gaytán et al., Nucleic Acids Research, (2002), 30(16), U.S. Pat. Nos. 5,264,563 and 7,175,996).

iii. Generation of Assembled Oligonucleotide Duplexes and Duplex Cassettes

Following oligonucleotide synthesis, synthetic oligonucleotides and/or duplexes generated from the oligonucleotides are used to generate duplexes, including intermediate duplexes and assembled duplexes, including assembled duplex cassettes. Synthetic oligonucleotides and/or duplexes from two or more, typically three or more, pools are assembled to form assembled duplexes. In one example, the assembled duplexes are large assembled duplexes. The large assembled duplexes can be generated by hybridization, polymerase reactions, amplification reactions, ligation, and/or combinations thereof.

Typically, the large assembled duplexes are greater than 50 or about 50 nucleotides in length, for example, greater than at or about 50, 55, 60, 65, 70, 75, 80, 85, 90, 95, 100, 150, 200, 250, 300, 350, 400, 450, 500, 600, 700, 800, 900, 1000, 1500, 2000 or more nucleotides in length. In one example, the large assembled duplexes contain the length of an entire coding region of a gene. Typically, the large assembled duplexes have one, typically more than one, for example, 2, 3, 4, 5, 6, 7, 8, 9, 10, 15, 20 or more variant portions. Typically the more than one variant portions are randomized portions. In one example, the assembled duplexes are assembled duplex cassettes, which can be directly ligated into vectors. In one example, assembled duplexes are cut with restriction endonucleases, to generate the assembled duplex cassettes, which then can be ligated into vectors.

In some of the provided approaches, oligonucleotide duplex cassettes are generated directly, without using a restriction digestion step, for example, by hybridizing complementary positive and negative strand synthetic oligonucleotides. An example of such an approach is used in random cassette mutagenesis and assembly (RCMA) described in related U.S. application No. [Attorney Docket No. 3800013-00031/1106] and International Application No. [Attorney Docket No. 3800013-00032/1106PC].

Briefly, in RCMA, assembled duplex cassettes, typically large assembled duplex cassettes, are generated by combining a plurality of oligonucleotide pools. Each assembled duplex cassette is made by hybridization and assembly of a plurality of positive and negative strand oligonucleotides with shared regions of complementarity. The approaches used in RCMA can be used to generate assembled duplex cassettes directly from synthetic oligonucleotides, without a restriction digestion step. The cassettes can be inserted directly into the vectors provided herein for reduced expression of the encodes polypeptides.

In other approaches, assembled duplexes are formed by hybridizing synthetic template oligonucleotides and synthetic oligonucleotide primers, followed by polymerase extension. In these approaches, the resulting assembled duplexes are used to generate duplex cassettes for insertion into vectors, for example, by cutting with restriction endonucleases. Exemplary of such an approach, used in oligonucleotide fill-in and assembly (OFIA; related U.S. application No. [Attorney Docket No. 3800013-00031/1106] and International Application No. [Attorney Docket No. 3800013-00032/1106PC]), a plurality of oligonucleotide template pools and oligonucleotide fill-in primer pools (which regions of complementarity to one another) are used in a plurality of fill-in reactions, whereby complementary strands are synthesized, thereby producing a plurality of pools of double-stranded duplexes, which then are digested with restriction endonucleases and assembled, to generate assembled duplexes. In one example, when the assembled duplexes contain restriction sites, the assembled duplexes then can be digested with one or more restriction endonucleases to create cassettes that can be inserted into the vectors provided herein for reduced expression of the encoded polypeptides.

In other examples, a combination of hybridization and polymerase reactions are used to generate the assembled duplexes. Exemplary of such an approach is used in duplex oligonucleotide ligation/single primer amplification (DOLSPA; described in related U.S. application No. [Attorney Docket No. 3800013-00031/1106] and International Application No. [Attorney Docket No. 3800013-00032/1106PC]. In this approach, a plurality of synthetic oligonucleotide pools (typically a combination of reference sequence oligonucleotide pools and variant oligonucleotide pools) are combined to assemble intermediate duplexes by hybridization and ligation. The intermediate duplexes then are used in an amplification reaction to form assembled duplexes. In one example of DOLSPA, the amplification reaction is a single-primer extension reaction using a non gene-specific primer. In another example, the amplification reaction is carried out using two primers, e.g. two gene-specific primers. As in other approaches, in one example, the assembled duplexes can be cut with restriction endonucleases to form assembled duplex cassettes, which can be ligated into the vectors provided herein for reduced expression of the encoded polypeptides.

Also exemplary of the combined approaches for generating assembled duplexes, Fragment Assembly and Ligation/Single Primer Amplification (FAL-SPA), described in related U.S. application No. [Attorney Docket No. 3800013-00031/1106] and International Application No. [Attorney Docket No. 3800013-00032/1106PC]. In this approach, pools of variant duplexes (typically randomized duplexes) (FIG. 3A), reference sequence duplexes (FIG. 3B), and scaffold duplexes (FIG. 3B) are generated simultaneously or in any order. In one example, the variant duplexes are generated by performing fill-in and/or amplification reactions, where synthetic variant template oligonucleotides (typically randomized template oligonucleotides) are incubated in the presence of oligonucleotide primers, under conditions whereby complementary strands are synthesized. Typically, the reference sequence and scaffold duplexes are generated by synthesizing complementary strands from the target polynucleotide or region thereof.

As illustrated in FIG. 3B, the scaffold duplexes contain regions of complementarity to variant (e.g. randomized) duplexes and reference sequence duplexes, and are used to facilitate ligation of polynucleotides from these two types of duplexes, to make pools of assembled polynucleotides, by bringing the polynucleotides in close proximity through hybridization via complementary regions. For this process, called fragment assembly and ligation (FAL) (FIG. 3C), the pools of variant duplexes, reference sequence duplexes and scaffold duplexes are incubated under conditions whereby polynucleotides from the duplexes hybridize through complementary regions, and whereby nicks are sealed, for example, by addition of a ligase, thereby forming assembled polynucleotides containing sequences of reference sequence duplexes and variant (e.g. randomized) duplexes.

Assembled duplexes then are generated by synthesizing complementary strands of the assembled polynucleotides, typically in a polymerase reaction, typically a single primer amplification (SPA) reaction (FIG. 3D), which uses a single primer pool to prime complementary strand synthesis from the 5′ ends of the assembled polynucleotides, thereby generating pools of assembled duplexes. In one example, as with the other methods described herein, the assembled duplexes then can be used to make assembled duplex cassettes, for example, for ligation into vectors.

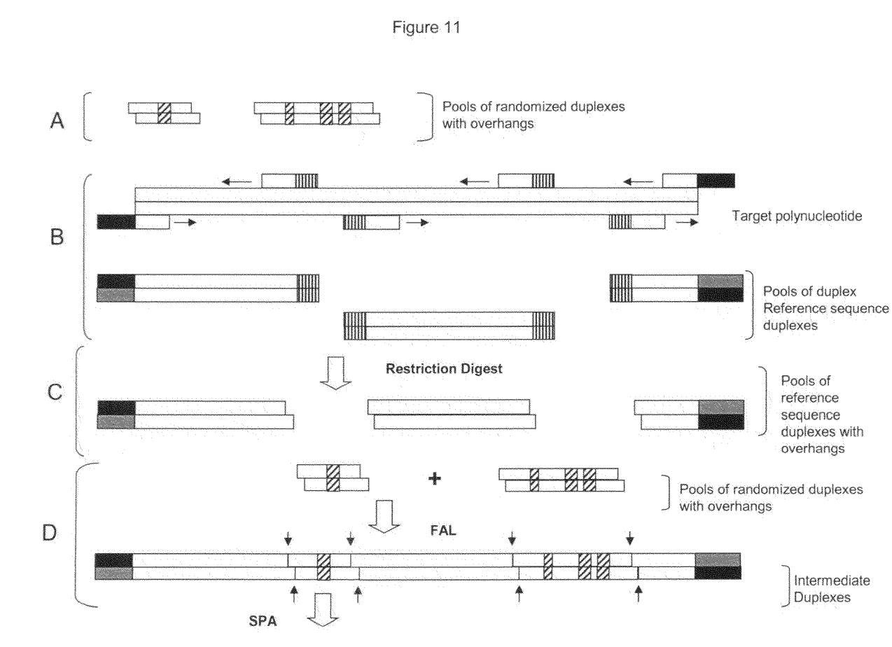

A modified variation of the FAL-SPA approach (mFAL-SPA) is illustrated in FIG. 11 and exemplified in Example 5, below. In mFAL-SPA, the pools of variant, e.g. randomized duplexes are designed so that the resulting duplexes contain one, typically two, restriction site overhangs, which are used for assembly with reference sequence duplexes in a subsequent step. Typically, the variant (e.g. randomized) duplexes are formed by hybridizing pools of positive strand oligonucleotides and pools of negative strand oligonucleotides under conditions whereby oligonucleotides in the pools hybridize through regions of complementarity.

Reference sequence duplexes are generated, such as in FAL-SPA. Typically, the reference sequence duplexes are generated by incubating target polynucleotide or region thereof with primers, each of which contains a sequence of nucleotides corresponding to a restriction endonuclease cleavage site (nucleotide sequences illustrated as filled grey and black boxes in FIG. 11B). In this example, a restriction endonuclease cleavage step (FIG. 11C) further is carried out following the generation of the reference sequence duplexes, generating overhangs, typically being a few nucleotides in length, e.g. 2, 3, 4, 5, 6, 7, or more nucleotides in length. Typically, the restriction site overhangs designed in the variant oligonucleotides are selected based on the restriction endonuclease site used in the primers, such that cleavage of the reference sequence duplexes with the restriction endonuclease produces overhangs that are compatible with the overhangs generated in the variant oligonucleotide duplexes. Exemplary of the restriction endonuclease cleavage site is a SAP-I cleavage site (GCTCTTC; SEQ ID NO: 44 (or the reverse complement, GAAGAGC; SEQ ID NO 45), which allows production of 3-nucleotide overhangs of a sequence near the site.

The pools of duplexes are combined in a fragment assembly and ligation (FAL) step to form pools of intermediate duplexes (FIG. 11D). Typically the pools of intermediate duplexes are assembled through the compatible overhangs. Assembled duplexes are generated using the intermediate duplexes are synthesized, e.g. in an amplification step, typically a single primer amplification (SPA) reaction, where a “single primer” (pool of identical primers) is used to prime complementary strand synthesis from the 5′ and the 3′ ends of the single strand fragments of the denatured intermediate duplex. In one example, as with the other methods described herein, the assembled duplexes then can be used to make assembled duplex cassettes, for example, for ligation into vectors.

iv. Ligation of the Assembled Duplex Cassettes into Vectors

After generation of duplex cassettes, the cassettes are inserted into the vectors provided herein, for amplification of the nucleic acids and reduced expression of the encoded polypeptides. The cassettes typically are inserted into the vectors using restriction digest and ligation, through restriction site overhangs generated in one or more of the previous steps. Typically, the vector into which a cassette is inserted contains all or part of the target polynucleotide.

### H. Domain Exchanged Libraries

Provided herein are domain exchanged libraries, including display libraries. The domain exchanged libraries provided herein can be generated using the methods, vectors and cells described herein. As described above, ny known methods for generating libraries containing variant polynucleotides and/or polypeptides can be used. For example, any method described herein and/or known to one of skill in the art, for example, methods described in U.S. Provisional Application, Attorney Docket No.: 119367-00014/p1106B, can be used to generate domain-exchanged antibody libraries. The libraries can be used in screening assays to select variant domain-exchanged antibodies from the library for any antigen, including, for example, any Candida antigen described herein. To facilitate screening, antibody libraries typically are screened using a display technique, such that there is a physical link between the individual molecules of the library (phenotype) and the genetic information encoding them (genotype). These methods include, but are not limited to, cell display, including bacterial display, yeast display and mammalian display, phage display (Smith, G. P. (1985) Science 228:1315-1317), mRNA display, ribosome display and DNA display.

a. Variant Libraries

i. Selecting Residues

Libraries can be generated by diversification of any one or more up to all residues in the CDR L1, L2, L3, H1, H2 and/or H3 of a template domain-exchanged antibodies. Diversification also can be effected in amino acid residues in the framework regions or hinge regions. One of skill in the art knows and can identify the CDRs and FR based on kabat or Chothia numbering (see e.g., Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). For example, diversification of any one or more up to all residues in 2G12 can be effected, for example, amino acid residues in the CDR H11 (amino acid residues 31-35 of SEQ ID NO:154); CDR H2 (amino acid residues 50-66 of SEQ ID NO:154); CDR H3 (amino acid residues 99-112 of SEQ ID NO:154); CDRL1 (amino acid residues 24-34 of SEQ ID NO:155); CDR L2 (amino acid residues 50-56 of SEQ ID NO:155) and/or CDR L3 (amino acid residues 89-97 of SEQ ID NO:155).

Exemplary of residues selected for diversification are those that are directly involved in antigen-binding. In one example, residues involved in antigen-binding can be identified empirically, for example, by mutagenesis experiments directly assessing binding to an antigen. In another example, residues involved in antigen-binding can be elucidated by analysis of crystal structures of the domain-exchanged binding molecule with the antigen or a related antigen or other antigen. For example, crystal structures of 2G12 complexed with various antigens can be used to elucidate and identify potential antigen-binding residues. It is contemplated that such residues may be involved in binding to diverse antigens, including Candida.

For example, based on crystal structure analysis of 2G12 binding to various antigens, exemplary antigen binding residues include, but are not limited to, L93 to L94 in CDR L3; H31, H32 and H33 in CDRH1; H52a in CDRH2; and H95, H96, H97, H98, H99, H100 in CDR H3, where residues are based on kabat numbering (Clarese et al. (2005) 300:2065). Other residues for diversification include L89, L90, L91, L92 and L95 in CDR L3; and H96, H100, H100a, H100c and H100d of CDRH3. For examples, exemplary of residues in the heavy chain for diversification include residues in the CDR H1 and CDR H3. For example, any one of amino acid residues H32, H33, H96, H100, H100a, H100c and H100d (corresponding to residues H32, H33, H100, H104, H105, H107 and H108 in SEQ ID NO:154) can be selected for diversification in generating a 2G12 heavy chain antibody library. In another example, exemplary of residues in the light chain for diversification include residues in the CDR3. For example, any one of amino acid residues L89 to L95 (corresponding to residues L89 to L95 in SEQ ID NO:155) can be selected for diversification in generating a 2G12 light chain antibody library.

### EXAMPLES

The following examples are included for illustrative purposes only and are not intended to limit the scope of the invention.

### Example 1

### Vector for Expressing Soluble and GeneIII-Fused AC-8

This Example describes a study conducted to demonstrate that introduction of an amber stop codon between a nucleic acid encoding an antibody target polynucleotide and a nucleic acid encoding a coat protein could yield expression of non-fusion (soluble) and fusion protein heavy chain polypeptides in host cells. Two vectors, each containing nucleic acid encoding a human anti-HSV-8 scFv antibody fragment (AC-8), an HA tag, and a bacteriophage cp3-encoding gene (gIII), where the nucleic acid encoding the antibody fragment and the gIII were separated by an amber stop codon (TAG). One vector, containing a G residue immediately 3′ of the amber stop codon, was obtained from The Scripps Research Institute (La Jolla, Calif.). This vector was sequenced through the antibody framework and into the start of gene III. This region of the vector had the nucleic acid sequence set forth in SEQ ID NO: 46.

For generation of the other vector, which contained a G residue immediately 3′ of the amber stop codon, the QuikChange Site-Directed Mutagenesis Kit (Stratagene, La Jolla Calif.) was used in PCR mutagenesis to replace the G immediately following the amber stop codon with an A, using conditions suggested by the supplier.

Approximately 250 ng of each vector then was used to transform non-amber suppressor, Top10 (Invitrogen™ Corporation, Carlsbad, Calif.) cells, and partial amber-suppressor, XL1-Blue cells. Individual transformed colonies were grown overnight at 37° C. in 3 mL of LB medium supplemented with 50 μg/mL ampicillin. The cultures were then diluted 10-fold into 3 mL of fresh media and grown at 37° C. to an optical density (OD) of 0.6.

1 mM IPTG then was added to half of the cultures. Duplicate cultures were grown in the absence of IPTG. The cultures then were grown at 30° C. for an additional 4 hours. The cells were collected by centrifugation at 3,000 rpm, for 15 minutes, and resuspended in 25 μL PBS.

The samples then were boiled in SDS loading buffer for 10 min and loaded on a 10% SDS-PAGE gel. Following gel electrophoresis, proteins were transferred to a 0.2 μm nitrocellulose membrane for 1 hr at 10V. The membrane was blocked with 5% non-fat dry milk in PBS containing 0.05% Tween for 1 hr at room temperature. Next, the membrane was incubated overnight at 4° C. with 1:2000 anti-HA-HRP (Roche Applied Science, Indianapolis, Ind.) in 5% non-fat dry milk in PBS containing 0.05% Tween. After washing the membrane 3 times, for 5 minutes each, with PBS containing 0.05% Tween, an enhanced chemiluminescent substrate (SuperSignal, Thermo Fisher Scientific, Rockford, Ill.) was added and the membrane was imaged. Density analysis was carried out on the images of the membranes, to determine relative intensities of bands corresponding to non-gene III-fused AC8 antibody versus gene III-fused AC8 antibody.

The results indicated that in the non-amber suppressor (Top10) cells, only non-gene III-fused AC8 heavy chain polypeptide was produced. In the partial amber-suppressor (XL1-Blue) cells, however, bands corresponding to the sizes of the AC8 and the AC8-gene III polypeptides were present. In the cultures that were grown in the presence of 1 mM IPTG, the expression of the AC8-gIII fusion relative to non-fusion AC8 was approximately 1:1, while in the cells that were not treated with IPTG, the ratio was approximately 1:2. The results of this study indicated that the provided methods and vectors can be used to express, from a single vector, two polypeptides: a soluble antibody chain and a fusion-protein containing the same antibody chain, each antibody chain encoded by a single genetic element.

### Example 2

### Design and Production of Vectors for Phage Display of Domain Exchanged Antibodies (e.g. Domain Exchanged Antibody Fragments)

After verifying that soluble and phage coat protein fusion protein antibody heavy chains could be expressed from the same genetic element by including an amber stop codon between the antibody nucleic acid and the coat protein nucleic acid, vectors were designed for phage display of domain exchanged antibodies using this method.

### Example 2A

### Construction of pCAL G13 and pCAL A1 Vectors

This Example describes the process by which two phagemid vectors (pCAL G13 (SEQ ID NO: 13) and pCAL G13 A1 (SEQ ID NO:14) were designed and generated. These vectors can be used for display of peptides, such as antibody polypeptides, particularly for display of domain exchanged antibody fragments. Vectors for display of particular exemplary domain exchanged antibodies are described in subsequent examples, below.

The pCAL G13 and pCAL G13 A1 vectors each contained a truncated (C-terminal) M13 phage gene III sequence and an amber stop codon (TAG), upstream of the gene III sequence. The pCAL G13 and pCAL G13 A1 vectors contained identical sequences, with the exception that the pCAL A1 vector contained a G-A substitution in the first nucleotide encoding the truncated gene III, compared to the pCAL G13 vector. The pCAL G13 vector is represented schematically in FIG. 7. These vectors were produced as described in the sub-sections below.

(i) Assembly of 539 Base-Pair Fragment with lacZ Promoter and Cloning Sites

In order to assemble a 539 base-pair (bp) fragment containing the lacZ promoter and cloning sites of each vector, the oligonucleotides listed in Table 5, below, were designed and ordered from Integrated DNA Technologies (IDT) (Coralville, Iowa). Each oligonucleotide contained a 5′ phosphate group. The oligonucleotides were reconstituted to 100 μM in TE pH 8.0 and further diluted to 20 μM in TE pH 8.0. 10 μL of each oligonucleotide was mixed with 1.4 μL 5M NaCl in a 141.4 μL volume. The mixture was incubated at 90° C. for 5 min on a dry heat block and slowly cool down to room temperature. The resulting assembled 539 by fragment contained the sequences of the oligonucleotides, and contained Sap I/Spe I restriction endonuclease site overhangs on 5′ and 3′ ends, respectively.

TABLE 5

Oligonucleotides used for the composition of lacZ promoter
and cloning sites for light chain and heavy chain.

| | | |
|---|---|---|
| Name | Sequence | SEQ ID NO |

| | | |
|---|---|---|
| pCAL_0 | AGCGGAAGAGCGCCCAATACGCAAACCGCCTCTCCCCGC | 47 |
| | GCGTTGGCCGATTCATTAATGCAGCTGGCAC | |
| pCAL_1 | GACAGGTTTCCCGACTGGAAAGCGGGCAGTGAGCGCAAC | 48 |
| | GCAATTAATGTGAGTTAGCTCACTCATTAGGCACCCCAG | |
| | GCTTTAC | |
| pCAL_2 | ACTTTATGCTTCCGGCTCGTATGTTGTGTGGAATTGTGAG | 49 |
| | CGGATAACAATTGAATTAAGGAGGATATAATTATGAAAT | |
| | ACCTGC | |
| pCAL_3 | TGCCGACCGCAGCCGCTGGTCTGCTGCTGCTCGCGGCCC | 50 |
| | AGCCGGCCATGGCCGCCGGTGCCTAACTCTGGCTGGTTTC | |
| | GCTACC | |
| pCAL_4 | GTAACCGGTTTAATTAATAAGGAGGATATAATTATGAAA | 51 |
| | AAGACAGCTATCGCGATTGCAGTGGCACTGGCTGGTTTC | |
| | GCTACCG | |
| pCAL_5 | TAGCCCAGGCGGCCGCACGCGTCTGGTTGAATCTGGTGG | 52 |
| | GGTCTGGAATTCTGCGATCGCGGCCAGGCCGGCCGCACC | |
| | ATCACCA | |
| pCAL_6 | TCACCATGGCGCATACCCGTACGACGTTCCGGACTACGC | 53 |
| | TTCTA | |
| pCAL_7 | CTAGTAGAAGCGTAGTCCGGAACGTCGTACGGGTATGCG | 54 |
| | CCATGGTGATGGTGATGGTGCGGCCGGCCTG | |
| pCAL_8 | GCCGCGATCGCAGAATTCCAGACCCCACCAGATTCAACC | 55 |
| | AGACGCGTGCGGCCGCCTGGGCTACGGTAGCGAAACCAG | |
| | CCAGTGC | |
| pCAL_9 | CACTGCAATCGCGATAGCTGTCTTTTTCATAATTATATCC | 56 |
| | TCCTTATTAATTAAACCGGTTACGGTAGCGAAACCAGCC | |
| | AGAGTT | |
| pCAL_10 | AGGCACCGGCGGCCATGGCCGGCTGGGCCGCGAGCAGC | 57 |
| | AGCAGACCAGCGGCTGCGGTCGGCAGCAGGTATTTCATA | |
| | ATTATATC | |
| pCAL_11 | CTCCTTAATTCAATTGTTATCCGCTCACAATTCCACACAA | 58 |
| | CATACGAGCCGGAAGCATAAAGTGTAAAGCCTGGGGTGC | |
| | CTAATG | |
| pCAL_12 | AGTGAGCTAACTCACATTAATTGCGTTGCGCTCACTGCCC | 59 |
| | GCTTTCCAGTCGGGAAACCTGTCGTGCCAGCTGCATTAAT | |
| | GAATC | |
| pCAL_13 | GGCCAACGCGCGGGGAGAGGCGGTTTGCGTATTGGGCGC | 60 |
| | TCTTCC | |

(ii) PCR Amplification of Gene III from M13 mp 18 with SpeIG3-F and PvuINheIG3-R Primers

For the amplification of gene III (G3) (G) (for production of the pCAL G13 vector) from M13 phage, a 5′ primer SpeIG3-F (having the sequence set forth in SEQ ID NO: 61 (GGTGGTGGTTCTGGTACTAGTTAGGAGGGTGGTG)) and a 3′ primer, PvulNheIG3-R (having the nucleic acid sequence set forth in SEQ ID NO: 62 (GGGAAGGGCGATCGTTAGCTAGCTTAAGACTCCTTATTACGCAGTATGTT AG), were ordered from IDT, and M13 mp18 RF1 DNA was ordered from New England Biolabs (NEB). The M13 mp18 DNA (100 nanograms (ng)/μL) was diluted in water to a concentration of 10 ng/μL and G3(G) was amplified with the above primers using Advantage HF2 DNA polymerase (Clontech) in the presence of its reaction buffer and dNTP mix in a 100 μL reaction volume. The PCR consisted of a denaturation step at 95° C. for 1 min, 5 cycles of denaturation at 95° C. for 5 seconds and annealing and extension at 72° C. for 1 min, and 30 cycles of denaturation at 95° C. for 5 seconds and annealing and extension at 68° C. for 1 min, followed by the incubation at 68° C. for 3 minutes. The PCR product was run on a 1% agarose gel and purified using Gel Extraction Kit (Qiagen).

To generate G3 (A) (for making the pCAL G 13 A1 vector) by introducing the G to A mutation in the first nucleotide encoding truncated gene III, a primer, SpeG3A-F (having the nucleic acid sequence set forth in SEQ ID NO: 63 (GGTGGTGGTTCTGGTACTAGTTAGAAGGGTGGTG)) was ordered from IDT. Two ng of the G3(G) product that was amplified above was used as a template for amplification of a mutant G3(A) fragment, by amplification with primers SpeG3A-F and PvuINheIG3-R. The amplification was carried out in a PCR, using Advantage HF2 DNA polymerase in the presence of its reaction buffer and dNTP in a 100 μL reaction volume. PCR was performed as above for the amplification of G3(G). The PCR product was run on a 1% agarose gel and purified using a Gel Extraction Kit (Qiagen).

The purified G3 (G) and G3 (A) products then were digested with Spe I and Pvu I restriction endonucleases, using the buffers and conditions recommended by the supplier. The digested products then were purified using PCR purification columns (Qiagen).

pBlueScript II KS(+) vector (Stratagene) then was digested with Sap I and Pvu I and run on a 0.7% agarose gel. Visualization of the gel revealed a 2419 fragment, which was purified using the Gel Extraction Kit.

(iii) Ligation into Vector and Transformation of Host Cells

Fifty nanograms (ng) of the 2419 by vector fragment, 50 ng of the 539 by lacZ promoter/coning site fragment and 30-40 ng of either G3(G) or G3(A) product (isolated after digestion with Spe I/Pvu I) then were ligated using T4 DNA ligase (NEB) with its reaction buffer at room temperature (20-25° C.) for at least 2 hrs.

For transformation of host cells, 1 μL of each ligation reaction (that for G3 (G) and G3 (A)) was electroporated into 80 μL of TOP10F′ cells (Invitrogen™ Corporation, Carlsbad, Calif.) at 2.5 kV in 0.2 cm gap cuvettes. The cells then were resuspended in 1 mL SOC medium. The cells were incubated at 37° C. for 1 hr; serial dilutions of the transformed bacteria then were made and the samples spread onto LB agar plates supplemented with 100 μg/mL ampicillin. The plates were incubated at 37° C. overnight.

To check insertion of the fragments into the vectors, colonies were picked from the plates and grown in culture plates with 1.2 mL of Super Broth (SB) medium containing 20 mM glucose and 50 μg/mL of ampicillin at 37° C. overnight shaking at 300 rpm. The culture plates then were centrifuged at 3000 rpm for 10 minutes. DNA was purified from the cell pellets using QIAprep 8 Turbo Miniprep Kit (Qiagen, Valencia, Calif.) according to the manufacturer's protocol. Because the vector, as constructed, contained Age I and Nhe I sites, the vector DNA was digested with these restriction endonucleases and run on an agarose gel. Visualization of the gel revealed an appropriately sized 753 by fragment in DNA from some clones, indicating that these clones contained vectors with the G3 insert. These 753 by fragments were isolated from the gel using a gel extraction kit (Qiagen) and sent for sequencing analysis to Eton Bioscience (San Diego, Calif.). Sequencing revealed that these clones contained pCAL G13 G3 and pCAL A1 vectors, containing the 753 by G3 (G) and G3 (A) inserts, respectively.

### Example 2B

### Generation of Vectors for Display of Domain Exchanged Antibody Fragments, 2G12 and 3-ALA 2G12

pCAL phagemid vectors produced as described in Example 2A, above, were used to generate vectors for display of two domain exchanged Fab fragments (2G12 and 3-ALA 2G12). As described in the following sub-sections, 2G12 vectors were generated containing nucleic acid encoding a 2G12 light chain fragment (V_{L} and CL), and a 2G12 heavy chain fragment (V_{H} and C_{H}1); and 3-ALA vectors were generated containing a 2G12 light chain fragment and a 3-Ala 2G12 mutant heavy chain fragment. The heavy chain-encoding polynucleotides in the vectors were directly upstream of an amber stop codon (TAG). This design of the vectors resulted in vectors for expression of 2G12 (or 3-ALA) heavy chain-gene III fusion polypeptide, and soluble 2G12 or 3-ALA heavy chain (V_{H}/C_{H}1) polypeptides from the same genetic element, which was used, as described in subsequent examples, for display of these domain exchanged antibodies on phage.

(i) 2G12 pCAL G13

The 2G12 pCAL G13 vector was made by inserting a nucleic acid encoding a light chain domain of the 2G12 antibody (SEQ ID NO: 64) and heavy chain domain of the same antibody (SEQ ID NO: 65) into the pCAL G13 vector (SEQ ID NO: 13), described in Example 2A, above, along with a sequence of nucleotides (SEQ ID NO: 66: TACCCGTACGACGTTCCGGACTACGCT) encoding an HA tag (SEQ ID NO: 67: YPYDVPDYA), as follows:

The 2G12 pCAL G13 vector was made by the following process. Polynucleotides encoding 2G12 heavy and light chains were amplified from a pET Duet vector, having the nucleic acid sequence set forth in SEQ ID NO: 68 and cloned into the pCAL G13 vector, which is described in Example 2A, above. Two primers (pCALVL-F: CCATGGCCGCCGGTGTTGTTATGACCCAGTCTCCGTC (SEQ ID NO: 69), and pCALCK-R: CTCCTTATTAATTAATTAGCATTCACCACGGTTGAAAG (SEQ ID NO: 70)) were used to amplify the light chain fragment and two heavy chain primers (pCALVH-F (SEQ ID NO: 71): GCCCAGGCGGCCGCAGAAGTTCAGCTGGTTGAATCTGGTG; and pCALCH-R: (SEQ ID NO: 72) CTGGCCGCGATCGCAGGCAAGATTTCGGTTCAACTTTCTTG) were used to amplify the heavy chain fragment, using conventional PCR. The products then were digested with SgrA I/Pac I and Not I/AsiS I and cloned into the pCAL G13 vector, described in Example 2A, above.

The resulting 2G12 pCAL G13 vector contained the nucleic acid sequence set forth in SEQ ID NO: 32

(GTGGCACTTTTCGGGGAAATGTGCGCGGAACCCCTATTTGTTTATTTTT
CTAAATACATTCAAATATGTATCCGCTCATGAGACAATAACCCTGATAAA
TGCTTCAATAATATTGAAAAAGGAAGAGTATGAGTATTCAACATTTCCGT
GTCGCCCTTATTCCCTTTTTTGCGGCATTTTGCCTTCCTGTTTTTGCTCA
CCCAGAAACGCTGGTGAAAGTAAAAGATGCTGAAGATCAGTTGGGTGCAC
GAGTGGGTTACATCGAACTGGATCTCAACAGCGGTAAGATCCTTGAGAGT
TTTCGCCCCGAAGAACGTTTTCCAATGATGAGCACTTTTAAAGTTCTGCT
ATGTGGCGCGGTATTATCCCGTATTGACGCCGGGCAAGAGCAACTCGGTC
GCCGCATACACTATTCTCAGAATGACTTGGTTGAGTACTCACCAGTCACA
GAAAAGCATCTTACGGATGGCATGACAGTAAGAGAATTATGCAGTGCTGC
CATAACCATGAGTGATAACACTGCGGCCAACTTACTTCTGACAACGATCG
GAGGACCGAAGGAGCTAACCGCTTTTTTGCACAACATGGGGGATCATGTA
ACTCGCCTTGATCGTTGGGAACCGGAGCTGAATGAAGCCATACCAAACGA
CGAGCGTGACACCACGATGCCTGTAGCAATGGCAACAACGTTGCGCAAAC
TATTAACTGGCGAACTACTTACTCTAGCTTCCCGGCAACAATTAATAGAC
TGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTA
GTTATCTACACGACGGGGAGTCAGGCAACTATGGATGAACGAAATAGACA
GATCGCTGAGATAGGTGCCTCACTGATTAAGCATTGGTAACTGTCAGACC
AAGTTTACTCATATATACTTTAGATTGATTTAAAACTTCATTTTTAATTT
AAAAGGATCTAGGTGAAGATCCTTTTTGATAATCTCATGACCAAAATCCC
TTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAA
ATACTGTCCTTCTAGTGTAGCCGTAGTTAGGCCACCACTTCAAGAACTCT
GTAGCACCGCCTACATACCTCGCTCTGCTAATCCTGTTACCAGTGGCTGC
TGCCAGTGGCGATAAGTCGTGTCTTACCGGGTTGGACTCAAGACGATAGT
TACCGGATAAGGCGCAGCGGTCGGGCTGAACGGGGGGTTCGTGCACACAG
CCCAGCTTGGAGCGAACGACCTACACCGAACTGAGATACCTACAGCGTGA
GCTATGAGAAAGCGCCACGCTTCCCGAAGGGAGAAAGGCGGACAGGTAT
CCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGAC
TTGAGCGTCGATTTTTGTGATGCTCGTCAGGGGGGCGGAGCCTATGGAA
AAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGT
ATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAAC
CGCCTCTCCCCGCGCGTTGGCCGATTCATTAATGCAGCTGGCACGACAGG
TTTCCCGACTGGAAAGCGGGCAGTGAGCGCAACGCAATTAATGTGAGTTA
GCTCACTCATTAGGCACCCCAGGCTTTACACTTTATGCTTCCGGCTCGTA
TGTTGTGTGGAATTGTGAGCGGATAACAATTGAATTAAGGAGGATATAAT
TATGAAATACCTGCTGCCGACCGCAGCCGCTGGTCTGCTGCTGCTCGCGG
CCCAGCCGGCCATGGCCGCCGGTGTTGTTATGACCCAGTCTCCGTCTACC
CTGTCTGCTTCTGTTGGTGACACCATCACCATCACCTGCCGTGCTTCTCA
GTCTATCGAAACCTGGCTGGCTTGGTACCAGCAGAAACCGGGTAAAGCTC
CGAAACTGCTGATCTACAAGGCTTCTACCCTGAAAACCGGTGTTCCGTCT
CGTTTCTCTGGTTCTGGTTCTGGTACCGAGTTCACCCTGACCATCTCTGG
TCTGCAGTTCGACGACTTCGCTACCTACCACTGCCAGCACTACGCTGGTT
ACTCTGCTACCTTCGGTCAGGGTACCCGTGTTGAAATCAAACGTACCGTT
GCTGCTCCGTCTGTTTTCATCTTCCCGCCGTCTGACGAACAGCTGAAATC
TGGTACCGCTTCTGTTGTTTGCCTGCTGAACAACTTCTACCCGCGTGAAG
CTAAAGTTCAGTGGAAAGTTGACAACGCTCTGCAGTCTGGTAACTCTCAG
GAATCTGTTACCGAACAGGACTCTAAAGACTCTACCTACTCTCTGTCTTC
TACCCTGACCCTGTCTAAAGCTGACTACGAAAAGCACAAAGTTTACGCTT
GCGAAGTTACCCACCAGGGTCTGTCTTCTCCGGTTACCAAATCTTTCAAC
CGTGGTGAATGCTAATTAATTAATAAGGAGGATATAATTATGAAAAAGA
CAGCTATCGCGATTGCAGTGGCACTGGCTGGTTTCGCTACCGTAGCCCAG
GCGGCCGCAGAAGTTCAGCTGGTTGAATCTGGTGGTGGTCTGGTTAAA
GCTGGTGGTTCTCTGATCCTGTCTTGCGGTGTTTCTAACTTCCGTATCT
CTGCTCACACCATGAACTGGGTTCGTCGTGTTCCGGGTGGTGGTCTGGA
ATGGGTTGCTTCTATCTCTACCTCTTCTACCTACCGTGACTACGCTGAC
GCTGTTAAAGGTCGTTTCACCGTTTCTCGTGACGACCTGGAAGACTTCG
TTTACCTGCAGATGCATAAAATGCGTGTTGAAGACACCGCTATCTACTA
CTGCGCTCGTAAAGGTTCTGACCGTCTGTCTGACAACGACCCGTTCGA
CGCTTGGGGTCCGGGTACCGTTGTTACCGTTTCTCCGGCGTCGACCAA
AGGTCCGTCTGTTTTCCCGCTGGCTCCGTCTTCTAAATCTACCTCTGGT
GGTACCGCTGCTCTGGGTTGCCTGGTTAAAGACTACTTCCCGGAACCG
GTTACCGTTTCTTGGAACTCTGGTGCTCTGACCTCTGGTGTTCACACCT
TCCCGGCTGTTCTGCAGTCTTCTGGTCTGTACTCTCTGTCTTCTGTTGT
TACCGTTCCGTCTTCTTCTCTGGGTACCCAGACCTACATCTGCAACGTT
AACCACAAACCGTCTAACACCAAAGTTGACAAGAAAGTTGAACCGAAAT
CTTGCCTGCGATCGCGGCCAGGCCGGCCGCACCATCACCATCACCATG
GCGCATACCCGTACGACGTTCCGGACTACGCTTCTACTAGTTAGGAGGGT
GGTGGCTCTGAGGGTGGCGGTTCTGAGGGTGGCGGCTCTGAGGGAGGCGG
TTCCGGTGGTGGCTCTGGTTCCGGTGATTTTGATTATGAAAAGATGGCAA
ACGCTAATAAGGGGGCTATGACCGAAAATGCCGATGAAAACGCGCTACAG
TCTGACGCTAAAGGCAAACTTGATTCTGTCGCTACTGATTACGGTGCTGC
TATCGATGGTTTCATTGGTGACGTTTCCGGCCTTGCTAATGGTAATGGTG
CTACTGGTGATTTTGCTGGCTCTAATTCCCAAATGGCTCAAGTCGGTGAC
GGTGATAATTCACCTTTAATGAATAATTTCCGTCAATATTTACCTTCCCT
CCCTCAATCGGTTGAATGTCGCCCTTTTGTCTTTGGCGCTGGTAAACCAT
ATGAATTTTCTATTGATTGTGACAAAATAAACTTATTCCGTGGTGTCTTT
GCGTTTCTTTTATATGTTGCCACCTTTATGTATGTATTTTCTACGTTTGC
TAACATACTGCGTAATAAGGAGTCTTAAGCTAGCTAACGATCGCCCTTCC
CAACAGTTGCGCAGCCTGAATGGCGAATGGGACGGGCCCTGTAGCGGCGC
ATTAAGCGCGGCGGGTGTGGTGGTTACGCGCAGCGTGACCGCTACACTTG
CCAGCGCCCTAGCGCCCGCTCCTTTCGCTTTCTTCCCTTCCTTTCTCGCC
ACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTA
GGGTGATGGTTCACGTAGTGGGCCATCGCCCTGATAGACGGTTTTTCGCC
CTTTGACGTTGGAGTCCACGTTCTTTAATAGTGGACTCTTGTTCCAAACT
GGAACAACACTCAACCCTATCTCGGTCTATTCTTTTGATTTATAAGGGAT
TTTGCCGATTTCGGCCTATTGGTTAAAAAATGAGCTGATTTAACAAAAAT
TTAACGCGAATTTTAACAAAATATTAACGCTTACAATTTAG).

In the vector sequence set forth above, the sequence of the nucleic acid encoding the light chain domain (SEQ ID NO: 64) is set forth in italics, and the sequence of the nucleic acid encoding the heavy chain domain (V_{H} and C_{H}1) (SEQ ID NO: 65) is set forth in bold. The 2G12 heavy and light chains encoded by these nucleic acids contained the sequences of amino acids set forth in SEQ ID NOS: 73 and 74, respectively.

(ii) 2G12 pCAL A1

An process identical to that used in section (i), above, was used to introduce the 2G12 sequence into the pCAL A1 vector (SEQ ID NO: 14) (also described in Example 2A, above), to produce a 2G12 pCAL A1 vector, having the nucleotide sequence set forth in SEQ ID NO: 34.

(iii) 3-Ala pCAL G13

A 3-Ala 2G12 pCAL G13 (3-Ala pCAL G13) vector (SEQ ID NO: 33) also was produced. This vector was identical to the 2G12 pCAL G13 vector, with the exception that the heavy chain domain in the vector contained three Alanine substitutions. The light chain domain in this vector was identical to the 2G12 light chain domain. To produce the vector (3-Ala pCAL G13) containing the sequence encoding the 3-Ala 2G12 mutant polypeptide, two sets of PCR amplifications were carried out, using the 2G12 pCAL G13 vector (SEQ ID NO: 32) as a template.

For the first reaction, pCALVH-F primer was used with another reverse primer (3Ala-R: TCGAACGGGTCCGCGTCCGCCGCACGGTCAGAACCTTTAC; SEQ ID NO: 75), and for the second reaction, the pCALCH-R primer was used with another forward primer (3Ala-F: GTTCTGACCGTGCGGCGGACGCGGACCCGTTCGACGCTTG; SEQ ID NO: 76). The products from these two reactions were gel-purified and an overlap PCR was performed with primer A (GCCCAGGCGGCCGCAGAAGTTCAG; SEQ ID NO: 77) and primer E (CCTTTGGTCGACGCCGGAGAAACGGTAACAACGGTACCCGGACCCCAAG CGTCGAACG; SEQ ID NO: 78). The product from the overlap PCR then was gel-purified and digested with Not I/Sal I and cloned back into 2G12 pCAL in the same restriction sites.

### Example 2C

### Generation of Vector for Display of Domain Exchanged Antibodies with Increased Stability/Reduced Toxicity: 2G12 pCAL IT* Vector

To reduce the toxicity of the domain exchanged Fab fragments expressed from the vectors, and thereby increase stability of the phagemids displaying the Fab fragments, the 2G12 pCAL IT* vector was generated, in which an additional amber stop codon (TAG) was introduced into each of the leader sequences upstream of the polynucleotides encoding the heavy and light chain fragments (see FIG. 9). This phagemid vector was made by modifying a 2G12 pCAL ITPO vector, which was derived from the 2G12 pCAL vector (as described below).

This vector can be used for repressed expression of the 2G12 Fab fragments in non-supE44 amber suppresser strains (such as, for example, NEB 10-beta cells and TOP10F′ cells), and modest expression in supE44 cells (e.g. XL1-Blue cells), for reduced expression and thus reduced toxicity of domain exchanged Fab fragments in amber-suppressor strains such as XL1-Blue.

(i). Generation of the 2G12 pCAL ITPO Vector

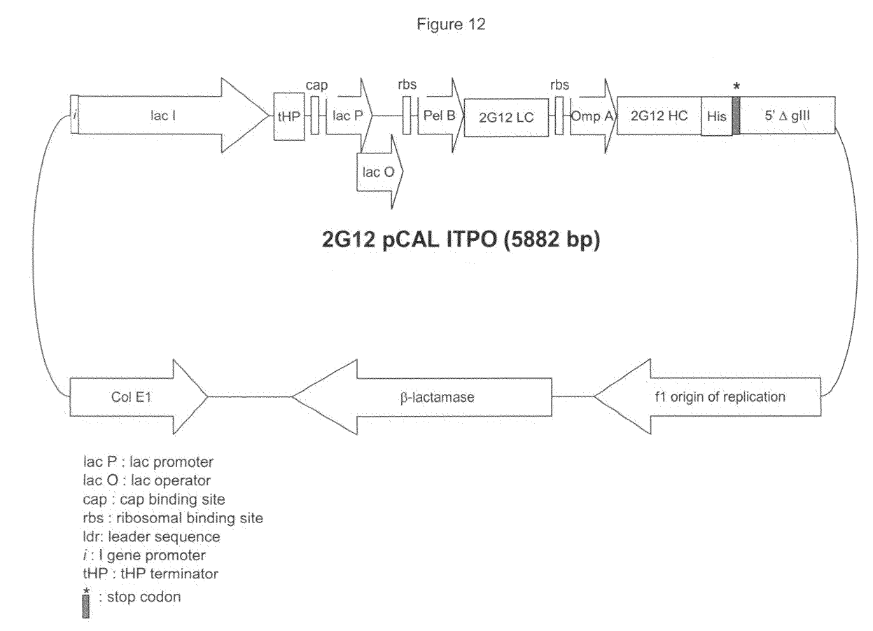

The 2G12 pCAL G13 vector (FIG. 8), having a nucleic acid sequence set forth in SEQ ID NO: 32, first was modified by replacement of the 5′-truncated lac I gene with the lac I gene promoter (i) and the entire lac I gene, tHP terminator, and lac promoter/operon gene to create the 2G12 pCAL ITPO vector (FIG. 12), having a nucleic acid sequence set forth in SEQ ID NO: 36.

Briefly, the lac I gene promoter and lac I gene were amplified using 10 ng of pET28a(+) AC8 scFv (SEQ ID NO: 79) as template DNA with 0.4 μM each of a LacITerm-F1 primer (SEQ ID NO: 80) and a LacITerm-R1 primer (SEQ ID NO: 81), 1 μL of Advantage® HF2 Polymerase Mix (Clontech) in 1× reaction buffer and dNTP mix in a 50 μL reaction volume. This amplification reaction was labeled PCR 1a.

The tHP terminator gene was amplified using 0.2 pmol of Term-R oligonucleotide (SEQ ID NO: 82) as a template with 0.4 μM of the LacITemr-F2 primer (SEQ ID NO: 83) and the TermPO-R primer (SEQ ID NO: 84) in the presence of 1 μL of Advantage® HF2 Polymerase Mix and its reaction buffer and dNTP mix in a 50 μL reaction volume. The amplification reaction was labeled PCR 1b.

The Lac promoter and operon gene was amplified using 10 ng of the 3Ala mutant of 2G12 in the pCAL G13 vector (SEQ ID NO: 33) as a template with 0.4 μM of the TermPO-F primer (SEQ ID NO: 85) and the SgrAIPelB-R primer (SEQ ID NO: 86) in the presence of 1 μL of Advantage® HF2 Polymerase Mix and its reaction buffer and dNTP mix in a 50 μL reaction volume (PCR 1c).

Each of the PCR amplifications (PCR 1a-c) included a denaturation step at 95° C. for 1 min followed by 30 cycles of denaturation at 95° C. for 5 seconds and annealing/extension at 68° C. for 1 min, and finished with incubation at 68° C. for 3 min.

The amplified products from the PCR 1a amplification (1195 base pairs (bp)) and the PCR 1c amplification (219 bp) were run on a 1% agarose gel and purified with a Gel Extraction Kit (Qiagen). The amplified product from the PCR 1b amplification was purified on a PCR purification column.

Two overlap PCR amplifications were then performed to join each of the products from the PCR 1a, b and c reactions. The first overlap amplification was performed by mixing 5 μL of PCR 1a and PCR 1b with 0.4 μM of LacITerm-F1 primer in the presence of 2 μL of Advantage® HF2 Polymerase Mix and its reaction buffer and dNTP mix in a 100 μL reaction volume. The second overlap amplification was performed by mixing 5 μL of PCR 1b and PCR 1c with 0.4 μM of SgrAIPelB-R primer in the presence of 2 μL of Advantage® HF2 Polymerase Mix and its reaction buffer and dNTP mix in a 100 μL reaction volume. Each of these reactions were performed using an initial denaturation step at 95° C. for 1 min, followed by 5 cycles of denaturation at 95° C. for 5 seconds and annealing/extension at 68° C. for 1 min. The two overlap reactions were then mixed in a third reaction with an initial denaturation step at 95° C. for 20 seconds, then 30 cycles of 95° C. for 5 seconds and annealing/extension at 68° C. for 1 min and 20 seconds, followed by a final extension step for 3 min incubation at 68° C.

The resulting amplified product (1443 bp) was run on a 1% agarose gel and purified with Gel Extraction Kit (Qiagen). The purified product was digested with Sap I/SgrA I and purified using PCR purification column. The 2G12 pCAL vector similarly was digested with Sap I/SgrA I to release the 5′-truncated lac I gene, and the vector DNA was gel purified using Gel Extraction Kit (Qiagen). The digested amplification product then was ligated into the vector DNA using T4 DNA ligase (Invitrogen) to produce the 2G12 pCAL ITPO vector (FIG. 12 and SEQ ID NO: 36) and transformed in XL1-Blue cells. Plasmid DNA was prepared by first inoculating colonies from the titration plates into 1.2 mL SuperBroth medium containing 50 μg/mL carbenicillin and 20 mM glucose. The culture plate was incubated overnight at 37° C. (shaken at 300 rpm). The DNA sequence of the resulting 2G12 pCAL ITPO vector (SEQ ID NO:36) was confirmed using the following primers: SeqCALTerm-F (SEQ ID NO: 87), SeqpCALTerm-R (SEQ ID NO: 88), SeqpCALIT-R (SEQ ID NO: 89) and SeqITPO-F2 (SEQ ID NO: 90).

(ii). Generation of the 2G12 pCAL IT* Vector

To generate the 2G12 pCAL IT* vector, the 2G12 pCAL ITPO vector was modified by introducing amber stop codons (TAG) at the 3′ end of the Pel B and Omp A bacterial leader sequences. The TAG amber stop codons were introduced to replace the wild-type CAG codon for glutamine.

Two PCR amplifications were performed using 10 ng 2G12 pCAL IPTO (SEQ ID NO: 36) as a template DNA, with either 400 nM of Kas I-F and AmbPelB-R primers (SEQ ID NOS: 91 and 92, respectively) or 400 nM of AmbPelB-F and AmbOmpA-R primers (SEQ ID NOS: 93 and 94, respectively), in the presence of 1 μL of Advantage® HF2 Polymerase Mix and its reaction buffer and dNTP mix in a 50 μL reaction volume. The PCR reactions were performed with an initial denaturation step at 95° C. for 1 min, followed by 30 cycles of denaturation at 95° C. for 5 seconds, annealing at 64° C. for 10 seconds, and extension at 68° C. for 1 min, followed by a final incubation at 68° C. for 3 min. The resulting amplified products (360 by and 777 bp, respectively) were run on a 1% agarose gel and purified with Gel Extraction Kit (Qiagen).

An overlap PCR amplification was performed using 4 μL of the gel-purified PCR fragments as template, with 400 nM of Kas I-F and AmbOmpA-R primers, in the presence of 4 μL of Advantage® HF2 Polymerase Mix, Advantage® HF2 reaction buffer, and dNTP mix, in a 200 μL reaction volume. The PCR reaction was performed with an initial denaturation step at 95° C. for 1 min, followed by 30 cycles of denaturation at 95° C. for 5 seconds and annealing/extension at 68° C. for 1 min, followed by a final incubation at 68° C. for 3 min. The resulting 1106 by amplified product was run on a 1% agarose gel and purified with Gel Extraction Kit (Qiagen).

Both the 2G12 pCAL ITPO vector and the purified PCR product were digested with Kas I/Not I. The vector DNA was run on a 0.7% agarose gel and the 4809 by fragment was purified with Gel Extraction Kit (Qiagen). The digested 1084 by PCR fragment was purified on a PCR purification column. The vector DNA and PCR product were ligated using 100 ng of vector DNA and 56 ng of PCR fragment with 1 μL of T4 DNA ligase (Invitrogen) and its reaction buffer in a 20 μL reaction volume at room temperature (˜25° C.) for 2 hrs or more. The ligated DNA was transformed into XL1-Blue cells (Stratagene) and spread onto LB agar plates with 100 μg/mL of carbenicillin and 20 mM glucose. 16 colonies from the plates were used to inoculate cultures of 1.2 mL SuperBroth medium containing 50 μg/mL carbenicillin and 20 mM glucose. The cultures were then incubated overnight at 37° C. (shaken at 300 rpm).

Plasmid DNA was purified using miniprep DNA columns (Qiagen) and DNA sequence of the resulting 2G12 pCAL IT* vector (FIG. 9) was confirmed using the following primers: SeqHCFR1-R (SEQ ID NO: 95), SeqpCAL-F (SEQ ID NO: 96), SeITPO-F2 (SEQ ID NO:90), and SeqITPO-F4 (SEQ ID NO: 97).

### Example 3

### Amplification of 2G12 and 3-Ala 2G12 Nucleic Acids in Host Cells and Expression of Domain Exchanged Fab Fragment-Gene III Fusion Proteins

To amplify nucleic acids and demonstrate that the vectors in Example 2B could be used to express domain exchanged Fab fragments, a partial amber suppressor bacterial host cell line (XL1-Blue) was transformed with the vectors. The vectors generated in Example 2A, above (pCAL A1 and pCAL G13), without inserts, also were transformed into the cells, for use as negative controls in subsequent assays.

1 μg (2 μL) of vector (e.g. 2G12 pCAL G13; 2G12 pCAL A1; 3-Ala pCAL G13; 3-Ala pCAL A1; pCAL A1 and pCAL G13) DNA was electroporated into 100 μL of electrocompetent XL1-Blue cells (Stratagene) at 1700 kV/0.1 cm (BioRad). The cells were resuspend in 3 mL SOC medium (Invitrogen™ Corporation). The mixture was incubated at 37° C. for 1 hour, with shaking at 250 rpm. 7 mL SB medium (30 g tryptone, 20 g yeast extract, 10 g MOPS in a 1 L volume in distilled water) was added to the culture, along with carbenicillin (at 20 μg/mL) and tetracycline (at 12.5 μg/mL).

To generate colonies, 0.01 μL and 0.001 μL aliquots of the mixture then were spread on LB agar plates, supplemented with 100 μg/mL of carbenicillin and 20 mM of glucose. The plates were incubated overnight at 37° C. Number of colonies was determined to evaluate transformation efficiency by multiplying the number of colonies by the culture volume and dividing by the plating volume (same units), using the following equation: [# colonies/plating volume×[culture volume)/microgram DNA]×dilution factor. For cells transformed with 2G12 pCAL A1 vector DNA, the efficiency was 9×10⁷ (cfu/microgram), for cells transformed with 2G12 pCAL G13, the efficiency was 1.6×10⁸ cfu/microgram, and for cells transformed with pCAL G13 empty vector, the efficiency was 7.1×10⁸ cfu/μg.

### Example 4

### Phage Display of Functional Domain Exchanged Antibodies

The study described in this example was carried out to demonstrate that XL1-Blue cells (which are phage display compatible) containing the domain exchanged antibody-encoding vectors could display domain exchanged antibodies on phage.

### Example 4A

### Inducing Production of Phage Expressing 2G12 Fab Fragments

After removal of aliquots for spreading on agar plates (Example 3), the remainder of the XL1-Blue cultures were incubated for 1 hour at 37° C., with shaking at 250 rpm, and added to 40 mL SB medium. Prior to the incubation, the concentration of carbenicillin was adjusted to 50 μg/mL and the concentration of tetracycline was adjusted to 12.5 μg/mL.

To induce phage production, 5×10¹¹ pfu of VCS M13 helper phage (Stratagene) then was added to the culture, which then was incubated for 2 hours at 37° C., with shaking at 250 rpm. Kanamycin was added, to a concentration of 70 μg/mL, and isopropyl-beta-D-thiogalactopyranoside (IPTG) (Acros Chemicals) was added, to a concentration of 1 mM, and the culture was incubated overnight at 30° C., with shaking at 250 rpm.

### Example 4B

### Phage Precipitation

The culture then was centrifuged at 4000 rpm for 15 min (4° C.). 32 mL of supernatant then was added to 8 mL of 20% polyethylene glycol 8000 (PEG8000; Sigma Catalog No. P5413) in 2.5 M NaCl solution (for a final concentration of 4% PEG8000, 0.5 M NaCl), while inverting, to mix thoroughly. This mixture was incubated on ice for 30 min to precipitate the phage.

To clear the phage, the mixture then was centrifuged at 12000×g for 30 minutes at 4° C. The supernatant was aspirated and the pellet was briefly dried (5 minutes). The precipitated phage then were resuspended in 2 mL phosphate buffered saline (PBS) containing 1% bovine serum albumin (BSA), and transferred to microcentrifuge tubes. The tubes were centrifuged at 14000 rpm for 5 min at 4° C. The resulting cleared phage suspensions were transferred to new microcentrifuge tubes.

### Example 4C

### Antigen Binding of Precipitated Phage

To demonstrate that the vectors and methods displayed functional domain exchanged antibodies, a binding assay was carried out on the cleared phage (phage transformed with 2G12 pCAL G13; 2G12 pCAL A1; empty pCAL G13; and empty pCAL A1) from Example 4B. For this process, 50 microliters of gp120 antigen (Strain JR-FL, Immune Technologies) diluted in PBS pH 7.4, was added to coat individual wells of a 96-well microtiter plate (Corning Costar, Catalog No. 3690, using a 50 microliter volume per well. Some wells were coated with ovalbumin (2 microgram per mL, 100 ng per well), as a control.

In each case, the antigen was coated onto the plate overnight, at 4° C. The coated plate then was washed 5 times with PBS/0.05% Tween-20. The plate then was blocked, using 135 microliters per well of 4% nonfat dry milk diluted in PBS, for one hour at 37° C. The block was discarded and the plate dried by tapping on paper towels.

A two-fold serial dilution was carried out by diluting the cleared phage from the previous step (dilutions carried out in 1% BSA in PBS), to generate the following dilutions of the phage: non-diluted; 1:2, 1:4, 1:8, 1:16, 1:32, 1:64, 1:128. Then, fifty microliters of each dilution was added to one of the wells of the coated and washed microtiter plate, which was incubated at 37° C. for 2 hours, with rocking.

The plate then was washed 5 times with PBS/0.5% Tween-20 (polysorbate 20). To detect phage displaying domain exchanged fragments that had specifically bound to the antigen coated on the plate, two separate enzyme linked immunosorbent assay (ELISA) reaction was carried out, detecting bound phage with either anti-HA antibody or anti-M13 (phage) antibody.

For this process, the wells were incubated with 50 μL of HRP-conjugated anti-HA (3F10) (1:1000)(Roche) or HRP-conjugated rabbit anti-M13 antibody (1:1000) in 1% BSA/PBS at 37° C. for 1 hr. The plates were washed 5 times, with PBS/0.05% Tween 20. The wells that contained anti-HA antibody were developed with 50 μL of TMB substrate kit (Pierce) and stopped with 50 μL of H₂SO₄. The plates were read at 450 nm. The wells that contained rabbit anti-M13 antibody were incubated with 50 μL of HRP-conjugated goat anti-rabbit IgG (H+L) (minimum cross-reactivity with human serum proteins) (Pierce) at 37° C. for 1 hr. The plates were washed 5 times, with PBS/0.05% Tween 20. The wells were developed with 50 μL of TMB substrate kit (Pierce) and stopped with 50 μL of H₂SO₄. The plates were read at 450 nm.

The results indicated that phage precipitated from the cells transformed with the 2G12 pCAL G13 and the 2G12 pCAL A1 vectors specifically bound, in a concentration-dependent manner, to the wells coated with gp120, but not the control wells, coated with ovalbumin. No specific binding was observed with empty vectors (pCAL G 13 and pCAL A1), with either antigen. These data confirmed that the provided methods can be used to display a functional fragment of a domain-exchange antibody (2G12) fragment on the surface of phage, and that the provided methods will be useful in phage display of domain-exchange antibody fragments, for example, in phage display libraries.

### Example 5

### Generation of a Nucleic Acid Library for Display of a Collection of Domain Exchanged Fab Fragments

To generate phage display libraries for selection of phage displayed domain exchanged antibodies, a nucleic acid library was generated by randomizing nucleotides encoding seven amino acids in the CDR 1 and CDR 3 regions of the 2G12 heavy chain. For this process, modified Fragment Assembly and Ligation/Single Primer Amplification (mFAL-SPA) (as described in U.S. application No. [Attorney Docket No. 3800013-00031/1106] and International Application No. [Attorney Docket No. 3800013-00032/1106PC]), was used to generate a collection of duplex cassettes containing randomized nucleic acids, with randomized positions within the 2G12 heavy chain-encoding nucleic acid. As described in subsections of this example, below, for the vectors described in Example 2B (2G12 pCAL) and Example 2C (2G12 pCAL IT*), nucleic acids encoding the wild-type 2G12 heavy chains were replaced with this collection of randomized cassettes, generating a nucleic acid library based on each vector. These libraries were used in “spike-in” experiments described in Examples below.

### Example 5A

### Randomization of CDRs 1 and 3 by Modified Fragment Assembly and Ligation/Single Primer Amplification (mFAL-SPA)

Modified Fragment Assembly and Ligation (mFAL-SPA), as described in U.S. application No. [Attorney Docket No. 3800013-00031/1106] and International Application No. [Attorney Docket No. 3800013-00032/1106PC], was used to generate nucleic acid libraries that could be used to make display libraries containing variant polypeptides with diversity in portions of the CDR1 and CDR3 of the heavy chain variable region of a 2G12 domain exchanged Fab target polypeptide. The 2G12 domain exchanged fab target polypeptide, which was randomized to create this diversity, contained a heavy chain having the amino acid sequence set forth in SEQ ID NO: 73, and a light chain having the amino acid sequence set forth in SEQ ID NO.: 74.

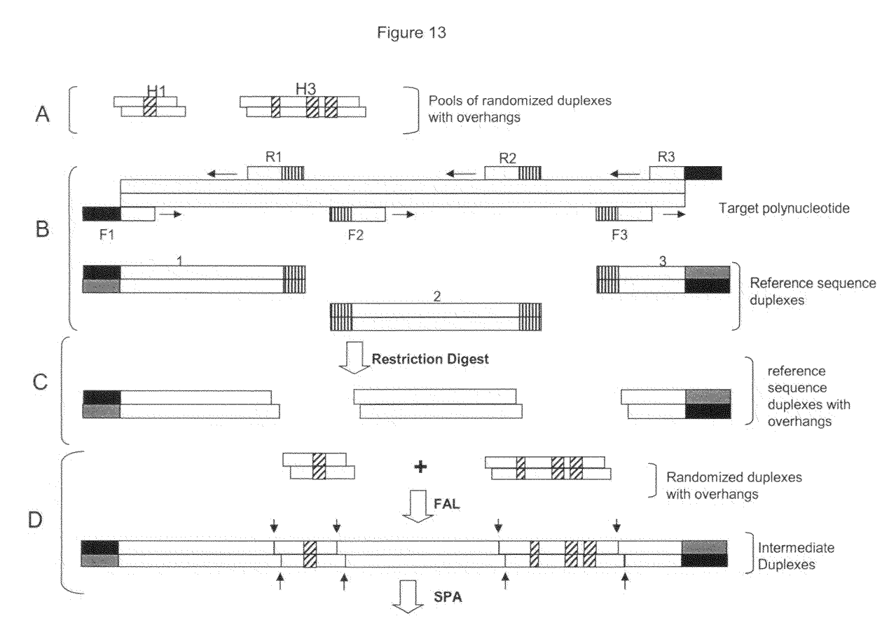

As illustrated schematically in FIG. 13, the mFAL-SPA process was used to diversify 7 amino acid positions in the 2G12 Fab by randomization of the 2G12 Heavy Chain CDR1 and CDR3, as follows.

(i) Generating Pools of Randomized Duplexes

Four pools of randomized oligonucleotides (H1F, H1R, H3F, and H3R) were designed and generated for use in forming two pools of randomized duplexes (H1 and H3; illustrated in FIG. 13A). The sequences of these randomized oligonucleotides are set forth in Table 6, below. Each oligonucleotide in each of these randomized pools was synthesized based on a reference sequence (which contained part of the native 2G12 heavy chain nucleotide sequence), but contained randomized portions, represented in bold type in Table 6 and as hatched boxes in FIG. 13. These randomized portions were synthesized using the NNK or NNT doping strategy. An NNK doping strategy minimizes the frequency of stop codons and ensures that each amino acid position encoded by a codon in the randomized portion could be occupied by any of the 20 amino acids. With this doping strategy, nucleotides were incorporated using an NKK pattern and a MNN pattern, during synthesis of the positive and negative strand randomized portions respectively, where N represents any nucleotide, K represents T or G and M represents A or C. An NNT strategy eliminates stop codons and the frequency of each amino acid is less biased but omits Q, E, K, M, and W.

The reference sequence used to design each pool of randomized oligonucleotides is listed in Table 6, below the sequence of the randomized oligonucleotide. The randomized portions also contained variant positions, where the nucleotide at the variant position was mutated compared to the reference sequence portion. These positions also are indicated in bold and are part of the randomized portions.

The randomized oligonucleotides were designed such that each oligonucleotide in each of the pools contained a region complementary to an oligonucleotide in another pool. Oligonucleotides in pool H1F were complementary to oligonucleotides in pool H1R, and oligonucleotides in pool H3F were complementary to oligonucleotides in pool H3R. The oligonucleotides in each pool further were designed, whereby, following hybridization of the pairs of oligonucleotides through these complementary regions, three nucleotide 5′-end overhangs would be generated, to facilitate ligation in subsequent steps (for example, see FIG. 13A). The nucleotides that would become the overhangs are indicated in italics in Table 6. The nucleotides in the randomized pools were labeled with 5′ phosphate groups.

In order to form the H1 duplex, 50 μL H1F (at 100 μM), 50 μL H1R (100 μM) and 1 μL NaCl were mixed, denatured at 95 C for 5 minutes, followed by slow cooling to 25° C. on a heat block covered with a Styrofoam® box. Similarly, to form the H3 duplex, 50 μL H3F (at 100 μM), 50 μL H1R (100 μM) and 1 μL NaCl were mixed, denatured at 95° C. for 5 minutes, followed by slow cooling to 25° C. on a heat block covered with a Styrofoam® box.

TABLE 6

| | | |
|---|---|---|
| Name | Sequence | SEQ ID NO: |

| | | |
|---|---|---|
| F1 | GCCGCTGTGCCATCGCTCAGTAACgcggccgcagaa | 98 |
| | gttcagctg | |
| R1 | GGCGGCGCTCTTCagttagaaacaccgcaagacaggatc | 99 |
| F2 | GGCGGCGCTCTTCtcgtgttccgggtggtggtctg | 100 |
| R2 | GGCGGCGCTCTTCagtagatagcggtgtcttcaacac | 101 |
| F3 | GGCGGCGCTCTTCgggtccgggtaccgttgttac | 102 |
| R3 | GCCGCTGTGCCATCGCTCAGTAACgtcgacgccgga | 103 |
| | gaaacggt | |
| H1F | AACTTCCGTATCTCTGCTNNTNNKATGAACTG | 104 |
| | GGTTCGT | |
| Reference | AACTTCCGTATCTCTGCTCACACCATGAACTG | 105 |
| sequence | GGTTCGT | |
| used to | | |
| design H1F | | |
| H1R | ACGACGAACCGAGTTCATMNNANNAGCAGAG | 106 |
| | ATACGGAA | |
| Reference | ACGACGAACCCAGTTCATGGTGTGAGCAGAG | 107 |
| sequence | ATACGGAA | |
| used to | | |
| design H1R | | |
| H3F | TACTACTGCGCTCGTAAANNKTCTGACCGTNN | 108 |
| | TNNKGACNNKNNKCCGTTCGACGCTTGG | |
| Reference | TACTACTGCGCTCGTAAAGGTTCTGACCGTCT | 109 |
| sequence | GTCTGACAACGACCCGTTCGACGCTTGG | |
| used to | | |
| design H3F | | |
| H3R | ACCCCAAGCGTCGAACGGMNNMNNGTCMNN | 110 |
| | ANNACGGTCAGAMNNTTTACGAGCGCAGTA | |
| Reference | ACCCCAAGCGTCGAACGGGTCGTTGTCAGAC | 111 |
| sequence | AGACGGTCAGAACCTTTACGAGCGCAGTA | |
| used to | | |
| design H3R | | |

ii. Generation of Reference Sequence Duplexes

PCR amplification was carried out to generate three reference sequence duplexes (1, 2, and 3, as illustrated in FIG. 13B). Duplexes in pool 1 were 125 nucleotides in length, duplexes in pool 2 were 196 nucleotides in length and duplexes in pool 3 were 76 nucleotides in length. For this process, three pools of forward oligonucleotide primers (F1, F2, F3) and three pools of reverse oligonucleotide primers (R1, R2, R3) were synthesized using the methods provided herein. The sequences of the primers in each pool are set forth in Table 6, above.

Each of the primers used to generate the reference sequence duplexes contained a 5′ sequence of nucleotides corresponding to a restriction endonuclease cleavage site. Four of the primers, R1, F2, R2 and F3, contained the sequence of nucleotides set forth in SEQ ID NO: 44 (GCTCTTC), which is the recognition site for the Sap I restriction endonuclease (within the grey portions in FIG. 13B). This enzyme cuts duplex polynucleotides to leave a 3-nucleotide overhang of any sequence at its 5′ end, beginning at one nucleotide in the 3′ direction from this recognition sequence. The restriction endonuclease recognition site is indicated in italics in Table 6, above, while the three-nucleotide overhang in each primer pool is indicated in bold. The oligonucleotides were designed such that the potential three nucleotide overhang of each primer pool was complementary to one of the three nucleotide overhangs generated in the randomized duplexes. The oligonucleotides were designed in this manner to facilitate ligation in a subsequent step.

Primers in the F1 pool contained a sequence of nucleotides corresponding to a Not I restriction endonuclease recognition site. Primers in the R3 pool contained a sequence of nucleotides corresponding to a Sal I restriction endonuclease site (the Sal I and Not I restriction sites are within the black portions in FIG. 13). These restriction endonuclease recognition sites facilitated ligation of the assembled duplexes into vectors in subsequent steps.

Further, one forward primer pool (F1), and one reverse primer pool (R3), contained a Region X (depicted in black in FIG. 13: identical in sequence within both primers), a non gene-specific sequence of nucleotides that is identical to the CALX24 primer (SEQ ID NO: 112) at the 5′ ends of the primers. Thus, the reference sequence duplexes 1 and 3, made with these primers/oligonucleotides, contained a sequence of nucleotides including Region X, and also a complementary Region Y. These regions served as templates for the primer CALX24, which was used in the subsequent single primer amplification (SPA) step, described below.

To form duplexes using these primers, the 2G12 pCAL vector containing the 2G12 target polynucleotide (SEQ ID NO: 33) was used as a template in three separate PCR amplifications. For these reactions, primer pair pools, F1/R1, F2/R2, and F3/R3, were used to amplify duplex pool 1, duplex pool 2, and duplex pool 3. For each reaction, 40 picomoles (pmol) of each primer of each primer, 20 nanograms (ng) of the vector template were incubated in the presence of 2 μl Advantage HF2 Polymerase Mix (Clonetech) and the corresponding 1× reaction buffer, and 1×dNTP in a 100 μL reaction volume. The PCR was carried out using the following reaction conditions: 1 minute denaturation at 95° C. followed by 30 cycles of 5 seconds of denaturation at 95° C., 10 seconds of annealing at 60° C., and 20 seconds of extension at 68° C., then 1 minute incubation at 68° C. The amplified fragments were gel-purified using a Gel Extraction Kit (Qiagen).

After amplification by PCR, 1.6-2 μg of each pool of reference sequence duplexes (1, 2 and 3) was digested, as illustrated in FIG. 13C, with 250 Units/mL Sap I (New England Biolabs, R0569M 10,000 Units/mL). The digested duplexes then were purified using a PCR purification column (Qiagen). The resulting digested duplexes were 108, 165 and 62 nucleobase pairs in length, respectively.

iii. Ligation of Digested Reference Sequence Duplexes and Randomized Duplexes to Form Intermediate Duplexes

As illustrated in FIG. 13D, the digested reference sequence duplexes and the randomized duplexes were hybridized and ligated to form intermediate duplexes. This process was carried out as follows. First, H1 and H3 pools were mixed at equimolar ((108 ng of 108 by duplexes, 39 ng of H1, 165 ng of 165 by duplexes, 60 ng of H3, and 62 ng of 62 by duplexes) in T4 DNA ligase buffer and ligated with 10 units of T4 DNA ligase, at room temperature (˜25° C.) overnight.

iv. Formation of Duplex Cassettes

Following the formation of the intermediate duplexes, a single primer amplification (SPA) reaction was used to generate amplified randomized assembled duplexes. Amplification was carried out using 50 μL of the intermediate duplexes and 1.2 μM CALX24 primer, in the presence of 50 μL Advantage HF2 Polymerase Mix and the corresponding 1× reaction buffer and 1×dNTP in a 2.5 mL reaction volume, using the same heating/cooling reaction conditions. The resulting collection of amplified assembled duplexes was column purified and gel purified. The assembled duplexes were 434 nucleotides in length. This process produced 60.8 μg of the assembled duplexes. The assembled duplexes were then digested with Sal I and Not I, to form assembled duplex cassettes, which could be ligated into vectors to form nucleic acid libraries.

### Example 5B

### Formation of 2G12 Nucleic Acid Libraries

Both the 2G12 pCAL IT* vector (SEQ ID NO: 35) and the 2G12 pCAL vector (SEQ ID NO: 32) were digested with Sal I and Not I. The DNA was run on a 0.7% agarose gel. The linearized pCAL IT* and pCAL vectors (without the original wild-type 2G12 insertions) were then purified using the Gel Extraction Kit (Qiagen). Each vector was ligated with the assembled duplex cassettes described above, to generate two libraries, each containing randomized 2G12 Fab encoding nucleic acid members. The two libraries contained the nucleic acids in the pCAL IT* vector and the pCAL vector, respectively.

### Example 6

### Antigen-Specific Selection of Phage Displaying Domain Exchanged Antibody

To demonstrate that the provided methods for phage display of domain exchanged antibodies can be used to select antigen-specific domain exchanged antibody fragments, panning studies were performed using the 2G12 pCAL G13 (SEQ ID NO: 32) and 3-ALA pCAL G13 (SEQ ID NO: 33) vectors described in Example 2B, above. In these studies, the gp120 antigen was used to select from among mixtures of phage-displayed domain exchanged antibodies encoded by these vectors. For example, as described in the subsections below, varying concentrations of a vector encoding the domain exchanged Fab fragment specific for the gp120 antigen (2G12 pCAL G13 (SEQ ID NO: 32), described in Example 2B) were spiked into a quantity of vector encoding a non-antigen specific domain exchanged Fab fragment (3-ALA pCAL G13 (SEQ ID NO: 33), described in Example 2B); the mixtures were used to transform cells for phage display and selection by multiple rounds of panning, to assess enrichment for the antigen-specific domain exchanged antibody fragment.

### Example 6A

### Transformation of Partial Amber Suppressor Host Cells with Vectors Encoding Domain Exchanged Fab Antibody Fragments

First, 1 microgram each of various phage display vector samples was used to transform host cells. One of the samples contained the 2G12 pCAL G13 vector alone (2G12 alone). Another contained the 3-ALA 2G12 pCAL G12 vector alone (3-ALA alone). Other samples contained mixtures of vectors, which were generated by adding (spiking in) 2G12 pCAL G13 vector to a sample containing 3-ALA pCAL G13 vector at four different dilutions, as follows: 10⁻³, 10⁻⁴, 10⁻⁵ and 10⁻⁶ micrograms of the 2G12 pCAL G13 were spiked, separately, into 1 microgram of 3-ALA pCAL G13 vector. 1 microgram of each diluted vector sample (2G12 alone, 3-ALA alone and each “spiked in” mixture) then was used to transform XL1-Blue MRF E. coli cells (Stratagene, La Jolla, Calif.) by electroporation. Cells then were incubated for one hour at 37° C., with shaking at 250 rpm, and the cultures supplemented with 50 μg/mL carbenicillin and 10 μg/mL tetracycline. The cells in culture then were infected with 10¹² VCSM13 helper phage (Stratagene) for an additional 4 hours, at 30° C.

### Example 6B

### Phage Precipitation

To precipitate phage particles, cells from each of the cultures described in Example 6A were centrifuged at 4000 rpm for 30 minutes, and 32 mL of the supernatant mixed with 8 mL of a 2.5 M sodium chloride (NaCl) solution containing 20% polyethylene glycol (Sigma #P5413-500 g). Each sample then was inverted ten times and incubated on ice for thirty minutes. The resulting samples, which contained precipitated phage, then were centrifuged at 13,000 rpm for twenty minutes at 4° C. The pellet containing the precipitated phage then was resuspended in 1 mL PBS containing 1% bovine serum albumin (BSA) and centrifuged at 13,500 rpm at 25° C., for 5 minutes. The supernatant of the 2G12 alone and 3-ALA alone samples were used in studies to assess display as described in Example 6C; the mixtures were used in panning (repeated selection and enrichment based on binding to antigen) as described in Example 6D.

### Example 6C

### Assessing Display and Specificity of Antibodies Following Transformation with 2G12 and 3-Ala Vectors

Prior to panning (see example 6D, below), an ELISA-based assay was used to analyze and verify expression and display of domain exchanged antibody produced by cells transformed with the 2G12 vector alone and the 3-ALA vector alone. For this assay, precipitated phage recovered after each vector transformation was captured onto wells of a microtiter plate that previously had been coated overnight at 4° C., with 100 ng/well (in PBS) of either gp120 JR-FL (Immune Technology Corp, New York, N.Y.) (gp120 capture) or anti-human F(ab′)2 MinX antibody (Goat Anti-Human IgG, F(ab′)2 fragment specific (min X Bov, Hrs, Ms Sr Prot) catalog number: 109 006 097) (anti-human capture) or chicken albumin (Sigma-Aldrich) (control). For this process, eleven two-fold dilutions (½; ¼; ⅛; 1/16; 1/32; 1/64; 1/128; 1/256; 1/512; 1/1024; 1/2048) of the precipitated phage were made. Each dilution was added to a coated and blocked well on the plates. The capture (binding of phage to antibody) was carried out for 2 hours at 37° C., with gentle rocking.

To remove unbound phage, the supernatant from each well was discarded and plates were washed with 150 microliters of PBS containing 0.05% Tween 20 (polysorbate 20). After washing, the presence of bound phage was detected using either 1:5000 anti-M13-p8 HRP (GE) (which bound the phage coat protein p8) or 1:1000 anti-HA (GE) (which bound the HA tag on the displayed antibody). The wells were developed with 50 μL of TMB substrate kit (Pierce) and stopped with 50 μL of H₂SO₄, according to conditions suggested by the supplier. Absorbance was read at 450 nm (A450). The results for the gp120 capture and anti-human capture are set forth in Table 7a (gp120 capture) and Table 7b (anti-human antibody capture), below. The column labeled “Input phage [cfu per well]” lists the corresponding cfu for each dilution of the respective precipitated phage.

TABLE 7a
ELISA data - plates coated with gp120; anti-M13 secondary

| Dilution of | 2G12 | | 3-ALA 1 | |
|---|---|---|---|---|
| precipitated | Input phage | | Input phage | |
| phage | [cfu per well] | A450 | [cfu per well] | A450 |
| 1/2 | 1.43E+11 | 1.576 | 1E+11 | 0.1555 |
| 1/4 | 7.13E+10 | 1.1465 | 5.00E+10 | 0.102 |
| 1/8 | 3.56E+10 | 0.85 | 2.50E+10 | 0.0715 |
| 1/16 | 1.78E+10 | 0.405 | 1.25E+10 | −0.0065 |
| 1/32 | 8.91E+09 | 0.199 | 6.25E+09 | −0.016 |
| 1/64 | 4.45E+09 | 0.0435 | 3.13E+09 | −0.037 |
| 1/128 | 2.23E+09 | 0.016 | 1.56E+09 | −0.03 |
| 1/256 | 1.11E+09 | −0.0095 | 7.81E+08 | −0.0235 |
| 1/512 | 5.57E+08 | −0.023 | 3.91E+08 | −0.0385 |
| 1/1024 | 2.78E+08 | −0.034 | 1.95E+08 | −0.038 |
| 1/2048 | 1.39E+08 | −0.039 | 9.77E+07 | −0.0415 |

TABLE 7b
ELISA data - plates coated with gp120; anti-M13 secondary

| Dilution of | 2G12 | | 3-ALA 1 | |
|---|---|---|---|---|
| precipitated | Input phage | | Input phage | |
| phage | [cfu per well] | A450 | [cfu per well] | A450 |
| 1/2 | 1.43E+11 | 1.3985 | 1E+11 | 1.441 |
| 1/4 | 7.13E+10 | 1.387 | 5.00E+10 | 1.4 |
| 1/8 | 3.56E+10 | 1.311 | 2.50E+10 | 1.3765 |
| 1/16 | 1.78E+10 | 1.1885 | 1.25E+10 | 1.211 |
| 1/32 | 8.91E+09 | 1.08 | 6.25E+09 | 1.0895 |
| 1/64 | 4.45E+09 | 0.869 | 3.13E+09 | 0.8285 |
| 1/128 | 2.23E+09 | 0.65 | 1.56E+09 | 0.591 |
| 1/256 | 1.11E+09 | 0.3995 | 7.81E+08 | 0.369 |
| 1/512 | 5.57E+08 | 0.24 | 3.91E+08 | 0.227 |
| 1/1024 | 2.78E+08 | 0.1265 | 1.95E+08 | 0.1385 |
| 1/2048 | 1.39E+08 | 0.0665 | 9.77E+07 | 0.0745 |

As evidenced by absorbance values listed in Tables 7a and 7b, the phage generated by transformation with the 2G12 vector and the phage generated by transformation with the 3-ALA vector exhibited a phage concentration-dependent binding in the anti-human capture study (where phage were incubated on wells coated with the anti-human antibody and detected with the anti-M13-HRP secondary). In contrast, however, only the phage generated by 2G12 vector transformation (and not that generated by the 3-ALA vector transformation) displayed specific binding to gp120 antigen in the gp120 capture study. Neither sample displayed any specific binding to the wells coated with albumin alone (not shown). These results indicated that the provided methods can be used for phage display and antigen-specific selection of domain exchanged antibodies.

### Example 6D

### Panning, Elution and Amplification

For panning (selection and enrichment based on ability to bind gp120 antigen), 50 microliters of phage solutions from samples generated in Example 6B were added to individual wells of a microtiter plate that had previously been coated with 1 microgram (per well) of gp120 antigen (Immune Technology Corp, New York, N.Y.) overnight at 4° C. The phage was incubated on the plate by incubation at 37° C. for 2 hours with gentle rocking. To remove unbound phage, the supernatant from each well was discarded and plates were washed with 150 microliters of PBS containing 0.05% Tween 20 (polysorbate 20). To elute phage that had bound to the antigen, 100 microliters of 0.1 M HCL (pH 2.2) was added to each well for 10 minutes. The solution (eluate) was removed from the wells by vigorous pipetting and transferred to a 1 mL Eppendorf tube containing 10 uL of 2M Tris-base (pH 9.0). This elution step was repeated and the resulting eluates containing the selected phage were pooled.

For amplification of the selected phage, 220 microliters of the pooled eluate was incubated with 10 mL XL1-Blue cells (having an O.D. between 0.3 and 0.6) for 20 minutes at room temperature (approximately 25° C.). The bacteria then were transferred to a 100 mL bottle containing 45 mL YT medium (5 g Bacto-yeast extract, 8 g Bacto-tryptone, 2.5 g NaCl, in dH₂0, total volume of 1 L), 20 mM glucose, 10 microgram/mL tetracycline and 20 microgram/mL carbenicillin, and incubated at 37° C., with shaking at 250 rpm. After 1 hour of incubation, the medium was supplemented with additional carbenicillin (for a final concentration of 50 micrograms/mL) and the cells incubated at 37° C. until the O.D. of the culture reached 0.3-0.6.

Following amplification, an iterative process was performed, whereby amplified phage from the cultures was isolated by precipitation, as described in the previous section, above, and used for a subsequent round of panning as described in this section above. With the samples generated from the mixtures containing spiked-in vectors, the iterative process was repeated for a total of three rounds of panning, to select for phage displaying antibody fragments that specifically bind to the gp120 antigen. Enrichment was analyzed as described in Example 6E, below.

### Example 6E

### Assessing Enrichment for Antigen-Specificity Following Transformation with Mixed (2G12/3-Ala) Vector Samples and Multiple Rounds of Panning

Enrichment of phage for those displaying antigen specific domain exchanged Fab was assessed following the third round of panning (Example 6D, above) for the samples where the 2G12 vector had been spiked into the 3-Ala vector samples at dilutions of 10⁻³, 10⁻⁴, and 10⁻⁵. For this process, XL1-Blue MRF cells were infected with the output (eluate) phage from the third panning round, and plated on agar plates supplemented with 100 μg/mL carbenicillin and 20 mM glucose. Individual colonies then were picked and used to inoculate 1 mL of SB medium containing 20 mM glucose, 50 μg/mL carbenicillin and 10 μg/mL tetracycline, in a 96 well plate.

The cultures then were incubated for sixteen hours at 37° C., with shaking at 300 rpm. 200 microliters from each well then were used to inoculate 1 mL fresh medium containing 1 mM IPTG and 50 μg/mL carbenicillin. After incubation for 4 hours at 30° C. with shaking at 300 rpm, the cells were lysed by freeze-thawing the plates two times in a dry ice/ethanol bath and then centrifuged at 4000 rpm for 30 minutes, at 4° C., to produce a cleared lysate.

The ELISA-based assay described in Example 6C, above, then was used to detect the presence of total antibody (Goat anti Human Fab MinX capture) and gp120-specific antibody (gp120 JR-FL capture). For this process, specific antibody that remained bound to the microtiter plates was detected using Goat Anti Human FabMin labeled with horse radish peroxidase (HRP) (Pierce, #31414) and a substrate, followed by reading of absorbance as described above.

Results indicated that the cumulative enrichment rates over three rounds for the 10⁻³, 10⁻⁴, and 10⁻⁵ dilutions were 583×, 1,875× and 2,083×, respectively. The “spiked” 2G12 antibody was not detected in the sample from the 1 to 10⁻⁶ dilution. These results indicated that the provided methods can be used to display domain exchanged antibodies on phage and to produce, select, and enrich for domain exchanged antibodies and fragments thereof in an antigen-specific manner. The vectors for phage display of domain exchanged antibodies can be used with the provided methods (e.g. as target polynucleotides) to generate collections of variant, for example, randomized, domain exchanged antibody polypeptides and to select variant antibodies from the collections, for example, based on ability to bind a particular antigen.

### Example 7

### Generation of Domain Exchanged Phage Display Libraries and Selection of Antigen-Specific Domain Exchanged Antibodies from the Libraries

The two nucleic acid libraries generated as described in Example 5B, above (the randomized 2G12 domain exchanged Fab-encoding nucleic acids in the pCAL IT* vectors (“the pCAL IT* library”) and the randomized 2G12 domain exchanged Fab-encoding nucleic acids in the pCAL vectors (“the pCAL library”) were used in spike-in experiments to assess the stability and enrichment of 2G12 Fabs using the 2G12 pCAL vector and 2G12 pCAL IT* vector, and thus the utility of these vectors, in particular the 2G12 pCAL IT* vector, for recovering the 2G12 Fab fragments in a library select antigen-specific domain exchanged antibodies. The phage libraries were subjected to sequential rounds of selection and the isolated phage were analyzed, such as by ELISA, to assess and compare the stability and enrichment of gp120-reactive phage from each library, and to demonstrate that phage display libraries generated using the provided vectors and methods could be used to display and isolate domain exchanged antibodies and fragments thereof.

### Example 7A

### Generation of Vector Mixture Libraries

Four distinct vector library mixtures were generated by adding (“spiking in”), separately, to 1 μg of “the pCAL library,” 10⁻³, 10⁻⁴, 10⁻⁶ and 10⁻⁸ μg of non-randomized 2G12 pCAL vector DNA. The resulting mixtures were labeled 2G12 pCAL 10⁻³; 2G12 pCAL 10⁻⁴; 2G12 pCAL 10⁻⁶; and 2G12 pCAL 10⁻⁸, respectively. Similarly, four distinct vector mixtures were generated by adding (“spiking in”), separately, to 1 μg of “the pCAL IT* library,” 10⁻³, 10⁴, 10⁻⁶ and 10⁻⁸ μg of non-randomized 2G12 pCAL IT* vector DNA. The resulting mixtures were labeled 2G12 pCAL IT* 10⁻³; 2G12 pCAL IT* 10⁻⁴; 2G12 pCAL IT* 10⁻⁶; and 2G12 pCAL IT* 10⁻⁸, respectively.

Additionally, a control mixture was generated, by adding (“spiking in”), separately, to 1 μg of “the pCAL library,” 10⁻³, 10⁻⁴, 10⁻⁶ and 10⁻⁸ μg of anti-HSV antibody (AC8)-encoding vector DNA (described in Example 1, herein; vector containing the nucleic acid having the nucleotide sequence set forth in SEQ ID NO: 46). The resulting mixtures were labeled AC-8 pCAL 10⁻³; AC-8 pCAL 10⁻⁴; AC-8 pCAL 10⁻⁶; and AC-8 pCAL 10⁻⁸, respectively.

### Example 7B

### Phage Display and Selection

As follows, each of the mixtures (libraries) were used to transform partial amber-suppressor XL1-Blue MRF′ cells for the first round of selection. Phage display was then induced and the phage were precipitated and selected by capturing with biotinylated antigen (gp120 for the 2G12 pCAL IT* and the 2G12 pCAL libraries, or HSV-1 gD for the AC-8 libraries) and incubation with streptavidin-coated magnetic beads. After washing of the beads, the bound phage were eluted. These phage were used to infect XL1-Blue MRF′ cells and the phagemid vector DNA was isolated for use in transforming XL1-Blue MRF′ cells to begin the next round of selection. This iterative process was continued for a total of 5 rounds to enrich for phage reactive with gp120 or HSV-1 gD. Following each round of selection, the phage were analyzed, such as by ELISA and determination of phage titers, to assess the stability and enrichment of reactive phage generated from either the pCAL IT* or pCAL vectors.

(i) Transformation of E. coli

Each of the twelve nucleic acid libraries (2G12 pCAL IT* 10⁻³, 10⁻⁴, 10⁻⁶ or 10⁻⁸; 2G12 pCAL 10⁻³, 10⁻⁴, 10⁻⁶ or 10⁻⁸; AC8 pCAL 10⁻³, 10⁴, 10⁻⁶ or 10⁻⁸) were individually transformed into XL1-Blue MRF′ cells (Stratagene). The following selection protocol was then used for each library. Briefly, frozen electrocompetent XL1-Blue MRF′ cells were thawed on ice before 1 μg of the pre-chilled DNA library was added to 100 μL cells in a pre-chilled electroporation cuvette. Following electroporation, 1000 μL of prewarmed 37° C. SOC media was added to resuspend and quench the cells. The cells were then transferred to a sterile 50 mL conical polypropylene tube. The SOC flush process was repeated two more times, resulting in a final volume of approximately 3 mL. A 10 μL aliquot was removed to calculate the electroporation efficiency, described in Example 7C(i), below. To the remaining cell suspension, 2YT medium was added to a final volume of 10 mL, and sterile glucose was added to a final concentration of 20 mM. The tubes were incubated for 1 hour at 37° C. on a shaker at 250 rpm. Following incubation, the cells were transferred to a 100 mL bottle and 2YT media was added to a final volume of 50 mL. Tetracycline [10 μg/mL final concentration], carbenicillin [50 μg/mL final concentration] and glucose (20 mM final concentration) also were added. The cells were then incubated for 2 hours at 37° C. on a shaker at 250 rpm, before being centrifuged at room temperature for 25 minutes at 4000 rpm to obtain a cell pellet.

(ii) Phagemid Expression

To induce phagemid expression, the cell pellet was resuspended in 2YT medium (containing 10 μg/mL tetracycline and 50 μg/mL carbenicillin) to a final volume of 30 mL per μg DNA electroporated). For cells containing the pCAL IT* vector, IPTG also was added to the medium to a final concentration of 1 mM. The cells were incubated at 30° C. for 1 hour, shaking at 250 rpm before VCSM13 helper phage was added at a multiplicity of infection (MOI) of 60:1. The cells were incubated at 30° C. for 8 hours, shaking at 300 rpm, before the temperature was lowered to 4° C. for incubation at 200 rpm until use.

(iii) Phage Precipitation

The cell culture was centrifuged for 30 minutes at 4000 rpm and 32 mL of the supernatant was transferred to a 50 mL centrifuge tube (Nalgene), to which 8 mL of 20% PEG, in 2.5 M NaCl, was added. The tube was then inverted 10 times and incubated on ice for 30 minutes, before the cells were centrifuged at 13,000 rpm for 30 minutes at 4° C. The supernatant was removed and the tube was inverted on a paper towel for 5-10 minutes to remove any excess media. The phage pellet was then resuspended in 2 mL PBS and aliquoted and transferred to sterile microcentrifuge tubes (Eppendorf). The tubes were centrifuged at 13,500 rpm for 5 minutes at 25° C. and the supernatant was transferred to a sterile microcentrifuge tube.

(iv) Phage Capture

To 1.5 mL phage in a microfuge tube, Tween 20 was added to a final concentration of 0.05%. The appropriate biotinylated antigen also was added to a final concentration of 41.6 nM. For the 2G12 pCAL and 2G12 pCAL IT* libraries, biotinylated gp120 (Strain JR-FL, Immune Technology Corp) was used as the capture antigen. Biotinylated HSV-1 gD (Vybion) was used as the capture Ag for the AC-8 pCAL libraries. The phage were then incubated for 2 hours at 37° C., rocking.

To prepare the magnetic beads for capture of the antigen-bound phage, 200 μL Dynabeads® M-280 Stretavidin (Invitrogen) in an microcentrifuge tube were washed 3 times by first applying the tube to the DynaMag2 magnet particle concentrator for 2 minutes to collect the beads at the bottom of the tube, removing the supernatant then washing the beads with 1 mL PBS by repeatedly pipetting. This process was repeated two more times for a total of 3 washes. The beads were then blocked by the addition of 2 ml blocking solution (3% bovine serum albumin (BSA) diluted in PBS) and incubating for 2 hours at 37° C. The beads were again concentrated using a DynaMag™-2 magnet and washed with 200 μL PBS.

To capture the antigen-bound phage, 200 μL of the washed beads were added to 1 mL of the phage/biotinylated antigen mix and the resulting mixture was incubated for 30 minutes at 37° C., rocking. To remove any unbound phage, the beads were washed with PBS/0.05% Tween 20 by concentrating the beads using the DynaMag2 magnet particle concentrator for 2 minutes and removing the supernatant, then washing the beads with 1 mL PBS/0.05% Tween 20. This process was repeated twice for a total of 3 washes. The supernatant was then removed.

(v) Phage Elution

To elute the phage from the bead pellet, 150 μl, 0.1 M HCl (pH 2.2) was added to the beads and the beads were incubated for 10 minutes at room temperature. The tube was vortexed repeatedly and pipetted to ensure maximal elution of the phage. The beads were removed using the magnet and the supernatant containing the eluted phage was transferred to a sterile microcentrifuge tube. The phage were then neutralized by the addition of 15 μL 2 M Tris base (pH 9) per 150 μL phage eluate. To the microcentrifuge tube containing the phage, 150 μL 0.1 M HCl (pH 2.2) was added and the tube was incubated for 5 minutes at room temperature before the phage were neutralized by the addition of 15 μl, 2 M Tris base (pH 9) per 150 μL phage eluate.

(vi) Infection of E. coli XL1-Blue MRF′ Cells

Chemically competent XL1-Blue MRF′ cells were streaked onto a Luria Broth (LB) agar plate containing 10 μg/mL tetracycline and incubated overnight at 37° C. Colonies were scraped off the plate and inoculated into 5 mL SB medium (30 g/L Bacto tryptone (Fisher), 20 g/L yeast extract (Fisher), 10 g/L MOPS (Fisher), pH: 7.0) containing 10 μg/mL tetracycline, and the culture was incubated at 37° C., 250 rpm until the OD 600 reached 1.0-2.0. The OD 600 was then adjusted to between 0.6 and 1.0 and 2.5 mL XL1-Blue MRF′ cells were infected with eluted phage (approximately 330 μL phage. The cells were incubated at room temperature for 30 minutes.

The infected XL1-Blue cells (2.5 mL) were then transferred to a bioassay tray (Corning) containing LB agar, 100 μg/mL carbenicillin and 100 mM glucose. The cells were spread evenly using a steril spreader and the tray was incubated at room temperature for 30 minutes. The tray was then inverted and placed in a 37° C. incubator for 12 hours.

(vii) DNA Purification

The cells were scraped from the plate and DNA was purified from the cells using a Qiafilter Midiprep Kit (Qiagen). Briefly, 25 mL 2YT media was spread onto the tray and the cells were gently scraped off and removed by pipetting. The cells were then centrifuged for 15 minutes at 5000-8000 rpm and the pellet was resuspended in 4 mL Buffer P1 of the Qiafilter Midiprep Kit (Qiagen). Buffer P2 (4 mL) was added and the solution was mixed by inversion before the lysis reaction was incubated for 5 minutes at room temperature. Precipitation was facilitated by adding 4 mL chilled Buffer P3. The lysate was then transferred to the barrel of the Qiafilter cartridge and incubated for 10 minutes at room temperature.

A Qiagen-tip 100 was equilibrated by applying 4 mL of Buffer QBT and allowing the column to empty by gravity flow. The cap from the Qiafilter Midi Cartridge outlet nozzle was removed and the plunger was inserted into the Qiafilter Midi Cartridge and the cell lysate was filtered into the previously equilibrated Qiagen-tip. The Qiagen-tip 100 was washed by applying 2×10 mL of Buffer QC before the DNA was eluted with 5 mL Buffer QF. The DNA was then precipitated by adding 3.5 mL (equivalent to 0.7 volumes) of room temperature isopropanol to the eluted DNA. The solution was mixed and centrifuged immediately at >15,000×g for 30 minutes at 4° C. The supernatant was decanted and the DNA pellet was washed with 2 mL room temperature 70% ethanol and again centrifuged at >15,000×g for 10 minutes at 4° C. The DNA pellet was air dried for 5-10 minutes and dissolved in TE buffer, pH 8.0, or 10 mM Tris-Cl, pH 8.5 to achieve a concentration of ≧125 ng/μL.

(viii) Repetition of the Process for Rounds 2-5.

The nucleic acid library DNA isolated in Example 7B(vii), above, was then used to transform XL1-Blue MRF′ cells and the process described in Example 7B(i) through Example 7B(vii), was repeated for a second round of screening. Following isolation of DNA, the process was again repeated until a total of 5 rounds of screening were performed. During each screening, the washing conditions for washing the phage-bound beads (Example 7B(iv)) were adjusted to increase stringency. Table 8 sets forth the wash conditions used in each round.

TABLE 8
Phage-bound bead wash conditions

| | No. of | |
|---|---|---|
| Round | washes | Description |
| 1 | 3 | Gentle washing steps: |
| | | Washing procedure is completed quickly and without |
| | | pipetting up and down vigorously. |
| 2 | 5 | Gentle washing steps: |
| | | Washing procedure is completed quickly and without |
| | | pipetting up and down vigorously. |
| 3 | 10 | Stringent washing steps: |
| | | Washing procedure is completed slowly and |
| | | pipetting is performed vigorously |
| 4-5 | 10 | Stringent washing steps: |
| | | Washing procedure is completed slowly and |
| | | pipetting is performed vigorously. Incubate |
| | | phage and biotinylated antigen in PBS/Tween wash for |
| | | 5 minute intervals, rocking at room temperature in |
| | | between each wash step. |

### Example 7C

### Analysis of Enrichment Using the Phage Libraries

The stability of the vectors and the enrichment of phage displaying antigen-specific 2G12 Fabs was assessed throughout the 5 round selection process described above. The various parameters analyzed included electroporation efficiencies (of the electroporations described in Example 7B(i)), input and output phagemid titers (i.e. before and after the phage capture described in Example 7B(iv)), and antigen-reactivity.

(i) Transformation Efficiencies

To determine the transformation efficiencies, a 10 μL aliquot of cells taken following electroporation (described in Example 7B(i), above), was used to prepare serial 10-fold dilutions. Into a 96-well plate, 90 μL SOC was added to the wells and the 10 μL cell aliquot was added to the first well. Serial 10-fold dilution were then prepared, resulting in 10⁻¹, 10⁻², 10⁻, 10⁻⁴, 10⁻⁵ and 10⁻⁶ dilutions. Seventy-five μL of the 10⁻³, 10⁻⁴, 10⁻⁵ and 10⁻⁶ dilutions were plated onto LB agar plates containing 100 μg/mL carbenicillin. The liquid was spread and the plate was allowed to dry before being inverted and placed in a 37° C. incubator overnight.

The number of transformants from the electroporation of cells with the nucleic acid libraries was calculated by multiplying the number of colonies on the plate by the culture volume and dividing by the plating volume, as set forth in the following equation:

[number of colonies/plating volume (μL)]×[culture volume (μL)/μg DNA]×dilution factor.

As demonstrated in Table 9, each electroporation resulted in over 10⁸ colonies per μg electroporated DNA.

TABLE 9
Transformation efficiency using each nucleic acid library

| | Titer (cfu/μg) | | | | |
|---|---|---|---|---|---|
| Library | Round 1 | Round 2 | Round 3 | Round 4 | Round 5 |
| AC8 pCAL | 2.64 × 10⁸ | 1.20 × 10⁹ | 1.92 × 10⁸ | ND | ND |
| [10⁻³] | | | | | |
| AC8 pCAL | 5.12 × 10⁸ | 2.50 × 10⁹ | 3.80 × 10⁸ | 1.00 × 10⁸ | ND |
| [10⁻⁴] | | | | | |
| AC8 pCAL | 8.96 × 10⁸ | 1.40 × 10⁹ | 2.20 × 10⁸ | 2.52 × 10⁸ | 3.70 × 10⁸ |
| [10⁻⁶] | | | | | |
| AC8 pCAL | 4.04 × 10⁸ | 3.00 × 10⁹ | 3.08 × 10⁸ | 2.44 × 10⁸ | 3.04 × 10⁸ |
| [10⁻⁸] | | | | | |
| 2G12 pCAL | 2.76 × 10⁸ | 1.60 × 10⁹ | 3.92 × 10⁸ | 1.32 × 10⁸ | ND |
| [10⁻³] | | | | | |
| 2G12 pCAL | 4.96 × 10⁸ | 1.40 × 10⁹ | 2.72 × 10⁸ | 1.28 × 10⁸ | ND |
| [10⁻⁴] | | | | | |
| 2G12 pCAL | 6.12 × 10⁸ | 1.30 × 10⁹ | 2.92 × 10⁸ | 6.80E+07 | 3.60 × 10⁸ |
| [10⁻⁶] | | | | | |
| 2G12 pCAL | 9.28 × 10⁸ | 2.40 × 10⁹ | 3.84 × 10⁸ | 1.00 × 10⁸ | 4.50 × 10⁸ |
| [10⁻⁸] | | | | | |
| 2G12 pCAL | 1.12 × 10⁸ | 1.30 × 10⁹ | 2.24 × 10⁸ | ND | ND |
| IT* [10⁻³] | | | | | |
| 2G12 pCAL | 1.92 × 10⁸ | 9.60 × 10⁸ | 3.00 × 10⁸ | 6.40 × 10⁷ | ND |
| IT* [10⁻⁴] | | | | | |
| 2G12 pCAL | 3.32 × 10⁸ | 1.20 × 10⁹ | 1.60 × 10⁸ | 4.44 × 10⁸ | 3.06 × 10⁸ |
| IT* [10⁻⁶] | | | | | |
| 2G12 pCAL | 3.64 × 10⁸ | 1.10 × 10⁹ | 7.40 × 10⁸ | 1.60 × 10⁸ | 3.68 × 10⁸ |
| IT* [10⁻⁸] | | | | | |

In addition to calculating the transformation efficiency, the input phagemid DNA (i.e. the phagemid DNA used for electroporation) at each round was digested with Pac I enzyme (New England Biolabs) to linearize the vector, and the vector was run on an agarose gel to visualize the abundance and quality of the DNA. Non-digested supercoiled DNA also was run on a gel. All of the phagemid vector DNA samples were observed to have the expected size with no degradation products.

(ii). Phagemid Titers

The titers of the phagemids before (input phage) and after (output phage) capture also were determined by titration and the percentage enrichment calculated. To determine the titer of input phage, 10 μL of input phage (obtained following precipitation and resuspension in PBS; see Example 7B(iii)) was added to 90 μL SOC and then diluted in series of 10-fold dilutions in SOC. One μL of each dilution was then added to 99 μL of XL1-Blue MRF′ cells and the phage was allowed to infect the cells for 15 minutes at room temperature, before 20 μL of the infected cells was plated onto LB agar plates containing 100 μg/mL carbenicillin. The plates were incubated overnight at 37° C. to obtain single colonies, which were then calculated to the phage titer (cfu/mL).

To determine the titer of the output phage, 10 μL of the XL1-Blue cells that had been infected with the eluted phage (see Example 7B(vi)) was added to 90 μL SOC and then diluted in series of 10-fold dilutions in SOC. Seventy-five μL of the diluted cells were then plated onto LB agar plates containing 100 μg/mL carbenicillin. The plates were allowed to dry for 15 minutes before being incubated overnight at 37° C. to obtain single colonies, which were then calculated to the phage titer (cfu/mL).

Table 10 sets forth the input and output phage titers and the % enrichment.

TABLE 10
Phagemid titers before and after capture

| | Phagemid titer (cfu/mL) | | Enrichment |
|---|---|---|---|
| Library | Input | Output | (%) |

Round 1

| | | | |
|---|---|---|---|
| AC8 pCAL [10⁻³] | 1.60E+12 | 3.16E+06 | 0.000198 |
| AC8 pCAL [10⁻⁴] | 2.00E+12 | 1.74E+06 | 0.000087 |
| AC8 pCAL [10⁻⁶] | 7.60E+11 | 1.80E+06 | 0.000237 |
| AC8 pCAL [10⁻⁸] | 4.16E+11 | 2.40E+06 | 0.000577 |
| 2G12 pCAL [10⁻³] | 4.96E+11 | 5.70E+06 | 0.001149 |
| 2G12 pCAL [10⁻⁴] | 3.20E+12 | 1.00E+07 | 0.000313 |
| 2G12 pCAL [10⁻⁶] | 4.00E+11 | 8.10E+06 | 0.002025 |
| 2G12 pCAL [10⁻⁸] | 2.80E+12 | 3.60E+06 | 0.000129 |
| 2G12 pCAL IT* [10⁻³] | 6.80E+11 | 3.09E+06 | 0.00045 |
| 2G12 pCAL IT* [10⁻⁴] | 1.28E+12 | 3.00E+06 | 0.00023 |
| 2G12 pCAL IT* [10⁻⁶] | 3.24E+12 | 8.25E+06 | 0.00026 |
| 2G12 pCAL IT* [10⁻⁸] | 1.20E+12 | 4.80E+06 | 0.0004 |

Round 2

| | | | |
|---|---|---|---|
| AC8 pCAL [10⁻³] | 2.80E+13 | 5.40E+07 | 0.000193 |
| AC8 pCAL [10⁻⁴] | 2.00E+13 | 2.30E+07 | 0.000115 |
| AC8 pCAL [10⁻⁶] | 2.80E+13 | 3.50E+06 | 0.000013 |
| AC8 pCAL [10⁻⁸] | 2.00E+13 | 6.20E+06 | 0.000031 |
| 2G12 pCAL [10⁻³] | 8.80E+12 | 5.20E+06 | 0.000059 |
| 2G12 pCAL [10⁻⁴] | 1.40E+13 | 2.40E+07 | 0.000171 |
| 2G12 pCAL [10⁻⁶] | 1.70E+13 | 1.04E+07 | 0.000061 |
| 2G12 pCAL [10⁻⁸] | 9.20E+12 | 2.14E+07 | 0.000233 |
| 2G12 pCAL IT* [10⁻³] | 2.10E+13 | 8.80E+06 | 0.000042 |
| 2G12 pCAL IT* [10⁻⁴] | 1.10E+13 | 5.64E+07 | 0.000513 |
| 2G12 pCAL IT* [10⁻⁶] | 2.90E+13 | 1.65E+07 | 0.000057 |
| 2G12 pCAL IT* [10⁻⁸] | 1.50E+13 | 3.22E+07 | 0.000215 |

Round 3

| | | | |
|---|---|---|---|
| AC8 pCAL [10⁻³] | 6.80E+13 | ND | ND |
| AC8 pCAL [10⁻⁴] | 2.80E+13 | 1.00E+06 | 0.000004 |
| AC8 pCAL [10⁻⁶] | 3.60E+13 | 2.30E+06 | 0.000006 |
| AC8 pCAL [10⁻⁸] | 6.40E+13 | 3.20E+06 | 0.000005 |
| 2G12 pCAL [10⁻³] | 2.80E+13 | 2.80E+06 | 0.00001 |
| 2G12 pCAL [10⁻⁴] | 6.40E+11 | 5.40E+06 | 0.000844 |
| 2G12 pCAL [10⁻⁶] | 5.60E+12 | 7.00E+06 | 0.000125 |
| 2G12 pCAL [10⁻⁸] | 3.20E+13 | 7.73E+06 | 0.000024 |
| 2G12 pCAL IT* [10⁻³] | 6.40E+13 | ND | ND |
| 2G12 pCAL IT* [10⁻⁴] | 4.00E+13 | 9.00E+06 | 0.000023 |
| 2G12 pCAL IT* [10⁻⁶] | 6.80E+13 | 2.60E+06 | 0.000004 |
| 2G12 pCAL IT* [10⁻⁸] | 2.40E+13 | 6.20E+06 | 0.000026 |

Round 4

| | | | |
|---|---|---|---|
| AC8 pCAL [10⁻³] | ND | ND | ND |
| AC8 pCAL [10⁻⁴] | 4.00E+12 | 1.45E+07 | 0.000363 |
| AC8 pCAL [10⁻⁶] | 3.60E+12 | 5.20E+06 | 0.000144 |
| AC8 pCAL [10⁻⁸] | 5.20E+12 | 2.70E+06 | 0.000052 |
| 2G12 pCAL [10⁻³] | ND | 3.60E+06 | ND |
| 2G12 pCAL [10⁻⁴] | 6.00E+12 | 2.60E+06 | 0.000043 |
| 2G12 pCAL [10⁻⁶] | 3.60E+12 | 2.69E+06 | 0.000075 |
| 2G12 pCAL [10⁻⁸] | 5.60E+12 | 3.70E+06 | 0.000066 |
| 2G12 pCAL IT* [10⁻³] | ND | ND | ND |
| 2G12 pCAL IT* [10⁻⁴] | 3.20E+12 | 7.40E+06 | 0.000231 |
| 2G12 pCAL IT* [10⁻⁶] | 4.40E+12 | 4.60E+06 | 0.000105 |
| 2G12 pCAL IT* [10⁻⁸] | 2.80E+12 | 3.70E+06 | 0.000132 |

Round 5

| | | | |
|---|---|---|---|
| AC8 pCAL [10⁻³] | ND | ND | ND |
| AC8 pCAL [10⁻⁴] | ND | ND | ND |
| AC8 pCAL [10⁻⁶] | 1.08E+13 | 9.20E+06 | 0.000085 |
| AC8 pCAL [10⁻⁸] | 4.40E+12 | 2.30E+07 | 0.000523 |
| 2G12 pCAL [10⁻³] | ND | ND | ND |
| 2G12 pCAL [10⁻⁴] | ND | ND | ND |
| 2G12 pCAL [10⁻⁶] | 1.24E+13 | 8.30E+05 | 0.000007 |
| 2G12 pCAL [10⁻⁸] | 8.00E+12 | 1.70E+06 | 0.000021 |
| 2G12 pCAL IT* [10⁻³] | ND | ND | ND |
| 2G12 pCAL IT* [10⁻⁴] | ND | ND | ND |
| 2G12 pCAL IT* [10⁻⁶] | 1.08E+13 | ND | ND |
| 2G12 pCAL IT* [10⁻⁸] | 4.80+12 | 1.80E+06 | 0.000038 |

ND = not done

(iii) ELISA Analysis of Fabs Displayed by Selected Phage

The stability and enrichment of gp120-specific Fabs displayed on phage from the various libraries was assessed by ELISA. Two ELISAs were performed, one to assess the reactivity of the phage on a polyclonal level, and the other to assess the reactivity of the phage on a monoclonal level. In the first assay (polyclonal), ELISAs were performed using an aliquot of the precipitated input phage obtained in Example 7B(iii). In the second assay (monoclonal), ELISAs were performed using cells lysates from individual colonies of XL1-Blue MRF′ cells that had been infected with the eluted phage. Reactivity of the displayed Fabs was tested against two different antigens to assess specificity: gp120 (Strain JR-FL, Immune Technologies), and HSV-1 gD (Vybion, Inc.). Goat anti-human IgG F(ab′)₂ fragment-specific antibodies (Jackson ImmunoResearch Laboratories, Inc) were used as a capture “antigen” to assess stability of the selected Fabs.

a. Polyclonal ELISA Analysis

To determine the reactivity of the phage on a polyclonal level, eluted phage from each round of selection were assayed by ELISA for reactivity with gp120 (Strain JR-FL, Immune Technologies), HSV-1 gD (Vybion, Inc.) and goat anti-human IgG F(ab′)₂ fragment specific antibodies (Jackson ImmunoResearch Laboratories, Inc). Ninety-six well ELISA plates were coated with antigen (gp120, HSV-1 gD or anti-human Fab) at 100 ng/50 μL (diluted in PBS)/well at 4° C. overnight. Following coating, the plates were washed twice with PBS/0.05% Tween 20 and then blocked with 4% non-fat dry milk in PBS at 37° C. for 2 hours. The plates were again washed twice with PBS/0.05% Tween 20. To each well, 50 μL of 1×10⁶, 1×10⁷, 1×10⁸, 1×10⁹, 1×10¹⁰, 1×10¹¹, 1×10¹², or 1×10¹³ cfu/well phage was added. The ELISA assay plate was incubated for a further 2 hours at 37° C. and the plates were washed 5 times with PBS/0.05% Tween 20 before 50 μL of ImmunoPure Goat Anti-Human IgG [F(ab′)2], Peroxidase Conjugated (Pierce:diluted 1:1000) was added to each well of the plates originally coated with HSV-gD or gp120, and anti-M13 HRP Conjugated (GE:diluted 1:5000) was added to each well of the plates originally coated with goat anti-human Fab. Following incubation for 1 hour at room temperature, the plate was washed 5 times with PBS/0.05% Tween 20 and 50 μL of TMB substrate (Pierce; prepared according to manufacturer's instructions) was added to each well and the plate was then incubated until a blue color developed. The reaction was stopped with the addition of 50 μL 1M H₂SO₄ and the optical density (O.D. 450 nm) of each well was determined.

It was observed that phage selected from the 2G12 pCAL IT* libraries had slightly increased reactivity with anti-human Fab antibodies compared to the phage selected from 2G12 pCAL libraries, indicating the expression from the pCAL IT* vectors increased stability of the Fabs. In addition, enrichment of gp120 reactive phage also was increased using the 2G12 pCAL IT* libraries compared to the 2G12 pCAL libraries, as indicated by higher OD values in ELISAs for these phage using gp120 as the capture antigen.

b. Monoclonal ELISA Analysis

To determine the reactivity of the phage on a monoclonal level, an aliquot of the XL1-Blue MRF′ cells that were infected with the eluted phage after each round of selection (see Example 7B(vi)) were first diluted and plated onto LB agar plates containing 100 μg/mL carbenicillin and incubated overnight at 37° C. to obtain single colonies. Individual colonies were then inoculated into a 96 deep well (1 mL volume) plate containing SB media containing 20 mM Glucose, 50 μg/mL carbenicillin and 10 μg/mL tetracycline. This parental plate was incubated for 16 hours at 37° C., shaking at 300 rpm. From each well of the parental plate, 200 μL of cell culture was inoculated into corresponding wells of a daughter plate that contained 1 mL/well SB media containing 20 mM glucose, 50 μg/mL carbenicillin and 10 μg/mL tetracycline. The parental plate was centrifuged at 3500 rpm for 30 minutes to pellet the cells and the pellets were stored at −20° C.

IPTG was added to each well of the daughter plate to a final volume of 1 mM. The daughter plate was incubated for 8 hours at 37° C., shaking at 300 rpm. The daughter plate was then frozen in a dry ice/ethanol bath and thawed to lyse the cells, before the lysate was cleared by centrifugation at 3500 rpm for 15 minutes. The supernatant was then extracted for analysis by ELISA.

Ninety-six well ELISA plates were coated with antigen at 100 ng/50 μL (diluted in PBS)/well at 4° C. overnight. Reactivity of the phage isolated from each colony was tested against two different antigens: gp120 (Strain JR-FL, Immune Technologies), HSV-1 gD (Vybion, Inc.). Goat anti-human IgG F(ab′)₂ fragment specific antibodies (Jackson ImmunoResearch Laboratories, Inc) also were used as a capture “antigen.” Following coating, the plates were washed twice with PBS/0.05% Tween 20 and then blocked with 135 μL/well 4% % non-fat dry milk in PBS at 37° C. for 2 hours. The plates were again washed twice with PBS/0.05% Tween 20. To each well, 50 μL of the bacterial cell lysate supernatant containing the phage was added, at a 1:2 dilution in PBS/0.05% Tween 20, to the ELISA assay plate and the plate was incubated for a further 2 hours at 37° C. The plate was washed 5 times with PBS/0.05% Tween 20 before 50 μl of ImmunoPure Goat Anti-Human IgG [F(ab′)2], Peroxidase Conjugated (Pierce:diluted 1:1000) was added to each well. Following incubation for 1 hour at room temperature, the plate was washed 5 times with PBS/0.05% Tween 20 and 50 μL of TMB substrate (Pierce; prepared according to manufacturers instructions) was added to each well and the plate was then incubated until a blue color developed. The reaction was stopped with the addition of 50 μL 1M H₂SO₄ and the optical density (O.D. 450 nm) of each well was determined. An OD 450 nm of greater than 0.5 indicated that the phage in that well (which were derived from a single colony) displayed Fabs that exhibited a positive reactivity for gp120. Tables 11-13 set forth the percentage of phage that displayed Fabs that bound gp120, anti-human Fab and HSV-1 gD, respectively after each round of selection.

It was observed that there was increased stability and enrichment of phage displaying 2G12 Fabs from phage display libraries generated using the 2G12 pCAL IT* phagemid vector libraries compared to those generated using the 2G12 pCAL phagemid vector libraries. For example, after the 4^{th} round of selection, 31% of phage generated from the 2G12 pCAL IT* [10⁻⁴] phagemid vector library reacted with gp120, compared to only 9% from the 2G12 pCAL [10⁻³] phagemid vector library (see Table 11). Further, the Fabs displayed on the phage from the 2G12 pCAL IT*libraries were recognized by the anti-human IgG [F(ab′)2] capture antibody at higher frequencies than the Fabs displayed on the phage from the 2G12 pCAL libraries. In particular, reactivity of Fabs displayed by phage from the 2G12 pCAL libraries with the anti-human IgG [F(ab′)2] capture antibody decreased as the selection rounds proceeded, indicating that the phagemids and/or Fabs were less stable than those from the 2G12 pCAL IT*libraries, which maintained high reactivity throughout the selection process (Table 12).

TABLE 11
Evaluation of gp120 antigen specific Fabs displayed by phage that
were selected after each round of capture

| | Number and percentage of gp120-specific phage following each | | | | | | | | | |
|---|---|---|---|---|---|---|---|---|---|---|
| | round of selection | | | | | | | | | |
| | Round 1 | | Round 2 | | Round 3 | | Round 4 | | Round 5 | |
| AC8 pCAL | ND | ND | 0/22 | 0% | ND | ND | ND | ND | ND | ND |
| [10⁻³] | | | | | | | | | | |
| AC8 pCAL | ND | ND | 0/22 | 0% | 0/22 | 0% | 0/44 | 0% | ND | ND |
| [10⁻⁴] | | | | | | | | | | |
| AC8 pCAL | ND | ND | 0/22 | 0% | 0/33 | 0% | 0/44 | 0% | 0/44 | 0% |
| [10⁻⁶] | | | | | | | | | | |
| AC8 pCAL | ND | ND | 0/22 | 0% | 0/33 | 0% | 0/88 | 0% | 0/44 | 0% |
| [10⁻⁸] | | | | | | | | | | |
| 2G12 pCAL | ND | ND | 0/22 | 0% | 0/22 | 0% | 2/22 | 9% | ND | ND |
| [10⁻³] | | | | | | | | | | |
| 2G12 pCAL | ND | ND | 0/22 | 0% | 0/22 | 0% | 0/22 | 0% | ND | ND |
| [10⁻⁴] | | | | | | | | | | |
| 2G12 pCAL | ND | ND | 0/22 | 0% | 0/22 | 0% | 0/22 | 0% | ND | ND |
| [10⁻⁶] | | | | | | | | | | |
| 2G12 pCAL | ND | ND | 0/22 | 0% | 0/22 | 0% | 0/22 | 0% | ND | ND |
| [10⁻⁸] | | | | | | | | | | |
| 2G12 pCAL | ND | ND | ND | ND | ND | ND | ND | ND | ND | ND |
| IT* [10⁻³] | | | | | | | | | | |
| 2G12 pCAL | ND | ND | 0/44 | 0% | 10/176 | 6% | 41/132 | 31% | ND | ND |
| IT* [10⁻⁴] | | | | | | | | | | |
| 2G12 pCAL | ND | ND | 0/44 | 0% | 0/44 | 0% | 0/44 | 0% | ND | ND |
| IT* [10⁻⁶] | | | | | | | | | | |
| 2G12 pCAL | ND | ND | 0/44 | 0% | 0/44 | 0% | 0/44 | 0% | 14/176 | 8% |
| IT* [10⁻⁸] | | | | | | | | | | |

TABLE 12
Evaluation of reactivity of Fabs displayed by phage that were selected
after each round of capture with anti-human Fab.

| | Number and percentage of phage that reacted with anti-human Fab | | | | | | | | | |
|---|---|---|---|---|---|---|---|---|---|---|
| | antibody following each round of selection | | | | | | | | | |
| | Round 1 | | Round 2 | | Round 3 | | Round 4 | | Round 5 | |
| AC8 pCAL | ND | ND | 21/22 | 95% | ND | ND | ND | ND | ND | ND |
| [10⁻³] | | | | | | | | | | |
| AC8 pCAL | ND | ND | 21/22 | 95% | 21/22 | 95% | 37/44 | 84% | ND | ND |
| [10⁻⁴] | | | | | | | | | | |
| AC8 pCAL | ND | ND | 21/22 | 95% | 27/33 | 81% | 40/44 | 91% | 30/44 | 68% |
| [10⁻⁶] | | | | | | | | | | |
| AC8 pCAL | ND | ND | 21/22 | 95% | 32/33 | 97% | 68/88 | 77% | 32/44 | 72% |
| [10⁻⁸] | | | | | | | | | | |
| 2G12 pCAL | ND | ND | 21/22 | 95% | 71/22 | 77% | 15/22 | 68% | ND | ND |
| [10⁻³] | | | | | | | | | | |
| 2G12 pCAL | ND | ND | 22/22 | 100% | 21/22 | 95% | 18/22 | 82% | ND | ND |
| [10⁻⁴] | | | | | | | | | | |
| 2G12 pCAL | ND | ND | 20/22 | 90% | 21/22 | 95% | 17/22 | 77% | ND | ND |
| [10⁻⁶] | | | | | | | | | | |
| 2G12 pCAL | ND | ND | 20/22 | 100% | 20/22 | 90% | 13/22 | 60% | ND | ND |
| [10⁻⁸] | | | | | | | | | | |
| 2G12 pCAL | ND | ND | ND | ND | ND | ND | ND | ND | ND | ND |
| IT* [10⁻³] | | | | | | | | | | |
| 2G12 pCAL | ND | ND | 44/44 | 100% | 172/176 | 97% | 132/132 | 100% | ND | ND |
| IT* [10⁻⁴] | | | | | | | | | | |
| 2G12 pCAL | ND | ND | 41/44 | 93% | 44/44 | 100% | 43/44 | 97% | ND | ND |
| IT* [10⁻⁶] | | | | | | | | | | |
| 2G12 pCAL | ND | ND | 44/44 | 100% | 42/44 | 95% | 41/44 | 93% | 170/176 | 97% |
| IT* [10⁻⁸] | | | | | | | | | | |

TABLE 13
Evaluation of HSV-1 gD antigen specific Fabs displayed by phage that
were selected after each round of capture.

| | Number and percentage of HSV-1 gD-specific phage following each | | | | | | | | | |
|---|---|---|---|---|---|---|---|---|---|---|
| | round of selection | | | | | | | | | |
| | Round 1 | | Round 2 | | Round 3 | | Round 4 | | Round 5 | |
| AC8 pCAL | ND | ND | 14/22 | 63% | ND | ND | ND | ND | ND | ND |
| [10⁻³] | | | | | | | | | | |
| AC8 pCAL | ND | ND | 0/22 | 0% | 1/22 | 5% | 28/44 | 64% | ND | ND |
| [10⁻⁴] | | | | | | | | | | |
| AC8 pCAL | ND | ND | 0/22 | 0% | 1/33 | 3% | 24/44 | 54% | 20/44 | 45% |
| [10⁻⁶] | | | | | | | | | | |
| AC8 pCAL | ND | ND | 0/22 | 0% | 0/33 | 0% | 18/88 | 20% | 23/44 | 52% |
| [10⁻⁸] | | | | | | | | | | |
| 2G12 pCAL | ND | ND | 0/22 | 0% | 0/22 | 0% | 0/22 | 0% | ND | ND |
| [10⁻³] | | | | | | | | | | |
| 2G12 pCAL | ND | ND | 0/22 | 0% | 0/22 | 0% | 0/22 | 0% | ND | ND |
| [10⁻⁴] | | | | | | | | | | |
| 2G12 pCAL | ND | ND | 0/22 | 0% | 0/22 | 0% | 0/22 | 0% | ND | ND |
| [10⁻⁶] | | | | | | | | | | |
| 2G12 pCAL | ND | ND | 0/22 | 0% | 0/22 | 0% | 0/22 | 0% | ND | ND |
| [10⁻⁸] | | | | | | | | | | |
| 2G12 pCAL | ND | ND | ND | ND | ND | ND | ND | ND | ND | ND |
| IT* [10⁻³] | | | | | | | | | | |
| 2G12 pCAL | ND | ND | 0/44 | 0% | 0/176 | 0% | 0/132 | 0% | ND | ND |
| IT* [10⁻⁴] | | | | | | | | | | |
| 2G12 pCAL | ND | ND | 0/44 | 0% | 0/44 | 0% | 0/44 | 0% | ND | ND |
| IT* [10⁻⁶] | | | | | | | | | | |
| 2G12 pCAL | ND | ND | 0/44 | 0% | 0/44 | 0% | 0/44 | 0% | 0/176 | 0% |
| IT* [10⁻⁸] | | | | | | | | | | |

### Example 8

### Design of Vectors for Generating Additional Domain-Exchange Antibody Fragment Variants

To generate various types of domain exchanged antibody fragments and assess their ability to assemble in periplasm for display on phage, multiple polynucleotide constructs were designed and generated. The constructs were designed to express various combinations of heavy and light chain regions of domain exchanged antibody, to form a plurality of domain exchanged antibody fragments (in addition to the domain exchanged Fab fragment), in the form of gene III fusion proteins, for phage display. The additional 2G12 antibody fragment fusion proteins encoded by the constructs are illustrated schematically in FIG. 2.

FIG. 2A schematically illustrates a phage displayed domain exchanged Fab fragment (illustrated as a cp3 fusion polypeptide) described in the examples above, as well as additional exemplary displayed domain exchanged fragments, all shown in the figure as parts of phage coat protein (cp3) fusions. These additional fragments, illustrated in FIGS. 2B-H, further contain covalent linkage of two heavy chains via a disulphide bond and/or via a peptide linker, and/or contain only variable heavy and light chains joined by peptide linkers, forming single chain fragments.

In addition to the 2G12 domain exchanged Fab fragment, a construct for expressing a 2G12 domain exchanged fragment-cp3 fusion polypeptide was carried out for each of the fragment types illustrated in FIG. 2.

### Example 8A

### 2G12 Fragments with Varying Configuration

Changes were made to the 2G12 domain exchanged Fab fragment to evaluate effects on stability of the domain exchanged configuration of the domain exchanged Fab molecule. For example, as shown in FIG. 2B, the domain exchanged Fab hinge fragment (encoded by the polynucleotide construct having the nucleic acid sequence set forth in SEQ ID NO: 38) was designed to include the amino acids making up the hinge region, providing cysteine residues that form a disulfide bridge between the two heavy chain domains, which could potentially further stabilize the domain exchanged configuration. As shown in FIG. 2C, the domain exchanged Fab Cys19 fragment (encoded by the polynucleotide construct having the nucleic acid sequence set forth in SEQ ID NO: 29) was identical to the domain exchanged Fab fragment, but contained an Isoleucine to cysteine mutation at position 19 of the heavy chain. This mutation was expected to induce formation of a disulfide bridge between the heavy chain variable regions, which was expected to stabilize the domain exchanged configuration at the heavy chain interface.

As shown in FIG. 2D, the 2G12 domain exchanged scFab ΔC2Cys19 fragment (encoded by the polynucleotide construct having the nucleic acid sequence set forth in SEQ ID NO: 30) contained the same isoleucine to cysteine mutation, but lacked the two cysteines responsible for formation of disulfide bridges between the C_{H} and C_{L} domains, and included two peptide linkers, covalently joining the heavy and light chains.

In addition to variation of the 2G12 Fab fragment, 2G12 domain exchanged single chain fragments were designed to assess expression, folding and/or domain exchanged configuration of antibodies other than the domain exchanged Fab fragment. As shown in FIG. 2E, the domain exchanged scFv tandem fragment (encoded by the polynucleotide construct having the nucleic acid sequence set forth in SEQ ID NO: 40) was a single-chain fragment containing two V_{H} and two V_{L} domains and no constant region domains. These four variable region domains were linked via peptide linkers, which was expected to ensure formation of a domain exchanged type configuration, which could potentially be used to display domain exchanged antibody on the surface of phage, even in the absence of an amber stop codon between the nucleic acid encoding the antibody and that encoding the gene III. By contrast, as shown in FIG. 2F, the scFv fragment (encoded by the polynucleotide construct having the nucleic acid sequence set forth in SEQ ID NO: 39) contained two single-chain molecules, each containing one V_{H} and one V_{L} domain, linked by a peptide linker, but no linker between the two V_{H} domains. As illustrated in FIG. 2G, the scFv hinge fragment (encoded by the polynucleotide construct having the nucleic acid sequence set forth in SEQ ID NO: 41) was identical to the scFv fragment, but further contained the amino acids of the hinge region, providing for disulfide bridge formation between the V_{H} domains. A variation of this fragment (scFv hinge ΔE, encoded by the polynucleotide construct having the nucleic acid sequence set forth in SEQ ID NO: 42) also was generated, which lacked the first amino acid (glutamate) in the hinge region. Finally, as illustrated in FIG. 2H, the scFv Cys19 fragment (encoded by the polynucleotide construct having the nucleic acid sequence set forth in SEQ ID NO: 31) was identical to the scFv fragment, but further contained the isoleucine to cysteine mutation at position 19 of the variable heavy chain. As noted above, this mutation was expected to induce formation of a disulfide bridge between the heavy chain variable regions, which was expected to stabilize the domain exchanged configuration at the heavy chain interface.

### Example 8B

### Generation of the Constructs Encoding the Fragments

(i): 2G12 scFv Tandem (VL-VH-VH-VL-6His-HA) Construct

The 2G12 scFv tandem construct (illustrated in FIG. 2E) was generated in a pET 28 vector (Novagen). As illustrated in FIG. 2E, the scFv tandem polynucleotide construct was designed with the following configuration: V_{L}-V_{H}-V_{H}-V_{L}-6His-HA, where V_{L} represents a nucleic acid encoding the light chain variable region of 2G12, V_{H} represents a nucleic acid encoding the heavy chain variable region of 2G12 antibody, 6His represents a nucleic acid encoding six histidine residues, and HA represents a nucleic acid encoding a hemagglutinin (HA) tag. The scFv tandem polynucleotide further contained a first linker (Linker 1) between the first V_{L} and V_{H} and the second V_{H} and V_{L}, and a second linker (Linker 2), between the two V_{H} domains. The nucleotide sequence of the pET 28 vector containing the nucleic acid encoding the 2G12 scFv tandem is set forth in SEQ ID NO: 40.

To generate the construct, the oligonucleotides listed in Table 14 were ordered from IDT.

TABLE 14

Oligonucleotides for Generation of the 2G12 Domain Exchanged
scFv tandem (VL-VH-VH-VL-6His-HA) construct

| | | |
|---|---|---|
| Oligonucleotide | | |
| Name | Sequence | SEQ ID NO: |

| | | |
|---|---|---|
| OmpA-F: | GTGGCACTGGCTGGTTTCGCTAC | 113 |
| VLL1-R: | GGAGGAAGATCCAGACGAACCACCTTTGATTTCAA | 114 |
| | CACGGGTACCCTG | |
| L1VH-F: | GGTGGCTCGGGCGGTGGTGGCGAAGTTCAGCTGGT | 115 |
| | TGAATCTGGTG | |
| VHL2-R: | CTGCTGCTGCTGCCGGATCCTCCCGGAGAAACGGT | 116 |
| | AACAACGGTAC | |
| L2VH-F: | GGCGGGAGCTCCGGCGGCGGAGAAGTTCAGCTGG | 117 |
| | TTGAATCTGGTG | |
| VHL1-R: | GGAGGAAGATCCAGACGAACCACCCGGAGAAACG | 118 |
| | GTAACAACGGTAC | |
| L1VL-F: | GGTGGCTCGGGCGGTGGTGGCGTTGTTATGACCCA | 119 |
| | GTCTCCGTC | |
| VLSfi-R: | GTGCTGGCCGGCCTGGCCTTTGATTTCAACACGGG | 120 |
| | TACCCTG | |
| Sfi6His-R: | GTGATGGTGCTGGCCGGCCTGGCCTTTG | 121 |
| Linker 1(+): (L1) | GGTGGTTCGTCTGGATCTTCCTCCTCTGGTGGCGGT | 15 |
| | GGCTCGGGCGGTGGTGGC | |
| Linker 1(−): (L1′) | GCCACCACCGCCCGAGCCACCGCCACCAGAGGCG | 122 |
| | GCAGATCCAGACGAACCACC | |
| Linker 2(+): (L2) | GGAGGATCCGGCAGCAGCAGCAGCGGCGGCGGCG | 17 |
| | GCGGGAGCTCCGGCGGCGGA | |
| Linker 2(−): (L2′) | TCCGCCGCCGGAGCTCCCGCCGCCGCCGCCGCTGC | 123 |
| | TGCTGCTGCCGGATCCTCC | |

Four first PCR amplifications (PRC1a-d) were carried out using the template and primers indicated in Table 15 below. For each reaction, the pET Duet vector containing the nucleotide encoding the 2G12 domain exchanged Fab fragment (SEQ ID NO: 124, was used as a template.

For each first PCR, 1 μL of template DNA and 1 μL of each primer were mixed with 1 μL of Advantage HF2 polymerase mix (Clontech) and 1× Advantage HF2 reaction buffer and dNTPs in 50 μL reaction volume. Each amplification was performed with 1 min denaturation at 95° C. and 30 cycles of denaturation at 95° C. for 5 seconds and annealing and extension at 68° C. for 1 min followed by an incubation at 68° C. for 3 minutes. The reaction then was cooled down to 4° C. Each PCR product then was run on a 1 agarose gel and purified using Gel Extraction Kit (Qiagen). The size of each product is indicated in Table 15 below.

TABLE 15
Template and Primers for First PCR Amplifications

| | PCR (product name) | | | |
|---|---|---|---|---|
| | PCR1a | PCR1b | PCR1c | PCR1d |
| template | pETDuet 2G12 | pETDuet 2G12 | pETDuet 2G12 | pETDuet 2G12 |
| | Fab (SEQ ID NO: | Fab (SEQ ID NO: | Fab (SEQ ID NO: | Fab (SEQ ID |
| | 124) | 124) | 124) | NO: 124) |
| 5′ primer(s) | OmpA-F (SEQ | L1 (SEQ ID NO: | L2 (SEQ ID NO: | L1 (SEQ ID |
| (20 μM) | ID NO: 113) | 15): L1VH-F | 17): L2VH-F | NO: 15): |
| | | (SEQ ID NO: | (SEQ ID NO: | L1VL-F (SEQ |
| | | 115) | 117) | ID NO: 119) |
| | | (10:1) | (10:1) | (10:1) |
| 3′ primer(s) | VLL1-R (SEQ ID | VHL2-R (SEQ ID | VHL1-R (SEQ | VLSfi-R (SEQ |
| (20 μM) | NO: 114): L1′ | NO: 116): L2′ | ID NO: 118): L1′ | ID NO: 120) |
| | (SEQ ID NO: | (SEQ ID NO: | (SEQ ID NO: | |
| | 122) (1:10) | 123) (1:10) | 122) (1:10) | |
| Product size | 411 | 446 | 444 | 390 |
| (base pairs | | | | |
| (bp)) | | | | |

Four second PCR (overlap PCR) amplifications then were carried out using the purified products from the first PCR amplifications as templates. The template and primers used in each of the reactions are indicated in Table 16 below. For the reactions, 16 μL total template mixture and 4 μL of each primer were mixed with 4 μL of Advantage HF2 polymerase mix and 1× Advantage HF2 reaction buffer and dNTPs in a 200 μL reaction volume. The amplification was performed with 1 min denaturation at 95° C. and 30 cycles of denaturation at 95° C. for 5 seconds and annealing and extension at 68° C. for 1 min followed by an incubation at 68° C. for 3 minutes. The reaction then was cooled down to 4° C. Each PCR product then was run on a 1% agarose gel and purified using Gel Extraction Kit (Qiagen). The size of each product is indicated in Table 16 below.

TABLE 16
Template and Primers for Second PCR Amplifications

| | PCR (product name) | | | |
|---|---|---|---|---|
| | PCR2a | PCR2b | PCR2c | PCR2d |
| template | PCR1a:PCR1b (1:1) | PCR1a:PCR1b | PCR1c:PCR1d | PCR1c:PCR1d |
| | | (1:1) | (1:1) | (1:1) |
| 5′ primer (20 μM) | OmpA-F | OmpA-F | L2 | L2 |
| | (SEQ ID NO: 113) | (SEQ ID NO: | (SEQ ID NO: | (SEQ ID NO: |
| | | 113) | 17) | 17) |
| 3′ primer (20 μM) | VHL2-R | L2′ | VLSfi-R | Sfi6His-R |
| | (SEQ ID NO: 116) | (SEQ ID NO: | (SEQ ID NO: | (SEQ ID NO: |
| | | 123) | 120) | 121) |
| Product size | 803 | 834 | 813 | 819 |
| (base pairs (bp)) | | | | |

The purified products from the second amplification reaction then were digested and ligated. The product from PCR2a was ligated to the product from PCR2c and the product from PCR2b was ligated to the product from PCR2d. For this process, the products were digested withBam HI restriction endonuclease and purified using a PCR purification column (Qiagen). The digested, purified products then were ligated with T4 DNA ligase (New England Biolabs). The resulting ligated polynucleotides (PCR2a/PCR2c and PCR2b/PCR2d) then were gel-purified and combined.

The combined polynucleotides then were digested with Sfi I (New England Biolabs) and purified using a PCR purification column. A pET28 vector (Novagen) containing AC8 scFv (SEQ ID NO: 79) was digested with Sfi I and gel purified (Qiagen). The Sfi I-digested polynucleotide described above then was inserted into the digested vector by ligation with T4 DNA ligase.

The resulting vector with the inserted polynucleotide then was used to transformed TOP10F′ cells (Invitrogen™ Corporation, Carlsbad, Calif.). The cells were titrated for colony formation on LB agar plates supplemented with 50 μg/mL kanamycin and 20 mM glucose. Following overnight growth at 37° C., individual colonies were picked and grown in 1.2 mL LB medium containing 50 μg/mL kanamycin at 37° C., overnight. DNA from the cultures then was prepared from the cultures using Qiagen miniprep DNA kit. Insertion of the polynucleotide was verified by digesting the DNA with Barn HI/Xho I (New England Biolabs) and visualization on a 1% agarose gel. The nucleotide sequence of the 2G12 scFv tandem (VL-VH-VH-VL-6His-HA) insert was verified by DNA sequencing.

(ii): 2G12 Domain Exchanged scFv (V_{L}-V_{H}) Construct

The 2G12 domain exchanged scFv construct (illustrated in FIG. 2F) was generated in a pET 28 vector (Novagen) by performing a PCR amplification using a PCR product from the procedure used to make the scFv tandem construct, described in Example 8B(i), as a template. As illustrated in FIG. 2F, the scFv polynucleotide construct was designed with the following configuration: V_{L}-V_{H}, where V_{L} represents a nucleic acid encoding the light chain variable region of 2G12, V_{H} represents a nucleic acid encoding the heavy chain variable region of 2G12 antibody. The scFv polynucleotide further contained a linker (Linker 1) between the V_{L} and V_{H}. The nucleotide sequence of the pET 28 vector containing the nucleic acid encoding the 2G12 scFv fragment is set forth in SEQ ID NO: 39.

To generate the scFv polynucleotide, a PCR amplification was carried out using 4 μL of PCR2a from the scFv tandem generation (described in Example 8B(i) above) as a template and 4 μL of primers (20 μM) OmpA-F (SEQ ID NO: 113; GTGGCACTGGCTGGTTTCGCTAC) and VHSfi-R (SEQ ID NO: 125, CCATGGTGATGGTGATGGTGCTGGCCGGCCTGGCCCGGAGAAACGGTAAC AACGGTAC). The PCR was carried out in the presence of 4 μL of Advantage HF2 polymerase mix and 1× Advantage HF2 reaction buffer and dNTP mix (Clontech) in a 200 μL reaction volume. The amplification was performed with 1 min denaturation at 95° C. and 30 cycles of denaturation at 95° C. for 5 seconds and annealing and extension at 68° C. for 1 min followed by an incubation at 68° C. for 3 minutes. The reaction then was cooled down to 4° C. The resulting 815 by polynucleotide was run on a 1% agarose gel and gel-purified using a Gel Extraction Kit (Qiagen).

The resulting scFv product then was ligated into the pET28 vector. For this process, the purified product was digested with Sfi I restriction endonuclease and purified over a PCR purification column (Qiagen). The purified digested product then was ligated into the pET28 vector that had been digested with Sfi I (described in Example 8B(i) above) using T4 DNA ligase (New England Biolabs® Inc.). The product from this ligation reaction was transformed into XL1-Blue cells (Statagene) and the cells titrated for colony formation on LB agar plates supplemented with 50 μg/mL kanamycin and 20 mM glucose. Following overnight growth at 37° C., individual colonies were picked and grown in 1.2 mL LB medium containing 50 μg/mL kanamycin, at 37° C. overnight, DNA from the cultures then was prepared from the cultures using Qiagen miniprep DNA kit. Correct insertion of the polynucleotide was verified by digesting the DNA with Xba I/Xho I (New England Biolabs) and visualization on a 1% agarose gel. The nucleotide sequence of the 2G12 scFv (V_{L}-V_{H}-) insert was verified by DNA sequencing.

(iii): scFv Cys19 Construct

The 2G12 scFv Cys19 construct (illustrated in FIG. 2H) was generated in a pET 28 vector (Novagen) by performing a PCR amplification using the scFv construct, described in Example 8B(i), as a template. As illustrated in FIG. 2H, the scFv Cys19 polynucleotide construct was identical to the scFv polynucleotide, with the exception that the encoded amino acid sequence contained a mutation at the 19^{th} residue of the V_{H} domain from isoleucine to cysteine. Thus, the scFv Cys19 polynucleotide had the following configuration: V_{L}-V_{H}, where V_{L} represents a nucleic acid encoding the light chain variable region of 2G12 and V_{H} represents a nucleic acid encoding the heavy chain variable region of 2G12 antibody, with a cysteine at position 19. The scFv polynucleotide further contained a linker (Linker 1; SEQ ID NO: 15) between the V_{L} and V_{H}. The nucleotide sequence of the pET 28 vector containing the nucleic acid encoding the 2G12 scFv Cys19 fragment is set forth in SEQ ID NO: 31.

Oligonucleotide primers used to construct the pET28 scFv Cys 19 were ordered from IDT. Their sequences are listed in Table 17 below.

TABLE 17
Oligonucleotide Primers for Construction of the
2G12 Domain Exchanged pET28 scFv Cys 19 Fragment

| | | SEQ |
|---|---|---|
| Oligonucleotide | | ID |
| name | Sequence | NO: |
| AgeI-F | CCCTGAAAACCGGTGTTCCGTCTC | 126 |
| Cys19- R | CACCGCAAGACAGGCACAGAGAACCACCAG | 127 |
| Cys19- F | CTGGTGGTTCTCTGTGCCTGTCTTGCGGTG | 128 |
| NcoI25- R | GGTATGCGCCATGGTGATGGTGATG | 129 |

Two first PCR amplifications (Cys a; Cys b) were carried out using the template and primers indicated in Table 18 below. As indicated in the table, for each reaction, the template was the pET28 2G12 domain exchanged scFv vector (SEQ ID NO: 39), generated as described in Example 8B(ii) above.

For each first PCR, 1 μL of template DNA (approximately 4 ng) and 1 μL of each primer were mixed with 1 μL of Advantage HF2 polymerase mix (Clontech) and 1× Advantage HF2 reaction buffer and dNTP mix in 50 μL reaction volume. Each amplification was performed with 1 min denaturation at 95° C. and 26 cycles of denaturation at 95° C. for 5 seconds and annealing and extension at 68° C. for 30 seconds followed by an incubation at 68° C. for 3 minutes. Then the reaction was cooled down to 4° C.

Each PCR product then was run on a 1% agarose gel and purified using Gel Extraction Kit (Qiagen). The size of each product is indicated in Table 18 below.

TABLE 18
Template and Primers for First PCR Amplifications

| | PCR (product name) | |
|---|---|---|
| | Cys a | Cys b |
| template | pET28 2G12 scFv [VL-VH] | pET28 2G12 scFv [VL- |
| | (SEQ ID NO: 39) | VH] (SEQ ID NO: 39) |
| 5′ primer | AgeI-F (SEQ ID NO: 126) | Cys19-F (SEQ ID NO: |
| | | 128) |
| 3′ primer | Cys19-R (SEQ ID NO: 127) | NcoI25-R (SEQ ID NO: |
| | | 129) |
| Product size | 288 | 372 |
| (bp) | | |

A second PCR amplification (Cys c; overlap PCR) was performed using the purified products from the first PCRs described above as templates and primers used in the first reactions. The templates and primers used in the second PCR amplification are indicated in Table 19 below. For this reaction, 4 μL of each template mix and 2 μL of each primer was mixed with 2 μL Advantage HF2 polymerase mix and 1× Advantage H2F reaction buffer and dNTP mix in a 100 μL reaction volume. The amplification was performed with 1 min denaturation at 95° C. and 30 cycles of denaturation at 95° C. for 5 seconds and annealing and extension at 68° C. for 1 min followed by an incubation at 68° C. for 3 minutes. Then the reaction was cooled down to 4° C. The product then was run on a 1% agarose gel, and purified using Gel Extraction Kit (Qiagen). The size of the product also is indicated in Table 19 below.

TABLE 19
Primers and Template for Second PCR Amplification

| | PCR |
|---|---|
| | (product name) |
| | Cys c |
| template | Cys a:Cys b (1:1) |
| 5′ | AgeI-F (SEQ ID NO: |
| | 126) |
| 3′ | NcoI25-R (SEQ ID NO: |
| | 129) |
| Product size | 630 |
| (base pairs) | |

The purified product then was digested and ligated into a pET28 vector. For this process, the product first was digested with Age I and Nco I (New England Biolabs) and purified using a PCR purification column. The digested fragment then was ligated into the pET28 vector containing the scFv polynucleotide (SEQ ID NO: 39, described in Example 8B(ii) above) digested with Age I/Nco I using T4 DNA ligase. The product from the ligation reaction was transformed into TOP10F′ cells (Invitrogen™ Corporation, Carlsbad, Calif.) and the cells titrated for colony formation on LB agar plates supplemented with 50 μg/mL kanamycin and 20 mM glucose. After overnight growth at 37° C., colonies were picked and grown in 1.2 mL LB medium containing 50 μg/mL kanamycin 37° C., overnight. DNA from the cultures was prepared using Qiagen miniprep DNA kit. Verification of correct insertion of the polynucleotide and the presence of cysteine in the 19th amino acid of heavy chain were confirmed by DNA sequence analysis.

(iv): scFv HingeΔE Construct

The scFv hinge ΔE polynucleotide (illustrated in FIG. 2G) was generated in the pET28 vector by carrying out PCR reactions using the pET28 vector containing the nucleotide encoding the 2G12 domain exchanged scFv fragment (SEQ ID NO: 39, described in Example 8B(ii) above) as a template. As shown in FIG. 2G and as described above, the 2G12 scFv hinge ΔE construct was designed to be identical to the scFv fragment, but further contained the nucleic acid encoding the hinge region (without the first glutamate residue), to promote disulfide bond formation between the two heavy chains. The nucleotide sequence of the pET 28 vector containing the nucleic acid encoding the 2G12 scFv hinge ΔE fragment is set forth in SEQ ID NO: 42.

The oligonucleotides listed in Table 20, below were ordered from IDT for the construction of the scFv hinge ΔE construct.

TABLE 20
Oligonucleotides for Construction of the 2G12
Domain Exchanged scFv hinge ΔE construct

| Primer/ | | SEQ |
|---|---|---|
| oligo | | ID |
| name | Sequence | NO: |
| AgeI- F | CCCTGAAAACCGGTGTTCCGTCTC | 126 |
| HingeVH- R | CGCAGCTTTTCGGCGGAGAAACGGTAACAAC | 130 |
| | GGTAC | |
| VHhinge- F | CCGTTTCTCCGCCGAAAAGCTGCGATAAAAC | 131 |
| | CCATACCTGCC | |
| HingeTemplate- F | GCTGCGATAAAACCCATACCTGCCCGCCGTG | 132 |
| | CCCGGGCCAG | |
| HingeTemplate- R | GATGGTGATGGTGCTGGCCGGCCTGGCCCGG | 133 |
| | GCACGGCGGGCAG | |
| NcoI38- R | GCGGCGCCATGGTGATGGTGATGGTGCTGGC | 134 |
| | CGGCCTG | |

Two first PCR amplifications (Hinge a; Hinge b) were carried out using the template and primers indicated in Table 21 below. As indicated in the table, for each reaction, the template was the pET28 2G12 domain exchanged scFv vector (SEQ ID NO: 39), generated as described in Example 8B(ii) above, or one of the template oligonucleotides listed in Table 20 above.

For each first PCR, 1 μL of template DNA (approximately 4 ng) and 1 μL of each primer were mixed with 1 μL of Advantage HF2 polymerase mix (Clontech) and 1× Advantage HF2 reaction buffer and dNTP mix in 50 μL reaction volume. Each amplification was performed with 1 min denaturation at 95° C. and 26 cycles of denaturation at 95° C. for 5 seconds and annealing and extension at 68° C. for 30 seconds followed by an incubation at 68° C. for 3 minutes. Then the reaction was cooled down to 4° C.

Each PCR product then was run on a 1% agarose gel and purified using Gel Extraction Kit (Qiagen). The size of each product is indicated in Table 21 below.

TABLE 21
Template and Primers for First PCR Amplifications

| | PCR (product name) | |
|---|---|---|
| | Hinge a | Hinge b |
| template | pET28 2G12 scFv [VL- | HingeTemplate-F |
| | VH] (SEQ ID NO: 39) | (SEQ ID NO: 131) and |
| | (approximately 4 ng) | HingeTemplate-R |
| | | (SEQ ID NO: 133) |
| | | (1 μM each) |
| 5′ primer | AgeI-F (SEQ ID NO: 126) | VHhinge-F |
| | | (SEQ ID NO: 131) |
| 3′ primer | HingeVH-R (SEQ ID NO: | NcoI38-R |
| | 130) | (SEQ ID NO: 134) |
| Product size (bp) | 600 | 94 |

A second PCR amplification (Hinge c; overlap PCR) was performed using the purified products from the first PCRs described above as templates and primers used in the first reactions. The templates and primers used in the second PCR amplification are indicated in Table 22 below. For this reaction, 4 μL of each template mix and 2 μL of each primer was mixed with 2 μL Advantage HF2 polymerase mix and 1× Advantage H2F reaction buffer and dNTP mix in a 100 μL reaction volume. The amplification was performed with 1 min denaturation at 95° C. and 30 cycles of denaturation at 95° C. for 5 seconds and annealing and extension at 68° C. for 1 min followed by an incubation at 68° C. for 3 minutes. The reaction then was cooled down to 4° C. The product then was run on a 1% agarose gel and purified using Gel Extraction Kit (Qiagen). The size of the product also is indicated in Table 22 below.

TABLE 22
Template and Primers for Second PCR Amplification

| | PCR (product name) |
|---|---|
| | Hinge c |
| template | Hinge a:Hinge b (1:1) |
| 5′ primer | AgeI-F (SEQ ID NO: 126) |
| 3′ primer | NcoI38-R (SEQ ID NO: 134) |
| Product size (bp) | 670 |

The purified product from the Hinge c PCR then was digested and inserted via ligation into the pET28 vector. For this process, the purified product was digested with Age I and Nco I enzymes (New England Biolabs) and purified using a PCR purification column. The digested fragment was ligated into the pET28 vector containing the domain exchanged scFv-encoding polynucleotide (SEQ ID NO: 39), described in Example 8B(ii) above, that had been digested with Age I/Nco I, using T4 DNA ligase (New England Biolabs® Inc.). The product from the ligation reaction then was used to transform TOP10F′ cells (Invitrogen™ Corporation, Carlsbad, Calif.) and the cells titrated for colony formation on LB agar plates containing 50 μg/mL kanamycin and 20 mM glucose. Following growth on the plates overnight at 37° C., colonies were picked and grown in 1.2 mL LB medium containing 50 μg/mL kanamycin at 37° C., overnight, and miniprep DNA was prepared using Qiagen miniprep DNA kit. Verification of correct insertion and presence of the hinge region was confirmed by sequencing the isolated DNA.

(v): scFv Hinge Construct

The scFv hinge polynucleotide (illustrated in FIG. 2G) was generated in the pET28 vector by carrying out PCR reactions using the pET28 vector containing the nucleotide encoding the 2G12 domain exchanged scFv fragment (SEQ ID NO: 39, described in Example 8B(ii) above) as a template. As shown in FIG. 2G and as described above, the 2G12 scFv hinge construct was designed to be identical to the scFv fragment, but further contained the nucleic acid encoding the hinge region (including the first glutamate residue), to promote disulfide bond formation between the two heavy chains. The nucleotide sequence of the pET 28 vector containing the nucleic acid encoding the 2G12 domain exchanged scFv hinge fragment is set forth in SEQ ID NO: 41.

The oligonucleotides listed in Table 23, below were ordered from IDT for the construction of the scFv hinge construct.

TABLE 23
Oligonucleotides for Construction of the Domain
Exchanged 2G12 scFv Hinge Construct

| | | SEQ |
|---|---|---|
| Primer/oligo | | ID |
| name | Sequence | NO: |
| AgeI- F | CCCTGAAAACCGGTGTTCCGTCTC | 126 |
| HingeVH(E)- R | CGCAGCTTTTCGGTTCCGGAGAAACGGTA | 135 |
| | ACAACGGTACCCGGAC | |
| VHhinge(E)- F | CCGTTTCTCCGGAACCGAAAAGCTGCGAT | 136 |
| | AAAACCCATACCTGCC | |
| HingeTemplate F - | GCTGCGATAAAACCCATACCTGCCCGCCG | 132 |
| | TGCCCGGGCCAG | |
| HingeTemplate- R | GATGGTGATGGTGCTGGCCGGCCTGGCCC | 133 |
| | GGGCACGGCGGGCAG | |
| NcoI25- R | GGTATGCGCCATGGTGATGGTGATG | 129 |

Two first PCR amplifications (Hinge(E) a; Hinge(E) b) were carried out using the template and primers indicated in Table 24 below. As indicated in the table, for each reaction, the template was the pET28 2G12 domain exchanged scFv vector (SEQ ID NO: 39), generated as described in Example 8B(ii) above, or one of the Hinge template oligonucleotides listed in Table 23 above.

For each first PCR, 1 μL of template DNA (approximately 4 ng) and 1 μL of each primer were mixed with 1 μL of Advantage HF2 polymerase mix (Clontech) and 1× Advantage HF2 reaction buffer and dNTP mix in 50 μL reaction volume. Each amplification was performed with 1 min denaturation at 95° C. and 26 cycles of denaturation at 95° C. for 5 seconds and annealing and extension at 68° C. for 30 seconds followed by an incubation at 68° C. for 3 minutes. The reaction then was cooled down to 4° C.

Each PCR product then was run on a 1% agarose gel and purified using Gel Extraction Kit (Qiagen). The size of each product is indicated in Table 24 below.

TABLE 24
First PCR Amplifications

| | PCR (product name) | |
|---|---|---|
| | Hinge(E) a | Hinge(E) b |
| template | pET28 2G12 scFv [VL-VH] | HingeTemplate-F |
| | (SEQ ID NO: 39) | (SEQ ID NO: 132) and |
| | (approximately 4 ng) | HingeTemplate-R |
| | | (SEQ ID NO: 133) |
| | | (1 μM each) |
| 5′ primer | AgeI-F | VHhinge(E)-F |
| | (SEQ ID NO: 126) | (SEQ ID NO: 136) |
| 3′ primer | HingeVH(E)-R | NcoI38-R |
| | (SEQ ID NO: 135) | (SEQ ID NO: 134) |
| product size (bp) | 603 | 97 |

A second PCR amplification (Hinge(E) c; overlap PCR) was performed using the purified products from the first PCRs described above as templates and primers used in the first reactions. The templates and primers used in the second PCR amplification are indicated in Table 25 below. For this reaction, 4 μL of each template mix and 2 μL of each primer was mixed with 2 μL Advantage HF2 polymerase mix and 1× Advantage H2F reaction buffer and dNTP mix in a 100 μL reaction volume. The amplification was performed with 1 min denaturation at 95° C. and 30 cycles of denaturation at 95° C. for 5 seconds and annealing and extension at 68° C. for 1 min followed by an incubation at 68° C. for 3 minutes. The reaction then was cooled down to 4° C. The product then was run on a 1% agarose gel and purified using Gel Extraction Kit (Qiagen). The size of the product also is indicated in Table 25 below.

TABLE 25
Second PCR Amplifications

| | PCR (product name) |
|---|---|
| | Hinge(E) c |
| template | Hinge(E) a:Hinge(E) b (1:1) |
| 5′ primer | AgeI-F (SEQ ID NO: 126) |
| 3′ primer | NcoI25-R (SEQ ID NO: 129) |
| Product size (bp) | 673 |

The purified product from the Hinge(E) c PCR then was digested and inserted via ligation into the pET28 vector. For this process, the purified product was digested with Age I and Nco I enzymes (New England Biolabs) and purified using a PCR purification column. The digested fragment was ligated into the pET28 vector containing the domain exchanged scFv-encoding polynucleotide (SEQ ID NO: 39), described in Example 8B(ii) above, that had been digested with Age I/Nco I, using T4 DNA ligase. The product from the ligation reaction then was used to transform TOP10F′ cells (Invitrogen™ Corporation, Carlsbad, Calif.) and the cells titrated for colony formation on LB agar plates containing 50 μg/mL kanamycin and 20 mM glucose. Following growth on the plates overnight at 37° C., colonies were picked and grown in 1.2 mL LB medium containing 50 μg/mL kanamycin at 37° C. overnight, and miniprep DNA was prepared using Qiagen miniprep DNA kit. Verification of correct insertion and presence of the hinge region was confirmed by sequencing the isolated DNA.

(vi): 2G12 Fab Cys19 Construct

The 2G12 Fab Cys19 construct (illustrated in FIG. 2C) was generated in a pET Duet vector (Novagen). As illustrated in FIG. 2C, the 2G12 Fab Cys19 polynucleotide construct was identical to the 2G12 Fab fragment, with the exception that the polynucleotide was mutated such that an isoleucine to cysteine substitution occurred at position 19 of the heavy chain amino acid sequence encoded by the construct; this mutation was made to promote formation of a disulfide bridge between the two heavy chain variable regions in the folded domain exchanged fragment. The 2G12 Fab Cys19 polynucleotide contained a linker (Linker 1; SEQ ID NO: 15) between the V_{L} and V_{H} encoding sequences. The nucleotide sequence of the pET Duet vector containing the nucleic acid encoding the 2G12 Fab Cys19 is set forth in SEQ ID NO: 29.

In addition to oligonucleotides listed elsewhere in this Example, the oligonucleotides listed in Table 26 below were ordered from IDT, for generation of the 2G12 Fab Cys19 construct.

TABLE 26
Oligonucleotides for Generating 2G12 Domain
Exchanged Fab Cys19

| Primer Name | Sequence | SEQ ID NO: |
|---|---|---|
| NdeIVH- F | GGAGATATACATATGAA | 137 |
| | ATACCTATTGCCTAC | |
| XhoIHA26- R | TACCAGACTCGAGCTAA | 138 |
| | GAAGCGTAG | |

Two first PCR amplifications (Fab Cys19 a and Fab Cys19 b) were carried out using the template and primers indicated in Table 27 below. For each reaction, the pET Duet vector containing the nucleotide encoding the 2G12 domain exchanged Fab fragment (SEQ ID NO: 124) was used as a template.

For each first PCR, 1 μL of template DNA (approximately 10 ng) and 1 μL of each primer were mixed with 1 μL of Advantage HF2 polymerase mix (Clontech) and 1× Advantage HF2 reaction buffer and dNTPs in 50 μL reaction volume. Each amplification was performed with 1 min denaturation at 95° C. and 26 cycles of denaturation at 95° C. for 5 seconds and annealing and extension at 68° C. for 30 seconds followed by an incubation at 68° C. for 3 minutes. The reaction then was cooled down to 4° C. Each PCR product then was run on a 1% agarose gel and purified using Gel Extraction Kit (Qiagen). The size of each product is indicated in Table 27 below.

TABLE 27
First PCR Amplifications

| | PCR (product name) | |
|---|---|---|
| | Fab Cys19 a | Fab Cys19 b |
| template | 2G12 Fab in pETDuet vector | 2G12 Fab in pETDuet |
| | (SEQ ID NO: 124) | vector (SEQ ID NO: |
| | | 124) |
| 5′ primer (20 μM) | NdeIVH-F (SEQ ID NO: 137) | Cys19-F |
| | | (SEQ ID NO: 128) |
| 3′ primer (20 μM) | Cys19-R | XhoIHA26-R |
| | (SEQ ID NO: 127) | (SEQ ID NO: 138) |
| Product size (bp) | 148 | 717 |

A second PCR amplification (Fab Cys19 c, an Overlap PCR) was performed using the purified products from the first PCR as templates. The primers/templates used in this second PCR are indicated in Table 28 below. For the reaction, 4 μL of template mix and 2 μL of each primer were mixed with 2 μL of Advantage HF2 polymerase mix in 1× Advantage H2F reaction buffer and dNTP in 100 μL reaction volume. The amplification was performed with 1 min denaturation at 95° C. and 30 cycles of denaturation at 95° C. for 5 seconds and annealing and extension at 68° C. for 1 min followed by an incubation at 68° C. for 3 minutes. The reaction then was cooled down to 4° C. The size of the product is indicated in Table 28 below. The product was run on a 1% agarose gel and purified by gel extraction.

TABLE 28
Second PCR Amplification

| | PCR (product name) |
|---|---|
| | Fab Cys19 c |
| template | Fab Cys a:Fab Cys b |
| | (1:1) |
| 5′ primer (20 μM) | NdeIVH-F |
| | (SEQ ID NO: 137) |
| 3′ primer (20 μM) | XhoIHA26-R |
| | (SEQ ID NO: 138) |
| Product size (bp) | 835 |

The purified product then was digested and inserted via ligation into the pETDuet 2G12 Fab vector. For this process, the product was digested with Nde I and Xho I enzymes (New England Biolabs) and purified using a PCR purification column. The digested product then was ligated into the pETDuet 2G12 Fab vector (SEQ ID NO: 231), that had been digested with Nde I/Xho I, using T4 DNA ligase. The product of this ligation reaction was used to transform TOP10F′ cells (Invitrogen™ Corporation, Carlsbad, Calif.) and the cells titrated for colony formation on LB agar plates supplemented with 100 μg/mL ampicillin and 20 mM glucose. Following overnight growth at 37° C., colonies were picked and grown in 1.2 mL LB medium containing 50 μg/mL ampicillin, overnight at 37° C., and DNA from the culture prepared using Qiagen miniprep DNA kit. The correct insertion of the 2G12 Fab Cys19 polynucleotide and the presence of the cysteine codon in the sequence at the position encoding the 19^{th} amino acid of the heavy chain were confirmed by DNA sequence analysis.

(vii): 2G12 Fab Hinge Construct

The 2G12 Fab hinge construct (illustrated in FIG. 2B) was generated in a pET Duet vector (Novagen). As illustrated in FIG. 2B, the 2G12 Fab hinge polynucleotide construct was identical to the 2G12 Fab fragment, with the exception that the construct further included the nucleic acid encoding the hinge region of the 2G12 antibody, thereby facilitating the formation of a disulfide bridge in the encoded fragment between the two heavy chains. The 2G12 Fab hinge polynucleotide contained a linker (Linker 1 SEQ ID NO: 15) between the V_{L} and V_{H} encoding sequences. The nucleotide sequence of the pET Duet vector containing the nucleic acid encoding the 2G12 Fab hinge fragment is set forth in SEQ ID NO: 38.

The oligonucleotides listed in Table 29 below were ordered from IDT, for generation of the 2G12 Fab hinge construct.

TABLE 29
Oligonucleotides for Generation of the Domain
Exchanged 2G12 Fab Hinge Construct

| Oligonucleotide name | sequence | SEQ ID NO: |
|---|---|---|
| HingeCH1-R | CAGGTATGGGTTTTATC | 139 |
| | GCAGCTTTTCGGTTC | |
| | AACTTTCTTGTC | |
| CH1Hinge-F | CCGAAAAGCTGCGATA | 140 |
| | AAACCCATACCTGCCC | |
| | GCCGTGC | |
| HingeHisTemplate-F | CCCATACCTGCCCGCC | 141 |
| | GTGCCCGCACCATCACCA | |
| | TCACCATGGCG | |
| HingeHisTemplate-R | GTCCGGAACGTCGTA | 142 |
| | CGGGTATGCGCCATGGT | |
| | GATGGTGATGGTGCG | |
| XhoIHA-R | ACCAGACTCGAGCT | 143 |
| | AAGAAGCGTAGTCCGGAA | |
| | CGTCGTACGGGTATG | |

Two first PCR amplifications (Fab hinge a and Fab hinge b) were carried out using the templates and primers indicated in Table 30 below. As indicated, for the Fab hinge a reaction, the pET Duet vector containing the nucleotide encoding the 2G12 domain exchanged Fab fragment (SEQ ID NO: 124) was used as a template.

For each first PCR, 1 μl of template DNA (approximately 10 ng) and 1 μL of each primer were mixed with 1 μL of Advantage HF2 polymerase mix (Clontech) in 1× Advantage HF2 reaction buffer and dNTPs in 50 μL reaction volume. The amplification of “Fab hinge a” was performed with 1 min denaturation at 95° C. and 30 cycles of denaturation at 95° C. for 5 seconds, annealing at 60° C. for 10 seconds, and extension at 68° C. for 30 seconds followed by an incubation at 68° C. for 3. The reaction then was cooled down to 4° C. The amplification of “Fab hinge b” was performed with 1 min denaturation at 95° C. and 26 cycles of denaturation at 95° C. for 5 seconds and annealing and extension at 68° C. for 30 seconds followed by an incubation at 68° C. for 3 minutes. The reaction then was cooled down to 4° C. Each PCR product then was run on a 1% agarose gel and purified using Gel Extraction Kit (Qiagen). The size of each product is indicated in Table 30 below.

TABLE 30
First PCR Amplifications

PCR (product name)

| | Fab hinge a | Fab hinge b |
|---|---|---|
| template | pETDuet 2G12 Fab | HingeHisTemplate-F |
| | (SEQ ID NO: 124) | (SEQ ID NO: 141) and |
| | | HingeHisTemplate-R |
| | | (SEQ ID NO: 142) |
| | | (0.2 μM each) |
| 5′ primer (20 μM) | NdeIVH-F | CH1hinge-F |
| | (SEQ ID NO: 137) | (SEQ ID NO: 140) |
| 3′ primer (20 μM) | HingeCH1-R | XhoIHA-R |
| | (SEQ ID NO: 139) | (SEQ ID NO: 143) |
| Product size (bp) | 774 | 111 |

A second PCR amplification (Fab hinge, an Overlap PCR) was performed using the purified products from the first PCR as templates. The primers/templates used in this second PCR are indicated in Table 31 below. For the reaction, 4 μL of template mix and 2 μL of each primer were mixed with 2 μL of Advantage HF2 polymerase mix in 1× Advantage H2F reaction buffer and dNTP in 100 μL reaction volume. The amplification was performed with 1 min denaturation at 95° C. and 30 cycles of denaturation at 95° C. for 5 seconds, annealing at 60° C. for 10 seconds, and extension at 68° C. for 30 seconds followed by an incubation at 68° C. for 3 minutes. The reaction then was cooled down to 4° C. The size of the product is indicated in Table 31 below. The product was run on a 1% agarose gel and purified by gel extraction.

TABLE 31
Second PCR Amplifications

| | PCR (product name) |
|---|---|
| | Fab hinge |
| template | Fab hinge a:Fab hinge b |
| | (1:1) |
| 5′ primer (20 μM) | NdeIVH-F |
| | (SEQ ID NO: 137) |
| 3′ primer (20 μM) | XhoIHA26-R |
| | (SEQ ID NO: 138) |
| Fragment size (bp) | 856 |

The purified product then was disgusted and inserted into the pETDuet vector containing 2G12 Fab. For this process, the purified product was digested with the Nde I and Xho I restriction endonucleases (New England Biolabs) and purified using a PCR purification column. The purified digested product then was ligated into the pETDuet vector containing the nucleotide encoding the 2G12 domain exchanged Fab fragment (SEQ ID NO: 124), that had been digested with Nde I/Xho I, using T4 DNA ligase.

The product of this ligation reaction then was transformed into TOP10F′ cells (Invitrogen™ Corporation, Carlsbad, Calif.) and the cells titrated for colony formation on LB agar plates supplemented with 100 μg/mL ampicillin and 20 mM glucose. Following overnight growth at 37° C., colonies were picked and grown in 1.2 mL LB medium containing 50 μg/mL ampicillin overnight at 37° C., and culture DNA prepared using Qiagen miniprep DNA kit. Verification of correct insertion of the product and the presence of the hinge region in the construct was carried out by sequencing the prepared DNA.

(viii): 2G12 scFab ΔC2 Cys19 Construct

The 2G12 scFab ΔC2 Cys19 construct (illustrated in FIG. 2D) was generated in a pET28 vector (Novagen). As illustrated in FIG. 2D, the 2G12 scFab ΔC2 Cys19 polynucleotide construct was identical to the 2G12 Fab Cys19 fragment, with the exception that the construct was mutated such that other amino acids were substituted for two cysteines in the encoded constant regions (removing the disulfide bridges between heavy and light chain) and a linker was added, linking the V_{H} and C_{L} domains. The nucleotide sequence of the pET 28 vector containing the nucleic acid encoding the 2G12 scFab ΔC2 Cys19 fragment is set forth in SEQ ID NO: 30.

The oligonucleotides listed in Table 32 below were ordered from IDT, for generation of the 2G12 scFab ΔC2 Cys19 construct. The BamHISacI(+) and SacIBamHI(−) oligonucleotides were generated with 5′ phosphate groups.

TABLE 32
Oligonucleotides for Generation of the Domain
Exchanged 2G12 scFab ΔC2 Cys19 Construct

| | | SEQ ID |
|---|---|---|
| Oligonucleotide Name | Sequence | NO: |
| XbaIVL-F | GGGGAATTGTGAGCGGATAAC | 144 |
| | AATTC | |
| BamHICK-R | CCGCCACCGGATCCACCACC | 145 |
| | AGATTCACCACGGTTGAAAGA | |
| | TTTGGTAACC | |
| SacIVH-F | GCGGTGGGAGCTCCGGTGAAG | 146 |
| | TTCAGCTGGTTGAATCTGGTG | |
| HingeCH1deltaC-R | CTGGCCGGCCTGGCCGCTGC | 147 |
| | TGCCAGATTTCGGTTCAACTT | |
| | TCTTGTCAAC | |
| NcoIHinge-R | GTATGCGCCATGGTGATGGT | 148 |
| | GATGGTGCTGGCCGGCCTGGCC | |
| | GCTG | |
| BamHISacI(+) | GATCCGGTGGCGGCAGCGAAG | 27 |
| | GTGGTGGCAGCGAAGGTGGCG | |
| | GTAGCGAAGGTGGCGGCAGCG | |
| | AAGGCGGCGGTAGCGGTGGG | |
| | AGCT | |
| SacIBamHI(−) | CCCACCGCTACCGCCGCCTT | 149 |
| | CGCTGCCGCCACCTTCGCTAC | |
| | CGCCACCTTCGCTGCCACC | |
| | ACCTTCGCTGCCGCCACCG | |

First, a light chain polynucleotide (scFab ΔC2 Cys19 LC) was generated by PCR amplification using the template and primers indicated in Table 33, below. The template was the pET Duet vector containing the 2G12 Fab polynucleotide (SEQ ID NO: 124). For the reaction, 1 μL template (approximately 10 ng) and 1 μL of each primer were mixed with 1 μL of Advantage HF2 polymerase mix in 1× Advantage HF2 reaction buffer and dNTP in a 50 μL reaction volume. The amplification was performed with 1 minute denaturation at 95° C. and 30 cycles of denaturation at 95° C. for 5 seconds, annealing at 60° C. for 10 seconds, and extension at 68° C. for 30 seconds followed by an incubation at 68° C. for 3 minutes. The reaction then was cooled down to 4° C. The size of the product is indicated in the Table 33, below. The product then was run on a 1% agarose gel and purified using a gel extraction kit.

TABLE 33
PCR Amplification of Light Chain Polynucleotide

| | PCR (product name) |
|---|---|
| | scFab ΔC2 Cys19 LC |
| template | 2G12 Fab in pETDuet vector |
| | (SEQ ID NO: 124) |
| 5′ primer (20 μM) | XbaIVL-F |
| | (SEQ ID NO: 144) |
| 3′ primer (20 μM) | BamHICK-R |
| | (SEQ ID NO: 145) |
| Product size (bp) | 795 |

The light chain product then was digested and inserted into the pET28 vector containing the 2G12 scFv tandem polynucleotide. For this process, the purified product was digested with Xba I and Bam HI restriction endonucleases (New England Biolabs®, Inc.) and purified using a PCR purification column. The digested product then was ligated into the pET28 vector containing the 2G12 domain exchanged scFv tandem polynucleotide (SEQ ID NO: 40), described in Example 8B(i) above, that had been digested with Xba I/Bam HI, using T4 DNA ligase.

The product of this ligation reaction was used to transform TOP10F′ cells (Invitrogen™ Corporation, Carlsbad, Calif.). The cells were titrated for colony formation on LB agar plates supplemented with 50 μg/mL kanamycin and 20 mM glucose. Following overnight growth at 37° C., colonies were picked and grown in 1.2 mL LB medium containing 50 μg/mL kanamycin, overnight at 37° C., and DNA from the cultures prepared using Qiagen miniprep DNA kit. Verification that the product had been correctly inserted into the vector was confirmed by DNA sequence analysis.

Next, a heavy chain polynucleotide (scFab μC2 Cys19HCl) was generated by PCR amplification using the template and primers indicated in Table 34, below. The template was the pET Duet vector containing the 2G12 Fab Cys 19 polynucleotide (SEQ ID NO: 29), described in Example 8B(vi), above. For the reaction, 1 μl of the template DNA (approximately 10 ng) was amplified with 1 μL of each primer in the presence of 1 μL of Advantage HF2 polymerase mix in 1× Advantage HF2 reaction buffer and dNTP in a 50 μL reaction volume. The amplified product was run on a 1% agarose gel and purified using a Gel Extraction kit.

TABLE 34
PCR Amplification of Heavy Chain Polynucleotide

| | PCR (product name) |
|---|---|
| | scFab μC2 Cys19 HC1 |
| template | 2G12 Fab Cys 19 in pETDuet vector |
| | (SEQ ID NO: 29) |
| 5′ primer (20 μM) | SacIVH-F |
| | (SEQ ID NO: 146) |
| 3′ primer (20 μM) | HingeCH1ΔC-R |
| | (SEQ ID NO: 147) |
| Product size (bp) | 716 |

Next, a second heavy chain fragment (scFab ΔC2 Cys19 HC2), was generated by PCR amplification, using the first heavy chain product as a template. The primers and template, as well as size of the product, are indicated in Table 35, below. For the reaction, 2 μL of purified scFab μC2 Cys19 HCl product from the previous step was amplified with 2 μL of each primer in the presence of 2 μL of Advantage HF2 polymerase mix and dNTP in 1× Advantage HF2 polymerase reaction buffer in a 100 μL reaction volume. The product was run on a 1% agarose gel and purified by Gel Extraction.

TABLE 35
PCR Amplification of Second Heavy Chain Polynucleotide

| | PCR (product name) |
|---|---|
| | scFab ΔC2 Cys19 HC2 |
| template | scFab ΔC2 Cys19 HC1 |
| 5′ primer (20 μM) | SacIVH-F |
| | (SEQ ID NO: 146) |
| 3′ primer (20 μM) | NcoIHinge-R |
| | (SEQ ID NO: 148) |
| Product size (bp) | 743 |

Next, a linker (GATCCGGTGGCGGCAGCGAAGGTGGTGGCAGCGAAGGTGGCGGTAGCGA AGGTGGCGGCAGCGAAGGCGGCGGTAGCGGTGGGAGCT, SEQ ID NO: 27), for insertion between the V_{H} and C_{L} domains was generated by mixing the BamHISacI(+) (SEQ ID NO: 27) and SacIBamHI(−) (SEQ ID NO: 149) oligonucleotides under conditions whereby they hybridized through complementary regions: in the presence of 50 mM NaCl, by denaturing at 90° C. for 5 min and slowly cooling down to ambient temperature (approximately 25° C.). The linker contained Sac I and BamH1 restriction site overhangs for ligation into the vector with the heavy chain.

Next, the heavy chain product (scFab ΔC2 Cys19 HC2) was digested and inserted into the pET28 vector into which the light chain fragment had been inserted as described in this subsection above. For this process, the light chain and the heavy chain product was digested with Sac I and Nco I restriction enzymes (New England Biolabs®, Inc.) and ligated, along with the linker prepared above, using T4 DNA ligase, into the pET28 vector into which the light chain had been introduced (described in this subsection above), that had been digested with Bam HI and Nco I.

The product of this ligation reaction was used to transform TOP10F′ cells (Invitrogen™ Corporation, Carlsbad, Calif.) and the cells titrated for colony formation on LB agar plates supplemented with 50 μg/mL kanamycin and 20 mM glucose. Following overnight growth at 37° C., colonies were picked and grown in 1.2 mL LB medium containing 50 μg/mL kanamycin, overnight at 37° C., and DNA from the culture was prepared using Qiagen miniprep DNA kit. The correct insertion of the fragment was confirmed by DNA sequence analysis.

(ix): Generation of Alternate Linker 2 Library for 2G12 scFv Tandem (VL-VH-VH-VL-6His-HA)

In addition to the original linker 2, used in generating the scFv tandem, detailed in Example 8B(i), above, which had 18 amino acids, the following oligonucleotides (listed in Table 36, below) were ordered from Integrated DNA Technologies (IDT) (Coralville, Iowa) to make a library of linkers with 16 to 20 amino acids. Each oligonucleotide contained a 5′ phosphate group.

TABLE 36
Oligonucleotides for Linker Library

| Oligo | | SEQ ID |
|---|---|---|
| name | Sequence | NO: |
| L216F | GATCCGGCAGCAGCAGCAGCGGCGGCGGGAGCT | 150 |
| L216R | CCCGCCGCCGCTGCTGCTGCTGCCG | 151 |
| L217F | GATCCGGCAGCAGCAGCAGCGGCGGCGGCGGGAGCT | 152 |
| L217R | CCCGCCGCCGCCGCTGCTGCTGCTGCCG | 153 |
| L219F | GATCCAGCGGCAGCAGCAGCAGCGGCGGCGGCGGCG | 154 |
| | GGAGCT | |
| L219R | CCCGCCGCCGCCGCCGCTGCTGCTGCTGCCGCTG | 155 |
| L220F | GATCCAGCGGCGGCAGCAGCAGCAGCGGCGGCGGC | 156 |
| | GGCGGGAGCT | |
| L220R | CCCGCCGCCGCCGCCGCTGCTGCTGCTGCCGCCGCTG | 157 |

Four linker oligonucleotide duplexes (L216, L217, L219, L220) were made by mixing 5′ oligonucleotides and 3′ oligonucleotides, as indicated in Table 37, below, under conditions whereby they formed duplexes by hybridizing through complementary regions: in the presence of 50 mM NaCl, by denaturing at 90° C. for 5 min and slowly cooling down to ambient temperature (approximately 25° C.).

TABLE 37
Linker Oligonucleotide Duplexes

| Linker name | | | |
|---|---|---|---|
| L216 | L217 | L219 | L220 |

| | | | | |
|---|---|---|---|---|
| 5′ oligonucleotide | L216F | L217F | L219F | L220F |
| (100 μM) | (SEQ ID | (SEQ ID NO: | (SEQ ID NO: | (SEQ ID NO: |
| | NO: 150) | 152) | 154) | 156) |
| 3′ oligonucleotide | L216R | L217R | L219R | L220R |
| (100 μM) | (SEQ ID | (SEQ ID NO: | (SEQ ID NO: | (SEQ ID NO: |
| | NO: 151) | 153) | 155) | 157) |
| Linker length | 16 | 17 | 19 | 20 |
| (amino acid | | | | |
| residues) | | | | |
| Nucleotide | GGAGGAT | GGAGGATCC | GGAGGATCC | GGAGGATCCA |
| sequence encoding | CCGGCAG | GGCAGCAGC | AGCGGCAGC | GCGGCGGCAG |
| linker | CAGCAGC | AGCAGCGGC | AGCAGCAGC | CAGCAGCAGC |
| | AGCGGCG | GGCGGCGGG | GGCGGCGGC | GGCGGCGGCG |
| | GCGGGAG | AGCTCCGGC | GGCGGGAGC | GCGGGAGCTC |
| | CTCCGGC | GGCGGA | TCCGGCGGC | CGGCGGCGGA |
| | GGCGGA | | GGA | |
| SEQ ID NO of | 19 | 21 | 23 | 25 |
| nucleotide sequence | | | | |
| encoding linker | | | | |
| SEQ ID NO of | 20 | 22 | 24 | 26 |
| amino acid | | | | |
| sequence of | | | | |
| polypeptide linker | | | | |

Each linker oligonucleotide duplex was inserted (via ligation using T4 DNA ligase into the pET28 vector containing the 2G12 scFv tandem polynucleotide (SEQ ID NO: 40), described in Example 8B(i) above, which had been cut with Barn HI and Sac I restriction endonucleases, thus partially replacing the sequence of the original Linker 2 in that construct.

### Example 8C

### Expression and Analysis of 2G12 Antibody Fragment Polypeptides in Bacterial Host Cells

(i) Polypeptide Expression

To evaluate expression of the various 2G12 domain exchanged polypeptide antibody fragments described in Example 8A from vectors generated as described in Example 8B, protein expression was induced in host cells transformed with the vectors. First, for protein expression of the 2G12 Fab fragment, 50 μL BL21 chemically competent E. coli cells were transformed with 100 ng of the pETDuet 2G12 domain exchanged Fab vector (SEQ ID NO: 124) and plated onto agar plates supplemented with kanamycin (30 ug/mL). Following overnight growth at 37° C., a single colony was picked and used to inoculate 50 mL of LB medium, supplemented with 30 ug/mL kanamycin. The culture was grown at 37° C., with shaking at 250 rpm, until the O.D. reached 0.6. To induce protein expression, 1 mM IPTG was added to the culture, which then was maintained at 30° C., with shaking at 250 rpm, overnight. The bacteria then were isolated by centrifugation (3000 rpm, 10 minutes) and resuspended in 1 mL PBS. To lyse the cells, the pellet was freeze-thawed three times in a dry ice/ethanol bath. The lysate then was centrifuged at 16,000×g for 20 minutes at 4° C. and the pellet discarded.

1 mL of the cleared supernatant then was separated on a Sephacryl S-200 HiPrep 16×60 size exclusion column (Amersham) by FPLC. Molecular weight standards (1 kb Plus DNA marker, Invitrogen™ Corporation, Carlsbad, Calif.) were used to determine molecular weight of the fraction proteins, by correlation with elution time. Protein from the fractions obtained from the column was tested for the presence 2G12 by ELISA binding against gp120, as described in Example 8D, below. Based on the molecular weight standards, it was determined that the fractions having reactivity in the ELISA binding assay with gp120 contained protein of an apparent size of approximately 92.5 Kda, the appropriate size of the 2G12 Fab fragment.

The same conditions and host cells were used to express other 2G12 fragments described in the above Examples. The results are listed in Table 38, below.

In Table 38, in the column labeled “Expression in E. coli,” a “++” indicates that the fragment was successfully expressed from the construct in bacterial host cells, using the conditions, methods and host cells described in this Example; a “−” indicates that the fragment was not successfully expressed in bacterial host cells using the conditions, methods and host cells described in this Example; and “NA” indicates that expression from this construct was not attempted.

As shown in Table 38, In addition to the 2G12 Fab fragment, the vectors containing nucleotide sequence encoding the domain exchanged 2G12 Fab hinge (SEQ ID NO: 38), 2G12 domain exchanged scFv tandem (SEQ ID NO: 40); 2G12 domain exchanged scFv (SEQ ID NO: 39) and the 2G12 domain exchanged scFv hinge E (SEQ ID NO: 41) fragments all were used to successfully express antibody fragments in bacterial cells, using the approach used to express the 2G12 Fab fragment. Expression of the 2G12 scFab ΔC2 Cys19 fragment in bacterial host cells was not attempted (indicated by ND in Table 38, below).

These data are expressed in Table 38. This table lists each 2G12 domain exchanged fragment (Fab, Fab hinge, Fab Cys19, scFabΔC2 Cys19, scFv tandem, scFv, scFv hinge and scFv Cys19) for which a construct was generated, as described in this and the previous Examples.

These data are exemplary, showing expression from particular constructs in a particular study with exemplary cell culture conditions and host cells and other parameters. Thus, the data are not comprehensive and are not meant to indicate that other constructs, including the constructs for which a “−” is listed in Table 38, cannot be used for expressing domain exchanged fragments in these or any other host cells under these or any other conditions.

TABLE 38
Expression of 2G12 Domain Exchange Fragments in Bacterial Host
Cells and Binding of the Expressed Antibodies to Antigen

| 2G12 Domain | | |
|---|---|---|
| Exchanged | Expression in | Binding to |
| Fragment | E. coli | gp120 |
| Fab | ++ | ++ |
| Fab Hinge | ++ | ++ |
| Fab Cys19 | − | − |
| scFabΔC² | ND | ND |
| Cys19 | | |
| scFv tandem | ++ | + |
| scFv | ++ | − |
| scFv hinge | ++ | + |
| scFv Cys19 | − | − |

(ii) Analysis of Antigen Specificity Using ELISA-Based Binding Assay

Polypeptides expressed from the host cells transformed with vectors described in Example 8C(i) were assessed in an ELISA-based antigen binding assay similar to the one described in Example 6C, above. Using this assay, the ability of each fragment to bind the 2G12 cognate antigen, gp120, was evaluated and compared to the ability of the 2G12 Fab fragment to bind the antigen. Polypeptides expressed from the AC8 scFv construct, described in Example 1, above were used as controls.

First, DNA (˜200 ng) from the various constructs was used to transform chemically competent BL21 (DE3) cells (Invitrogen™ Corporation, Carlsbad, Calif., Carlsbad, Calif.). Single colonies of the transformants were grown overnight at 37° C. in LB media containing the appropriate antibiotic (Fab constructs: 50 μg/mL ampicillin; ScFv constructs: 25 μg/mL kanamycin), to allow secretion of domain exchanged fragments expressed from the constructs into the culture supernatant. The cultures then were centrifuged at 3,000 rpm for 15 min. The cell pellets were resuspended in 1 mL PBS and subjected to five freeze-thaw cycles. Insoluble material was removed by centrifugation at 14,000 rpm for 20 min.

The resulting PBS solutions contained the domain exchanged antibody fragments that were secreted into the supernatant during overnight growth, as well as antibodies harbored within the cells.

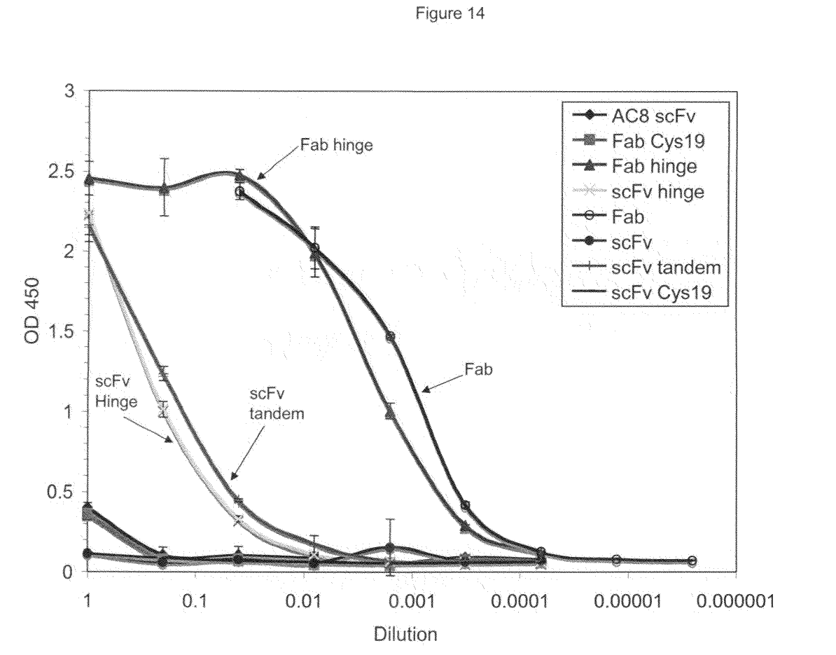

In order to demonstrate that the expressed fragments could bind the 2G12 antigen, gp120, the ELISA-based assay such as described in Example 6C was performed on the PBS solutions containing the fragments. Briefly, gp120-coated plates were incubated with serially diluted solutions of the polypeptide-containing PBS solutions from the previous step (1:5 serial dilutions), using the same binding conditions as described in Example 6C, above. Each sample was added to the plate in triplicate. Following binding, the plates were washed 10× with PBS containing 0.05% Tween to remove unbound proteins. Bound antibody fragments were detected using HRP-conjugated anti-HA, followed by a substrate, which was detected by taking absorbance readings, as described in Example 6C above. The data are summarized in Table 38, above and in FIG. 14.

In Table 38, in the column labeled “Binding to gp120,” “++” indicates that polypeptides from a particular sample bound strongly to the gp120 antigen as assessed using these experimental conditions; “+” indicates that polypeptides from a particular sample bound moderately well to the gp120 antigen as assessed using these experimental conditions; and “−” indicates that the polypeptides from a particular sample exhibited weak binding (no detectable absorbance compared to control level) to the gp120 antigen as assessed using these experimental conditions.

As shown in Table 38, under these experimental conditions, the polypeptides recovered from the cells transformed with the 2G12 domain exchanged Fab and the 2G12 domain exchanged Fab hinge constructs (vectors having the nucleotide sequences set forth in SEQ ID Nos: 124 and 38, respectively) exhibited strong binding to gp120, while the polypeptides recovered from the cells transformed with the domain exchanged 2G12 scFv tandem and 2G12 scFv hinge constructs (vectors having the nucleotide sequences set forth in SEQ ID Nos: 40 and 41, respectively), exhibited moderate binding (absorbance values less than half those for the Fab and Fab hinge proteins at comparable dilutions), and that the polypeptides recovered from the Fab Cys19, scFv Cys19 and scFv constructs exhibited weak binding (no detectable absorbance over that observed for polypeptides from the control sample (AC8 scFv)). FIG. 14 shows a graph, where the Y axis represents absorbance at 450 nm and the X axis represents dilution of the solution containing the antibody fragments. The binding curves for the domain exchanged fragments that exhibited moderate or strong binding to gp120 are labeled on the graph, with arrows pointing to the appropriate curve. The lack of detectable binding in the Fab Cys19 and scFv Cys19 samples likely was due to poor protein expression from these constructs under particular conditions as described in Example 8C(i) above.

These data are exemplary, showing binding of polypeptides from particular samples in a particular study with exemplary cell culture conditions, host cells, reagents and other parameters. Thus, the data are not comprehensive and are not meant to indicate that other constructs, including the constructs for which a “−” is listed in Table 38, cannot be used to express domain exchanged fragments that bind cognate antigen in these or any other host cells under these or any other conditions and parameters.

### Example 8D

### Phage Display of the Fragments

Example 2B, above, describes the generation of phage display 2G12 pCAL G13 vector for phage display of the 2G12 Fab fragment. Example 4, above, describes the successful expression of the 2G12 domain exchanged fragment, using this vector, as part of a gene III fusion protein on phage surface. Examples 4B and 4C and describe precipitation of phage displaying the 2G12 Fab fragment, and verification of its ability to specifically bind gp120 antigen using the ELISA-based assay on precipitated phage. Further, as described in Examples 6 and 7, panning was used to selectively enrich for antigen binding (2G12) version of the Fab fragment when a vector encoding this fragment was spiked in to a mixture of vector encoding a non-binding (3-Ala) Fab fragment, and the mixture was used to transform cells and display phage (Example 6), and when it was spiked in to a randomized nucleic acid library containing randomized 2G12 variant-encoding nucleic acids, and the mixture used to transform cells and induce phage display (Example 7). These results indicate that the provided compositions and methods can be used to generate domain exchanged antibodies displayed on phage, including phage display libraries of domain exchanged antibodies and fragments thereof (such as fragments described in Example 8), and to select domain exchanged antibodies from the libraries having particular properties, such as ability to bind to a particular antigen.

### Example 9

### Generation of the 2G12 3Ala LC pCAL IT* Vector

The 2G12 pCAL IT* vector was further modified by the introduction of three alanine amino acid substitutions in the light chain CDR3 of 2G12. The modification of the 2G12 pCAL IT* vector was carried out using overlapping PCR mutagenesis and cloning at the SgrAI and Pad sites of the 2G12 pCAL IT* vector to produce the 2G12 3Ala LC pCAL IT* vector (SEQ ID NO:174).

TABLE 39
2G12 3Ala LC pCAL IT* primers

| | | | SEQ ID |
|---|---|---|---|
| Name | nt | Sequences | NO |
| 2G12LCF1 | 42 | GCCGCTGTGCCATCGCTCAGTAACcaattgaattaaggagga | 324 |
| 2G12LCR1 | 35 | ggcggcgctcttcTAGCGAAGTCGTCGAACTGCAG | 325 |
| 2G12ALCF2 | 54 | GCTACCTACCACTGCCAGCACGCCGCGGGTGCGGCCGC | 326 |
| | | GACCTTCGGTCAGGGT | |
| 2G12ALCR2 | 54 | GGTACCCTGACCGAAGGTCGCGGCCGCACCCGCGGCGT | 327 |
| | | GCTGGCAGTGGTAGGT | |
| 2G12LCF3 | 35 | ggcggcgctcttcTACCCGTGTTGAAATCAAACGT | 328 |
| 2G12LCR3 | 48 | GCCGCTGTGCCATCGCTCAGTAACTTAATTAATTAGCATT | 171 |
| | | CACCACGG | |

The 2G12ALCF2 and 2G12ALCR2 primers contain a 5′ phosphate.

The 2G12ALCF2 and 2G12ALCR2 primers contain three codons (underlined and bold in Table 39 above) that mutate two tyrosines and one serine to alanine. In order to form the CDRL3 3ALA duplex, 50 μL 2G12ALCF2 (100 μM) and 50 μL 2G12ALCR2 (100 μM) were mixed with 1 μL of 5M NaCl. The mixture was denatured at 95° C. for 5 min and slowly cooled to ambient temperature (25° C.) on a heat block covered with a Styrofoam® box to allow duplex formation.

PCR amplification was carried out to generate two 2G12 light chain fragment duplexes. Duplexes in pool 1 (LC1) were 387 nucleotides in length, and duplexes in pool 2 (LC3) were 388 nucleotides in length. For this process, two pools of forward oligonucleotide primers (2G12LCF1 and 2G12LCF3) and two pools of reverse oligonucleotide primers (2G12LCR1 and 2G12LCR3) were synthesized. The sequences of the primers in each pool are set forth in Table 39, above.

Two of the primers, 2G12LCR1 and 2G12LCF3, contained a 5′ sequence of nucleotides corresponding to a SapI restriction endonuclease cleavage site (GCTCTTC) (SEQ ID NO: 180). This enzyme cuts duplex polynucleotides to leave a 3-nucleotide overhang of any sequence at its 5′ end, beginning at one nucleotide in the 3′ direction from this recognition sequence. The restriction endonuclease recognition site is indicated in italics in Table 39, above, while the three-nucleotide overhang in each primer pool is indicated in bold. The oligonucleotides were designed such that the potential three nucleotide overhang of each primer pool was complementary to one of the three nucleotide overhangs generated in the light chain fragment duplexes. The oligonucleotides were designed in this manner to facilitate ligation in a subsequent step.

Primers in the 2G12LCF1 pool contained a sequence of nucleotides corresponding to a MfeI restriction endonuclease recognition site. Primers in the 2G12LCR3 pool contained a sequence of nucleotides corresponding to a PacI restriction endonuclease site (the MfeI and PacI restriction sites are indicated in bold in Table 39). These restriction endonuclease recognition sites facilitated ligation of the assembled duplexes into vectors in subsequent steps.

Further, the forward primer pool 2G12LCF1 and the reverse primer pool 2G12LCR3 contained a non gene-specific sequence region that is identical to the CALX24 primer (SEQ ID NO:112) at the 5′ ends of the primers. Thus, the reference sequence duplexes LC1 and LC3, generated by PCR with these primers/oligonucleotides, contained a duplex of these regions at each end of the reference sequence duplex. These regions served as templates for the primer CALX24, which was used in the subsequent single primer amplification (SPA) step, described below.

To form duplexes using these primers, the 2G12 pCAL IT* vector was used as a template in three separate PCR amplifications. For these reactions, primer pair pools, 2G12LCF1/2G12LCR1 and 2G12LCF3/2G12LCR3, were used to amplify duplex pool LC1 and duplex pool LC3 (Table 40). For each reaction, 4 μL of each primer, 4 μL of the 2G12 pCAL IT* vector template incubated in the presence of 4 μL Advantage HF2 Polymerase Mix (Clontech), 20 μL of 10c HF2 reaction buffer, 20 μL of 10×dNTP mixture, 144 μL PCR grade water in a 200 μL reaction volume. The PCR was carried out using the following reaction conditions: 1 minute denaturation at 95° C., followed by 30 cycles of 5 seconds of denaturation at 95° C., 10 seconds of annealing at 50° C., and 30 seconds of extension at 68° C., then finishing with a 3 minute incubation at 68° C. The amplified fragments were gel-purified using a Gel Extraction Kit (Qiagen) according to the manufacturer's instruction. The purified products were run on 1% agarose gel and each fragment was gel-purified with Gel Extraction Kit (Qiagen) according to the manufacturer's instruction.

TABLE 40
Primer pairs for duplex pools

| Fragment | LC1 | LC3 |
|---|---|---|
| 5′ primer | 2G12LCF1 | 2G12LCF3 |
| 3′ primer | 2G12LCR1 | 2G12LCR3 |
| Size (bp) | 384 | 388 |

After amplification by PCR, 2 μg of LC1 (384 bp) and LC3 (388 bp) were digested with SapI (New England Biolabs). The digested fragments were purified with PCR purification column (Qiagen) according to the manufacturer's instruction.

The digested light chain duplexes and the 3ALA duplex were hybridized and ligated to form intermediate duplexes. This process was carried out as follows. The 3ALA duplex was mixed in equimolar amounts with both reference duplexes, LC1 and LC3, in the presence of 5×T4 DNA ligase buffer and ligated with T4 DNA Ligase in a 20 μL volume, at room temperature (˜25° C.) overnight. The reaction was purified with PCR purification column and run on 1% agarose gel and each fragment was gel purified (Qiagen) according to the manufacturer's instruction.

Following the formation of the intermediate duplexes, a single primer amplification (SPA) reaction was used to generate amplified randomized assembled duplexes. Amplification was carried out using 2 μL of the intermediate duplex and 1.2 μL CALX24 primer (100 μmol), in the presence of 2 μL Advantage HF2 Polymerase Mix, 10 μL 10×HF2 buffer, 10 μL 10×dNTP, 74.8 μL of PCR grade water in a 100 μL reaction volume. The PCR was carried out using the following reaction conditions: denaturation at 95° C. for 1 min, followed by 30 cycles of denaturation at 95° C. for 5 seconds, annealing and extension at 68° C. for 1 min, then finished with an incubation at 68° C. for 3 min. The resulting amplified assembled duplex was column purified with a PCR purification column (Qiagen) and run on 1% agarose gel and purified with Gel Extraction Kit (Qiagen) according to the manufacturer's instruction.

The 3ALA LC duplex cassette was digested with SgrAI and PacI restriction enzymes and purified over a PCR purification column (Qiagen), according to the manufacturer's instruction. The vector DNA, 2G12 pCAL IT*, also was digested with SgrAI and PacI, run on a 0.7% agarose gel, and purified using Gel Extraction Kit (Qiagen). The SgrAI/PacI digested vector and 3ALA LC duplex cassette were ligated in the presence of T4 DNA ligase (Invitrogen) and 5× ligation reaction buffer (Invitrogen) in a 20 μL reaction volume at ambient temperature (22-25° C.) overnight.

The ligated DNA was electroporated into NEB 10-beta cells (New England Biolabs) at 2000 V/0.1 cm and titrated onto LB agar plates containing 100 μg/mL of carbenicillin and 20 mM glucose. Single colonies were selected and amplified. Miniprep DNA were analyzed by DNA sequencing and the clone SP2 was selected for Maxiprep DNA preparation from a single bacterial colony on a LB agar plate containing 100 μg/mL of carbenicillin and 20 mM glucose.

### Example 10

### Generation of Variant 2G12 Nucleic Acid Libraries for Display of Collections of Variant 2G12 Domain Exchanged Fab Fragments

To generate phage display libraries for selection of phage displayed domain exchanged antibodies that have an increased affinity for C. albicans, nucleic acid libraries were generated by randomizing nucleotides encoding four of the nine amino acids in the CDR3 region of the 2G12 light chain. Specifically, the libraries were designed to randomized the four sequential amino acid residues A, G, Y, and S of the light chain CDR3 QHYAGYSAT (SEQ ID NO: 162). The nucleic acid libraries can be used to make phage display libraries containing variant polypeptides with diversity in portions of the CDR3 of the light chain variable region of a 2G12 domain exchanged Fab target polypeptide.

Two methods of randomization were employed. The first method used overlap PCR mutagenesis with Single Primer Amplification, which involved PCR amplification of overlapping segments of the 2G12 light chain using randomized nucleic acid primers, which contain randomized positions within the 2G12 light chain CDR3 encoding region. The second method employed modified Fragment Assembly and Ligation/Single Primer Amplification (mFAL-SPA) (as described in U.S. application No. (Attorney Docket No.: 3800013-00031/1106)), which involved generating a collection of duplex cassettes containing randomized nucleic acids, which have randomized positions within the 2G12 light chain CDR3 encoding region. Both methods are described in detail below.

As described in subsections of this example below, the nucleic acid encoding the 2G12 light chain in the 2G12 3Ala LC pCAL IT* vector described in Example 9 was replaced with either the randomized PCR fragments produced by overlap PCR mutagenesis or the collection of randomized cassettes produced by the mFAL-SPA method to generate the nucleic acid libraries.

A. Randomization of 2G12 Light Chain CDR3 by Overlap PCR Mutagenesis/Single Primer Amplification

Overlap PCR generally involves PCR amplification of two or more overlapping segments of the gene of interest that can be subsequently recombined using an overlap fill-in reaction to reconstitute the full length gene. The process can be used to randomize a region of the gene by using oligonucleotide primers in the PCR amplification step which contain randomized nucleotides in addition to the nucleotides complementary to the template. Overlap PCR mutagenesis and Single Primer Amplification was used to diversify four amino acid positions in the 2G12 Fab by randomization of the 2G12 light chain CDR3 as follows.

1. Generation Overlapping Segments by PCR

Three nucleic acid libraries were generated by overlap PCR. For each library, a set of two overlapping segments of the 2G12 light chain were generated by PCR amplification. The oligonucleotide primers employed for the PCR amplifications are shown in Table 41.

A first segment, containing the nucleic acid encoding the CDR1, CDR2 and the first three amino acids of the CDR3 of the wild-type 2G12 light chain, was amplified as described below with a first oligonucleotide primer complementary to a region directly upstream of the 2G12 light chain in the 2G12 3Ala LC pCAL IT* vector (2G12LCF (SEQ ID NO: 165)) and a second oligonucleotide primer complementary to the region encoding several amino acids upstream of the CDR3 and the first three amino acids of the CDR3 (L3R (SEQ ID NO: 166)). This first segment does not contain any mutations relative to wild-type 2G12 and was used for all three libraries. The sequences of the primers used to amplify the first segment are set forth in Table 41. A MfeI restriction site (CAATTG) (SEQ ID NO: 172; shown in bold in Table 41) was designed in the 2G12LCF oligonucleotide to facilitate ligation of the library into vectors in subsequent steps. The underlined portion of the 2G12LCF oligonucleotide shown in Table 41 indicates a non gene-specific sequence that is identical to the CALX24 primer (SEQ ID NO: 112), which was used for the single primer amplification step described below.

A second segment, containing the nucleic acid encoding the entire CDR3 region of the 2G12 light chain and light chain constant region (C_{L}) was amplified as described below using a first oligonucleotide primer selected from those set forth in Table 41 containing randomized nucleotides in the light CDR3 region and a second oligonucleotide primer complementary to a region encoding the C-terminus of the 2G12 light chain (2G12LCR (SEQ ID NO: 171)). A PacI restriction site (TTAATTAA) (SEQ ID NO: 173; shown in bold in Table 41) was designed in the 2G12LCR oligonucleotide to facilitate to facilitate ligation of the library into vectors in subsequent steps. The underlined portion of the 2G12LCR oligonucleotide shown in Table 41 indicates a non gene-specific sequence that is identical to the CALX24 primer (SEQ ID NO: 112), which was used for the single primer amplification step described below.

Three pools of randomized oligonucleotides (AGYS, AGYS+1, and AGYS+2) were designed and generated for use in PCR amplification. The sequences of these randomized oligonucleotides are set forth in Table 41, below. Each oligonucleotide in each of these randomized pools was synthesized based on a reference sequence (which contained part of the native 2G12 light chain CDR3 nucleotide sequence), but contained randomized portions, represented in underlined type in Table 41. The CDR3 region is represented in bold type. The reference wild-type 2G12 sequence used to design the AGYS, AGYS+1, and AGYS+2 pools of randomized oligonucleotides is listed in Table 41. The region encoding the light chain CDR3 is indicated in bold.

The randomized portions of the oligonucleotides were synthesized using the NNK or NNT doping strategy. An NNK doping strategy minimizes the frequency of stop codons and ensures that each amino acid position encoded by a codon in the randomized portion could be occupied by any of the 20 amino acids. With this doping strategy, nucleotides were incorporated using an NKK pattern and a MNN pattern, during synthesis of the positive and negative strand randomized portions respectively, where N represents any nucleotide, K represents T or G, and M represents A or C. An NNT strategy eliminates stop codons and the frequency of each amino acid is less biased but omits Q, E, K, M, and W. The nucleotides in the randomized pools were labeled with 5′ phosphate groups.

TABLE 41
PCR mutagenesis/Single Primer Amplification Primers

| | | | SEQ ID |
|---|---|---|---|
| Name | nt | Sequences | NO |
| 2G12LCF | 41 | GCCGCTGTGCCATCGCTCAGTAACaattgaattaaggagga | 165 |
| L3R | 20 | ATAGTGCTGGCAGTGGTAGG | 166 |
| 2G12 | 55 | CTACCTACCACTGCCAGCACTACGCTGGTTACTCT | 167 |
| reference | | GCTACCTTCGGTCAGGGTAC | |
| sequence | | | |
| AGYS | 55 | CTACCTACCACTGCCAGCACTATNNKNNKNNKNNK | 168 |
| | | GCTACCTTCGGTCAGGGTAC | |
| AGYS + 1 | 58 | CTACCTACCACTGCCAGCACTATNNKNNKNNKNNKNNK | 169 |
| | | GCTACCTTCGGTCAGGGTAC | |
| AGYS + 2 | 61 | CTACCTACCACTGCCAGCACTATNNKNNKNNKNNKNNKNNK | 170 |
| | | GCTACCTTCGGTCAGGGTAC | |
| 2G12LCR | 48 | GCCGCTGTGCCATCGCTCAGTAACTTAATTAATTAGCATTCAC | 171 |
| | | CACGG | |

The 2G12LCF, L3R and 2G12LCR primers were purified by HPLC. The AGYS, AGYS + 1 and AGYS + 2 primers contain a 5′ phosphate.

PCR amplification of the overlapping segments was performed using the primer pairs shown in Table 42. Each fragment was amplified using 10 ng of 2G12 3Ala LC pCAL IT* (SEQ ID NO: 174) (10 μL of 100 ng/μL stock) as a template with 10 μL of 20 μM 5′ and 3′ primers listed in Table 42 below in the presence of 10 μL of Advantage® HF2 Polymerase Mix (Clontech), 50 μL of 10×HF2 reaction buffer (Clontech), 50 μL of 10×dNTP mixture, and 360 μL of PCR grade water in a 500 μL reaction volume.

Each of the PCR amplifications (PCR 1a, 1b, 1b+1, 1b+2) included a denaturation step at 95° C. for 1 min, followed by 20 cycles of denaturation at 95° C. for 5 seconds, at 50° C. for 10 seconds, and extension at 68° C. for 30 seconds, and finished with incubation at 68° C. for 1 min.

TABLE 42
PCR primers and resulting fragment sizes

| Fragment | PCR1a | PCR1b | PCR1b + 1 | PCR1b + 2 |
|---|---|---|---|---|
| 5′ primer | 2G12LCF | AGYS | AGYS + 1 | AGYS + 2 |
| 3′ primer | L3R | 2G12LCR | 2G12LCR | 2G12LCR |
| Size (bp) | 390 | 427 | 430 | 433 |

The amplified products from the PCR reactions were purified on a single PCR purification column (Qiagen). The purified products were run on 1% agarose gel and each fragment was gel-purified with Gel Extraction Kit (Qiagen) according to the manufacturer's instructions.

A. 2. Overlap Fill-In Reaction

The overlapping segments generated from the PCR amplifications were rejoined to produce the nucleic acid library encoding full-length light chains, which contain the randomized CDR3 regions. The full-length nucleic acids were reconstructed by denaturation of the PCR amplified segments, annealing of the overlapping the nucleic acid, followed by an overlap fill-in reaction. Each library was constructed using 50 μl, of PCR1 Mix as shown in Table 43 for each library, 2 μL of Advantage® HF2 Polymerase Mix (Clontech), 10 μL of 10×HF2 reaction Buffer, 10 μL of 10×dNTP mixture, and 28 μL of PCR grade water in a 100 μL reaction volume. The calculated volumes for each of the PCR samples used in the fill-in reactions is shown in Table 43.

Each of the overlap reactions (AGYS, AGYS+1, and AGYS+2) included a denaturation step at 95° C. for 1 min, followed by 40 cycles of denaturation at 95° C. for 5 seconds, annealing at 60° C. for 10 seconds, and extension at 68° C. for 1 min, and finished with incubation at 68° C. for 3 min. The amplified products were run on 1% agarose gel and each fragment was purified with Gel Extraction Kit (Qiagen) according to the manufacturer's protocol.

TABLE 43
Calculated volumes for PCR samples

| | Length | Amount needed for | Volume for |
|---|---|---|---|
| | of PCR | reaction: | fill-in |
| | fragment | 6.08 pmol (μg) | reaction |
| | (bp) | (3.64 × 10¹² molecules) | (μL) |
| PCR1a | 390 | 1.560 | 26.85 |
| PCR1b | 427 | 1.708 | 16.03 |
| PCR1b+1 | 430 | 1.720 | 10.23 |
| PCR1b+2 | 433 | 1.732 | 12.42 |

TABLE 44
PCR1 Mix for Overlap Reactions

| | Library | | |
|---|---|---|---|
| | AGYS | AGYS + 1 | AGYS + 2 |
| PCR1a (μL) | 26.85 | 26.85 | 26.85 |
| PCR1b (μL) | 16.03 | 0 | 0 |
| PCR1b + 1 (μL) | 0 | 10.23 | 0 |
| PCR1b + 2 (μL) | 0 | 0 | 12.42 |
| PCR grade water | 7.12 | 12.92 | 10.73 |
| (μL) | | | |
| Total (μL) | 50 | 50 | 50 |
| Size of combined | 794 | 797 | 800 |
| fragment (bp) | | | |

B. 3. Single Primer Amplification (SPA)

SPA was performed by mixing 244 μL of PCR grade water, 50 μL of 10×HF2 buffer, 50 μL of 10×dNTP, 6 μL of CALX24 primer (100 μm) (SEQ ID NO: 21), 140 μL of each overlap fill-in reaction (AGYS, AGYS+1 or AGYS+2), and 10 μL of Advantage® HF2 Polymerase Mix in a 500 μL reaction volume.

Each of the SPA reactions included a denaturation step at 95° C. for 1 min, followed by 20 cycles of denaturation at 95° C. for 5 seconds, annealing and extension at 68° C. for 1 min, and finished with incubation at 68° C. for 3 min. The amplified products were column purified and run on 1% agarose gel and purified with Gel Extraction Kit (Qiagen).

5. Formation of the Variant 2G12 Nucleic Acid Libraries

Five μg of each library (AGYS, AGYS+1 or AGYS+2) was digested with MfeI and PacI restriction enzymes and purified over a PCR purification column (Qiagen). The vector DNA, 2G12 3Ala LC pCAL IT* (60 μg), also was digested with MfeI and PacI, run on a 0.7% agarose gel, and the 5139 by vector fragment was purified using Gel Extraction Kit (Qiagen).

The MfeI/PacI digested vector and library fragments were ligated in the presence of 10 μL T4 DNA ligase (10 units) (Invitrogen) and 5× ligation reaction buffer (Invitrogen) in a 200 μL reaction volume at ambient temperature (22-25° C.) overnight. The ng and pmol amounts of the vector and library fragments used in the ligation reactions are shown in Table 45.

TABLE 45
Amounts of vector and library fragments used in ligation reactions

| Library | Amount | AGYS | AGYS + 1 | AGYS + 2 |
|---|---|---|---|---|
| Vector | ng | 1066.77 | 1066.77 | 8139.06 |
| | pmol | 0.316 | 0.315 | 2.405 |
| Fragment | ng | 385.58 | 387.142 | 2965.63 |
| | pmol | 0.789 | 0.790 | 6.026 |

C. 6. Transformation

The ligation reactions were purified over PCR purification column (Qiagen) and electroporated into NEB 10-beta cells (New England Biolabs) at 2000 V in cuvettes with 0.1 cm gap. The cells were resuspended in SOC medium and incubated at 37° C. for 1 hr. Thirty mL of SuperBroth medium containing 20 μg/mL of carbenicillin and 20 mM of glucose were added to the culture and titrated on to LB agar plates containing 100 μg/mL of carbenicillin and 20 mM of glucose. The cells were incubated at 37° C. for 1 hr and added to 200 mL of SuperBroth medium with 50 μg/mL of carbenicillin and 20 mM of glucose. The culture was incubated overnight at 37° C. Maxiprep DNA was prepared from the overnight culture using HiSpeed Maxiprep Kit (Qiagen) according to the manufacturer's protocol.

The size of each library was 3.64×10⁸ for AGYS, 2.84×10⁸ for AGYS+1, and 1.59×10⁹ for AGYS+2.

B. Randomization of 2G12 Light Chain CDR3 by Modified Fragment Assembly and Ligation/Single Primer Amplification (mFAL-SPA)

The Modified Fragment Assembly and Ligation (mFAL-SPA) method, as described in U.S. application No. (Attorney Docket No.: 119367-00031/1106), also was employed to generate nucleic acid libraries which are diversified at the same four amino acid positions (A, G, Y, S), in the light chain CDR3 of 2G12 Fab. The details of this method are as follows.

1. Generation of Pools of Randomized Duplexes

Six pools of randomized oligonucleotides (AGYS, SYGA, AGYS+1, SYGA+1, AGYS+2, and SGYA+2) were designed and generated for use in forming three pools of randomized duplexes (DO, DO+1, and DO+2). The sequences of these randomized oligonucleotides are set forth in Table 46, below. Each oligonucleotide in each of these randomized pools was synthesized based on a reference sequence (which contained part of the wild-type 2G12 light chain CDR3 nucleotide sequence), but contained randomized portions, represented in underlined type in Table 46 for oligonucleotides AGYS, SYGA, AGYS+1, SYGA+1, AGYS+2, and SGYA+2. The region encoding the light chain CDR3 region in these oligonucleotides is represented in bold type. The randomized portions were synthesized using the NNK or NNT doping strategy as described above for the overlap PCR mutagenesis. The reference wild-type 2G12 sequence used to design the AGYS, SYGA, AGYS+1, SYGA+1, AGYS+2, and SGYA+2 pools of randomized oligonucleotides also is listed in Table 46. The region encoding the light chain CDR3 is indicated in bold.

The randomized oligonucleotides were designed such that each oligonucleotide in each of the pools contained a region complementary to an oligonucleotide in another pool. For example, oligonucleotides in pool AGYS were complementary to oligonucleotides in pool SYGA, oligonucleotides in pool AGYS+1 were complementary to oligonucleotides in pool SYGA+1, and oligonucleotides in pool AGYS+2 were complementary to oligonucleotides in pool SYGA+2. The oligonucleotides in each pool further were designed, whereby, following hybridization of the pairs of oligonucleotides through these complementary regions, two nucleotide 5′-end overhangs would be generated, to facilitate ligation in subsequent steps. The nucleotides that become the 5′-end overhangs are indicated in italics in Table 46 for oligonucleotides AGYS, SYGA, AGYS+1, SYGA+1, AGYS+2, and SGYA+2. The nucleotides in the randomized pools were labeled with 5′ phosphate groups.

TABLE 46
Primers for mFAL-SPA

| | | | SEQ |
|---|---|---|---|
| | | | ID |
| Name | nt | Sequences | NO |
| 2G12LCF | 41 | GCCGCTGTGCCATCGCTCAGTAACaattgaattaaggagga | 165 |
| L1R | 34 | gggcggcgctcttcG [symbol] CGAAGTCGTCGAACTG | 175 |
| 2G12 | 55 | CTACCTACCACTGCCAGCACTACGCTGGTTACTCT | 167 |
| reference | | GCTACCTTCGGTCAGGGTAC | |
| sequence | | | |
| AGYS | 55 | [symbol] CCTACCACTGCCAGCACTATNNKNNKNNKNNK | 168 |
| | | GCTACCTTCGGTCAGGGTAC | |
| SYGA | 55 | [symbol] GTACCCTGACCGAAGGTAGCMNNMNNMNNMNN | 176 |
| | | ATAGTGCTGGCAGTGGTAGG | |
| AGYS + 1 | 58 | [symbol] CCTACCACTGCCAGCACTATNNKNNKNNKNNKNNK | 169 |
| | | GCTACCTTCGGTCAGGGTAC | |
| SYGA + 1 | 58 | [symbol] GTACCCTGACCGAAGGTAGCMNNMNNMNNMNNMNNATA | 177 |
| | | GTGCTGGCAGTGGTAGG | |
| AGYS + 2 | 61 | [symbol] CCTACCACTGCCAGCACTATNNKNNKNNKNNKNNKNNK | 170 |
| | | GCTACCTTCGGTCAGGGTAC | |
| SYGA + 2 | 61 | [symbol] GTACCCTGACCGAAGGTAGCMNNMNNMNNMNNMNNMNN | 178 |
| | | ATAGTGCTGGCAGTGGTAGG | |
| L2F | 34 | gggcggcgctcttcC [symbol] TGTTGAAATCAAACGT | 179 |
| 2G12LCR | 48 | GCCGCTGTGCCATCGCTCAGTAACTTAATTAATTAGCATTCAC | 171 |
| | | CACGG | |

The 2G12LCF, L1R and 2G12LCR primers were purified by HPLC. The AGYS, SYGA, AGYS + 1, SYGA + 1, AGYS + 2, and SYGA + 2 primers contain a 5′ phosphate.

In order to form the DO, DO+1, and DO+2 randomized duplexes, 50 μL oligonucleotide 1 (at 100 μM) and 50 μL oligonucleotide 2 (see Table 46) (100 μM) as set forth in Table 47 for each reaction were mixed with 1 μL of 5M NaCl. The mixture was denatured at 95° C. for 5 min and slowly cooled to ambient temperature (25° C.) on a heat block covered with a Styrofoam® box to allow duplex oligonucleotide (DO) formation.

TABLE 47
Oligonucleotide pairings for generation of
randomized duplexes

| | DO | DO + 1 | DO + 2 |
|---|---|---|---|
| Oligonucleotide 1 | AGYS | AGYS + 1 | AGYS + 2 |
| Oligonucleotide 2 | SGYA | SGYA + 1 | SGYA + 2 |
| Size (bp) | 55 | 58 | 61 |

2. Generation of Reference Sequence Duplexes by PCR

PCR amplification was carried out to generate two reference sequence duplexes (LC1 and LC2). Duplexes in pool 1 (LC1) were 385 nucleotides in length, and duplexes in pool 2 (LC2) were 387 nucleotides in length. For this process, two pools of forward oligonucleotide primers (2G12LCF and L2F) and two pools of reverse oligonucleotide primers (L1R and 2G12LCR) were synthesized. The sequences of the primers in each pool are set forth in Table 46, above.

Two of the primers, L1R and L2F, used to generate the reference sequence duplexes contained a 5′ sequence of nucleotides corresponding to a SapI restriction endonuclease cleavage site (GCTCTTC) (SEQ ID NO: 180). This enzyme cuts duplex polynucleotides to leave a 3-nucleotide overhang of any sequence at its 5′ end, beginning at one nucleotide in the 3′ direction from this recognition sequence. The restriction endonuclease recognition site is indicated in italics in Table 46, above, while the three-nucleotide overhang in each primer pool is indicated in bold. The oligonucleotides were designed such that the potential three nucleotide overhang of each primer pool was complementary to one of the three nucleotide overhangs generated in the randomized duplexes. The oligonucleotides were designed in this manner to facilitate ligation in a subsequent step.

Primers in the 2G12LCF pool contained a sequence of nucleotides corresponding to a MfeI restriction endonuclease recognition site. Primers in the 2G12LCR pool contained a sequence of nucleotides corresponding to a PacI restriction endonuclease site (the MfeI and PacI restriction sites are indicated in bold in Table 46). These restriction endonuclease recognition sites facilitated ligation of the assembled duplexes into vectors in subsequent steps.

Further, the forward primer pool 2G12LCF and the reverse primer pool 2G12LCR contained a non gene-specific sequence region that is identical to the CALX24 primer (SEQ ID NO:112) at the 5′ ends of the primers. Thus, the reference sequence duplexes LC1 and LC2, generated by PCR with these primers/oligonucleotides, contained a duplex of these regions at each end of the reference sequence duplex. These regions served as templates for the primer CALX24, which was used in the subsequent single primer amplification (SPA) step, described below.

To form duplexes using these primers, the 2G12 3Ala LC pCAL IT* vector was used as a template in three separate PCR amplifications. For these reactions, primer pair pools, 2G12LCF/L1R and L2F/2G12LCR, were used to amplify duplex pool LC1 and duplex pool LC2 (Table 48). For each reaction, 200 picomoles (pmol) of each primer (10 μL), 1 microgram (μg) of the 2G12 3Ala LC pCAL IT* vector template (10 μL of 100 ng/μL stock) were incubated in the presence of 10 μL Advantage HF2 Polymerase Mix (Clontech), 50 μL of 10c HF2 reaction buffer, 50 μL of 10×dNTP mixture, 360 μL PCR grade water in a 500 μL reaction volume. The PCR was carried out using the following reaction conditions: 1 minute denaturation at 95° C., followed by 20 cycles of 5 seconds of denaturation at 95° C., 10 seconds of annealing at 50° C., and 30 seconds of extension at 68° C., then finishing with a 1 minute incubation at 68° C. The amplified fragments were gel-purified using a Gel Extraction Kit (Qiagen) according to the manufacturer's instruction. The purified products were run on 1% agarose gel and each fragment was gel-purified with Gel Extraction Kit (Qiagen) according to the manufacturer's instruction.

TABLE 48
Primer pairs for duplex pools

Fragment

| | LC1 | LC2 |
|---|---|---|
| 5′ primer | 2G12LCF | L2F |
| 3′ primer | L1R | 2G12LCR |
| Size (bp) | 385 | 387 |

After amplification by PCR, 20 pmoles of LC1 (385 bp) and LC2 (387 bp) were digested with SapI (New England Biolabs). The digested fragments were purified with PCR purification column (Qiagen) according to the manufacturer's instruction.

3. Ligation of Digested Reference Sequence Duplexes and Randomized Duplexes to Form Intermediate Duplexes

The digested reference sequence duplexes and the randomized duplexes were hybridized and ligated to form intermediate duplexes. This process was carried out as follows. Three ligation reactions, one for each randomized duplex (DO, DO+1, and DO+2), were prepared. Each randomized duplex (DO, DO+1, or DO+2) was mixed in equimolar amounts (5.19 picomoles) with both reference duplexes, LC1 and LC2, in the presence of 80 μL 5×T4 DNA ligase buffer and ligated with 20 units of T4 DNA Ligase in a 400 μL volume, at room temperature (˜25° C.) overnight. The reaction was purified with PCR purification column and run on 1% agarose gel and each fragment was gel purified (Qiagen) according to the manufacturer's instruction.

4. Formation of Duplex Cassettes by Single Primer Amplification

Following the formation of the intermediate duplexes, a single primer amplification (SPA) reaction was used to generate amplified randomized assembled duplexes. Amplification was carried out using 140 μL of the intermediate duplex (LC1/DO/LC2, LC1/DO+1/LC2, or LC1/DO+2/LC2) and 6 μL CALX24 primer (100 μmol), in the presence of 10 μL Advantage HF2 Polymerase Mix, 50 μL 10×HF2 buffer, 50 μL 10×dNTP, 244 μL of PCR grade water in a 500 μL reaction volume. The PCR was carried out using the following reaction conditions: denaturation at 95° C. for 1 min, followed by 20 cycles of denaturation at 95° C. for 5 seconds, annealing and extension at 68° C. for 1 min, then finished with an incubation at 68° C. for 3 min.

The resulting collections of amplified assembled duplexes were column purified with a PCR purification column (Qiagen) and run on 1% agarose gel and purified with Gel Extraction Kit (Qiagen) according to the manufacturer's instruction. Each duplex cassette LC1/DO/LC2, LC1/DO+1/LC2, and LC1/DO+2/LC2 represents the AGYS, AGYS+1 and AGYS+2 libraries, respectively.

5. Formation of the Variant 2G12 Nucleic Acid Libraries

Five μg of each library (AGYS, AGYS+1 or AGYS+2) was digested with MfeI and Pad restriction enzymes and purified over a PCR purification column (Qiagen), according to the manufacturer's instruction. The vector DNA, 2G12 3Ala LC pCAL IT* (60 μg), also was digested with MfeI and PacI, run on a 0.7% agarose gel, and the 5139 by vector fragment was purified using Gel Extraction Kit (Qiagen). Each vector was ligated with the assembled duplex cassettes described above, to generate three libraries, each containing randomized 2G12 Fab encoding nucleic acid members.

The MfeI/PacI digested vector and library fragments were ligated in the presence of 10 μL T4 DNA ligase (10 units) (Invitrogen) and 5× ligation reaction buffer (Invitrogen) in a 200 μL reaction volume at ambient temperature (22-25° C.) overnight. The ng and pmol amounts of the vector and library fragments used in the ligation reactions is shown in Table 49.

TABLE 49
Amounts of vector and library fragments used in ligation reactions

| Library | Amount | AGYS | AGYS + 1 | AGYS + 2 |
|---|---|---|---|---|
| Vector | ng | 1066.77 | 1066.77 | 8139.06 |
| | pmol | 0.316 | 0.315 | 2.405 |
| Fragment | ng | 385.58 | 387.142 | 2965.63 |
| | pmol | 0.789 | 0.790 | 6.026 |

D. 6. Transformation

The ligation reactions were purified over PCR purification column (Qiagen) and electroporated into NEB 10-beta cells (New England Biolabs) at 2000 V in cuvettes with 0.1 cm gap. The cells were resuspended in SOC medium and incubated at 37° C. for 1 hr. Thirty mL of SuperBroth medium containing 20 μg/mL of carbenicillin and 20 mM of glucose were added to the culture and titrated on to LB agar plates containing 100 μg/mL of carbenicillin and 20 mM of glucose. The cells were incubated at 37° C. for 1 hr and added to 200 mL of SuperBroth medium with 50 μg/mL of carbenicillin and 20 mM of glucose. The culture was incubated overnight at 37° C. Maxiprep DNA was prepared from the overnight culture using HiSpeed Maxiprep Kit (Qiagen) according to the manufacturer's protocol.

The size of each library was 3.15×10⁸ for AGYS, 3.98×10⁸ for AGYS+1, and 1.59×10⁹ for AGYS+2.

### Example 11

### Preparation of Formalin-Fixed Candida albicans Cells

Formalin fixed C. albicans cells were prepared for use as the C. albicans target antigen for phage selection. A starter culture was first prepared by inoculation of 10 mL of YPD medium with a single colony of C. albicans (Cat. No. 10231, ATCC, stored at −20° C.). The cells were cultured at 37° C. with shaking at 170 rpm for 24 hours, before 500 μL of culture was removed and transferred into 10 mL of fresh YPD medium. This was repeated to generate 10 individual cultures, which were incubated at 37° C. with agitation at 170 rpm for 24 hours. The C. albicans cells were centrifuged at 4000 rpm for 10 minutes and the cell pellet was resuspended in 1×PBS. This washing step was repeated twice before the cell pellet was fixed in 1% formalin (diluted in 1×PBS). The cells were incubated with shaking for 30 minutes at room temperature before being centrifuged at 4000 rpm for 10 minutes. The cell pellet was resuspended in 1×PBS. The cells were washed twice more with PBS before being counted using a hemocytometer. The C. albicans cell density was adjusted to 1×10⁸ cells per mL, and the cells were aliquoted into 1 mL stocks and stored at −20° C. or −80° C. The fixed C. albicans cells were thawed on ice prior to use before each round of selection.

### Example 12

### Selection of Domain Exchanged Antibodies Specific for Candida albicans

Diversified 2G12-derived domain exchanged antibodies having specificity for Candida albicans were selected using phage display techniques. Each 2G12 library generated as described in Example 10 was introduced into electrocompetent DH5α VCSM13 dsDNA CL F-cells for expression on the surface of the cells in phagemids. The phage were then screened for specificity for C. albicans using formalin-fixed C. albicans cells as the target antigen. The selection protocol is described in general below.

A. Preparation of Electrocompetent DH5α VCSM13 dsDNA CL F-Cells

To generate the electrocompetent DH5α VCSM13 dsDNA CL F-cells for subsequent use in the display of phage, doublestranded DNA from VCSM13 helper phage was purified before being transformed into DH5α cells. These cells were then treated to become electrocompetent.

1. Purification of VCSM13 Helper Phage dsDNA

Doublestranded DNA from VCSM13 helper phage was purified using the Qiafilter Midiprep or Maxiprep Kit (Qiagen), per the manufacturer's instructions. Briefly, a colony of XL1-Blue MRF′ cells (Stratagene) was transferred into 10 ml of Superbroth (SB) media (30 g Bacto tryptone, 20 g Yeast extract, 10 g MOPS, in 1 liter water, pH 7.0) in a 50-ml conical tube. Tetracycline was added to a final concentration of 10 μg/mL, and the culture was incubated with shaking at 37° C. until an OD600 of 0.3 was reached (corresponding approximately to 2.5×10⁸ cells/mL). The culture was scaled up to between 50 and 100 mL, tetracycline was added to a final concentration of 10 μg/mL. For culture volumes of approximately 50-100 mL, the Qiagen Qiafilter Midiprep was used for purification. For culture volumes of approximately 200 mL, the Qiagen Qiafilter Maxiprep was used for purification.

VCSM13 helper phage (Stratagene) were added to the culture at a multiplicity of infection (MOI) of 10:1 (phage-to-cells ratio). The culture was incubates at 37° C. (without agitation) for 15 minutes to allow the phage to attach to the cells, before being incubated for a further hour with shaking at 37° C. Kanamycin was added to the culture at a final concentration of 25 μg/mL, and the culture was incubated with shaking at 37° C. for a further 8 hours. The cell debris was pelleted by centrifugation and the supernatant was transferred to a fresh conical tube. The pellet was stored at either −20° C. or −80° C. until required. The titer of the supernatant was determined and typically found to be between 7.5×10¹⁰ and 1×10¹² pfu/mL.

The cell pellet was resuspended in 4 mL of Buffer P1 if a Midiprep was being used for purification, or 10 mL of Buffer P1 if a Maxiprep was being used for purification. The DNA was purified as per the manufacturer's instructions. Following elution from the Qiagen-tip 100 (if a Midiprep kit was used) or Qiagen-tip 500 (if a Maxiprep kit was used), the VCSM13 DNA was precipitated by the addition of 0.7 volumes of room temperature isopropanol and centrifugation at >15,000×g for 60 minutes at 4° C. The DNA pellets were washed with 2 mL or 5 mL (for Midiprep or Maxiprep purifications, respectively) of 70% ethanol and centrifuged at >15,000×g for 10 minutes at 4° C. The VCSM13 DNA pellet was air dried for 5-10 minutes and dissolved in a suitable volume of TE buffer, pH 8.0, or 10 nM Tris-Cl, pH 8.5. The concentration of VCSM13 DNA was then measured.

2. Preparation of Electrocompetent VCSM13 DH5α Cell Line

To prepare the electrocompetent VCSM13 DH5α cell line, sterile SOC was first pre-heated to 37° C. and the electroporator settings were adjusted to: 2000V [20 kV/cm field strength], resistance to 200Ω, capacitance to 25 μF. Electroporation cuvettes (0.1 centimeter gap) were pre-chilled at −20° C. and transferred to an ice bucket prior to use. Electrocompetent ElectroMax DH5α-E cells (Invitrogen) were thawed on ice before 100 ng of the purified VCSM13 DNA was added to the cells. The cells were then incubated for 5 minutes on ice and transferred from the 1.5 mL tube into each pre-chilled electroporation cuvette. To avoid arcing and to ensure optimal DNA entry, a 2-5% volume of DNA to cell ratio typically was used. The electroporation cuvettes were tapped gently until the mixture of cells and DNA settled flush with the bottom of the cuvette, and any external water or condensation on the cuvette was wiped away. The sample was pulsed once and the cuvette was quickly removed and 1000 μl of pre-warmed SOC media was added to the cells.

The cells were then transferred to a sterile 50 mL conical polypropylene tube, and the remaining cells in the cuvette were flushed twice more with 1 mL, so that the cells were resuspended in a final volume of 3 mL SOC media. Superbroth media was added to the cells for a final volume of 10 mL, and the cells were incubated at 37° C. with shaking at 250 rpm, for 1 hour. (To calculate the transformation efficiency, 90 μl of SOC was aliquoted into an ELISA dilution plate, and 10 μL of the cells (DH5α cells with VCSM13 DNA) was add to the top well and a 6 step, 10-fold dilution series was prepared. Seventy-five μL of the diluted cells were plated on LB agar/kanamycin plates (LB agar with 25 μg/mL kanamycin and 20 mM D-glucose), and the liquid was allowed to dry for a minimum of 15 minutes before being incubated at 37° C. overnight).

After the 1 hour incubation, 0.5 mL of the DH5α VCSM13 dsDNA CL F-cells were inoculated into a 500 mL flask containing 50 mL of SB media, and kanamycin was added to a final concentration of 25 μg/mL. The flask was incubated at 37° C. overnight with shaking at 250 rpm. Ten mL of the overnight culture was added to 1 L of SB in a 2 L flask, and kanamycin was added to a final concentration of 25 μg/mL. The cells were grown at 37° C. with shaking at 250 rpm until the culture reached an OD 600 of approximately 0.6-0.7, so that the cells were harvested at early to mid-log phase (cell density of approximately 4-5×10⁷ cells/mL). The cells were chilled on ice for approximately 20 minutes, and kept in an ice/water bath for the subsequent steps. All containers used in the subsequent steps also were chilled before adding any cells.

The DH5α VCSM13 dsDNA CL F-cells were transferred to three large centrifuge bottles and centrifuged at 4000×g for 20 min at 4° C. The supernatant was decanted and the cells remaining in the bottle were placed on ice. The cell pellets were then resuspended in 10 mL of ice cold 10% glycerol, and the bottles were then filled with approximately 400 mL of ice cold 10% glycerol. The cells were again centrifuged at 4000×g for 20 min at 4° C., the supernatant was decanted, and the cells remaining in the bottle were placed on ice. The cell pellets were resuspended in 10 mL of ice cold 10% glycerol, and another approximately 400 mL 10% glycerol was added to fill the bottle before the cells were again centrifuged at 4000×g for 20 min at 4° C. The supernatant was removed and the cells were resuspended in approximately 25 mL ice cold 10% glycerol and transferred to a pre-chilled 50 mL falcon tube. The cells were pelleted by centrifugation at 4000 rpm for 30 minutes and the supernatant was carefully removed. The final cell pellet was resuspended in 4-5 mL ice cold 10% glycerol, having a concentration of about 1-3×10¹⁰ cells/mL. The resulting electrocompetent DH5α VCSM13 dsDNA CL F-cells were aliquoted in 100 μL volumes into several pre-chilled sterile 1.5 mL tubes, on ice, before being frozen in a dry ice/ethanol bath or in liquid nitrogen and stored at −80° C.

B. Phage Display and Selection of Domain-Exchanged Antibodies Specific for C. albicans.

1. Electroporation of 2G12 Library DNA into DH5α VCSM13 dsDNA CL F-Cells and Library Expansion.

The six libraries generated in Example 10 were individually electroporated and screened. For electroporation of 2G12 library DNA into electrocompetent DH5α VCSM13 dsDNA CL F-cells, the electroporator settings were adjusted as follows: 2000V (20 kV/cm field strength), resistance to 200Ω, and capacitance to 25 μF. The electroporation cuvettes (0.1 centimeter gap) were pre-chilled at −20° C. and transferred to an ice bucket until use. Electrocompetent DH5α VCSM13 dsDNA CL F-cells (prepared as described in Example 11.A.1, above) were thawed on ice. Pre-chilled 2G12 library DNA was then added to the cells and incubated on ice for 5 minutes. Typically, 100 ng of library DNA in 2-5 μL was added to 100 μL of cells. The volume of cells and amount of DNA added was dependent upon the scale of the electroporation. For a mini electroporation, 100-500 ng DNA was added to 100-500 μL cells, which resulting in approximately 1×10⁸ to 1×10⁹ cfu. For a midi electroporation, 500-1000 ng DNA was added to 500-1000 μL cells, resulting in approximately 1×10⁹ to 1×10¹⁰ cfu. For a maxi electroporation, 1500-3000 ng DNA was added to 1500-3000 μL cells, resulting in greater that 1×10¹⁰ cfu. One hundred μL of the cells, premixed with the library DNA, was then added to each electroporation cuvette, which was tapped gently until the cell mixture settled flush with the bottom of the cuvette. Thus, for a mini electroporation, there were 1-5 cuvettes; for a midi electroporation, there were 5-10 cuvettes; and for a maxi electroporation, there were 15-30 cuvettes. Any external water or condensation on the cuvette was removed before the samples were pulsed once.

The cuvettes were removed and 1000 μl of prewarmed (37° C.) SOC media was added to resuspend and quench the cells. The cells were transferred to a sterile 50 mL conical polypropylene tube, and the SOC flush process was repeated two more times, resulting in 3 mL of cells from each electroporation cuvette. 2YT medium (containing 16 g Bacto tryptone, 10 g Yeast extract and 5 g NaCl per liter) was added to the cells in each tube to a final volume of 10 mL per tube. Sterile glucose was then added to a final concentration of 20 mM. The cells were incubated at 37° C. with shaking at 250 rpm for 1 hour. (To calculate the transformation efficiency, 90 μl of SOC was aliquoted into an ELISA dilution plate, and 10 μL of the cells (DH5α VCSM13 dsDNA CL F-cells with library DNA) was added to the top well and a 6 step, 10-fold dilution series was prepared. Seventy-five μL of the diluted cells were plated on LB agar/carbenicillin plates (LB agar with 100 μg/mL carbenicillin and 20 mM D-glucose), and the liquid was allowed to dry for a minimum of 15 minutes before being incubated at 37° C. overnight).

Following the 1 hour incubation, the cells were transferred to a 100 mL bottle and 2YT media was added to a final volume of 50 mL before kanamycin (final concentration of 25 μg/mL) and carbenicillin (final concentration of 50 μg/mL) also were added for library expansion. For every 100 nanograms of library DNA electroporated (i.e. for every electroporation cuvette), a separate culture bottle with 50 mL 2YT final volume was used (i.e. for a mini electroporation, there was 1-5×50 mL 2YT; for a midi electroporation, there was 5-10×50 mL 2YT; and for a maxi electroporation, there was 15-30×50 mL 2YT). The library was then expanded by incubation of the cells at 37° C. with shaking at 250 rpm for 2 hours.

2. Phagemid Expression

Following the library expansion, the cell suspension was centrifuged at room temperature for 25 minutes at 4000 rpm and the cell pellet was resuspended in 2YT media to a final volume of such that the OD595 of the bacterial culture was 0.3. Kanamycin was added to a final concentration of 25 μg/mL, carbenicillin was added to a final concentration of 50 μg/mL, and IPTG was added to a final concentration of 1 mM (for variations of the protocol in which pCAL libraries rather than pCAL IT* libraries are used, IPTG is not added). The cells were incubated at 30° C., 300 rpm for 9 hours, then incubated at 4° C. with shaking at 200 rpm until needed.

3. Phage Precipitation and Preparation for Capture

To precipitate the phage, the cultures bottles containing the expressed phage were removed from the 4° C. incubator and centrifuged at 4000 rpm for 30 minutes. Thirty-two mL of the supernatant was transferred to a 50 mL Nalgene centrifuge tube and 8 mL of 20% PEG with 2.5M NaCl was added (a ratio of 4:1 supernatant:20% PEG with 2.5M NaCl). The tube was inverted 10 times before being incubated on ice for 30 minutes. The centrifuge tube was spun at 13,000 rpm for 30 minutes at 4° C., and the supernatant was pour off. The tube was inverted on a paper towel for 5-10 minutes to remove any excess media. The phage pellet on the bottom of the tube was carefully resuspended (without any bubbles forming) in 1000 μL 1×PBS if a mini electroporation was originally performed, 3750 μL 1×PBS if a midi electroporation was originally performed, or 10000 μL 1×PBS if a maxi electroporation was originally performed. The resuspended phage were transferred to an appropriate number of sterile 1.5 mL microcentrifuge tubes, which were centrifuged at 13,500 rpm, at 25° C. for 5 minutes to pellet cell debris. Finally, supernatant containing the resuspended phage was mixed at a 1:1 ratio with 8% nonfat dry milk (NFDM; reconstituted in 1×PBS) for a final concentration of 4% NFDM. Any unused supernatant was transferred to a sterile 1.5 mL microcentrifuge tube.

4. Phage Capture

An appropriate amount of phage (1000 μL for a mini scale selection; 5000 μL for a midi scale selection; 15000 μL for a maxi scale selection), was added to an 1.5 mL tube or 50 mL conical tube (depending on the scale of selection). The phage were then mixed with Tween20 to a final concentration of 0.05% Tween20, and 1×10⁸ formalin-fixed C. albicans cells. The mixture was then incubated with rocking for 2 hours at 37° C. The C. albicans cells were washed by centrifugation at 4000 rpm for 5 minutes, removal of the supernatant, and resuspension in 1500 μL, 5000 μL or 15000 μL PBS/0.05% Tween20 (for mini, midi and maxi scale selections, respectively). The washing procedure was repeated four times for a total of 5 washes.

5. Phage Elution

To elute the phage, 150 μL, 500 μL or 1000 μL of 0.1M glycine, pH 2.2 (for a mini, midi or maxi scale selection, respectively), was added to the cells and incubated for 10 minutes at room temperature. The tube was vortexed repeatedly to ensure complete elution of all of the phage. After centrifugation to pellet the cells, the glycine containing the eluted phage was transferred to a sterile 1.5 mL tube and was neutralized with the addition of 15 μL, 50 μL, or 100 μL of 2M Tris base, pH 9.0 (for a mini, midi or maxi scale selection, respectively).

The phage were then used to infect 2.5 mL, 7.5 mL or 15 mL (for a mini, midi or maxi scale selection, respectively) of XL1-Blue MRF′ cells (OD600 of 0.6-1.5). The cells were incubated for 30 minutes at room temperature. The cells were spread on a Corning bioassay tray (LB agar containing 100 μg/mL carbenicillin, 100 mM D-glucose), with 2.5 mL cells per tray. The tray was incubated at room temperature for 30 minutes before being incubated at 37° C. for 12 hours.

6. DNA Purification and Further Rounds of Selection

After the 12 hour incubation, the cells were scraped from the tray and DNA was purified using a Qiagen DNA purification kit according to the manufacturers instructions. Additional rounds of selection were then performed by electroporating the purified DNA into the electrocompetent DH5α VCSM13 dsDNA CL F-cells, and proceeding with the phage expression, precipitation, capture and elution, as described above. To wash the phage-bound cells (from Example 12.B.4, above) in the subsequent selection rounds, the following wash conditions were used: Round 2: 5 washes as described for the first round; Round 3; 10 washes with vigorous vortexing and pipetting the cells up and down; Rounds 4-8; 10 washes with vigorous vortexing and pipetting the cells up and down, including a 5 minute incubation at room temperature with rocking between each wash.

### Summary of Library Screening

Table 50 below summarizes the screening for the various CDRL3 libraries generated in Example 10.

TABLE 50
Library Screening Summary

Overlap PCR Libraries
Library

| | AGYS/ | | |
|---|---|---|---|
| | QHYA | AGYS + 1 | AGYS + 2 |
| # | 5 | 6 | 6 |
| Rounds | | | |
| Clones | 400 | 400 | 400 |
| Tested by | from round 4 | from round 5 | from round 5 |
| ELISA | | | |
| Clones | 400 | 400 | 400 |
| Tested by | from round 5 | from round 6 | from round 6 |
| ELISA | | | |

### Example 13

### Preparation of Candida albicans and Control Antigen for ELISA Screening of Fabs

C. albicans cells were prepared for use as the C. albicans target antigen for ELSA screening of the Fab polyclonal pre-selected library isolated in the phage display screening described in Example 12. A starter culture was first prepared by inoculation of 10 mL of YPD medium with a single colony of C. albicans (Cat. No. 10231, ATCC). The cells were cultured at 37° C. with shaking at 170 rpm for 24 hours, before 500 μL of culture was removed and transferred into 10 mL of fresh YPD medium. The culture was then diluted 1:3 in YPD medium and plated at 100 μL per well of the ELISA microplate (Reacti-Bind White Opaque 96-well plate). The plate was sealed with Qiagen tape pad and incubated at 37° C. for 8-16 hours. Following incubation, the plate was washed 5 times with PBS with 0.05% Tween20. Finally, the ELISA plate was blocked with 250 μL of 4% NFDM-PBS at 37° C. for 2 hours and then used in the ELISA assay described in Example 14.

ELISA plates containing chicken albumin or goat anti-human Fab were also prepared for negative controls. 100 ng of chicken albumin or goat anti-human Fab (100 μL, diluted in PBS) was added to each well of an ELISA microplate (Reacti-Bind White Opaque 96-well plate). The ELISA plate was incubated overnight with rocking at 4° C. Following incubation, the plate was washed 5 times with PBS with 0.05% Tween20. Finally, the ELISA plate was blocked with 250 μL of 4% NFDM-PBS at 37° C. for 2 hours and then used in the ELISA assay described in Example 14.

### Example 14

### ELISA Screening of Fab Candidates for Binding to Candida albicans

The polyclonal library DNA that was pre-selected by phage display in Example 12 was then further screened for identification of single Fab clones that bind to C. albicans. A summary of the number of clones and the round from which they were selected for the various libraries that were screened in Example 12 is shown in Table 50 above. One nanogram of library DNA prepared using a Qiagen Qiafilter according to the manufacturer's instructions was transformed into electrocompetent DH5 Alpha E (F-) cells (Invitrogen). The transformed cells were plated onto LB agar plates containing 100 μg/mL carbenicillin and 100 mM glucose to obtain single colonies. The culture plates were inverted and incubated at 37° C. for 14-16 hours.

Individual colonies were then inoculated into a 96 deep well (1 mL volume) parental microplate containing 1.2 mL SB media containing 50 μg/ml carbenicillin and 20 mM glucose. The parental plate was incubated at 37° C. with shaking at 300 rpm for 12-14 hours.

Following incubation of the parental microplate cultures, a 96 deep well daughter microplate was prepared with 1.0 mL SB culture containing 50 μg/ml carbenicillin and 1 mM IPTG. 200 μL of supernatant from the parental plate was transferred to the daughter plate. The parental plate was centrifuged at 4000 rpm for 20 minutes, and the supernatant was discarded. The parental plate was stored at −80° C. The parental plate was saved for later preparation of DNA and sequence analysis after clonal target antigen recognition to C. albicans was determined. For induction of antibody expression, the daughter plate was incubated at 30° C. with shaking at 300 rpm for 8 hours. The daughter plate was were then stored at −80° C., overnight.

The following day, the daughter plate was removed from −80° C. storage and subjected to three freeze/thaw cycles of 37° C. water bath for 5 minutes, followed by incubation in a dry ice ethanol bath for 5 minutes to lyse the cells. The microplate then was spun at 4000 rpm in the tabletop centrifuge for 30 minutes to clear the lysate. The soluble, freeze thawed antibodies (supernatants) from the daughter plate were then diluted 1:1 with 8% NFDM-1×PBS+0.1% Tween20 buffer into a 96 well dilution plate.

ELISA plates were coated with C. albicans, chicken albumin and goat anti-human Fab as described above in Example 12. The 4% NFDM-PBS blocking solution was discarded and the ELISA plates were washed two times with 1×PBS+0.05% Tween20 wash buffer. 100 μl of the diluted antibodies from the daughter plate was transferred from the 96 well dilution plate to the ELISA plates containing the C. albicans, chicken albumin and goat anti-human Fab. Dilutions of the 2G12 IgG antibody were employed as a positive control. For the negative control, several wells received no primary antibody. The ELISA plates were then incubated at 37° C. for 1 hour with rocking. Following antibody incubation, the media from the ELISA plate wells was discarded to remove unbound antibody and the plates were washed 10 times with 1×PBS+0.05% Tween20 wash buffer.

For detection of Fab antibody binding, an anti-human Fab secondary antibody (Goat Anti Human Fab MinX (Pierce, 31414)) was employed. 100 μl of the diluted secondary antibody (1:50,000, diluted according to manufacturers instructions, using 4% NFDM-1×PBS+0.05% Tween20 as dilution buffer) was added to each well of the ELISA plates. The ELISA plates were then incubated at 37° C. for 1 hour with rocking. Following incubation, the secondary antibody solution was discarded to remove unbound antibody and the ELISA plates were washed 5 times with 1×PBS+0.05% Tween20 wash buffer. 50 μl of Supersignal ELISA Femtomax Sensitivity Substrate solution was added to each ELISA plate well. The ELISA plates were then read by measuring luminescence (relative light units (RLU)) using a Biotek Synergy2 luminometer. Positive hits were identified as wells that had greater than 10 times the relative light units (RLU) over background. Clones with RLU values less than 10 times over background and were not selected for follow up. Background was calculated by averaging the values from control wells without primary antibody.

Antibody 2G12 was diluted from concentrations of 0.0001 to 25 μg/mL and tested for binding to goat anti-Human Fab to generate a standard curve. Using linear regression analysis obtained from the standard curve, estimated working concentrations of the antibody lysates were calculated and values were expressed in nanograms per mL. Specific binding of clones to C. albicans was normalized for antibody expression (RLU) per nanogram of antibody.

DNA from positive clones identified in the screen was prepared using the stored parental plates and sequence analysis was performed. Sequencing was performed for both the heavy and light chains of positive clones. A summary of the clones identified in the screen are shown in Tables 52 and 53. The approximate affinities for selected Fabs are set forth in Table 51 below. Of these clones, 10 were selected for further study (see Table 55 below).

TABLE 51
Affinities of selected Fabs

| | | Approximate Affinity | |
|---|---|---|---|
| Fab | CDRL3 | (method of determination) | |
| 4F8 | QHYKEWRAS | 9 | μg/mL (FACS) |
| 1F8 | QHYLPFNAT | unknown | |
| 1H12 | QHYMPYRAS | 9 | μg/mL (luminescent ELISA) |
| A4F10 | QHYTDHYGAT | 1 | μg/mL (luminescent ELISA) |
| A1G7 | QHYTDHRGAT | 1.5 | μg/mL (luminescent ELISA) |
| P2H12 | QHYTDHHGAT | 2 | μg/mL (luminescent ELISA) |

TABLE 52

AGYS CDRL3 Mutants Identified by ELISA as
binding to C. Albicans

| | | | | | | |
|---|---|---|---|---|---|---|
| K--- | R--- | M | Q | V/S--- | I/L--- | E/H--- |

| | | | | | | | |
|---|---|---|---|---|---|---|---|
| EWR | QHYKEW | | | | | | |
| | RAS | | | | | | |
| | (SEQ ID | | | | | | |
| | NO: 181) | | | | | | |
| EWS | QHYKEW | QHYREWS | | | | | |
| | SAT | AT | | | | | |
| | (SEQ ID | (SEQ ID | | | | | |
| | NO: 182) | NO: 183) | | | | | |
| EWW | QHYKEW | QHYREWW | | | | | |
| | WAT | AT | | | | | |
| | (SEQ ID | (SEQ ID | | | | | |
| | NO: 185) | NO: 186) | | | | | |
| SWS | | | | | | QHYLSWS | |
| | | | | | | AT | |
| | | | | | | (SEQ ID | |
| | | | | | | NO: 187) | |
| AWS | | | | | | QHYLAWS | |
| | | | | | | AT | |
| | | | | | | (SEQ ID | |
| | | | | | | NO: 184) | |
| PFN | QHYKPF | QHYRPFN | QHYMPFN | QHYQPFN | | QHYLPFN | QHYEPFN |
| | NAT | AT | AT | AT | | AT | AT |
| | (SEQ ID | (SEQ ID | (SEQ ID | (SEQ ID | | (SEQ ID | (SEQ ID |
| | NO: 188) | NO: 189) | NO: 190) | NO: 191) | | NO: 192) | NO: 193) |
| PFE | QHYKPF | QHYRPFE | | | | | |
| | EAT | AT (SEQ | | | | | |
| | (SEQ ID | ID | | | | | |
| | NO: 194) | NO: 195) | | | | | |
| PFQ | QHYKPF | QHYRPFQ | | QHYQPFQ | | QHYIPFQ | |
| | QAT | AT | | AT | | AT | |
| | (SEQ ID | (SEQ ID | | (SEQ ID | | (SEQ ID | |
| | NO: 196) | NO: 197) | | NO: 198) | | NO: 199) | |
| PFS | QHYKPF | QHYRPFS | QHYQPFS | | QHYVPFS | | QHYHPFS |
| | SAS | AT | AT | | AT | | AT |
| | (SEQ ID | (SEQ ID | (SEQ ID | | (SEQ ID | | (SEQ ID |
| | NO: 200) | NO: 201) | NO: 202) | | NO: 203) | | NO: 204) |
| PFH | QHYKPF | Q | Q | | | | QHYEPFH |
| | HAT | HYRPFHA | HYMPFHAT | | | | AT |
| | (SEQ ID | T (SEQ | (SEQ ID | | | | (SEQ ID |
| | NO: 205) | ID | NO: 207) | | | | NO: 208) |
| | | NO: 206) | | | | | |
| PFR | Q | | | | QHYVPFR | | QHYEPFR |
| | HYKPFR | | | | AT | | AT |
| | AT | | | | (SEQ ID | | (SEQ ID |
| | (SEQ ID | | | | NO: 210) | | NO: 211) |
| | NO: 209) | | | | | | |
| PFA | Q | | | | QHYVPFA | QHYIPFA | |
| | HYKPFA | | | | AT | AT | |
| | AT | | | | (SEQ ID | (SEQ ID | |
| | (SEQ ID | | | | NO: 213) | NO: 214) | |
| | NO: 212) | | | | | | |
| PFD | Q | | Q | | | | |
| | HYKPFD | | HYMPFDAT | | | | |
| | AT | | (SEQ ID | | | | |
| | (SEQ ID | | NO: 216) | | | | |
| | NO: 215) | | | | | | |
| PFK | | | Q | | | | |
| | | | HYMPFKAT | | | | |
| | | | (SEQ ID | | | | |
| | | | NO: 217) | | | | |
| PFT | | | Q | | | | |
| | | | HYMPFTAT | | | | |
| | | | (SEQ ID | | | | |
| | | | NO: 218) | | | | |
| PFP | | | Q | | | | |
| | | | HYMPFPAT | | | | |
| | | | (SEQ ID | | | | |
| | | | NO: 219) | | | | |
| PFW | | | | Q | Q | | |
| | | | | HYQPFWAT | HYSPFWAT | | |
| | | | | (SEQ ID | (SEQ ID | | |
| | | | | NO: 220) | NO: 221) | | |
| | | | Q | | | | |
| | | | HYMPYRAS | | | | |
| | | | (SEQ ID | | | | |
| | | | NO: 222) | | | | |
| PYR | Q | | Q | Q | | Q | Q |
| | HYKPYR | | HYMPYRAT | HYQPYRAT | | HYLPYRAT | HYEPYRAT |
| | AT | | (SEQ ID | (SEQ ID | | (SEQ ID | (SEQ ID |
| | (SEQ ID | | NO: 224) | NO: 225) | | NO: 226) | NO: 227) |
| | NO: 223) | | | | | | |
| PYD | Q | | | | | | |
| | HYKPYD | | | | | | |
| | AT | | | | | | |
| | (SEQ ID | | | | | | |
| | NO: 228) | | | | | | |
| PYS | Q | | | | | | |
| | HYKPYS | | | | | | |
| | AT | | | | | | |
| | (SEQ ID | | | | | | |
| | NO: 229) | | | | | | |
| PYV | | | | Q | | | |
| | | | | HYQPYVAT | | | |
| | | | | (SEQ ID | | | |
| | | | | NO: 230) | | | |
| PYK | | | | | | | Q |
| | | | | | | | HYEPYKAT |
| | | | | | | | (SEQ ID |
| | | | | | | | NO: 231) |
| PYQ | | | | | | Q | |
| | | | | | | HYLPYQAS | |
| | | | | | | (SEQ ID | |
| | | | | | | NO: 232) | |

TABLE 53
AGYS + 1 CDRL3 Mutants
Identified by ELISA as binding to C. Albicans

| CDRL3 | SEQ ID NO |
|---|---|
| QHYRPHTGAT | 233 |
| QHYTAHDGAT | 234 |
| QHYTAHRGAT | 235 |
| QHYRAHTGAT | 236 |
| QHYTAHTGAT | 237 |
| QHYTDHHGAT | 238 |
| QHYTDHKGAT | 239 |
| QHYTDHRGAT | 240 |
| QHYTDHYGAT | 241 |

### Example 15

### Generation of IgG

In this example, Fab antibodies identified in Example 14 above were converted into IgGs by cloning into either the 2G12 pCALM 8 His mammalian expression vector (SEQ ID NO:336) or the 2G12 pDR12 mammalian expression vector (SEQ ID NO:337), both of which contained the 2G12 heavy chain (set forth in SEQ ID NOS:334 and 335, respectively). Primers specific to the 5′ and 3′ end of the light chain of 2G12 were generated. The primers additionally contained sequences for restriction sites to allow cloning into each respective vector.

A. Cloning into pCALM Mammalian Expression Vector

Primers 2G12IgGLC-F and 2G12IgGLC-R (set forth in Table 54 below) were used to amplify the light chains of Fabs 1H12, 4F8 and 1F8. XhoI (2G12IgGLC-F) and EcoRI (2G12IgGLC-R) restriction sites are shown in bold in Table 54 below. For each reaction, each variant DNA (100 ng) was mixed with 20 pmoles of 2G12IgGLC-F and 20 pmoles of 2G12IgGLC-R and incubated in the presence of 1 μL Advantage HF2 Polymerase Mix (Clontech), 5 μL of 10c HF2 reaction buffer, 5 μL of 10×dNTP mixture and PCR grade water to a final reaction volume of 50 μL. The PCR was carried out using the following reaction conditions: 1 minute denaturation at 95° C., followed by 30 cycles of 5 seconds of denaturation at 95° C., 10 seconds of annealing at 60° C., and 30 seconds of extension at 68° C., then finishing with a 3 minute incubation at 68° C. The amplified fragments (735 bp) were gel-purified using a Gel Extraction Kit (Qiagen) according to the manufacturer's instruction. The purified products were run on 1% agarose gel and each fragment was gel-purified with Gel Extraction Kit (Qiagen) according to the manufacturer's instruction.

The gel-purified fragments were digested with XhoI and EcoRI and subsequently ligated into the similarly digested 2G12 pCALM 8 His mammalian expression vector in the presence of T4 DNA ligase.

B. Cloning into pDR12 Mammalian Expression Vector

Primers 2G12HindIIILC-F1, 2G12HindIIILC-F2 and 2G12EcoRILC-R (set forth in Table 54 below) were used to amplify the light chains of Fabs 1H12, A2A12, P2H12, A1E8, A1G7, A4F10, A5G10, P4H12, and P1F9. HindIII and EcoRI restriction sites are shown in bold in Table 54 below. For each reaction, each variant DNA (diluted 1:100 in Buffer EB) was mixed with 20 pmoles of 2G12HindIIILC-F1, 2 pmoles of 2G12HindIIILC-F2 and 20 pmoles of 2G12EcoRILC-R and incubated in the presence of 1 μL Advantage HF2 Polymerase Mix (Clontech), 5 μL of 10c HF2 reaction buffer, 5 μL of 10×dNTP mixture and PCR grade water to a final reaction volume of 50 μL. The PCR was carried out using the following reaction conditions: 1 minute denaturation at 95° C., followed by 30 cycles of 5 seconds of denaturation at 95° C., 10 seconds of annealing at 60° C., and 30 seconds of extension at 68° C., then finishing with a 3 minute incubation at 68° C. The amplified fragments (735 bp) were gel-purified using a Gel Extraction Kit (Qiagen) according to the manufacturer's instruction. The purified products were run on 1% agarose gel and each fragment was gel-purified with Gel Extraction Kit (Qiagen) according to the manufacturer's instruction.

The gel-purified fragments were digested with HindIII and EcoRI and subsequently ligated into the similarly digested 2G12 pDR12 mammalian expression vector in the presence of T4 DNA ligase.

TABLE 54
2G12 IgG Light Chain Primers

| | | | SEQ |
|---|---|---|---|
| | | | ID |
| Name | nt | Sequences | NO |
| 2G12IgGLC-F | 42 | GGTCCCTGGCTCGAGTGAGGTTGTTAT | 329 |
| | | GACCCAGTCTCCGTC | |
| 2G12IgGLC-R | 44 | CCTGGTACCGAATTCTTAGCATTCACC | 330 |
| | | ACGGTTGAAAGATTTGG | |
| 2G12HindIIILC- | 63 | GTAAGCAAGCTTATGGACATGAGAGT | 331 |
| F1 | | GCCTGCACAGCTGCTGGGACTGC | |
| | | TGCTGCTGTGGCTG | |
| 2G12HindIIILC- | 62 | GGACTGCTGCTGCTGTGGCTGCCAGGCG | 332 |
| F2 | | CCAAGTGCGACGTTGTTATGACCCAGTCT | |
| | | CCGTC | |
| 2G12EcoRILC-R | 46 | CGCTACGAATTCTCAGCATTCACCA | 333 |
| | | CGGTTGAAAGATTTGGTAACC | |

TABLE 55
CDRL3s and library screened selected for
conversion to IgGs

| | | SEQ ID NO | SEQ ID NO | Library |
|---|---|---|---|---|
| IgG | CDRL3 | (CDRL3) | (VL) | Screened |
| 1H12 | QHYMPYRAS | 222 | 283 | AGYS |
| 1F8 | QHYLPFNAT | 192 | 253 | AGYS |
| 4F8 | QHYKEWRAS | 181 | 242 | AGYS |
| A1E8 | QHYTDHKGAT | 239 | 300 | AGYS + 1 |
| A1G7 | QHYTDHRGAT | 240 | 301 | AGYS + 1 |
| P1F9 | QHYRAHTGAT | 236 | 297 | AGYS + 1 |
| A2A12 | QHYTAHTGAT | 237 | 298 | AGYS + 1 |
| P2H12 | QHYTDHHGAT | 238 | 299 | AGYS + 1 |
| A4F10 | QHYTDHYGAT | 241 | 302 | AGYS + 1 |
| P4H12 | QHYTAHRGAT | 235 | 296 | AGYS + 1 |
| A5G10 | QHYRPHTGAT | 233 | 294 | AGYS + 1 |

### Example 16

### Characterization of 2G12 Variants with Improved Affinity for C. albicans

In this example the IgGs generated in Example 15 were assayed for their ability to bind to C. alibicans and various other Candida species, namely C. krusei, C. tropicalis, and C. glabrata by both FACS assay and ELISA.

A. C. albicans Binding by FACS Assay

Selected IgG antibodie's generated in Example 15 were tested for their ability to bind C. albicans by FACS assay. The C. albicans cells were prepared as follows. A starter culture was first prepared by inoculation of 10 mL of YPD medium with a single colony of C. albicans (Cat. No. 10231, ATCC). The cells were cultured at 37° C. with shaking at 170 rpm for 24 hours and subsequently washed 2× with PBS. The cells were fixed by incubating in 1% formaldehyde in PBS for 30 min at room temperature. Following fixation, the cells were washed 2× in PBS, resuspended in fresh PBS and counted (cells/mL).

Approximately 1×10⁶ C. albicans cells in PBS were transferred to each well of a 96-well deep well plate. The plate was subsequently centrifuged to pellet the cells and the supernatant was removed. The cells were then resuspended in 125 μL of 2% BSA in PBS (a 1:5 dilution of a 10% stock solution). The IgG antibodies were serially diluted in PBS (from a concentration of 0.1 to 200 nM). 125 μL each dilution was added to each well (final concentration of 1% BSA). 125 μL of PBS was added to control wells. The plate was then centrifuged for 30 seconds to pool the liquid at the bottom of the wells followed by incubation for 1 hour at room temperature with shaking.

Following incubation, the plate was centrifuged for 5 minutes at 5000 rpm to pellet the cells. The supernatant was removed by inverting the plate and the cells were washed 2× with 1 mL PBS. The cells were resuspended in 250 μL 1% BSA in PBS containing 5 μg/mL secondary antibody (anti human IgG, Alexa fluor 488, Invitrogen). The plate was then centrifuged for 30 seconds to pool the liquid at the bottom of the wells followed by incubation for 1 hour at room temperature with shaking while shielded from all light. Following incubation, the plate was centrifuged for 5 minutes at 5000 rpm to pellet the cells. The supernatant was removed by inverting the plate and the cells were washed 2× with 1 mL PBS. The cells were resuspended in 200 μL PBS. FACS was performed in a FL-1 channel, using the sample that contained only PBS as a control.

The data is shown in Table 56 below, which sets forth the antibody and concentration at 50% maximum binding. 2G12 LC 3ALA (SEQ ID NO:307) which contains three alanine mutations in light chain CDRL3 does not show appreciable binding to C. albicans. Wildtype 2G12 binds at about 150 nM while CDRL3 mutants 1H12 (QHYMPYRAS, SEQ ID NO:222), 1F8 (QHYLPFNAT, SEQ ID NO:192) and 4F8 (QHYKEWRAS, SEQ ID NO:181) all have from 10- to 30-fold increased binding affinity to C. albicans. 2G12 Polymun (Cat. No. AB002, Polymun Scientific) binds at approximately 500 nM. The difference in affinity between 2G12 and 2G12 Polymun is due to the fact the 2G12 contains IgG aggregates (approximately 8-10% aggregates) which increase the affinity for binding to C. albicans.

TABLE 56
Binding to C. albicans by FACS

| Antibody (IgG) | [50% Max] |
|---|---|
| 2G12 LC 3ALA | N/D |
| 2G12 Polymun | 500 nM |
| 2G12 | 150 nM |
| 1H12 | 5.2 nM |
| 1F8 | 15.1 nM |
| 4F8 | 9.4 nM |

B. C. krusei, C. tropicalis, and C. glabrata Binding by FACS Assay

Selected IgGs were analyzed for their ability to bind to C. albicans, C. krusei, C. tropicalis, and C. glabrata by FACS assay. The C. krusei, C. tropicalis, and C. glabrata used in the assay were clinical isolates. The assay was performed as described in Example 16.A. above. The antibodies were tested at concentrations between 0.1 and 1000 nM. 2G12 Polymun (Cat. No. AB002, Polymun Scientific) was used as a control. The antibodies that were tested are set forth in Table 57. Antibodies A1E8, A1G7, A2A12, P2H12, A4F10, and A5G10 bind C. albicans and C. krusei with an affinity of approximately 50 nM. Antibodies A1E8, A1G7, A2A12, P2H12, A4F10, and A5G10 bind C. tropicalis with an affinity between approximately 50-100 nM. Antibodies A1E8, A1G7, A2A12, P2H12, A4F10, and A5G10 bind C. glabratas do not show appreciable binding at the tested antibody concentrations. 2G12 Polymun does not show appreciable binding to any of the isolates. Selected affinities for the various Candida isolates are set forth in Table 58 below. CDRL3 mutants 1H12 and P1F9 bind to all 4 isolates with low nanomolar affinity.

TABLE 57
IgGs screened for binding to C. albicans,
C. krusei, C. tropicalis, and C. glabrata
by FACS

| | | SEQ ID NO | SEQ ID NO | Library |
|---|---|---|---|---|
| IgG | CDRL3 | (CDRL3) | (VL) | Screened |
| 2G12 | QHYAGYSAT | 162 | — | N/A |
| Polymun | | | | |
| 1H12 | QHYMPYRAS | 222 | 283 | AGYS |
| A1E8 | QHYTDHKGAT | 239 | 300 | AGYS + 1 |
| A1G7 | QHYTDHRGAT | 240 | 301 | AGYS + 1 |
| P1F9 | QHYRAHTGAT | 236 | 297 | AGYS + 1 |
| A2A12 | QHYTAHTGAT | 237 | 298 | AGYS + 1 |
| P2H12 | QHYTDHHGAT | 238 | 299 | AGYS + 1 |
| A4F10 | QHYTDHYGAT | 241 | 302 | AGYS + 1 |
| P4H12 | QHYTAHRGAT | 235 | 296 | AGYS + 1 |
| A5G10 | QHYRPHTGAT | 233 | 294 | AGYS + 1 |

TABLE 58
Selected affinities for C. albicans, C. krusei, C. tropicalis,
and C. glabrata

| | C. albicans | | C. krusei | | C. tropicalis | C. glabrata | |
|---|---|---|---|---|---|---|---|
| 2G12 | ~500 | nM | ~1000 | nM | n/a | n/a | |
| Polymun | | | | | | | |
| 1H12 | 10 | nM | 17 | nM | 23 nM | 12 | nM |
| P1F9 | 23 | nM | 27 | nM | 51 nM | 102 | nM |

| P4H12 | N/D | N/D | 21 nM | N/D | |
|---|---|---|---|---|---|
| A5G10 | N/D | N/D | N/D | 72 | nM |

C. C. albicans ELISA Binding Assay

Select IgG antibodies generated in Example 15 were tested for their ability to bind C. albicans by ELISA assay. Binding was detected by detecting a colorimetric change (absorbance at 450 nm) or by detecting bioluminescence.

General Procedure

The C. albicans cells were prepared as follows. A starter culture was first prepared by inoculation of 10 mL of YPD medium with a single colony of C. albicans (Cat. No. 10231, ATCC). The cells were cultured at 37° C. with shaking at 170 rpm for 24 hours. A coating culture was prepared by transferring 500 μL of starter culture into 10 mL of YPD medium. The cells were cultured at 37° C. with shaking at 170 rpm for 24 hours.

Following incubation, the coating culture was diluted 1:3 in YPD medium and plated in a 96-well plate (see Table 59 below). A negative control plate was prepared by coating with chicken albumin (Sigma) at a concentration of 2 μg/mL in PBS. The plates were sealed with Qiagen tape pad and incubated at 37° C. overnight. Following overnight incubation, the plates were washed 5× with PBS containing 0.05% Tween20. The plates were then blocked with 4% NFDM in PBS (see Table 59 below) and incubated at 37° C. for 2 hours. Following blocking, the plates were washed 2× with PBS containing 0.05% Tween20.

The IgGs to be tested were serially diluted in 4% NFDM in PBS with 0.05% Tween20 and each dilution series was transferred to a C. albicans coated plate and an chicken albumin coated plate. 4% NFDM in PBS with 0.05% Tween20 was added to one well of each plate for a “secondary only” control. The plates were sealed with Qiagen tape pad and incubated at 37° C. for 2 hours. Following incubation, the plates were washed 5× with PBS containing 0.05% Tween20. Goat anti-Human Fab MinX secondary antibody (Cat. No. 31414, Pierce) was added to each well according to the dilutions and amounts listed in Table 59 below. The plates were sealed with Qiagen tape pad and incubated at 37° C. for 1 hour. Following incubation, the plates were washed 5× with PBS containing 0.05% Tween20.

TABLE 59
Summary of volume of reagents used in ELISA

| | | | | Goat |
|---|---|---|---|---|
| | | | | anti-Human |
| | Coating | Block | | Fab MinX |
| | C. Albicans | (4% NFDM | | Secondary |
| Assay | Cells | in PBS) | IgG | Antibody |
| Colorimetric | 50 μL | 130 μL | 50 μL | 1:1000 |
| | | | | dilution |
| | | | | 50 μL |
| Luminescent | 100 μL | 250 μL | 100 μL | 1:50000 |
| | | | | dilution |
| | | | | 100 μL |

Detection

Colorimetric: Add 50 μL TMB Substrate (Cat. No. 34021, Pierce) to each well and incubate for 5-10 minutes. Stop the reaction by adding 50 μL 1.0 N. H₂SO₄ and read the absorbance at 450 nm using an ELISA plate reader.

Luminescence: Add 50 μL Supersignal ELISA Femtomax Sensitivity Substrate (Pierce) to each well. Measure the luminescence (RLU, relative light units) using a Biotek Synergy2 luminometer.

Results

Selected IgGs were analyzed for their ability to bind to C. albicans using colorimetric detection. The antibodies were tested at concentrations between 0.0001 and 500 nM. 2G12 Polymun (Cat. No. AB002, Polymun Scientific) and 2F5 Polymun (Cat. No. AB0001, Polymune Scientific) were used as controls. The data is set forth in Table 60 below. Antibody 2F5 Polymun, a monoclonal antibody that binds HIV gp120, did not bind to C. albicans. 2G12 Polymun bound with a 50% Max concentration of 76.3 nM while 2G12 had a 8-fold higher affinity. The difference in affinity between 2G12 and 2G12 Polymun is due to the fact the 2G12 contains IgG aggregates which increase the affinity for binding to C. albicans. CDRL3 mutants 1H12, 1F8 and 4F8 all bind with a 50% Max concentration of approximately 1 nM.

TABLE 60
Binding to C. albicans by ELISA

| Antibody (IgG) | CDRL3 | [50% Max] | |
|---|---|---|---|
| 2F5 Polymun | — | N/D | |
| 2G12 Polymun | QHYAGYSAT | 76.3 | nM |
| 2G12 | QHYAGYSAT | 9.7 | nM |
| 1H12 | QHYMPYRAS | 0.4 | nM |
| 1F8 | QHYLPFNAT | 0.9 | nM |
| 4F8 | QHYKEWRAT | 1.3 | nM |

The antibodies listed in Table 57 above were tested for their ability to bind C. albicans by ELISA using both colorimetric and luminescent detection. The antibodies were tested at concentrations between 0.05 and 700 nM. 2G12 Polymun (Cat. No. AB002, Polymun Scientific) and Fab AC8 were used as controls. Selected data is set forth in Table 61 below. Negative control Fab AC8 did not bind to C. albicans. 2G12 Polymun did not show appreciable binding by luminescence and bound with an EC50 of approximately 500 nM using colorimetric detection. CDRL3 mutant 1H12 had the highest affinity of all the antibodies tested. Antibodies A1E8, A1G7, P1F9, A2A12, P2H12, A4F10, P4H12 and A5G10 all bind C. albicans with the same affinity, between 3.2 and 19 nM.

TABLE 61
Binding to C. albicans by ELISA

| | | Colorimetric | | Luminescent | |
|---|---|---|---|---|---|
| IgG | CDRL3 | ELISA | | ELISA | |
| 2G12 | QHYAGYSAT | ~500 | nM | N/A | |
| Polymun | | | | | |
| 1H12 | QHYMPYRAS | 0.82 | nM | 6.1 | nM |
| P1F9 | QHYRAHTGAT | 3.2 | nM | 19 | nM |

Since modifications will be apparent to those of skill in this art, it is intended that this invention be limited only by the scope of the appended claims.

1. A genetic package, comprising a domain exchanged antibody, wherein:
the domain exchanged antibody is fused to a genetic package display protein, whereby the domain exchanged antibody is displayed on the genetic package; and
a domain exchanged antibody comprises:
a first variable heavy chain (V_{H}) domain, a second variable heavy chain (V_{H}′) domain, a first variable light chain (V_{L}) domain and a second variable light chain (V_{L}′) domain, or functional regions thereof; and
an interface is formed between the V_{H} domain and the V_{H}′ domain.

2. The genetic package of claim 1, wherein:
the V_{H}′ domain interacts with the V_{L} domain; and
the V_{H} domain interacts with the V_{L}′ domain.

3. The genetic package of claim 1, wherein the domain exchanged antibody contains one or more of:
a peptide linker that joins the V_{H} domain and the V_{L}′ domain;
a peptide linker that joins the V_{H}′ domain and the V_{L} domain; and
a peptide linker that joins the V_{H}′ domain and the V_{H} domain.

4. The genetic package of claim 1, wherein the genetic package display protein is fused to one of the V_{H} domain, V_{H}′ domain, V_{L} domain and the V_{L}′ domain.

5. The genetic package of claim 1, wherein the domain exchanged antibody further comprises a first constant heavy chain (C_{H}) domain, a second constant heavy chain (C_{H}′) domain, a first constant light chain (C_{L}) domain and a second constant light chain (C_{L}′), or functional regions thereof.

6. The genetic package of claim 5, wherein:
the V_{H} domain and C_{H} domain are linked, thereby forming a V_{H}-C_{H} chain, or are linked by a peptide linker to form a chain;
the V_{H}′ domain and C_{H}′ domain are linked, thereby forming a V_{H}′-C_{H}′ chain, or are linked by a peptide linker to form a chain;
the V_{L} domain and C_{L} domain are linked, thereby forming a V_{L}-C_{L} chain, or are linked by a peptide linker to form a chain; and
the V_{L}′ domain and C_{L}′ domain are linked, thereby forming a V_{L}′-C_{L}′ chain, or are linked by a peptide linker to form a chain.

7. The genetic package of claim 5, wherein the domain exchanged antibody contains a peptide linker that joins the V_{H} domain and the C_{L}′ domain and a peptide linker that joins the V_{H}′ domain and the C_{L} domain.

8. The genetic package of claim 5, wherein the genetic package display protein is fused to one or more of the C_{H} domain, C_{H}′ domain C_{L} domain and the C_{L}′ domain.

9. The genetic package of claim 1, wherein:
the V_{H} domain and the V_{H}′ domain or functional regions thereof have identical amino acid sequences; and/or
the V_{L} domain and the V_{L}′ domain or functional regions thereof have identical amino acid sequences.

10. The genetic package of claim 5, wherein:
the C_{H} domain and the C_{H}′ domain or functional regions thereof have identical amino acid sequences; and/or
the C_{L} domain and the C_{L}′ domain or functional regions thereof have identical amino acid sequences.

11. The genetic package of claim 1 that further comprises a hinge region.

12. The genetic package of claim 11, wherein the hinge region is connected to one or more of the C_{H} domain, C_{H}′ domain, V_{H} domain, and V_{H}′ domain.

13. The genetic package of claim 11, wherein the domain exchanged antibody contains one or more hinge region disulfide bonds.

14. The genetic package of claim 1, wherein the domain exchanged antibody contains intra-chain disulfide bonds.

15. The genetic package of claim 1, wherein the domain exchanged antibody contains a disulfide bond between an amino acid in the V_{H} domain and an amino acid in the V_{H}′ domain.

16. The genetic package of claim 1 that is a phage.

17. The genetic package of claim 16, wherein the phage is a bacteriophage, selected from among: Ff, M13, fd, and fl.

18. The genetic package of claim 1 that specifically binds to an antigen selected from among: carbohydrates, polysaccharides, proteoglycans, lipids, proteins, nucleic acids and glycolipids.

19. The genetic package of claim 18, wherein the antigen is expressed on an infectious agent selected from among any one or more of microbes, viruses, bacteria, yeast, fungi, prions and drug-resistant infectious agents.

20. The genetic package of claim 1, wherein the domain exchanged antibody specifically binds an antigen other than HIV gp120.

21. The genetic package of claim 1, wherein the domain exchanged antibody is 2G12 or is a modified 2G12.

22. The genetic package of claim 1 that contains modifications at one or more amino acid residue positions in any one or more of: a heavy chain CDR1, a heavy chain CDR2, a heavy chain CDR3, a light chain CDR1, a light chain CDR2 and a light chain CDR3, compared to the 2G12 antibody.

23. The genetic package of claim 22, wherein the domain exchanged antibody contains modifications at one or more amino acid residues selected from among H31, H32, H33, H52, H95, H96, H97, H98, H99, H100, H100a, H100c, H100d, L89, L90, L91, L92, L93, L94 and L95, based on Kabat numbering.

24. The genetic package of claim 1, wherein the domain exchanged antibody is selected from among a domain exchanged Fab fragment, a domain exchanged scFv fragment, a domain exchanged single chain Fab (scFab) fragment, a domain exchanged scFv tandem fragment, a domain exchanged scFv hinge fragment and a domain exchanged Fab hinge fragment.

25. A composition, comprising a plurality of the genetic packages of claim 1.

26. A collection of genetic packages, comprising:
genetic packages displaying domain exchanged antibody polypeptides.

27. A vector, comprising:
a nucleic acid encoding a heavy chain variable region (V_{H}) domain of a domain exchanged antibody, or a functional region thereof;
a nucleic acid encoding a genetic package display protein, wherein the nucleic acid encoding the genetic package display protein is 3′ of the nucleic acid encoding the V_{H} domain or functional region thereof; and
a stop codon, wherein the stop codon is located between the nucleic acid encoding the V_{H} domain or functional region thereof and the nucleic acid encoding the display protein.

28. The vector of claim 27, wherein the stop codon is selected from among: an amber stop codon (UAG or TAG), an ochre stop codon (UAA or TAA) and an opal stop codon (UGA or TGA).

29. The vector of claim 27, further comprising an additional nucleic acid, selected from among:
a nucleic acid encoding a light chain variable region (V_{L}) domain or functional region thereof;
a nucleic acid encoding a heavy chain constant region (C_{H}) domain or functional region thereof, and
a nucleic acid encoding a light chain constant region (C_{L}) domain or functional region thereof.

30. The vector of claim 27, wherein the nucleic acid encoding the V_{H} domain or functional region thereof, the nucleic acid encoding the genetic package display protein, and the stop codon are operably linked to a promoter, such that upon initiation of transcription from the vector, an mRNA transcript is produced, wherein the mRNA transcript encodes the V_{H} domain or functional region thereof, the genetic package display protein, and includes an RNA stop codon.

31. A vector, comprising:
two nucleic acids encoding heavy chain variable region (V_{H}) domains of a domain exchanged antibody or functional regions thereof;
a nucleic acid encoding a genetic package display protein, wherein the nucleic acid encoding the genetic package display protein is 3′ of the nucleic acids encoding the V_{H} domains or functional regions thereof, and
a nucleic acid encoding a peptide linker, wherein:
the two nucleic acids encoding V_{H} domains or functional regions thereof encode identical V_{H} domains or functional regions; and
the nucleic acid encoding the peptide linker is between the two nucleic acids encoding V_{H} domains or functional regions thereof.

32. The vector of claim 31, wherein the nucleic acid(s) encoding the peptide linker(s) contains nucleic acid having the nucleotide sequence set forth in any of SEQ ID NOS: 15, 17, 19, 21, 23, 25 and 27.

33. The vector of claim 31, wherein the nucleic acids encoding the V_{H} domains or functional regions thereof, the nucleic acid encoding the genetic package display protein, and the nucleic acid encoding the peptide linker(s), are operably linked to a promoter, such that upon initiation of transcription from the vector, an mRNA transcript is produced that contains nucleic acid encoding the V_{H} domains or functional regions thereof, the genetic package display protein, and the peptide linker(s).

34. A vector, comprising:
a nucleic acid encoding a heavy chain variable region (V_{H}) domain of a domain exchanged antibody or a functional region thereof;
a nucleic acid encoding a genetic package display protein, wherein the nucleic acid encoding the genetic package display protein is 3′ of the nucleic acid encoding the V_{H} domain or region thereof, and
a nucleic acid encoding a dimerization domain, wherein:
the nucleic acid encoding the dimerization domain is located between the nucleic acid encoding the V_{H} domain or functional region thereof and the nucleic acid encoding the display protein.

35. The vector of claim 34, further comprising a stop codon, located between the nucleic acid encoding the dimerization domain and the nucleic acid encoding the display protein.

36. The vector of claim 34, wherein the antibody is a domain exchanged antibody selected from a full-length antibody or an antigen-binding fragment thereof.

37. A vector, comprising:
a nucleic acid encoding an antibody heavy chain variable region (V_{H}) domain or a functional region thereof;
a nucleic acid encoding a genetic package display protein, wherein the nucleic acid encoding the genetic package display protein is 3′ of the nucleic acid encoding the antibody heavy chain variable region (V_{H}) domain or functional region thereof; and
a stop codon between the nucleic acid encoding the V_{H} domain or functional region thereof and the nucleic acid encoding the display protein, wherein:
the vector does not encode an antibody hinge region or functional region thereof;
the vector does not encode a leucine zipper or a GCN4 zipper domain; and
upon introduction of the vector into host cell that produces a genetic package and upon expression of the encoded V_{H} protein or functional region thereof, an antibody containing two copies of the V_{H} domain or functional region thereof, is displayed on the genetic package.

38. The vector of claim 37, wherein the antibody is a domain exchanged antibody selected from a full-length antibody or an antigen-binding fragment thereof.

39. A nucleic acid molecule, comprising:
a nucleic acid encoding a first leader peptide;
a nucleic acid encoding a first polypeptide, wherein the nucleic acid encoding the first leader peptide is operably linked to the nucleic acid encoding the first polypeptide for secretion thereof;
a nucleic acid encoding a genetic package display protein, wherein the nucleic acid encoding the genetic package display protein is 3′ of the nucleic acid encoding first polypeptide; and
two stop codons; wherein
the first stop codon is located in the nucleic acid encoding the first leader peptide or the nucleic acid encoding the first polypeptide; and
the second stop codon is located between the nucleic acid encoding the first polypeptide and the nucleic acid encoding the display protein.

40. The nucleic acid molecule of claim 39, wherein the nucleic acids encoding the first leader peptide, first polypeptide and genetic package display protein are operably linked to a promoter, whereby, upon initiation of transcription from the nucleic acid molecule, a single mRNA transcript that contains nucleic acids encoding the first leader peptide, the first polypeptide and the genetic package display protein is produced.

41. The nucleic acid molecule of claim 39, wherein the nucleic acid encoding the first polypeptide encodes an antibody or functional region thereof.

42. The nucleic acid molecule of claim 39, wherein the nucleic acid encoding the first polypeptide encodes a domain exchanged antibody or functional region thereof.

43. The nucleic acid molecule of claim 39, wherein the nucleic acid encoding the first polypeptide encodes an antibody domain selected from among:
a heavy chain variable region (V_{H}) domain or functional region thereof;
a light chain variable region (V_{L}) domain or functional region thereof;
a heavy chain constant region (C_{H}) domain or functional region thereof;
and a light chain constant region (C_{L}) domain or functional region thereof.

44. The nucleic acid molecule of claim 39, wherein the nucleic acid encoding the first polypeptide encodes two or more antibody domains.

45. The nucleic acid molecule of claim 42, further comprising:
a nucleic acid encoding a second leader peptide;
a nucleic acid encoding second polypeptide, wherein the nucleic acid encoding the second leader peptide is operably linked to the nucleic acid encoding the first polypeptide for secretion thereof; and
a third stop codon; wherein
the third stop codon is located in the nucleic acid encoding the second leader peptide or the nucleic acid encoding the second polypeptide.

46. The nucleic acid molecule of claim 45, wherein the nucleic acids encoding the second leader peptide, second polypeptide, first leader peptide, first polypeptide, and genetic package display protein are operably linked to a promoter, whereby, upon initiation of transcription from the nucleic acid molecule, a single mRNA transcript that contains nucleic acids encoding the second leader peptide, second polypeptide, first leader peptide, first polypeptide and genetic package display protein is produced.

47. The nucleic acid molecule of claim 45, wherein the nucleic acid encoding the second polypeptide encodes an antibody or functional region thereof.

48. The nucleic acid molecule of claim 45, wherein the nucleic acid encoding the second polypeptide encodes a domain exchanged antibody or functional region thereof.

49. The nucleic acid molecule of claim 45, wherein the nucleic acid encoding the second polypeptide encodes an antibody domain selected from among:
a heavy chain variable region (V_{H}) domain or functional region thereof;
a light chain variable region (V_{L}) domain or functional region thereof;
a heavy chain constant region (C_{H}) domain or functional region thereof; and
a light chain constant region (C_{L}) domain or functional region thereof.

50. The nucleic acid molecule of claim 45, wherein one or more additional stop codons are located in one or more of the nucleic acids encoding the first leader peptide, first polypeptide, second leader peptide and second polypeptide.

51. The nucleic acid molecule of claim 39 that contains an additional 1, 2, 3, 4, 5, 6, 7, 8, 9, 10 or more stop codons.

52. The nucleic acid molecule of claim 39, wherein the stop codons are each selected from among: an amber stop codon (UAG or TAG), an ochre stop codon (UAA or TAA) and an opal stop codon (UGA or TGA).

53. The nucleic acid molecule of claim 51, wherein the stop codons are each selected from among: an amber stop codon (UAG or TAG), an ochre stop codon (UAA or TAA) and an opal stop codon (UGA or TGA).

54. A nucleic acid molecule, comprising:
a nucleic acid encoding a first leader peptide;
a nucleic acid encoding a first polypeptide, wherein the nucleic acid encoding the first leader peptide is operably linked to the nucleic acid encoding the first polypeptide for secretion thereof;
a nucleic acid encoding a second leader peptide;
a nucleic acid encoding a second polypeptide, wherein the nucleic acid encoding the second leader peptide is operably linked to the nucleic acid encoding the second polypeptide for secretion thereof;
a nucleic acid encoding a genetic package display protein, wherein the nucleic acid encoding the genetic package display protein is 3′ of the nucleic acid encoding first polypeptide; and
two stop codons; wherein
the first stop codon is located in the nucleic acid encoding the first leader peptide; and
the second stop codon is located in the nucleic acid encoding the second leader peptide.

55. The nucleic acid molecule of claim 54, wherein the nucleic acid encoding the second leader peptide, second polypeptide, first leader peptide, first polypeptide and genetic package display protein are operably linked to a promoter, whereby, upon initiation of transcription from the nucleic acid molecule, a single mRNA transcript that contains nucleic acids encoding the second leader peptide, second polypeptide, first leader peptide, first polypeptide and genetic package display protein is produced.

56. The nucleic acid molecule of claim 54, wherein the nucleic acid encoding the first polypeptide encodes an antibody or functional region thereof.

57. The nucleic acid molecule of claim 56, wherein the antibody is a domain exchanged antibody or functional region thereof.

58. The nucleic acid molecule of claim 54, wherein the nucleic acid encoding the second polypeptide encodes a domain exchanged antibody or functional region thereof.

59. The nucleic acid molecule of claim 54, wherein the stop codons are each selected from among: an amber stop codon (UAG or TAG), an ochre stop codon (UAA or TAA) and an opal stop codon (UGA or TGA).

60. The nucleic molecule of claim 39, wherein the nucleic acid encoding the first leader peptide encodes a bacterial leader peptide.

61. The nucleic molecule of claim 54, wherein the nucleic acid encoding the first leader peptide encodes a bacterial leader peptide.

62. The nucleic molecule of claim 45, wherein the nucleic acid encoding the second leader peptide encodes a bacterial leader peptide.

63. The nucleic molecule of claim 54, wherein the nucleic acid encoding the second leader peptide encodes a bacterial leader peptide.

64. The nucleic acid molecule of claim 39, wherein the genetic package display protein is a bacteriophage coat protein.

65. The nucleic acid molecule of claim 54, wherein the genetic package display protein is a bacteriophage coat protein.

66. The nucleic acid molecule acid of claim 56 that encodes a full length domain exchanged 2G12 antibody or modified 2G12 antibody.

67. The nucleic acid molecule of claim 56, wherein the antibody is selected from among domain exchanged Fab fragments, domain exchanged scFv fragments, domain exchanged scFv tandem fragments, domain exchanged single chain Fab (scFab) fragments, domain exchanged scFv hinge fragments and domain exchanged Fab hinge fragments.

68. The nucleic acid molecule of claim 39 comprising a sequence of nucleotides set forth in SEQ ID NO:35.

69. The nucleic acid molecule of claim 39 that comprises a vector.

70. The nucleic acid molecule of claim 54 that comprises a vector.

71. A library, comprising a plurality of nucleic acid molecules of claim 39.

72. A library, comprising a plurality of nucleic acid molecules of claim 54.

73. (canceled)

74. A method for producing a first polypeptide, comprising:
introducing into a cell a nucleic acid molecule of claim 39; and
culturing the cell under conditions whereby the first polypeptide is expressed, wherein the cell is a partial suppressor cell.

75. A method for producing a first polypeptide, comprising:
introducing into a cell a nucleic acid molecule of claim 54; and
culturing the cell under conditions whereby the first polypeptide is expressed, wherein the cell is a partial suppressor cell.

76. The method of claim 74, wherein:
the nucleic acid molecule contains the second stop codon;
the second stop codon is an amber stop codon; and
the cell is a partial amber suppressor cell.

77. The method of claim 75, wherein:
the nucleic acid molecule contains the third stop codon;
the third stop codon is an amber stop codon; and
the cell is a partial amber suppressor cell.

78. The method of claim 76, wherein expression of the encoded first polypeptide results in a fusion polypeptide that comprises the first polypeptide fused to the genetic package display protein, and a non-fusion polypeptide that comprises the first polypeptide without the genetic package display protein.

79. The method of claim 77, wherein expression of the encoded first polypeptide results in a fusion polypeptide that comprises the first polypeptide fused to the genetic package display protein, and a non-fusion polypeptide that comprises the first polypeptide without the genetic package display protein.

80. The method of claim 76, wherein the first polypeptide is a domain exchanged antibody or functional region thereof.

81. The method of claim 77, wherein the first polypeptide is a domain exchanged antibody or functional region thereof.

82. The method of claim 76, wherein the domain exchanged antibody is 2G12.

83. The method of claim 77, wherein the domain exchanged antibody is 2G12.

84. A domain exchanged antibody, comprising a modification at an amino acid position, based on Kabat numbering, selected from among H31, H32, H33, H52, H95, H96, H97, H98, H99, H100, H100a, H100c, H100d, L89, L90, L91, L92, L93, L94 and L95, wherein the modification is with reference to the amino acid residue at the corresponding position in domain exchanged antibody 2G12.

85. The domain exchanged antibody of claim 84, wherein the amino acid modification is at an amino acid position selected from among H32, H33, H96, H100, H100a, H100c, H100d, L92, L93, L94 and L95, based on Kabat numbering.

86. The domain exchanged antibody of claim 84, that is a modified 2G12 domain exchanged antibody.

87. The domain exchanged antibody of claim 85, wherein the unmodified 2G12 domain exchanged antibody comprises a light chain having a sequence of amino acids set forth in SEQ ID NO:159, and a heavy chain having a sequence of amino acids set forth in SEQ ID NO:308.

88. The domain exchanged antibody of claim 86, wherein the modifications are amino acid replacements in the variable heavy chain at positions H100, H100a and H100c by Kabat numbering.

89. The domain exchanged antibody of claim 88, wherein the amino acid replacements are replacement with an alanine.

90. The domain exchanged antibody of claim 86, wherein the modifications are amino acid replacements in the variable light chain at positions L91, L94 and L95 by Kabat numbering.

91. The domain exchanged antibody of claim 90, wherein the amino acid replacements are replacement with an alanine.

92. The domain exchanged antibody of claim 84 that is a domain exchanged antibody fragment.

93. The domain exchanged antibody of claim 92, wherein the domain exchanged antibody fragment is selected from among a domain exchanged 8 Fab fragment, a domain exchanged scFv fragment, a domain exchanged single chain Fab (scFab) fragment, a domain exchanged scFv tandem fragment, a domain exchanged scFv hinge fragment and a domain exchanged Fab hinge fragment.

94. The domain exchanged antibody of claim 84, comprising a heavy chain having a sequence of amino acids set forth in SEQ ID NO: 306.

95. The domain exchanged antibody of claim 84, comprising a light chain having a sequence of amino acids set forth in SEQ ID NO: 307 or 322.

96. The domain exchanged antibody of claim 84, comprising a V_{H} domain having a sequence of amino acids set forth in SEQ ID NO: 161.

97. The domain exchanged antibody of claim 84, comprising a V_{L} domain having a sequence of amino acids set forth in SEQ ID NO: 305 or 321.

98. A collection, comprising a plurality of domain exchanged antibodies of claim 84.

99. The collection of claim 98, wherein domain exchanged antibodies are 2G12 antibodies.
